(19) United States
(12) Patent Application Publication
(10) Pub. No.: US 2026/0151398 A1
(43) Pub. Date: Jun. 4, 2026

(54) COMPOUNDS FOR REDUCING CHOLESTEROL AND TREATING LIVER AND KIDNEY DISEASE

(71) Applicant: McMaster University, Hamilton (CA)

(72) Inventors: Jakob Magolan, Grimsby (CA); Richard Austin, Ancaster (CA); Paul Lebeau, St. Catherines (CA); Jae Hyun Byun, Oakville (CA); Paul Saliba, Oakville (CA); Matthew Sguazzin, Hamilton (CA)

(73) Assignee: McMaster University, Hamilton, ON (CA)

(21) Appl. No.: 18/686,147

(22) PCT Filed: Aug. 26, 2022
(86) PCT No.: PCT/CA2022/051295

§ 371 Date: Feb. 23, 2024

Related U.S. Application Data
(60) Provisional application No. 63/260,595, filed on Aug. 26, 2021.

(51) Int. Cl. A61K 31/522; A61K 45/06; A61P 13/12; C07D 473/04; C07D 473/08

(52) U.S. Cl. CPC A61K 31/522; A61K 45/06; A61P 13/12; C07D 473/04; C07D 473/08

(57) ABSTRACT

This application relates to compounds such as compounds of Formula (I) that block SREBP2-induced hepatic PCSK9 expression, compositions comprising these compounds and methods of use thereof; for example, for treating diseases, disorders or conditions treatable by blocking SREBP2 activation and/or PCSK9 gene expression.

### CROSS REFERENCE TO RELATED APPLICATIONS

The present application claims the benefit of priority from U.S. provisional patent application Ser. No. 63/260,595 filed on Aug. 26, 2021, the contents of which are incorporated herein by reference in their entirety.

### FIELD

The present application relates to compounds that block SREBP2-induced hepatic PCSK9 expression, compositions comprising these compounds and methods of use thereof for example, for treating diseases, disorders or conditions treatable by blocking SREBP2 activation and/or PCSK9 gene expression, alone or in combination with other pharmaceuticals.

### BACKGROUND

Increased levels of circulating low density lipoprotein-cholesterol (LDLc) are linked to the development of cardiovascular disease (CVD). Despite the approval of several therapies that lower LDLc, many patients fail to reach their LDL lowering goal due to intolerance, adverse events or simply the high cost of medications. An important regulator of LDLc is the sterol regulatory element-binding protein 2 (SREBP2) which is an endoplasmic reticulum (ER)-resident transcription factor. SREBP2 is activated by reductions in intracellular cholesterol and loss of ER Ca²⁺, which then triggers translocation of SREBP2 from the ER to the Golgi where it is cleaved by S1 and S2 proteases. Upon this cleavage, the released transcriptional portion of SREBP2 migrates to the nucleus where it acts as a transcription factor to induce the cholesterol regulatory genes including the proprotein convertase subtilisin/kexin type 9 (PCSK9), the low-density lipoprotein receptor (LDLR) and HMG-CoA reductase (HMGR) (Horton, J. D., et al., 2003). Recent advancements in the therapies available for the management of dyslipidemia and CVD have led to the characterization of PCSK9 as a hepatocyte-secreted circulating factor capable of enhancing the degradation of cell-surface LDLR (Abifadel, M., et al., 2003; Seidah, N. G., et al., 2003; Benjannet, S., et al., 2004; Maxwell, K. N., et al., 2004). By extension, PCSK9 also reduces the ability of metabolically active tissues, like the liver, to remove excess LDLc from the blood. Based on these seminal discoveries, anti-PCSK9 antibodies are now available to patients at high risk of CVD with great success, yielding an unprecedented 60-70% reduction of LDLc levels (Sabatine, M. S., et al., 2017). Although efficacious, the high cost of manufacturing fully human anti-PCSK9 antibodies and/or need for subcutaneous administration poses a limit to their availability to patients worldwide (Robinson, J. G., et al., 2019). Such circumstances warrant the need for additional studies examining the molecular mechanisms that modulate the expression and secretion of PCSK9 from hepatocytes, in order to develop novel and more cost-effective therapies.

The background herein is included solely to explain the context of the application. This is not to be taken as an admission that any of the material referred to was published, known, or part of the common general knowledge as of the priority date.

### SUMMARY

In the present application, it has been demonstrated that clinically relevant concentrations of caffeine (CF) suppress SREBP2 transcriptional activation in liver hepatocytes, thereby leading to a reduction of PCSK9 in both mice and humans. Using structure/activity relationships (SAR), several xanthine derivatives were shown to have heightened antagonism against SREBP2 and PCSK9, compared to caffeine. Overall, these studies characterize the mechanism by which caffeine impacts the expression of genes well-known to impact CVD risk. The xanthine-based compounds of the present application have been shown herein to reduce PCSK9 activity and therefore are potential therapeutics for treating diseases, disorders or conditions caused and/or exacerbated by increased PCSK9 function or activity.

Therefore, in accordance with an aspect of the present application, there is provided a compound of Formula (Ia):

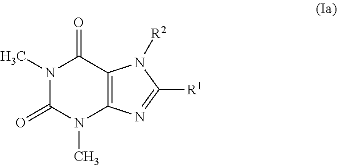

or a pharmaceutically acceptable salt, solvate and/or prodrug thereof;
wherein
R¹ is selected from
   (i) phenyl optionally substituted with one to five substituents independently selected from CN, halo, NH₂, OH, NO₂, C₁₋₆alkyl, C₁₋₆fluoroalkyl, C₁₋₆alkyleneOH, C₁₋₆ alkyleneNH₂, C₁₋₆alkyleneNH(C₁₋₆alkyl), C₁₋₆alkyleneN(C₁₋₆alkyl)(C₁₋₆alkyl), X¹—C₁₋₆ alkyl, X¹—C₁₋₆fluoroalkyl, CO₂H and C(O)H, and substituted with one or two of C₅₋₆heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (ii) 9-10-membered bicyclic aryl wherein the second ring is phenyl or C₅₋₆cycloalkyl and the 9-10 membered bicyclic aryl is optionally substituted with one or more substituents independently selected from ═O, CN, halo, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iii) monocyclic C₃₋₆cycloalkyl or C₅₋₆cycloalkenyl optionally substituted with one or more substituents independently selected from halo, CN, NH₂, OH, NO₂, C₁₋₆alkyl, C₁₋₆ fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one or more substituents independently selected from halo, CN NH₂, OH, NO₂, C₁₋₆alkyl, C₁₋₆ fluoroalkyl, C₁₋₆alkyleneOH, C₁₋₆alkyleneNH₂, C₁₋₆alkyleneNH(C₁₋₆alkyl), C₁₋₆ alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (v) monocyclic C₅₋₆heterocycloalkyl optionally substituted with one or more substituents independently selected from halo, CN, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vi) 8-10-membered bicyclic heterocycloalkyl wherein the second ring is C₅₋₆ heterocycloalkyl, phenyl, C₅₋₆cycloalkyl or C₅₋₆heteroaryl and the 8-10-membered bicyclic heterocycloalkyl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vii) monocyclic C₅₋₆heteroaryl, optionally substituted with one or more substituents independently selected from halo, CN, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆ fluoroalkyl, X²—C₁₋₆alkyl, X²—C₁₋₆fluoroalkyl and NH₂; and
   (viii) 8-10-membered bicyclic heteroaryl wherein the second ring C₅₋₆heterocycloalkyl, phenyl, C₅₋₆cycloalkyl or C₅₋₆heteroaryl and the 8-10-membered bicyclic heteroaryl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₅fluoroalkyl and NH₂;
R² is selected from H, C₁₋₆alkyl and C₁₋₆alkyl substituted with one or more substituents independently selected from OH and halo; and
X¹ and X² are independently selected from O, NH, N(C₁₋₆alkyl), N(C₁₋₆fluoroalkyl), C(O), C(O)O, OC(O), C(O)NH, C(O)N(C₁₋₆alkyl), C(O)N(C₁₋₆fluoroalkyl), NHC(O), N(C₁₋₆alkyl)C(O), N(C₁₋₆fluoroalkyl)C(O), S, S(O) and SO₂;
provided that R¹ is not:
(a) phenyl monosubstituted with unsubstituted phenyl, unsubstituted pyridinyl and unsubstituted pyrazolyl;
(b) unsubstituted thiophenyl or thiophenyl substituted with Br, NO₂, CN, CH₃, C(O)H, C(O)CH₃ or OCH₃;
(c) unsubstituted furanyl or furanyl substituted with Cl, Br, CH₃, CF₃, C(O)H or phenyl;
(d) unsubstituted pyrimidinyl, unsubstituted pyridinyl or pyridinyl monosubstituted with NHCH₃, Br, CH₃ or pyridinyl;
(e)

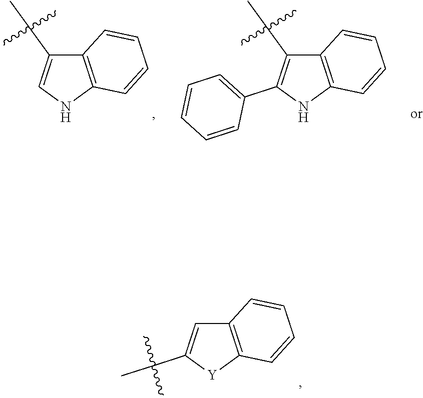

wherein Y is NH, O or S, and when Y is O, the phenyl ring is unsubstituted or mono-substituted with OCH₃ or Cl; and
(f)

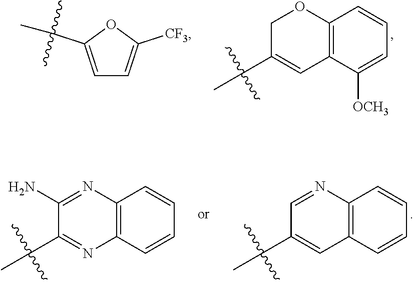 [SMILES: *c1ccc(C(F)(F)F)o1 ; *C1=Cc2c(OC)cccc2OC1 ; *c1nc2ccccc2nc1N ; *c1cnc2ccccc2c1]

Also provided is a compound selected from the compounds listed in Table 1 or a pharmaceutically acceptable salt, solvate and/or prodrug thereof.

Also provided is a pharmaceutical composition comprising one or more compounds disclosed herein, such as compounds of Formula Ia and the compounds listed in Table 1, or a pharmaceutically acceptable salt, solvate and/or hydrate thereof, and one or more pharmaceutically acceptable carriers.

In some embodiments, the compositions disclosed herein further comprise one or more cholesterol lowering agents.

In accordance with another aspect of the present application, there is also provided a method of treating a disease, disorder or condition treatable by blocking SREBP2 activation and/or PCSK9 gene expression, the method comprising administering a therapeutically effective amount of one or more compounds of Formula (I), or a pharmaceutically acceptable salt, solvate and/or prodrug thereof, to a subject in need thereof:

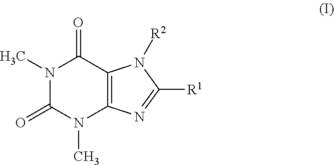

wherein
R¹ is selected from
   (i) phenyl substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆ alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₅ fluoroalkyl and NH₂;
   (ii) 9-10-membered bicyclic aryl wherein the second ring is phenyl or C₅₋₆cycloalkyl and the 9-10 membered bicyclic aryl is optionally substituted with one or more substituents independently selected from ═O, halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iii) monocyclic C₃₋₆cycloalkyl or C₅₋₆cycloalkenyl optionally substituted with one or more substituents independently selected from halo, CN, NH₂, OH, NO₂, C₁₋₆alkyl, C₁₋₆ fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iv) 8-10-membered bicyclic cycloalkyl or cycloalkenyl wherein the second ring is C₅₋₆ heterocycloalkyl, phenyl, C₅₋₆cycloalkyl or C₅₋₆heteroaryl and the 8-10-membered bicyclic cycloalkyl or cycloalkenyl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (v) monocyclic C₅₋₆heterocycloalkyl optionally substituted with one or more substituents independently selected from halo, CN, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆ fluoroalkyl, X²—C₁₋₆alkyl, X²—C₁₋₆fluoroalkyl and NH₂;
   (vi) 8-10-membered bicyclic heterocycloalkyl wherein the second ring is C₅₋₆ heterocycloalkyl, phenyl, C₅₋₆cycloalkyl or C₅₋₆heteroaryl and the 8-10-membered bicyclic heterocycloalkyl is optionally substituted with one or more substituents independently selected from halo, CN, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vii) monocyclic C₅₋₆heteroaryl, optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, X¹-phenyl, X¹-phenyl, C₁₋₆alkylene-phenyl, X¹—C₁₋₆ alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂; and
   (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆ alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆ fluoroalkyl and NH₂;
R² is selected from H, C₁₋₆alkyl and C₁₋₆alkyl substituted with one or more substituents independently selected from OH and halo; and
X¹ and X² are independently selected from O, NH, N(C₁₋₆alkyl), N(C₁₋₆fluoroalkyl), C(O), C(O)O, OC(O), C(O)NH, C(O)N(C₁₋₆alkyl), C(O)N(C₁₋₆fluoroalkyl), NHC(O), N(C₁₋₆alkyl)C(O), N(C₁₋₆ fluoroalkyl)C(O), S, S(O) and SO₂.

In some embodiments, the one or more compounds of Formula (I), or pharmaceutically acceptable salt, solvate and/or prodrug thereof, is a compound of Formula (Ia) and/or a compound selected from Table 1, or pharmaceutically acceptable salt, solvate and/or prodrug thereof.

In some embodiments, the one or more compounds of Formula (I), Formula (Ia) and compound selected from Table 1, or pharmaceutically acceptable salt, solvate and/or prodrug thereof, for use in the treatment methods of the application is selected from the compounds listed in Table 2 or a pharmaceutically acceptable salt, solvate and/or prodrug thereof.

In some embodiments, the disease, disorder or condition is caused and/or exacerbated by increased SREBP2 and/or PCSK9 function or activity.

In some embodiments, the disease, disorder or condition is elevated cholesterol levels, liver disease or chronic kidney disease.

In some embodiments, the therapeutically effective amount of the one or more compounds is administered in combination with one or more other therapeutic agents.

Also provided is a method of blocking SREBP2 activation and/or PCSK9 gene expression in a cell, either in a biological sample or in a subject, comprising administering a therapeutically effective amount of one or more compounds of Formula (I), or a pharmaceutically acceptable salt, solvate and/or prodrug thereof, to a cell in need thereof.

Further provided is a method of increasing endoplasmic reticulum calcium levels in a cell, either in a biological sample or in a subject, comprising administering a therapeutically effective amount of one or more compounds of Formula (I), or a pharmaceutically acceptable salt, solvate and/or prodrug thereof, to a cell in need thereof.

Also provided is a method of lowering serum LDL cholesterol levels comprising administering a therapeutically effective amount of one or more compounds of Formula (I), or a pharmaceutically acceptable salt, solvate and/or prodrug thereof, to a subject in need thereof.

In accordance with another aspect of the present application, there is also provided method of treating or preventing a disease, disorder or condition treatable by lowering serum LDL cholesterol levels comprising administering a therapeutically effective amount of one or more compounds of Formula (I), or a pharmaceutically acceptable salt, solvate and/or prodrug thereof, to a subject in need thereof.

In some embodiments, serum LDL cholesterol levels are lowered compared to pre-dose serum LDL cholesterol levels in the subject.

In some embodiments, the therapeutically effective amount of the one or more compounds is administered in combination with one or more other therapeutic agents.

In some embodiments, the one or more other therapeutic agents elevates serum LDLR cholesterol levels.

In some embodiments, the one or more other therapeutic agents lowers serum LDL cholesterol levels.

In some embodiments, the one or more other therapeutic agents is a statin.

In some embodiments, the statin is selected from the group consisting of atorvastatin, fluvastatin, lovastatin, mevastatin, pitavastatin, pravastatin, rosuvastatin and simvastatin, and combinations thereof.

Also provided is a use of one or more compounds disclosed herein or a composition disclosed herein for blocking SREBP2 activation and/or PCSK9 gene expression.

Further provided is a use of one or more compounds disclosed herein or a composition disclosed herein for treating a disease, disorder or condition treatable by blocking SREBP2 activation and/or PCSK9 gene expression.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

Certain embodiments of the application will now be described in greater detail with reference to the attached drawings in which:

FIG. 1 shows caffeine (CF) blocks PCSK9 expression and secretion in hepatocytes: (A) HuH7 cells were treated with established inducers of PCSK9 expression, thapsigargin (TG) (100 nM) or U18 (10 μM), in the presence or absence of CF (200 μM) for 24 h. PCSK9 expression was assessed via immunoblot analysis; (B, C, D) PCSK9 expression was also assessed in PMH and PHH, as well as in HepG2 cells treated with CF and TG via real-time PCR; (E, F, G) PCSK9 ELISAs were carried on the medium harvested from CF-treated HuH7, HepG2, PMHs and PHH (*, p<0.05); (H) Coomassie blue staining of electrophoretically resolved medium harvested from CF-treated cells served to examine the effect of CF on total secreted protein levels; (I) Secreted PCSK9 levels from HepG2 cells treated with an escalating dose of CF. (J, K) PCSK9 expression and secretion was then assessed in HepG2 cells treated in the presence and absence of CF and a blocker of transcription, ActD. (L)ELISAs were also used to measure secreted PCSK9 levels in CF-treated HepG2 cells (1 mM) transfected with a CMV-driven PCSK9 vector. Values are presented as the mean and error bars as SD. *, p<0.05.

FIG. 2 shows CF blocks SREBP2 activation in hepatocytes: (A) The effect of CF (200 μM) on SREBP2 and SREBP1 mRNA expression was examined in PMH in the presence and absence of TG (100 nM), an established activator of SREBPs—the downstream product of SREBP2 transcriptional activity, HMGR, was also examined; (B, C) The inhibitory effect of CF on SREBP2 was also examined in PHH and HepG2 cells. (D) CF-mediated SREBP1 inhibition was also examined in PMH (*, p<0.05). (E, F, G) HuH7 cells were transfected with a reporter construct encoding a sterol-regulatory element-driven green fluorescent protein (SRE-GFP). Cells were subsequently treated with CF (200 μM) and/or TG (100 nM) 24 h later. GFP and nuclear (n)SREBP2 expression was examined via immunoblot analysis. GFP expression was also assessed via immunofluorescent staining, which was quantified using ImageJ. (H) The cellular localization of SREBP2 in CF- and TG-treated HuH7 cells was also examined via immunofluorescent staining. Nuclei containing activated SREBP2 are indicated by white arrows—values are presented as the mean and error bars as SD; *, p<0.05; scale bars: (G) 100 μm; (H) 20 μm.

FIG. 3 shows CF does not affect CMV-driven PCSK9 expression: The expression of the hepatocyte nuclear factor 1α was assessed in hepatocytes treated with CF in the presence or absence of the ER stress-inducing agents TG.

FIG. 4 shows CF blocks PCSK9 expression in a manner independent of AMPK: (A) The livers of CF-treated mice (100 mg/kg-8 hours) were examined for phosphorylated (p)AMPK and a downstream target of its activation, pACC. (B, C) CF-mediated inhibition of PCSK9 was tested in primary hepatocytes isolated from AMPKβ^{-/-} via ELISA and real time PCR. (D, E) The effect of CDN on PCSK9 expression and secretion was also examined in AMPKβ^{-/-} hepatocytes.

FIG. 5 shows CF increases cytosolic Ca²⁺ levels in cultured hepatocytes: Immortalized HuH7 cultured hepatocytes were loaded with the cytosolic Ca²⁺ dye, fura-2-AM, and subsequently treated with CF over a 24-hour time-course (*, p<0.05).

FIG. 6 shows ER Ca²⁺ modulates PCSK9 expression and secretion: (A) HuH7 cells were either transfected with a FRET-based ER-resident Ca²⁺ sensor, D1ER, or pre-loaded with the low-affinity Ca²⁺ indicator, Mag-Fluo-4. Cells were subsequently treated with either TG (100 nM), CDN (100 μM) or CF (200 μM) for 24 h. Fluorescence intensity was measured using a fluorescent spectrophotometer and visualized using a fluorescent microscope. (B) HuH7 cells were pre-treated with either CF or vehicle for 24 h and loaded with the high-affinity Ca²⁺ dye, Fura-2-AM. Exposure of cells to a high dose of TG (1 mM) induced a spontaneous depletion of ER Ca²⁺ (*, p<0.05 vs. vehicle-treated). (C) The expression of an ER-resident Ca²⁺ binding protein, calnexin, was examined in CF- and TG-treated HuH7 cells using immunoblots. (D, E, F, G) PCSK9, SREBP2 and GRP78 mRNA expression was assessed in HuH7 cells treated with a variety of ER Ca²⁺ modulators including: ryanodine receptor agonist (ryanodine 10 nM), ryanodine receptor antagonist (ryanodine 10 μM), SERCA pump activator CDN (100 μM) and IP3R antagonist 2 APB (50 μm), in the presence and absence of TG (100 nM) for 24 h. (H) mRNA transcript levels were also examined in HuH7 cells treated with SERCA pump inhibitors, TG (100 nM) and CPA (10 μM). (I, J, K) The effect of pharmacologic agents and plasmid-derived CMV-driven proteins, known to affect ER Ca²⁺ levels, on secreted PCSK9 levels was then examined using ELISAs. (L, M) Secreted PCSK9 levels were also examined in TG- and U18-treated cells. (N) The effect of CF on secreted PCSK9 levels was also examined in cells incubated in Ca²⁺-deficient medium. Values are presented as the mean and error bars as SD. *, p<0.05. Scale bars; 200 μm.

FIG. 7 shows ER Ca²⁺ regulates the interaction between GRP78 and SREBP2: (A) HuH7 cultured human hepatocytes were treated with control agents TG (100 nM), which causes ER Ca²⁺ depletion, or CDN (100 μM), a compound known to increase ER Ca²⁺ levels. The effect of CF (200 μM) was also assessed. 24 h after treatment, a co-immunoprecipitation (IP) for GRP78 was carried out. Protein loading was normalized to GRP78 and relative co-immunoprecipitated SREBP2 was examined via immunoblots (IB). (B) The effect of CF, CDN and TG on the retention of ER-resident pre-mature SREBP2, and on the activated nuclear SREBP2 (nSREBP2), was also assessed via IB. (C, D, E) To confirm the role of GRP78 in CF-mediated PCSK9 inhibition, mRNA transcript and secreted protein levels were examined in HepG2 cells exposed to siRNA targeted against GRP78 (siGRP78). (F) Knockdown of GRP78 was confirmed via immunoblot analysis. (G) ER stress markers were assessed in PHH treated with CF 200 μM CDN (10 μM) via real-time PCR. (H) The effect of CF on reactive oxygen species production, resulting from the treatment of TG (100 nM), was also assessed in HuH7 cells (*, p<0.05 vs. vehicle-treated). (I) ER stress-induced amyloid deposition was examined using the fluorescent stain, Thioflavin-T. (J, K) HuH7 cells were transfected with the ER activated indicator plasmid encoding an ER stress-inducible FLAG-sXBP1. Staining intensity was using ImageJ software. (L) Model in which Ca²⁺ promotes the GRP78-mediated sequestration of SREBP2 in the ER. Values are presented as the mean and error bars as SD. (*, p<0.05).

FIG. 8 shows CF protects against ER stress in cultured hepatocytes: (A, B) The effect of CF on ER stress marker expression in HuH7 cells was examined via immunoblots and real time PCR. (C) 12-week old C57BL/6J mice were treated with CDN 50 mg/kg and assessed for LDLR expression via immunohistochemical staining.

FIG. 9 shows CF reduces chaperone expression and blocks hepatic PCSK9 expression in mice (12-week-old male C57BL/6J mice were fasted and treated with CF (50 mg/kg) for 8 h prior to sacrifice; n=6): (A, B) Plasma PCSK9 and triglyceride levels were measured using an ELISA and colorimetric assays, respectively. (C) The time-dependence of CF on plasma PCSK9 levels was also determined using an ELISA (n=5). (D) The livers of these mice were assessed for cell-surface expression of LDLR and CD36, as well as ER stress markers GRP78 and GRP94 via immunohistochemical staining. (E) Staining was quantified using ImageJ software. (F, G) The expression of the LDLR, as well as ER stress markers (GRP78, PERK and IRE1α) as well as cholesterol-regulatory genes (PCSK9, HMGR, SREBP1 and SREBP2) were also examined using immunoblots and real-time PCR. (H) 12-week-old male C57BL/6J mice were treated with a single subcutaneous injection of an inhibitor of PCSK9, alirocumab (30 mg/kg), for 10 days (n=10). (H-I) LDLR expression was assessed using immunohistochemistry and immunoblots. (J) The mRNA expression of SREBP2, PCSK9 and the LDLR was assessed via RT-PCR. Values are presented as the mean and error bars as SD. *, p<0.05. Scale Bars; 50 μm.

FIG. 10 shows CF reduces plasma PCSK9 levels in mice via oral gavage: 12-week-old male C57BL/6J mice were fasted and treated with CF (20 mg/kg) of up to 48 hours prior to sacrifice (n=5). Plasma PCSK9 levels were measured at different time points using an ELISA (First bar at each time point show results using saline. Second bar at each time point shows results using caffeine). Values are presented as the mean and error bars as SD. (*, p<0.05).

FIG. 11 shows CF increases hepatic LDL uptake in a PCSK9-dependent manner: (A) The expression of PCSK9-regulated receptors, LDLR and CD36, was examined in CF-treated cultured hepatocytes (200 μM). (B) The uptake and intracellular accumulation of fluorescently-labelled DiI-LDL was examined in cells treated with CF in the presence or absence of the PCSK9-inducer U18, using a fluorescent spectrophotometer. (C) The effect of CF treatment (200 μM) on DiI-labelled LDL uptake was also examined in PCSK9 shRNA knockdown cells (NS, non-significant). (D) Immunofluorescent staining of cell-surface LDLR was carried out in live CF pre-treated HepG2 cells (200 μM). Cellular DiI-LDL accumulation was also visualized in CF-treated HepG2 cells using a fluorescent microscope. (E) Expression of the LDLR in CF-treated HuH7 (first set of three bars) and HepG2 (second set of three bars) cells was measured via RT-PCR. (F-G) The uptake of DiI-LDL was quantified in HepG2 cells transfected with siRNA targeted against CD36; knockdown was confirmed via immunoblotting. (H) DiI-LDL was also quantified in HepG2 cells treated with SSO (X), a blocker of CD36. Pcsk9+/+ and Pcsk9−/− mice were treated with either PBS-vehicle or CF, as well as fluorescently-labelled DiI-LDL (1 μg/kg). (I) Hepatic cell-surface LDLR expression was assessed via IHC and immunofluorescent staining. (J) Hepatic and serum DiI-LDL fluorescence intensity was quantified using a fluorescent spectrophotometer and visualized using a fluorescent spectrophotometer. (K) Hepatic cell-surface LDLR expression was assessed via immunohistochemistry (DAPI: first column; LDLR: third column; DiI-LDL: second column, merge: forth column). (L, M) Native LDLc was also examined in 18-week-old male C57BL/6J mice treated with CF (30 mg/kg) every 24 hours for 14 days via ELISA of the surrogate marker ApoB; serum PCSK9 levels were also assessed via ELISA (n=10). Values are presented as the mean and error bars as SD. *, p<0.05; NS, non-significant. Scale bars; (D) 10 μm; (I) 50 μm; (K) 100 μm.

FIG. 12 shows CF reduces plasma PCSK9 levels in healthy volunteers: (A, B) Healthy subjects between the ages of 22 and 45 were administered 400 mg (˜5 mg/kg) of CF following a 12-h fasting period. Plasma PCSK9 levels were measured before administration, as well as 2- and 4-h following administration (n=12 and n=5, respectively). (C) PCSK9 levels were also measure in a group of individuals (n=5) that were not administered CF to control for the 2 additional h of fasting time during the experiment. Differences between groups were determined using a paired Student's t-test and error bars are presented as SD.

FIG. 13 shows characterization of novel xanthine-derived compounds as PCSK9 inhibitors in exemplary embodiments of the application: (A, B) HepG2 cells were treated with increasing doses of CF metabolites, paraxanthine and theobromine, as well as xanthine derivatives PSB603, 8CD and 8CC. Secreted PCSK9 levels were assessed using ELISAs and mRNA transcript via real-time PCR. (C, D) Cells were treated with CF, as well as Compound No. Ja-8 (denoted as 1812) and Compound No. Ia-1 (denoted as 1820). Secreted PCSK9, as well as PCSK9 mRNA and SREBP2 mRNA were assessed in these cells. (E) The cytotoxicity of these agents was examined using an LDH assay. (F) HepG2 cells were treated with CF, Compound No. Ia-8 (10 μM) and Compound No. Ia-1 (10 μM) and assessed for cell-surface LDLR expression via immunofluorescent staining; staining intensities were quantified using ImageJ software. (G) The uptake of DiI-LDL was also quantified in these cells using a spectrophotometer. Values are presented as the mean and error bars as SD. *, p<0.05 vs. vehicle; NS, non-significant.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art. It is also to be understood that the terminology used herein is for the purpose of describing particular aspects only and is not intended to be limiting.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The abbreviation, “e.g.” is derived from the Latin exempli gratia and is used herein to indicate a non-limiting example. Thus, the abbreviation “e.g.” is synonymous with the term “for example.”

The term “and/or” as used herein means that the listed items are present, or used, individually or in combination. In effect, this term means that “at least one of” or “one or more” of the listed items is used or present. The term “and/or” with respect to salts, solvates and/or prodrugs thereof means that the compounds of the application exist as individual salts, solvates and prodrugs, as well as a combination of, for example, a salt of a solvate of a compound of the application.

The term “compound of the application” or “compound of the present application” and the like as used herein refers to a compound of Formula (I), or a pharmaceutically acceptable salt, solvate and/or prodrug thereof, and a compound of Formula (Ia), or a pharmaceutically acceptable salt, solvate and/or prodrug thereof and compounds listed in Table 1 or a pharmaceutically acceptable salt, solvate and/or prodrug thereof.

The term “composition of the application” or “composition of the present application” and the like as used herein refers to a composition comprising one or more compounds of the application.

The term “suitable” as used herein means that the selection of the particular compound or conditions would depend on the specific synthetic manipulation to be performed or composition to be prepared, the identity of the molecule(s) to be transformed and/or the specific use for the compound, but the selection would be well within the skill of a person trained in the art.

The present description refers to a number of chemical terms and abbreviations used by those skilled in the art. Nevertheless, definitions of selected terms are provided for clarity and consistency.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are non soluble in aqueous solutions.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₆alkyl means an alkyl group having 1, 2, 3, 4, 5 or 6 carbon atoms.

The term “bicyclic” as used herein refers to a ring system comprising a first ring fused to a second ring, wherein the first ring comprises the point of attachment to the remaining portion of the molecule.

The term “cycloalkyl,” as used herein, whether it is used alone or as part of another group, means a saturated carbocycle. The number of carbon atoms that are possible in the referenced cycloalkyl group are indicated by the numerical prefix “Cₙi-2”. For example, the term C₃₋₈cycloalkyl means a cycloalkyl group having 3, 4, 5, 6, 7 or 8 carbon atoms. The term “bicyclic cycloalkyl” as used herein refers to a cyclic ring system in which the first ring is a cycloalkyl.

The term “cycloalkenyl” as used herein, whether it is used alone or as part of another group, means an unsaturated carbocycle containing 1 or more double bonds. The number of carbon atoms that are possible in the referenced cycloalkyl group are indicated by the numerical prefix “Cₙ₁₋ₙ₂”. For example, the term C₃₋₆alkyl means a cycloalkyl group having 3, 4, 5, 6, 7 or 8 carbon atoms. The term “bicyclic cycloalkenyl” as used herein refers to a bicyclic ring system in which the first ring is a cycloalkenyl.

The term “heterocycloalkyl” as used herein, whether it is used alone or as part of another group, refers to cyclic groups containing at least one non-aromatic ring in which one or more of the atoms are a heteroatom and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom and the remaining atoms are C. The term “bicyclic heterocycloalkyl” as used herein refers to a bicyclic ring system in which the first ring is a heterocycloalkyl.

The term “aryl” as used herein, whether it is used alone or as part of another group, refers to cyclic groups containing at least one aromatic ring. The term “bicyclic aryl” as used herein refers to a bicyclic ring system in which the first ring is an aryl.

The term “heteroaryl” as used herein, whether it is used alone or as part of another group, refers to cyclic groups containing at least one heteroaromatic ring in which one or more of the atoms are a heteroatom and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom. Heteroaryl groups are optionally benzofused. The term “bicyclic heteroaryl” as used herein refers to a bicyclic ring system in which the first ring is a heteroaryl.

A first ring being “fused” with a second ring means the first ring and the second ring share two adjacent atoms there between.

The term “fluoroalkyl” refers to an alkyl group wherein one or more, including all, available hydrogens are substituted with fluoro.

The terms “halo” or “halogen” as used herein, whether it is used alone or as part of another group, refers to a halogen atom and includes fluoro, chloro, bromo and iodo.

The term “available”, as in “available hydrogen atoms” or “available atoms” refers to atoms that would be known to a person skilled in the art to be capable of replacement by a substituent.

The term “cell” as used herein refers to a single cell or a plurality of cells and includes a cell either in a cell culture or in a subject.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted into an active drug.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. Treatment methods comprise administering to a subject a therapeutically effective amount of one or more of the compounds of the application and optionally consist of a single administration, or alternatively comprise a series of administrations.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition, or manifesting a symptom associated with a disease, disorder or condition.

As used herein, the term “effective amount” or “therapeutically effective amount” means an amount of a compound, or one or more compounds, of the application that is effective, at dosages and for periods of time necessary to achieve the desired result.

The expression “inhibiting PCSK9” as used herein refers to inhibiting, blocking and/or disrupting SREBP2-induced PCSK9 expression in a hepatic cell, whether direct or indirect. The inhibiting, blocking and/or disrupting causes a therapeutic effect.

By “inhibiting, blocking and/or disrupting” it is meant any detectable inhibition, block and/or disruption in the presence of a compound compared to otherwise the same conditions, except for in the absence in the compound.

The term “disease, disorder or condition caused and/or exacerbated by increased PCSK9 function or activity” means that the disease, disorder or condition to be treated is affected by, modulated by and/or has some biological basis, either direct or indirect, that includes PCSK9 function or activity. These diseases, disorders or conditions respond favourably when PCSK9 activity or function associated with the disease, disorder or condition is inhibited by one or more of the compounds or compositions of the application.

The term “administered” as used herein means administration of a therapeutically effective amount of a compound, or one or more compounds, or a composition of the application to a cell either in cell culture or in a subject.

It will be understood that any component defined herein as being included may be explicitly excluded by way of proviso or negative limitation, such as any specific compounds or method steps, whether implicitly or explicitly defined herein.

Although methods and materials similar or equivalent to those described herein can be used in the practice or testing of this application, suitable methods and materials are described below.

### II. Compounds, Compositions and Methods of the Application

In the present application a variety of in vitro and in vivo models were used to determine the mechanism by which caffeine affects PCSK9 expression and circulating LDL cholesterol levels. Using structure activity relationships, the development of xanthine derivatives, with increased potency for PCSK9 inhibition compared to caffeine itself is also described. Pre-clinical findings were subsequently confirmed in a cohort of healthy volunteers. Herein, it is demonstrated that caffeine increases hepatic endoplasmic reticulum (ER) Ca²⁺ levels to block the activation of the transcription factor responsible for the regulation of PCSK9, namely sterol regulatory element-binding protein 2 (SREBP2). Evidence to support that caffeine and the xanthine compounds of the application increase the expression and activity of cell-surface hepatic LDLR and improve hepatocyte-mediated LDL cholesterol (LDLc) clearance is also provided. Collectively these findings highlight ER Ca²⁺ as a master regulator of SREBP2 activation and identify a mechanism by which caffeine, as well as other Ca²⁺ modulating agents, affect circulating LDL cholesterol levels to reduce cardiovascular disease risk.

Ca²⁺ release channels, ER-resident chaperones, and buffer proteins play a major role in ER Ca² regulation. The maintenance of ER Ca²⁺ levels is helpful for Ca²⁺-dependent chaperones to promote the proper folding of newly synthesized polypeptides. Thus, loss of ER Ca²⁺ has been linked to an increased accumulation of ER luminal proteins, triggering the unfolded protein response (UPR), which attempts to increase the protein folding capacity of the ER. Thus, any physiological conditions that alter the state of ER Ca²⁺ can impair chaperone activity, leading to an accumulation of misfolded proteins in the ER. This state, known as ER stress, contributes to hepatic injury in liver diseases such as non-alcoholic fatty liver disease (NAFLD), non-alcoholic steatohepatitis NASH, viral hepatitis, and hepatocellular carcinoma (Kaplowitz, N., et al., 2007; Dara, L. and Kaplowitz, N. 2011; Jo, H., et al., 2013; Yeganeh, B., et al., 2015; Shuda, M., et al., 2003). In addition, both UPR activation and the loss of ER Ca²⁺ are reported to activate SREBP2, an important regulator of LDL cholesterol, through its translocation to the nucleus. Ultimately, this induces cholesterol regulatory genes including PCSK9, the low-density lipoprotein receptor (LDLR) and HMG-CoA reductase (HMGR), which further exacerbates the progression of liver diseases, such as NAFLD and NASH (Moslehi, A., et al., 2018; Wang, L., et al., 2018; Lebeau, P., et al., 2019). ER stress also evokes the development of chronic kidney disease (CKD) (Ajoolabady, A. et al., 2021), which has been shown to be ameliorated by SREBP inhibition (Mustafa, M., et al., 2016).

1,3,7-Trimethylxanthine, or caffeine (CF), is best known as a stimulant alkaloid of the central nervous system found in various plants and is commonly found in coffee or tea. The majority of published literature demonstrates that the average adult consumes between 400 and 600 mg/day and organizations like Health Canada and the Food and Drug Administration conclude that such doses are not negatively associated with toxicity, cardiovascular effects, bone status, calcium imbalance, behavior, incidence of cancer or effects on male fertility (Turnbull, D., et al., 2017). On the contrary, accumulating evidence now suggests that moderate to high levels of CF (>600 mg/day), consumed daily in the form of non-alcoholic beverages, are associated with a protective outcome on the cardiovascular system (Turnbull, D., et al., 2017; Ding, M., et al., 2014). Although biochemical studies have shown that CF increases intracellular Ca²⁺ levels and induces vasodilation of the vascular endothelium via release of nitric oxide (Zucchi, R. and Ronca-Testoni, S., 1997; Echeverri, D., et al., 2010), a cellular process known to be cardioprotective (Khazaei, M., et al., 2008), molecular mechanisms supporting clinical evidence are currently lacking.

Herein, it has been found that CF increases intracellular Ca²⁺ levels and attenuates ER stress. In hepatocytes, the data herein strongly suggest that CF increases cytosolic and ER Ca²⁺ levels following a 24-h exposure. It was also found that CF protects against TG-induced ER stress in cultured hepatocytes and reduces the expression of a variety of ER chaperones in the livers of mice. Further disclosed herein is the finding that ER Ca²⁺ levels serve to fine-tune the peptide binding capacity of GRP78, thereby affecting the ER retention of its binding partners, such as pre-mature SREBP2, from the ER.

It was also observed that CF induced the protein expression of the LDLR and increased LDL uptake in cultured hepatocytes. Given that SREBP2 regulates de novo expression of PCSK9 and the LDLR, the observed induction of cell-surface LDLR in the face of SREBP2 inhibition likely occurs in response to the loss of circulating PCSK9 levels. Because SREBP2 also induces the expression of HMGR, it is also possible that CF reduces circulating LDL cholesterol levels via inhibition of HMGR-mediated de novo cholesterol synthesis. In contrast to statins (which block HMGR, induce SREBP2 activity and increase circulating PCSK9 levels), these findings demonstrate that CF blocks SREBP2 and PCSK9. Thus, with reduced circulating PCSK9 levels, it is unlikely that HMGR activity is increased in response to CF.

### (a) Novel Compounds

In some embodiments, the present application includes a compound of Formula (Ia):

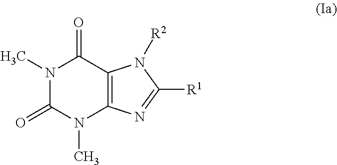

or a pharmaceutically acceptable salt, solvate and/or prodrug thereof;
wherein
R¹ is selected from
   (i) phenyl optionally substituted with one to five substituents independently selected from halo, CN, NH₂, OH, NO₂, C₁₋₆alkyl, C₁₋₆fluoroalkyl, C₁₋₆alkyleneOH, C₁₋₆ alkyleneNH₂, C₁₋₆alkyleneNH(C₁₋₆alkyl), C₁₋₆alkyleneN(C₁₋₆alkyl)(C₁₋₆alkyl), X¹—C₁₋₆ alkyl, X¹—C₁₋₆fluoroalkyl, CO₂H and C(O)H, and substituted with one or two of C₅₋₆ heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (ii) 9-10-membered bicyclic aryl wherein the second ring is phenyl or C₅₋₆cycloalkyl and the 9-10-membered bicyclic aryl is optionally substituted with one or more substituents independently selected from ═O, halo, CN, NH₂, OH, NO₂, C₁₋₆alkyl, C₁₋₆ fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iii) monocyclic C₃₋₆cycloalkyl or C₅₋₆cycloalkenyl optionally substituted with one or more substituents independently selected from halo, CN, NH₂, OH, NO₂, C₁₋₆alkyl, C₁₋₆ fluoroalkyl, C₁₋₆alkyleneOH, C₁₋₆alkyleneNH₂, C₁₋₆alkyleneNH(C₁₋₆alkyl), C₁₋₆ alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iv) 8-10-membered bicyclic cycloalkyl or cycloalkenyl wherein the second ring is C₅₋₆ heterocycloalkyl, phenyl, C₅₋₆cycloalkyl or C₅₋₆heteroaryl and the 8-10-membered bicyclic cycloalkyl or cycloalkenyl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (v) monocyclic C₅₋₆heterocycloalkyl optionally substituted with one or more substituents independently selected from halo, CN, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 5 groups being optionally substituted with one or more substituents independently selected from halo, OH, C₁₋₆alkyl, C₁₋₆ fluoroalkyl, X²—C₁₋₆alkyl, X²—C₁₋₆fluoroalkyl and NH₂;
   (vi) 8-10-membered bicyclic heterocycloalkyl wherein the second ring is C₅₋₆ heterocycloalkyl, phenyl, C₅₋₆cycloalkyl or C₅₋₆heteroaryl and the 8-10-membered bicyclic heterocycloalkyl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vii) monocyclic C₅₋₆heteroaryl, optionally substituted with one or more substituents independently selected from halo, CN, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 5 groups being optionally substituted with one or more substituents independently selected from halo, OH, C₁₋₆alkyl, C₁₋₆ fluoroalkyl, X²—C₁₋₆alkyl, X²—C₁₋₆fluoroalkyl and NH₂; and
   (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₅fluoroalkyl and NH₂;
R² is selected from H, C₁₋₆alkyl and C₁₋₆alkyl substituted with one or more substituents independently selected from OH and halo; and
X¹ and X² are independently selected from O, NH, N(C₁₋₆alkyl), N(C₁₋₆fluoroalkyl), C(O), C(O)O, OC(O), C(O)NH, C(O)N(C₁₋₆alkyl), C(O)N(C₁₋₆fluoroalkyl), NHC(O), N(C₁₋₆alkyl)C(O), N(C₁₋₆fluoroalkyl)C(O), S, S(O) and SO₂;
provided that R¹ is not:
   (a) phenyl monosubstituted with unsubstituted phenyl, unsubstituted pyridinyl and unsubstituted pyrazolyl
   (b) unsubstituted thiophenyl or thiophenyl substituted with Br, NO₂, CN, CH₃, C(O)H, C(O)CH₃ or OCH₃;
   (c) unsubstituted furanyl or furanyl substituted with Cl, Br, CH₃, CF₃, C(O)H or phenyl;
   (d) unsubstituted pyrimidinyl, unsubstituted pyridinyl or pyridinyl monosubstituted with NHCH₃, Br, CH₃ or pyridinyl;
   (e)

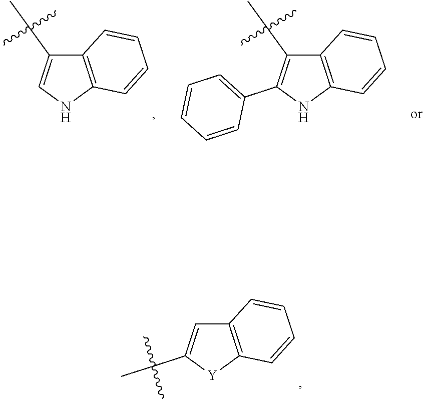

wherein Y is NH, O or S, and when Y is O, the phenyl ring is unsubstituted or mono-substituted with OCH₃ or Cl; and
(f)

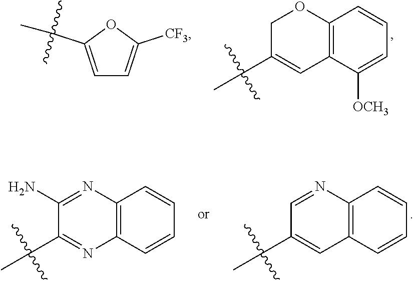 [SMILES: *c1ccc(C(F)(F)F)o1 ; *C1=Cc2c(OC)cccc2OC1 ; *c1nc2ccccc2nc1N ; *c1cnc2ccccc2c1]

In accordance with a further aspect of the present application, there is provided a compound of Formula (Ia):

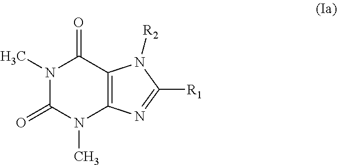

or a pharmaceutically acceptable salt, solvate and/or prodrug thereof;
wherein
R¹ is selected from
   (i) Phenyl optionally substituted with one to five substituents independently selected from halo, NH₂, OH, NO₂, C₁₋₆alkyl, C₁₋₆fluoroalkyl, C₁₋₆alkyleneOH, C₁₋₆alkyleneNH₂, C₁₋₆ alkyleneNH(C₁₋₆alkyl), C₁₋₆alkyleneN(C₁₋₆alkyl)(C₁₋₆alkyl), X¹—C₁₋₆alkyl, X¹—C₁₋₆ fluoroalkyl, CO₂H and C(O)H, and substituted with one or two of C₅₋₆heteroaryl, C₅₋₆ heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (ii) bicyclic C₉₋₁₀-aryl wherein the second ring in the bicyclic C₉₋₁₀aryl is phenyl or C₅₋₆ cycloalkyl and the C₉₋₁₀-aryl is optionally substituted with one or more substituents independently selected from ═O, halo, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iii) monocyclic C₃₋₆cycloalkyl or C₅₋₆cycloalkenyl optionally substituted with one or more substituents independently selected from halo, NH₂, OH, NO₂, C₁₋₆alkyl, C₁₋₆ fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₅fluoroalkyl and NH₂;
   (iv) bicyclic C₅₋₁₀cycloalkyl or C₅₋₁₀cycloalkenyl wherein the second ring in the bicyclic C₅₋₁₀cycloalkyl or C₅₋₁₀cycloalkenyl is C₅₋₆heterocycloalkyl, phenyl, C₅₋₆cycloalkyl or C₅₋₆heteroaryl and the C₅₋₁₀cycloalkyl or C₅₋₁₀cycloalkenyl is optionally substituted with one or more substituents independently selected from halo, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (v) monocyclic C₅₋₆heterocycloalkyl optionally substituted with one or more substituents independently selected from halo, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vi) bicyclic C₅₋₁₀heterocycloalkyl wherein the second ring in the bicyclic heterocycloalkyl is C₅₋₆heterocycloalkyl, phenyl, C₅₋₆cycloalkyl or C₅₋₆heteroaryl and the C₉₋₁₀heterocycloalkyl is optionally substituted with one or more substituents independently selected from halo, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vii) monocyclic C₅₋₆heteroaryl, optionally substituted with one or more substituents independently selected from halo, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂; and
   (viii) bicyclic C₅₋₁₀heteroaryl wherein the second ring in the bicyclic heteroaryl is C₅₋₆ heterocycloalkyl, phenyl, C₅₋₆cycloalkyl or C₅₋₆heteroaryl and the bicyclic C₉₋₁₀heteroaryl is optionally substituted with one or more substituents independently selected from halo, NH₂, OH, NO₂, C₁₋₆alkyl, C₁₋₆fluoroalkyl, C₁₋₆alkyleneOH, C₁₋₆ alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₈heteroaryl, C₅₋₈heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
R² is selected from H, C₁₋₆alkyl and C₁₋₆alkyl substituted with one or more substituents independently selected from OH and halo; and
X¹ and X² are independently selected from O, NH, N(C₁₋₆alkyl), N(C₁₋₆fluoroalkyl), C(O), C(O)O, OC(O), C(O)NH, C(O)N(C₁₋₆alkyl), C(O)N(C₁₋₆fluoroalkyl), NHC(O), N(C₁₋₆alkyl)C(O), N(C₁₋₆fluoroalkyl)C(O), S, S(O) and SO₂;
provided that R¹ is not:
(a) phenyl monosubstituted with unsubstituted phenyl, unsubstituted pyridinyl and unsubstituted pyrazolyl;
(b) unsubstituted thiophenyl or thiophenyl substituted with Br, NO₂, CN, CH₃, C(O)H, C(O)CH₃ or OCH₃;
(c) unsubstituted furanyl or furanyl substituted with Cl, Br, CH₃, CF₃, C(O)H or phenyl;
(d) unsubstituted pyridinyl or pyridinyl monosubstituted with NHCH₃, Br, CH₃ or pyridinyl;
(e)

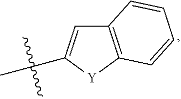

wherein Y is NH, O or S, and when Y is O, the phenyl ring is unsubstituted or mono-substituted with OCH₃ or Cl; and
(f)

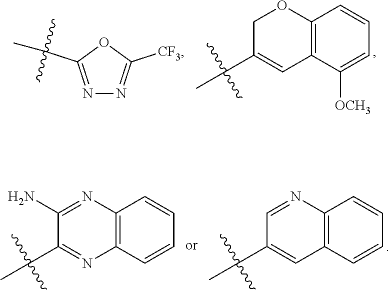

In some embodiments, R¹ is monocyclic C₅₋₆heteroaryl, optionally substituted with one to four substituents, one to three substituents, one to two substituents or one substituent independently selected from F, Cl, Br, F, NH₂, OH, NO₂, C₁₋₆alkyl, C₁₋₆fluoroalkyl, C₁₋₆alkylOH, C₁₋₆alkylNH₂, X¹—C₁₋₆alkyl, X¹—C₁₋₆fluoroalkyl, CO₂H, C(O)H, C₅₋₆heteroaryl, C₅₋₆heterocycloalkyl, phenyl, C₃₋₆cycloalkyl and C₃₋₆cycloalkenyl, the latter 5 groups being optionally substituted with one to four substituents, one to three substituents, one to two substituents or one substituent independently selected from F, Cl, Br, F, OH, C₁₋₄alkyl, C₁₋₄fluoroalkyl, X²—C₁₋₄alkyl, X²—C₁₋₄fluoroalkyl and NH₂, wherein X¹ and X² are independently selected from O, NH, N(C₁₋₄ alkyl), N(C₁₋₄fluoroalkyl), C(O), C(O)O, OC(O), C(O)NH, NHC(O), C(O)N(C₁₋₄alkyl), C(O)N(C₁₋₄fluoroalkyl), N(C₁₋₄alkyl)C(O), N(C₁₋₄fluoroalkyl)C(O), S, S(O) and SO₂. In some embodiments, R¹ is monocyclic C₅₋₆heteroaryl, optionally substituted with one to four substituents, one to three substituents, one to two substituents or one substituent independently selected from F, Cl, Br, F, NH₂, OH, NO₂, C₁₋₆alkyl, C₁₋₆fluoroalkyl, C₁₋₆alkylOH, C₁₋₆alkylNH₂, X¹—C₁₋₆alkyl, X¹—C₁₋₆fluoroalkyl, CO₂H, and C(O)H, wherein X¹ is selected from O, NH, N(CH₃), N(CF₃), C(O), C(O)O, OC(O), C(O)NH, NHC(O), C(O)N(CH₃), C(O)N(CF₃), N(CH₃)C(O), N(CF₃)C(O), S, S(O) and SO₂.

In some embodiments, R¹ is (i) phenyl optionally substituted with one to five substituents independently selected from halo, CN, NH₂, OH, NO₂, C₁₋₄alkyl, C₁₋₄fluoroalkyl, C₁₋₄ alkyleneOH, C₁₋₄alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H and C(O)H, and substituted with one or two of C₅₋₆ heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 5 groups being optionally substituted with one or more substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (i) phenyl optionally substituted with one to five substituents independently selected from halo, CN, NH₂, OH, NO₂, C₁₋₄alkyl, C₁₋₄fluoroalkyl, C₁₋₄alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), X¹—C₁₋₄alkyl, X¹—C₁₋₄fluoroalkyl, CO₂H and C(O)H, and substituted with one or two of C₅₋₆heteroaryl, C₅₋₆heterocycloalkyl, phenyl, X¹-phenyl, C₁₋₂alkylene-phenyl, X¹—C₁₋₂ alkylenephenyl, C₃₋₆cycloalkyl and C₃₋₆cycloalkenyl, the latter 5 groups being optionally substituted with one to five substituents independently selected from halo, OH, C₁₋₄alkyl, C₁₋₄ fluoroalkyl, X²—C₁₋₄alkyl, X²—C₁₋₄fluoroalkyl and NH₂.

In some embodiments, X¹ in (i) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (i) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (i) is selected from O, C(O), C(O)O, OC(O), C(O)NH, NHC(O), S and SO₂

In some embodiments, X² in (i) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (i) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (i) is selected from O, NH, C(O), C(O)O, OC(O), C(O)NH, NHC(O), S, S(O) and SO₂. In some embodiments, X² in (i) is selected from O, NH and C(O). In some embodiments, X² in (i) selected from O and C(O).

In some embodiments, X¹ in (i) is selected from O, C(O), C(O)O, OC(O), C(O)NH, NHC(O), and SO₂ and X² in (i) is selected from O and C(O), and R¹ is (i) phenyl optionally substituted with one to four substituents independently selected from F, Cl, Br, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), O—C₁₋₂alkyl, O—C₁₋₂fluoroalkyl, NHC(O)—C₁₋₂alkyl, NHC(O)C₁₋₄fluoroalkyl, C(O)C₁₋₄alkyl, C(O)C₁₋₄fluoroalkyl C(O)OC₁₋₄alkyl, C(O)OC₁₋₄fluoroalkyl, OC(O)C₁₋₂alkyl, OC(O)C₁₋₄fluoroalkyl, SO₂C₁₋₄alkyl, SO₂C₁₋₄fluoroalkyl, CO₂H, C(O)H, and substituted with one or two of C₅₋₆heteroaryl, C₅₋₆heterocycloalkyl, phenyl, O-phenyl, C(O)-phenyl, C₁₋₂alkylene-phenyl, O—C₁₋₂alkylenephenyl, C(O)—C₁₋₂alkylenephenyl, C₃₋₆cycloalkyl and C₃₋₆cycloalkenyl, the latter 10 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂.

In some embodiments, R¹ is (i) phenyl substituted with one to four substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, NHC(O)CH₃, NHC(O)CF₃, C(O)CH₃, C(O)CF₃, OC(O)CH₃, OC(O)CF₃, SO₂CH₃, SO₂CF₃, CO₂H, C(O)H, and substituted with one or two of C₅₋₆heteroaryl, C₅₋₆ heterocycloalkyl, phenyl, O-phenyl, C(O)-phenyl, CH₂-phenyl, O—CH₂phenyl, C(O)— CH₂pheny, C₃₋₆cycloalkyl and C₃₋₆cycloalkenyl, the latter 10 groups being optionally substituted with one to four substituents independently selected from F, Cl, OH, CH₃, CF₃, CH₃O, CF₃O, and NH₂.

In some embodiments, R¹ is (i) phenyl substituted with one or two substituents independently selected from C₅₋₆heteroaryl, C₅₋₆heterocycloalkyl, phenyl, O-phenyl, C(O)-phenyl, CH₂-phenyl, O-CH₂phenyl, C(O)—CH₂pheny, C₃₋₆cycloalkyl and C₃₋₆cycloalkenyl, the latter 10 groups being optionally substituted with one to four substituents independently selected from F, Cl, OH, CH₃, CF₃, CH₃O, CF₃O, and NH₂. In some embodiments, R¹ is phenyl substituted with one or two substituents independently selected from C₅₋₆heteroaryl, phenyl, O-phenyl, C(O)-phenyl, O—CH₂phenyl and C(O)—CH₂phenyl, the latter 6 groups being optionally substituted with one to three substituents independently selected from F, Cl, OH, CH₃, CF₃, CH₃O, CF₃O, and NH₂.

In some embodiments, R¹ is (ii) 9-10-membered bicyclic aryl wherein the second ring is phenyl or C₅₋₆cycloalkyl and the 9-10 membered bicyclic aryl is optionally substituted with one to five substituents independently selected from ═O, halo, CN, NH₂, OH, NO₂, C₁₋₄alkyl, C₁₋₄ fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₆alkyl), X¹—C₁₋₄alkyl, X¹—C₁₋₄fluoroalkyl, CO₂H, C(O)H, C₅₋₈heteroaryl, C₅₋₆ heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (ii) 9-10-membered bicyclic aryl wherein the second ring is phenyl or C₅₋₆cycloalkyl and the 9-10 membered bicyclic aryl is optionally substituted with one or more substituents independently selected from ═O, halo, CN, NH₂, OH, NO₂, C₁₋₄alkyl, C₁₋₄ fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₆alkyl), X¹—C₁₋₄alkyl, X¹—C₁₋₄fluoroalkyl, CO₂H, C(O)H, C₅₋₆heteroaryl, C₅₋₆ heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one or more substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (ii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (ii) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (ii) is selected from O, C(O), C(O)O, OC(O), C(O)NH, NHC(O), S and SO₂ In some embodiments, X¹ in (ii) is O,

In some embodiments, X² in (ii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (ii) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (ii) is selected from O, NH, C(O), C(O)O, OC(O), C(O)NH, NHC(O), S, S(O) and SO₂. In some embodiments, X² in (ii) is selected from O, NH and C(O). In some embodiments, X² in (ii) is O.

In some embodiments, X¹ in (ii) is O and X² in (ii) is O, and R¹ is (ii) 9-10-membered bicyclic aryl wherein the second ring is phenyl or C₅₋₆cycloalkyl and the 9-10 membered bicyclic aryl is optionally substituted with one to four substituents independently selected from ═O, halo, CN, NH₂, OH, NO₂, C₁₋₄alkyl, C₁₋₄fluoroalkyl, C₁₋₂alkyleneOH, C₁₋₂alkyleneNH₂, C₁₋₂ alkyleneNH(C₁₋₄alkyl), C₁₋₂alkyleneN(C₁₋₄alkyl)(C₁₋₆alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₄alkylene-phenyl, O—C₁₋₄alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄ fluoroalkyl, O—C₁₋₄alkyl, O—C₁₋₄fluoroalkyl and NH₂.

In some embodiments, X¹ in (ii) is O and X² in (ii) is O, and R¹ is (ii) 9-10-membered bicyclic aryl wherein the second ring is phenyl or C₅₋₆cycloalkyl and the 9-10 membered bicyclic aryl is optionally substituted with one to three substituents independently selected from ═O, F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂phenyl, O CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂.

In some embodiments, R¹ is (ii) 9-10-membered bicyclic aryl wherein the second ring is phenyl or C₅₋₆cycloalkyl and the 9-10 membered bicyclic aryl is optionally substituted with one to three substituents independently selected from ═O, F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H and C(O)H. In some embodiments, R¹ is (ii) 9-10-membered bicyclic aryl wherein the second ring is phenyl or C₅₋₆cycloalkyl and the 9-10 membered bicyclic aryl is optionally substituted with one to three substituents independently selected from ═O, F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H and C(O)H.

In some embodiments, R¹ is (ii) 9-10-membered bicyclic aryl wherein the second ring is phenyl and 9-10 membered bicyclic aryl is optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H and C(O)H. In some embodiments, R¹ is (ii) 9-10-membered bicyclic aryl wherein the second ring is phenyl and 9-10 membered bicyclic aryl is optionally substituted with one to three substituents independently selected from F, Cl, Br and CH₃. In some embodiments, R¹ is (ii) 9-10-membered bicyclic aryl wherein the second ring is C₅₋₆cycloalkyl and the 9-10 membered bicyclic aryl is optionally substituted with one to three substituents independently selected from ═O, F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H and C(O)H. In some embodiments, R¹ is (ii) 9-10-membered bicyclic aryl wherein the second ring is C₅₋₆cycloalkyl and the 9-10 membered bicyclic aryl is optionally substituted with one to three substituents independently selected from ═O and F, Cl, Br, CH₃. In some embodiments, R¹ is (ii) 9-10-membered bicyclic aryl wherein the second ring is C₅₋₆ cycloalkyl and the 9-10 membered bicyclic aryl is optionally substituted with one or two ═O.

In some embodiments, R¹ is (ii) 9-10-membered bicyclic aryl wherein the second ring is phenyl or C₅₋₆cycloalkyl and the 9-10 membered bicyclic aryl is optionally substituted with one to three substituents independently selected from, C₅₋₆heteroaryl, C₅₋₆heterocycloalkyl, phenyl, O-phenyl, CH₂phenyl, O CH₂phenyl, C₃₋₆cycloalkyl and C₃₋₆cycloalkenyl, the latter 8 groups being optionally substituted with one to five substituents independently selected from halo, OH, C₁₋₄ alkyl, C₁₋₄fluoroalkyl, O—C₁₋₄alkyl, O—C₁₋₄fluoroalkyl and NH₂.

In some embodiments, R¹ is (iii) monocyclic C₃₋₆cycloalkyl or C₅₋₆cycloalkenyl optionally substituted with one to five substituents independently selected from halo, CN, NH₂, OH, NO₂, C₁₋₄alkyl, C₁₋₄fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆ heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (iii) monocyclic C₃₋₆cycloalkyl or C₅₋₆cycloalkenyl optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂ alkyleneNH(C₁₋₆alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₆ fluoroalkyl, X²—C₁₋₄alkyl, X²—C₁₋₄fluoroalkyl and NH₂

In some embodiments, X¹ in (iii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (iii) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (iii is selected from O, C(O), C(O)O, OC(O), C(O)NH, NHC(O), S and SO₂ In some embodiments, X¹ in (iii) is O.

In some embodiments, X² in (iii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (ii) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (iii) is selected from O, NH, C(O), C(O)O, OC(O), C(O)NH, NHC(O), S, S(O) and SO₂. In some embodiments, X² in (iii) is selected from O, NH and C(O). In some embodiments, X² in (iii) is O.

In some embodiments, X¹ in (iii) is O and X² in (iii) is O, and R¹ is (iii) monocyclic C₃₋₆cycloalkyl or C₅₋₆cycloalkenyl optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₄ alkylene-phenyl, O—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₆fluoroalkyl, O—C₁₋₄alkyl, O—C₁₋₄fluoroalkyl and NH₂ In some embodiments, R¹ is (iii) monocyclic C₅₋₆cycloalkyl or C₅₋₆cycloalkenyl optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆ heterocycloalkyl, phenyl, O-phenyl, C₁₋₄alkylene-phenyl, O—C₁₋₄alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂ In some embodiments, R¹ is (iii) monocyclic C₅₋₆cycloalkyl optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆ heterocycloalkyl, phenyl, O-phenyl, C₁₋₄alkylene-phenyl, O—C₁₋₄alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₆fluoroalkyl, O—C₁₋₄alkyl, O—C₁₋₄fluoroalkyl and NH₂ In some embodiments, R¹ is (iii) monocyclic C₅₋₆cycloalkenyl optionally substituted with one to three substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂ CO₂H, C(O)H, C₅₋₆heteroaryl, C₅₋₆ heterocycloalkyl, phenyl, O-phenyl, C₁₋₄alkylene-phenyl, O—C₁₋₄alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂

In some embodiments, R¹ is (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one o five substituents independently selected from halo, CN, NH₂, OH, NO₂, C₁₋₄alkyl, C₁₋₄fluoroalkyl, C₁₋₄alkyleneOH, C₁₋₄alkyleneNH₂, C₁₋₄alkyleneNH(C₁₋₆alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆ heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one to five substituents independently selected from halo, CN, NH₂, OH, NO₂, C₁₋₄alkyl, C₁₋₄fluoroalkyl, C₁₋₂ alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₂alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (iv) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (iv) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (iv) is selected from O, C(O), C(O)O, OC(O), C(O)NH, NHC(O), S and SO₂ In some embodiments, X¹ in (iv) is O,

In some embodiments, X² in (iv) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (iv) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (iv) is selected from O, NH, C(O), C(O)O, OC(O), C(O)NH, NHC(O), S, S(O) and SO₂. In some embodiments, X² i in (iv) is selected from O, NH and C(O). In some embodiments, X² in (iv) is O.

In some embodiments, X¹ in (iv) is O and X² in (iv) is O, and R¹ is (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₂alkylene-phenyl, O—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄ fluoroalkyl, O—C₁₋₄alkyl, O—C₁₋₄fluoroalkyl and NH₂.

In some embodiments, X¹ in (iv) is O and X² in (iv) is O, and R¹ is (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O-CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂.

In some embodiments, R¹ is (iv) 8-10-membered bicyclic cycloalkyl wherein the second ring is C₅₋₆heterocycloalkyl, phenyl, C₅₋₆cycloalkyl or C₅₋₆heteroaryl and the 8-10-membered bicyclic cycloalkyl is optionally substituted with one to five substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O— CH₂phenyl, C₃₋₆cycloalkyl and C₃₋₆cycloalkenyl, the latter 8 groups being optionally substituted with one to five substituents independently selected from halo, OH, C₁₋₄alkyl, C₁₋₄ fluoroalkyl, O—C₁₋₄alkyl, O—C₁₋₄fluoroalkyl and NH₂. In some embodiments, R¹ is (iv) 8-10-membered bicyclic cycloalkyl wherein the second ring is phenyl or C₅₋₆heteroaryl and the 8-10-membered bicyclic cycloalkyl is optionally substituted with one to five substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O— CH₂phenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (iv) 8-10-membered bicyclic cycloalkyl wherein the second ring is phenyl and the 8-10-membered bicyclic cycloalkyl or cycloalkenyl is optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O—CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄ fluoroalkyl, O—C₁₋₄alkyl, O—C₁₋₄fluoroalkyl and NH₂.

In some embodiments, R¹ is (iv) 8-10-membered bicyclic cycloalkenyl wherein the second ring is C₅₋₆heterocycloalkyl, phenyl, C₅₋₆cycloalkyl or C₅₋₆heteroaryl and the 8-10-membered bicyclic cycloalkenyl is optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O— CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (iv) 8-10-membered bicyclic cycloalkenyl wherein the second ring is phenyl or C₅₋₆heteroaryl and the 8-10-membered bicyclic cycloalkenyl is optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂CO₂H, C(O)H, C₅₋₆heteroaryl, C₅₋₆heterocycloalkyl, phenyl, O-phenyl, CH₂-phenyl, O—CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (iv) 8-10-membered bicyclic cycloalkenyl wherein the second ring is phenyl and the 8-10-membered bicyclic cycloalkenyl is optionally substituted with one to three substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O—CH₂phenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (iv) 8-10-membered bicyclic cycloalkenyl wherein the second ring is phenyl.

In some embodiments, R¹ is (v) monocyclic C₅₋₆heterocycloalkyl optionally substituted with one to five substituents independently selected from halo, CN, NH₂, OH, NO₂, C₁₋₄ alkyl, C₁₋₄fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄ fluoroalkyl and NH₂. In some embodiments, R¹ is (v) monocyclic C₅₋₆heterocycloalkyl optionally substituted with one to five substituents independently selected from halo, CN, NH₂, OH, NO₂, C₁₋₄ alkyl, C₁₋₄fluoroalkyl, C₁₋₂alkyleneOH, C₁₋₂alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₄alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄ fluoroalkyl and NH₂.

In some embodiments, X¹ in (v) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (v) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (v) is selected from O, C(O), C(O)O, OC(O), C(O)NH, NHC(O), S and SO₂ In some embodiments, X¹ in (v) is O,

In some embodiments, X² in (v) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (v) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (v) is selected from O, NH, C(O), C(O)O, OC(O), C(O)NH, NHC(O), S, S(O) and SO₂. In some embodiments, X² in (v) is selected from O, NH and C(O). In some embodiments, X² in (v) is O.

In some embodiments, X¹ in (v) is O and X² in (v) is O, and R¹ is (v) monocyclic C₅₋₆heterocycloalkyl optionally substituted with one to five substituents independently selected from F, Br, Cl, CN, NH₂, OH, NO₂, C₁₋₄alkyl, C₁₋₄fluoroalkyl, C₁₋₂alkyleneOH, C₁₋₂alkyleneNH₂, C₁₋₄alkyleneNH(C₁₋₄alkyl), C₁₋₄alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₄alkylene-phenyl, O—C₁₋₂ alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄ fluoroalkyl, O—C₁₋₄alkyl, O—C₁₋₄fluoroalkyl and NH₂. In some embodiments, R¹ is (v) monocyclic C₅₋₆heterocycloalkyl optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₄ alkylene-phenyl, O—C₁₋₂alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂.

In some embodiments, the monocyclic C₅₋₆heterocycloalkyl in R¹ is selected from tetrahydrofuranyl, tetrahydrothiophenyl, pyrrolidinyl, imidazolidinyl, pyrazolidinyl, 2-oxopiperazinyl, 2-oxopiperdinyl, 2-oxopyrrolidinyl, isoxthiolidinyl, thiazolidinyl, isothiazolidinyl, piperidinyl, dihydropyranyl, tetrahydropyranyl, diazinanyl (e.g, piperazinyl), piperidinyl, morpholinyl, thiomorpholinyl, thiomorpholinyl sulfoxide, dioxanyl and dithianyl each of which is optionally substituted with one to three substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₄alkylene-phenyl, O—C₁₋₂ alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄ fluoroalkyl, O—C₁₋₄alkyl, O—C₁₋₄fluoroalkyl and NH₂. In some embodiments, the monocyclic C₅₋₆ heterocycloalkyl in R¹ is dihydropyranyl.

In some embodiments, R¹ is (vi) 8-10-membered bicyclic heterocycloalkyl wherein the second ring is C₅₋₆heterocycloalkyl, phenyl, C₅₋₆cycloalkyl or C₅₋₆heteroaryl and the 8-10-membered bicyclic heterocycloalkyl is optionally substituted with one to five substituents independently selected from halo, CN, NH₂, OH, NO₂, C₁₋₄alkyl, C₁₋₄fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₄ alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (vi) 8-10-membered bicyclic heterocycloalkyl wherein the second ring is C₅₋₆heterocycloalkyl, phenyl, C₅₋₆cycloalkyl or C₅₋₆heteroaryl and the 8-10-membered bicyclic heterocycloalkyl is optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₆ heteroaryl, C₅₋₆heterocycloalkyl, phenyl, X¹-phenyl, C₁₋₂alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (vi) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (vi) is selected from O, C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S and SO₂. In some embodiments, X¹ in (vi) is selected from O, C(O), C(O)O, OC(O), C(O)NH, NHC(O), S and SO₂ In some embodiments, X¹ in (vi) is O,

In some embodiments, X² in (vi) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (vi) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (v) is selected from O, NH, C(O), C(O)O, OC(O), C(O)NH, NHC(O), S, S(O) and SO₂. In some embodiments, X² in (vi) is selected from O, NH and C(O). In some embodiments, X² in (vi) is O.

In some embodiments, X¹ in (vi) is O and X² in (vi) is O, and R¹ is (vi) 8-10-membered bicyclic heterocycloalkyl wherein the second ring is C₅₋₆heterocycloalkyl, phenyl, C₅₋₆ cycloalkyl or C₅₋₆heteroaryl and the 8-10-membered bicyclic heterocycloalkyl is optionally substituted with one to five substituents independently selected from halo, CN, NH₂, OH, NO₂, C₁₋₄ alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆ heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (vi) 8-10-membered bicyclic heterocycloalkyl wherein the second ring is C₅₋₆heterocycloalkyl, phenyl, C₅₋₆cycloalkyl or C₅₋₆heteroaryl and the 8-10-membered bicyclic heterocycloalkyl is optionally substituted with one to four substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₂ alkylene-phenyl, O—C₁₋₂alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (vi) 9-10-membered bicyclic heterocycloalkyl wherein the second ring is C₅₋₆heterocycloalkyl, phenyl, C₅₋₆cycloalkyl or C₅₋₆heteroaryl and the 9-10-membered bicyclic heterocycloalkyl is optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄ fluoroalkyl and NH₂. In some embodiments, R¹ is (vi) 9-10-membered bicyclic heterocycloalkyl wherein the second ring is phenyl and the 9-10-membered bicyclic heterocycloalkyl is optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H, C(O)H, and phenyl, the phenyl being optionally substituted with one to three substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (vi) 9-10-membered bicyclic heterocycloalkyl wherein the second ring is phenyl and the 9-10-membered bicyclic heterocycloalkyl is optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H, C(O)H, and phenyl, the phenyl being optionally substituted with one to three substituents independently selected from halo, OH, C₁₋₄alkyl, C₁₋₄ fluoroalkyl, O—C₁₋₄alkyl, O—C₁₋₄fluoroalkyl and NH₂.

In some embodiments, R¹ is (vi) 9-10-membered bicyclic heterocycloalkyl wherein the second ring is phenyl and the 9-10-membered bicyclic heterocycloalkyl is optionally substituted with one o three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H, C(O)H, and phenyl, the phenyl being optionally substituted with one to three substituents independently selected from halo, OH, C₁₋₄alkyl, C₁₋₄fluoroalkyl, O—C₁₋₄alkyl, O—C₁₋₄fluoroalkyl and NH₂.

In some embodiments, R¹ is (vii) monocyclic C₅₋₆heteroaryl, optionally substituted with one to five substituents independently selected from halo, CN, NH₂, OH, NO₂, C₁₋₄alkyl, C₁₋₄fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆ heterocycloalkyl, phenyl, X¹-phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄ fluoroalkyl and NH₂. In some embodiments, R¹ is (vii) monocyclic C₅₋₆heteroaryl, optionally substituted with one to five substituents independently selected from halo, CN, NH₂, OH, NO₂, C₁₋₄ alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, X¹-phenyl, C₁₋₂alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (vii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (vii) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (vii) is selected from O, C(O), C(O)O, OC(O), C(O)NH, NHC(O), S and SO₂ In some embodiments, X¹ in (vii) is selected from O and S.

In some embodiments, X² in (vii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (vii) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (vii) is selected from O, NH, C(O), C(O)O, OC(O), C(O)NH, NHC(O), S, S(O) and SO₂. In some embodiments, X² in (vii) is selected from O, NH and C(O). In some embodiments, X² in (vii) is O.

In some embodiments, X¹ in (vii) is selected from O and S and X² in (vii) is O and R¹ is (vii) monocyclic C₅₋₆heteroaryl, optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), O—C₁₋₄alkyl, S—C₁₋₄alkyl, O—C₁₋₄fluoroalkyl, S—C₁₋₄fluoroalkyl, CO₂H, C(O)H, C₅₋₆heteroaryl, C₅₋₆ heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄ fluoroalkyl and NH₂. In some embodiments, R¹ is (vii) monocyclic C₅₋₆heteroaryl, optionally substituted with one to four substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, CH₃S, CF₃S, CH₂OH, CH₂NH₂, CO₂H, C(O)H, C₅₋₆heteroaryl, C₅₋₆ heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄ fluoroalkyl and NH₂. In some embodiments, R¹ is (vii) monocyclic C₅₋₆heteroaryl, optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CH₃S, CF₃S, CH₂OH, CH₂NH₂, CO₂H, C(O)H, C₅₋₆heteroaryl, C₅₋₆heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂.

In some embodiments, the monocyclic C₅₋₆heteroaryl in R¹ is selected from furanyl, thiophenyl, pyridyl, triazolyl, pyrrolyl, tetrazolyl, imidazolyl, pyrazolyl, oxazolyl, thiazolyl, pyrimidinyl, isoxazolyl and isothiazolyl, optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CH₃S, CF₃S, CH₂OH, CH₂NH₂, CO₂H, C(O)H, C₅₋₆heteroaryl, C₅₋₆heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂. In some embodiments, the monocyclic C₅₋₆heteroaryl in R¹ is selected from thiophenyl, pyridyl, pyrazolyl, oxazolyl, pyrimidinyl, and isothiazolyl, optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CH₃S, CF₃S, CH₂OH, CH₂NH₂, CO₂H, C(O)H, C₅₋₆heteroaryl, C₅₋₆ heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄ fluoroalkyl and NH₂. In some embodiments, the monocyclic C₅₋₆heteroaryl in R¹ is selected from pyridyl, and pyrimidinyl optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CH₃S, CF₃S, CH₂OH, CH₂NH₂, CO₂H, C(O)H, C₅₋₆heteroaryl, C₅₋₆ heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄ fluoroalkyl and NH₂.

In some embodiments, R¹ is (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one to five substituents independently selected from halo, CN, NH₂, OH, NO₂, C₁₋₆alkyl, C₁₋₄fluoroalkyl, C₁₋₄alkyleneOH, C₁₋₄ alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄ alkyl, C₁₋₄fluoroalkyl, X²—C₁₋₄alkyl, X²—C₁₋₄fluoroalkyl and NH₂. In some embodiments, R¹ is (viii) 8-10-membered bicyclic heteroaryl wherein the second ring is C₅₋₆heterocycloalkyl, phenyl, C₅₋₆ cycloalkyl or C₅₋₆heteroaryl and the 8-10-membered bicyclic heteroaryl is optionally substituted with one to five substituents independently selected from halo, CN, NH₂, OH, NO₂, C₁₋₆alkyl, C₁₋₄fluoroalkyl, C₁₋₄alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆ heterocycloalkyl, phenyl, X¹-phenyl, C₁₋₂alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (viii) is selected from O, NH, N(C₁₋₄alkyl), N(C₁₋₄fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (viii) is selected from O, C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O) and SO₂. In some embodiments, X¹ in (viii) is selected from O, C(O), C(O)O, OC(O), C(O)NH, NHC(O), and SO₂ In some embodiments, X¹ in (viii) is O,

In some embodiments, X² in (viii) is selected from O, NH, N(C₁₋₄alkyl), N(C₁₋₄fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (viii) is selected from O, NH, C(O), C(O)O, OC(O), C(O)NH, NHC(O), N(C₁₋₄alkyl)C(O), N(C₁₋₄fluoroalkyl)C(O), S, S(O) and SO₂. In some embodiments, X² in (viii) is selected from O, NH, C(O), C(O)O, OC(O), C(O)NH, NHC(O), S, S(O) and SO₂. In some embodiments, X² in (viii) is selected from O, NH and C(O). In some embodiments, X² in (viii) is O.

In some embodiments, X¹ in (viii) is selected from O and S and X² in (viii) is O and R¹ is (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one to five substituents independently selected from halo, CN, NH₂, OH, NO₂, C₁₋₆alkyl, C₁₋₄fluoroalkyl, C₁₋₄alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₈heteroaryl, C₅₋₆ heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one to four substituents independently selected F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to four substituents independently selected from halo, OH, C₁₋₄ alkyl, C₁₋₄fluoroalkyl, O—C₁₋₄alkyl, O—C₁₋₄fluoroalkyl and NH₂. In some embodiments, R¹ is (viii) 9-10-membered bicyclic heteroaryl wherein the second ring is C₅₋₆heterocycloalkyl, phenyl, C₅₋₆ cycloalkyl or C₅₋₆heteroaryl and the 9-10-membered bicyclic heteroaryl is optionally substituted with one to three substituents independently selected F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, C(CF₃)₃, CH(CH₃)₂O, CH₃CH₂O, CH₃O, CF₃O, CHF₂O, CH₂OH, CH₂NH₂, CO₂H, C(O)H, C₅₋₆heteroaryl, C₅₋₆ heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂.

In some embodiments, the 9-10-membered bicyclic heteroaryl in R¹ is selected from benzofuranyl, indolyl, isoindolyl, benzodioxolyl, benzothiazolyl quinolinyl, isoquinolinyl and benzothiophenyl each of which is optionally substituted with one to three substituents independently selected F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, C(CF₃)₃, CH(CH₃)₂O, CH₃CH₂O, CH₃O, CF₃O, CHF₂O, CH₂OH, CH₂NH₂, CO₂H, C(O)H, C₅₋₆heteroaryl, C₅₋₆heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂.

In some embodiments, each C₅₋₆heteroaryl in the substituents on R¹ is independently selected from furanyl, thiophenyl, pyridyl, triazolyl, pyrrolyl, tetrazolyl, imidazolyl, pyrazolyl, oxazolyl, thiazolyl, pyrimidinyl, isoxazolyl and isothiazolyl.

In some embodiments, the C₅₋₆heteroaryl in the substituents on R¹ in (i) is selected from triazolyl and pyridyl. In some embodiments, the triazole is a 1, 2, 4 triazolyl. In some embodiments, the substituent C₅₋₆heteroaryl in R¹ in (i) is pyridyl.

In some embodiments, the C₅₋₆heterocycloalkyl in the substituents on R¹ is independently selected from tetrahydrofuranyl, tetrahydrothiophenyl, pyrrolidinyl, imidazolidinyl, pyrazolidinyl, 2-oxopiperazinyl, 2-oxopiperdinyl, 2-oxopyrrolidinyl, isoxthiolidinyl, thiazolidinyl, isothiazolidinyl, piperidinyl, dihydropyranyl, tetrahydropyranyl, diazinanyl (e.g, piperazinyl), piperidinyl, morpholinyl, thiomorpholinyl, thiomorpholinyl sulfoxide, dioxanyl and dithianyl.

In some embodiments, each C₃₋₆cycloalkyl in the substituents on R¹ is independently selected from, cyclopropyl, cyclobutyl, cyclopentyl and cyclohexyl,

In some embodiments, each C₃₋₆cycloalkenyl in the substituents on R¹ is independently selected from, cyclopropenyl, cyclobutenyl, cyclopentenyl and cyclohexenyl.

In some embodiments, R² is selected from H, C₁₋₄alkyl and C₁₋₄alkyl substituted with one or more substituents independently selected from OH and halo. In some embodiments, R² is selected from H, CH₃, CF₃, CF₂H, CH₃O, CF₃O, CHF₂O and CH₂OH. In some embodiments, R² is H. In some embodiments, R² is CH₃.

In some embodiments, “one or more” is one to five. In some embodiments, “one or more” is one to four. In some embodiments, “one or more” is one to three. In some embodiments, “one or more” is “one or two”.

In some embodiments, the compound of Formula (Ia) is selected from:

| Compound No. | Structure |
|---|---|
| Ia-4 | 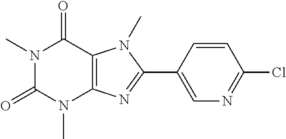 [SMILES: Cn1c(=O)c2c(nc(-c3ccc(Cl)nc3)n2C)n(C)c1=O] |
| Ia-5 | 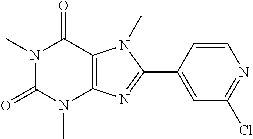 [SMILES: Cn1c(=O)c2c(nc(-c3ccnc(Cl)c3)n2C)n(C)c1=O] |
| Ia-6 | 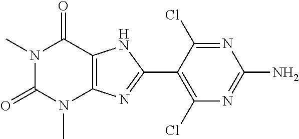 [SMILES: Cn1c(=O)c2[nH]c(-c3c(Cl)nc(N)nc3Cl)nc2n(C)c1=O] |
| Ia-7 | 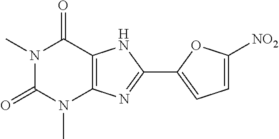 [SMILES: Cn1c(=O)c2[nH]c(-c3ccc([N+](=O)[O-])o3)nc2n(C)c1=O] |
| Ia-9 | 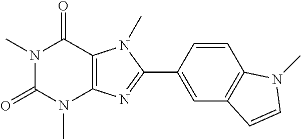 |
| Ia-10 | 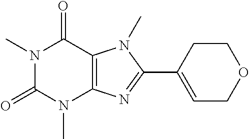 [SMILES: Cn1c(=O)c2c(nc(C3=CCOCC3)n2C)n(C)c1=O] |
| Ia-12 | 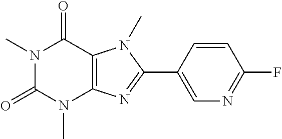 [SMILES: Cn1c(=O)c2c(nc(-c3ccc(F)nc3)n2C)n(C)c1=O] |
| Ia-13 | 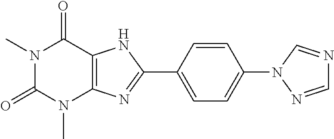 [SMILES: Cn1c(=O)c2[nH]c(-c3ccc(-n4cncn4)cc3)nc2n(C)c1=O] |
| Ia-15 | 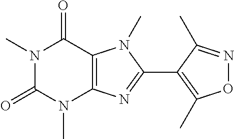 [SMILES: Cc1noc(C)c1-c1nc2c(c(=O)n(C)c(=O)n2C)n1C] |
| Ia-16 | 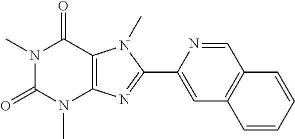 [SMILES: Cn1c(=O)c2c(nc(-c3cc4ccccc4cn3)n2C)n(C)c1=O] |
| Ia-17 | 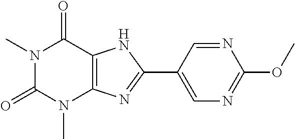 |
| Ia-19 | 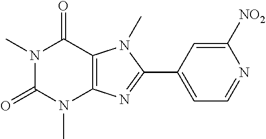 [SMILES: Cn1c(=O)c2c(nc(-c3ccnc([N+](=O)[O-])c3)n2C)n(C)c1=O] |
| Ia-21 | 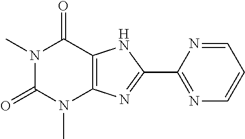 [SMILES: Cn1c(=O)c2[nH]c(-c3ncccn3)nc2n(C)c1=O] |
| Ia-26 | 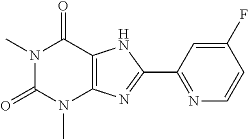 [SMILES: Cn1c(=O)c2[nH]c(-c3cc(F)ccn3)nc2n(C)c1=O] |
| Ia-27 | 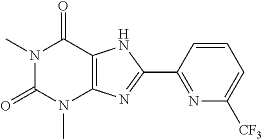 [SMILES: Cn1c(=O)c2[nH]c(-c3cccc(C(F)(F)F)n3)nc2n(C)c1=O] |
| Ia-28 | 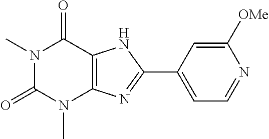 [SMILES: COc1cc(-c2nc3c([nH]2)c(=O)n(C)c(=O)n3C)ccn1] |
| Ia-34 | 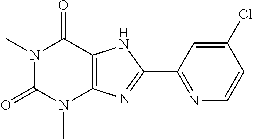 [SMILES: Cn1c(=O)c2[nH]c(-c3cc(Cl)ccn3)nc2n(C)c1=O] |
| Ia-35 | 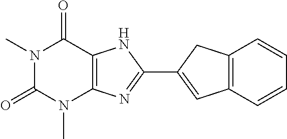 [SMILES: Cn1c(=O)c2[nH]c(C3=Cc4ccccc4C3)nc2n(C)c1=O] |
| Ia-37 | 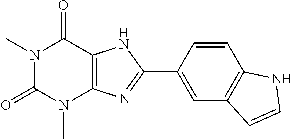 [SMILES: Cn1c(=O)c2[nH]c(-c3ccc4[nH]ccc4c3)nc2n(C)c1=O] |
| Ia-38 | 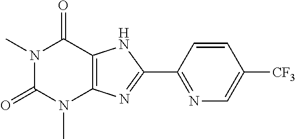 [SMILES: Cn1c(=O)c2[nH]c(-c3ccc(C(F)(F)F)cn3)nc2n(C)c1=O] |
| Ia-39 | 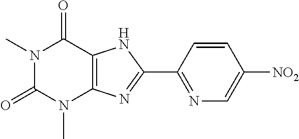 [SMILES: Cn1c(=O)c2[nH]c(-c3ccc([N+](=O)[O-])cn3)nc2n(C)c1=O] |
| Ia-40 | 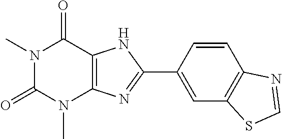 [SMILES: Cn1c(=O)c2[nH]c(-c3ccc4ncsc4c3)nc2n(C)c1=O] |
| Ia-42 | 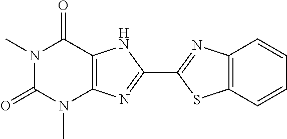 [SMILES: Cn1c(=O)c2[nH]c(-c3nc4ccccc4s3)nc2n(C)c1=O] |
| Ia-44 | 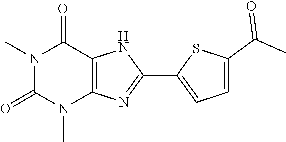 [SMILES: CC(=O)c1ccc(-c2nc3c([nH]2)c(=O)n(C)c(=O)n3C)s1] |
| Ia-45 | 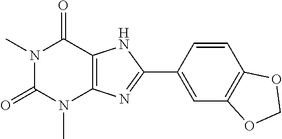 [SMILES: Cn1c(=O)c2[nH]c(-c3ccc4c(c3)OCO4)nc2n(C)c1=O] |
| Ia-47 | 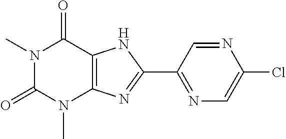 [SMILES: Cn1c(=O)c2[nH]c(-c3cnc(Cl)cn3)nc2n(C)c1=O] |
| Ia-50 | 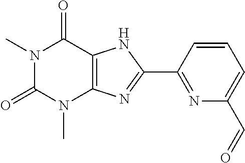 [SMILES: Cn1c(=O)c2[nH]c(-c3cccc(C=O)n3)nc2n(C)c1=O] |
| Ia-51 | 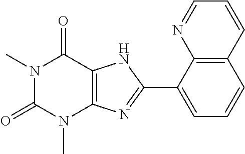 [SMILES: Cn1c(=O)c2[nH]c(-c3cccc4cccnc34)nc2n(C)c1=O] |
| Ia-57 | 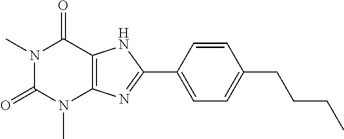 [SMILES: CCCCc1ccc(-c2nc3c([nH]2)c(=O)n(C)c(=O)n3C)cc1] |
| Ia-68 | 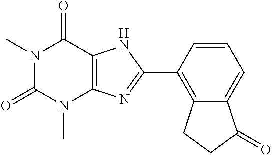 [SMILES: Cn1c(=O)c2[nH]c(-c3cccc4c3CCC4=O)nc2n(C)c1=O] |
| Ia-77 | 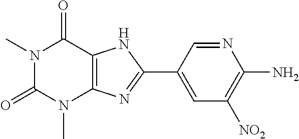 [SMILES: Cn1c(=O)c2[nH]c(-c3cnc(N)c([N+](=O)[O-])c3)nc2n(C)c1=O] |
| Ia-78 | 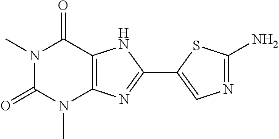 [SMILES: Cn1c(=O)c2[nH]c(-c3cnc(N)s3)nc2n(C)c1=O] |
| Ia-79 | 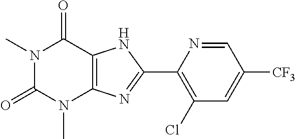 [SMILES: Cn1c(=O)c2[nH]c(-c3ncc(C(F)(F)F)cc3Cl)nc2n(C)c1=O] |
| Ia-80 | 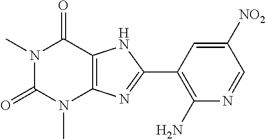 [SMILES: Cn1c(=O)c2[nH]c(-c3cc([N+](=O)[O-])cnc3N)nc2n(C)c1=O] |
| Ia-81 | 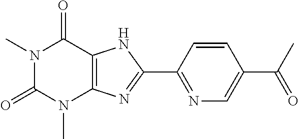 [SMILES: CC(=O)c1ccc(-c2nc3c([nH]2)c(=O)n(C)c(=O)n3C)nc1] |
| Ia-82 | 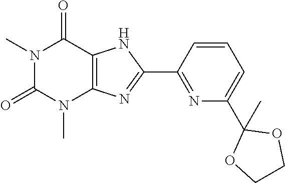 [SMILES: Cn1c(=O)c2[nH]c(-c3cccc(C4(C)OCCO4)n3)nc2n(C)c1=O] |

or a pharmaceutically acceptable salt, solvate and/or prodrug thereof.

In some embodiments, the present application also includes the following novel compounds

| Compound No. | Structure |
|---|---|
| Ia-1 | 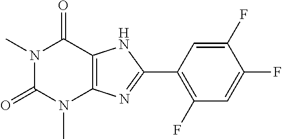 [SMILES: Cn1c(=O)c2[nH]c(-c3cc(F)c(F)cc3F)nc2n(C)c1=O] |
| Ia-2 | 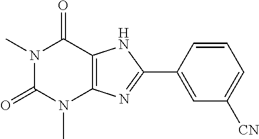 [SMILES: Cn1c(=O)c2[nH]c(-c3cccc(C#N)c3)nc2n(C)c1=O] |
| Ia-3 | 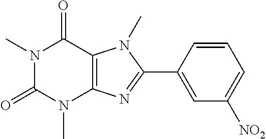 [SMILES: Cn1c(=O)c2c(nc(-c3cccc([N+](=O)[O-])c3)n2C)n(C)c1=O] |
| Ia-4 | 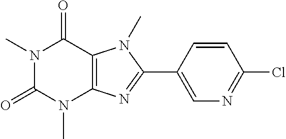 [SMILES: Cn1c(=O)c2c(nc(-c3ccc(Cl)nc3)n2C)n(C)c1=O] |
| Ia-5 | 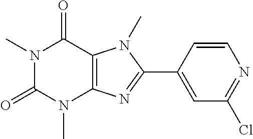 [SMILES: Cn1c(=O)c2c(nc(-c3ccnc(Cl)c3)n2C)n(C)c1=O] |
| Ia-6 | 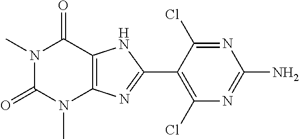 [SMILES: Cn1c(=O)c2[nH]c(-c3c(Cl)nc(N)nc3Cl)nc2n(C)c1=O] |
| Ia-7 | 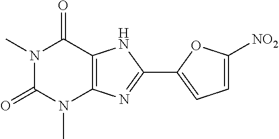 [SMILES: Cn1c(=O)c2[nH]c(-c3ccc([N+](=O)[O-])o3)nc2n(C)c1=O] |
| Ia-8 | 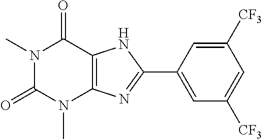 [SMILES: Cn1c(=O)c2[nH]c(-c3cc(C(F)(F)F)cc(C(F)(F)F)c3)nc2n(C)c1=O] |
| Ia-9 | 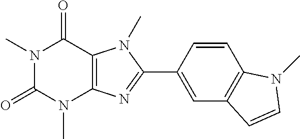 |
| Ia-10 | 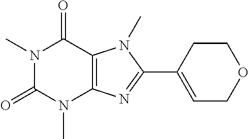 [SMILES: Cn1c(=O)c2c(nc(C3=CCOCC3)n2C)n(C)c1=O] |
| Ia-11 | 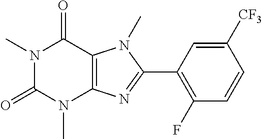 [SMILES: Cn1c(=O)c2c(nc(-c3cc(C(F)(F)F)ccc3F)n2C)n(C)c1=O] |
| Ia-12 | 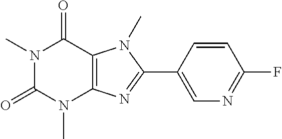 [SMILES: Cn1c(=O)c2c(nc(-c3ccc(F)nc3)n2C)n(C)c1=O] |
| Ia-13 | 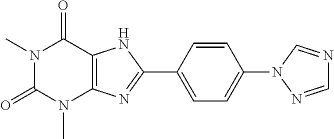 [SMILES: Cn1c(=O)c2[nH]c(-c3ccc(-n4cncn4)cc3)nc2n(C)c1=O] |
| Ia-14 | 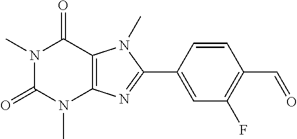 [SMILES: Cn1c(=O)c2c(nc(-c3ccc(C=O)c(F)c3)n2C)n(C)c1=O] |
| Ia-15 | 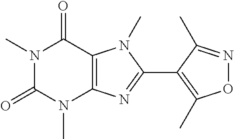 [SMILES: Cc1noc(C)c1-c1nc2c(c(=O)n(C)c(=O)n2C)n1C] |
| Ia-16 | 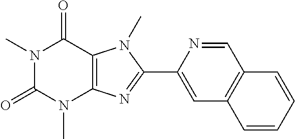 [SMILES: Cn1c(=O)c2c(nc(-c3cc4ccccc4cn3)n2C)n(C)c1=O] |
| Ia-17 | 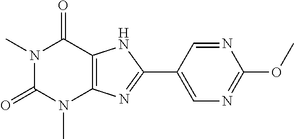 |
| Ia-18 | 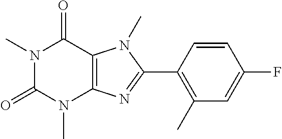 [SMILES: Cc1cc(F)ccc1-c1nc2c(c(=O)n(C)c(=O)n2C)n1C] |
| Ia-19 | 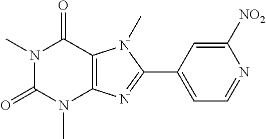 [SMILES: Cn1c(=O)c2c(nc(-c3ccnc([N+](=O)[O-])c3)n2C)n(C)c1=O] |
| Ia-20 | 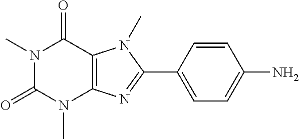 [SMILES: Cn1c(=O)c2c(nc(-c3ccc(N)cc3)n2C)n(C)c1=O] |
| Ia-21 | 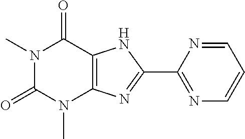 [SMILES: Cn1c(=O)c2[nH]c(-c3ncccn3)nc2n(C)c1=O] |
| Ia-22 | 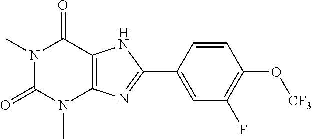 [SMILES: Cn1c(=O)c2[nH]c(-c3ccc(OC(F)(F)F)c(F)c3)nc2n(C)c1=O] |
| Ia-23 | 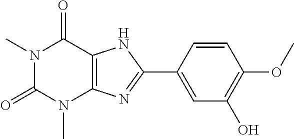 |
| Ia-24 | 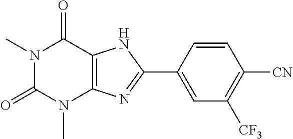 [SMILES: Cn1c(=O)c2[nH]c(-c3ccc(C#N)c(C(F)(F)F)c3)nc2n(C)c1=O] |
| Ia-25 | 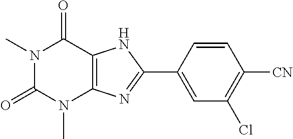 [SMILES: Cn1c(=O)c2[nH]c(-c3ccc(C#N)c(Cl)c3)nc2n(C)c1=O] |
| Ia-26 | 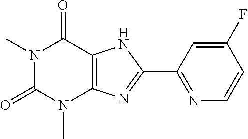 [SMILES: Cn1c(=O)c2[nH]c(-c3cc(F)ccn3)nc2n(C)c1=O] |
| Ia-27 | 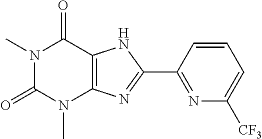 [SMILES: Cn1c(=O)c2[nH]c(-c3cccc(C(F)(F)F)n3)nc2n(C)c1=O] |
| Ia-28 | 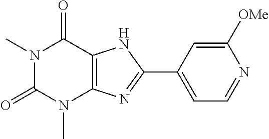 [SMILES: COc1cc(-c2nc3c([nH]2)c(=O)n(C)c(=O)n3C)ccn1] |
| Ia-29 | 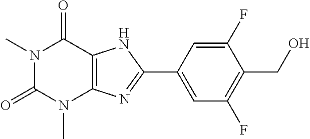 [SMILES: Cn1c(=O)c2[nH]c(-c3cc(F)c(CO)c(F)c3)nc2n(C)c1=O] |
| Ia-31 | 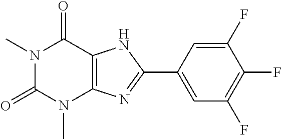 [SMILES: Cn1c(=O)c2[nH]c(-c3cc(F)c(F)c(F)c3)nc2n(C)c1=O] |
| Ia-32 | 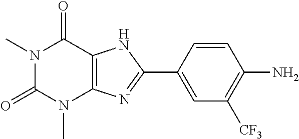 [SMILES: Cn1c(=O)c2[nH]c(-c3ccc(N)c(C(F)(F)F)c3)nc2n(C)c1=O] |
| Ia-33 | 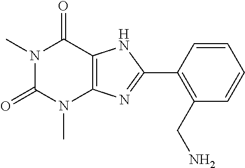 [SMILES: Cn1c(=O)c2[nH]c(-c3ccccc3CN)nc2n(C)c1=O] |
| Ia-34 | 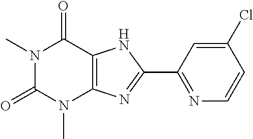 [SMILES: Cn1c(=O)c2[nH]c(-c3cc(Cl)ccn3)nc2n(C)c1=O] |
| Ia-35 | 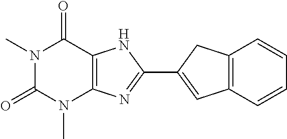 [SMILES: Cn1c(=O)c2[nH]c(C3=Cc4ccccc4C3)nc2n(C)c1=O] |
| Ia-36 | 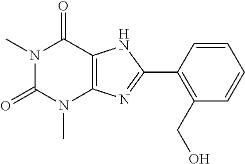 [SMILES: Cn1c(=O)c2[nH]c(-c3ccccc3CO)nc2n(C)c1=O] |
| Ia-37 | 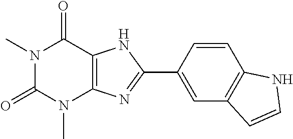 [SMILES: Cn1c(=O)c2[nH]c(-c3ccc4[nH]ccc4c3)nc2n(C)c1=O] |
| Ia-38 | 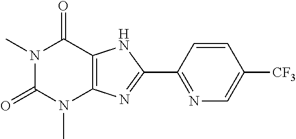 [SMILES: Cn1c(=O)c2[nH]c(-c3ccc(C(F)(F)F)cn3)nc2n(C)c1=O] |
| Ia-39 | 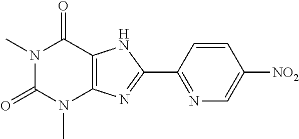 [SMILES: Cn1c(=O)c2[nH]c(-c3ccc([N+](=O)[O-])cn3)nc2n(C)c1=O] |
| Ia-40 | 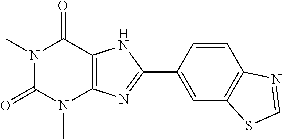 [SMILES: Cn1c(=O)c2[nH]c(-c3ccc4ncsc4c3)nc2n(C)c1=O] |
| Ia-41 | 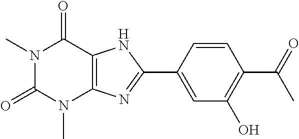 [SMILES: CC(=O)c1ccc(-c2nc3c([nH]2)c(=O)n(C)c(=O)n3C)cc1O] |
| Ia-42 | 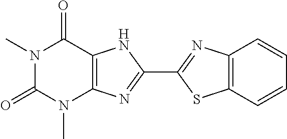 [SMILES: Cn1c(=O)c2[nH]c(-c3nc4ccccc4s3)nc2n(C)c1=O] |
| Ia-43 | 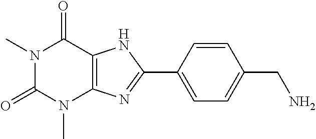 [SMILES: Cn1c(=O)c2[nH]c(-c3ccc(CN)cc3)nc2n(C)c1=O] |
| Ia-44 | 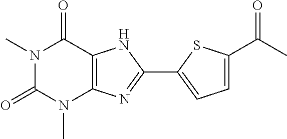 [SMILES: CC(=O)c1ccc(-c2nc3c([nH]2)c(=O)n(C)c(=O)n3C)s1] |
| Ia-45 | 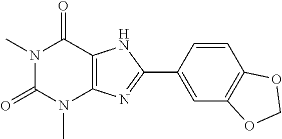 [SMILES: Cn1c(=O)c2[nH]c(-c3ccc4c(c3)OCO4)nc2n(C)c1=O] |
| Ia-47 | 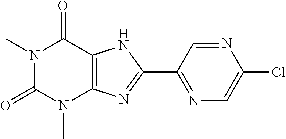 [SMILES: Cn1c(=O)c2[nH]c(-c3cnc(Cl)cn3)nc2n(C)c1=O] |
| Ia-50 | 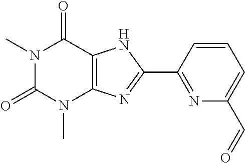 [SMILES: Cn1c(=O)c2[nH]c(-c3cccc(C=O)n3)nc2n(C)c1=O] |
| Ia-51 | 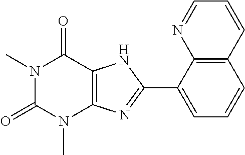 [SMILES: Cn1c(=O)c2[nH]c(-c3cccc4cccnc34)nc2n(C)c1=O] |
| Ia-53 | 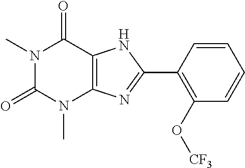 [SMILES: Cn1c(=O)c2[nH]c(-c3ccccc3OC(F)(F)F)nc2n(C)c1=O] |
| Ia-55 | 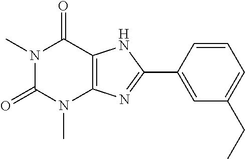 [SMILES: CCc1cccc(-c2nc3c([nH]2)c(=O)n(C)c(=O)n3C)c1] |
| Ia-56 | 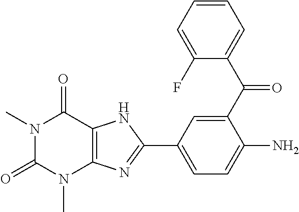 [SMILES: Cn1c(=O)c2[nH]c(-c3ccc(N)c(C(=O)c4ccccc4F)c3)nc2n(C)c1=O] |
| Ia-57 | 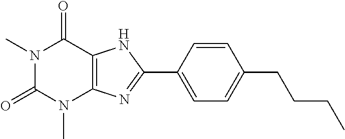 [SMILES: CCCCc1ccc(-c2nc3c([nH]2)c(=O)n(C)c(=O)n3C)cc1] |
| Ia-58 | 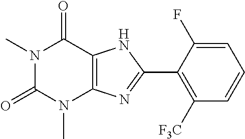 [SMILES: Cn1c(=O)c2[nH]c(-c3c(F)cccc3C(F)(F)F)nc2n(C)c1=O] |
| Ia-59 | 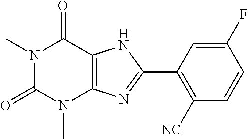 [SMILES: Cn1c(=O)c2[nH]c(-c3cc(F)ccc3C#N)nc2n(C)c1=O] |
| Ia-60 | 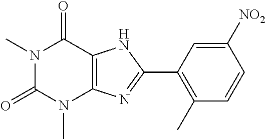 [SMILES: Cc1ccc([N+](=O)[O-])cc1-c1nc2c([nH]1)c(=O)n(C)c(=O)n2C] |
| Ia-61 | 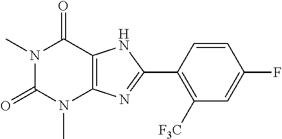 [SMILES: Cn1c(=O)c2[nH]c(-c3ccc(F)cc3C(F)(F)F)nc2n(C)c1=O] |
| Ia-62 | 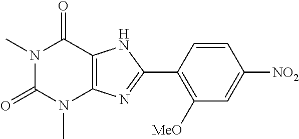 [SMILES: COc1cc([N+](=O)[O-])ccc1-c1nc2c([nH]1)c(=O)n(C)c(=O)n2C] |
| Ia-63 | 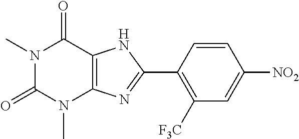 [SMILES: Cn1c(=O)c2[nH]c(-c3ccc([N+](=O)[O-])cc3C(F)(F)F)nc2n(C)c1=O] |
| Ia-64 | 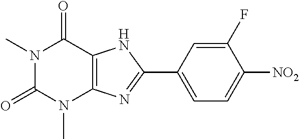 [SMILES: Cn1c(=O)c2[nH]c(-c3ccc([N+](=O)[O-])c(F)c3)nc2n(C)c1=O] |
| Ia-65 | 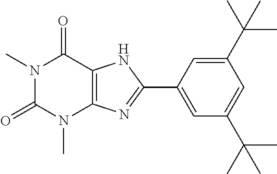 [SMILES: Cn1c(=O)c2[nH]c(-c3cc(C(C)(C)C)cc(C(C)(C)C)c3)nc2n(C)c1=O] |
| Ia-66 | 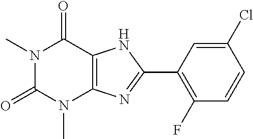 [SMILES: Cn1c(=O)c2[nH]c(-c3cc(Cl)ccc3F)nc2n(C)c1=O] |
| Ia-67 | 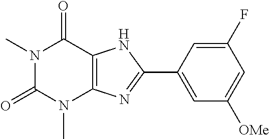 [SMILES: COc1cc(F)cc(-c2nc3c([nH]2)c(=O)n(C)c(=O)n3C)c1] |
| Ia-68 | 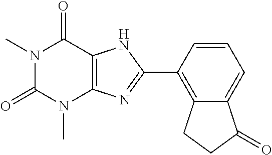 [SMILES: Cn1c(=O)c2[nH]c(-c3cccc4c3CCC4=O)nc2n(C)c1=O] |
| Ia-69 | 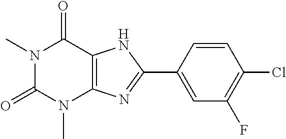 [SMILES: Cn1c(=O)c2[nH]c(-c3ccc(Cl)c(F)c3)nc2n(C)c1=O] |
| Ia-70 | 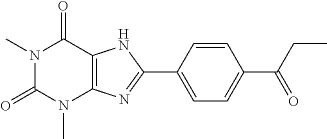 [SMILES: CCC(=O)c1ccc(-c2nc3c([nH]2)c(=O)n(C)c(=O)n3C)cc1] |
| Ia-71 | 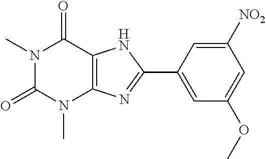 [SMILES: COc1cc(-c2nc3c([nH]2)c(=O)n(C)c(=O)n3C)cc([N+](=O)[O-])c1] |
| Ia-72 | 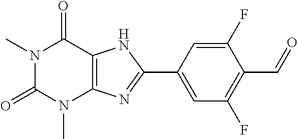 [SMILES: Cn1c(=O)c2[nH]c(-c3cc(F)c(C=O)c(F)c3)nc2n(C)c1=O] |
| Ia-73 | 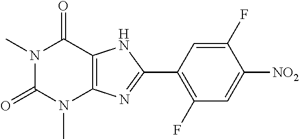 [SMILES: Cn1c(=O)c2[nH]c(-c3cc(F)c([N+](=O)[O-])cc3F)nc2n(C)c1=O] |
| Ia-74 | 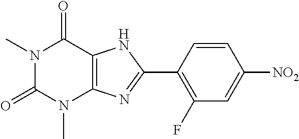 [SMILES: Cn1c(=O)c2[nH]c(-c3ccc([N+](=O)[O-])cc3F)nc2n(C)c1=O] |
| Ia-75 | 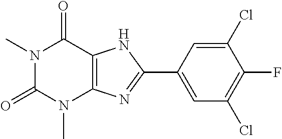 [SMILES: Cn1c(=O)c2[nH]c(-c3cc(Cl)c(F)c(Cl)c3)nc2n(C)c1=O] |
| Ia-76 | 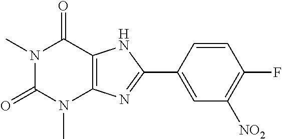 [SMILES: Cn1c(=O)c2[nH]c(-c3ccc(F)c([N+](=O)[O-])c3)nc2n(C)c1=O] |
| Ia-77 | 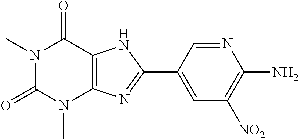 [SMILES: Cn1c(=O)c2[nH]c(-c3cnc(N)c([N+](=O)[O-])c3)nc2n(C)c1=O] |
| Ia-78 | 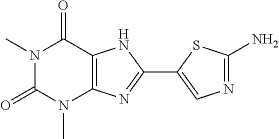 [SMILES: Cn1c(=O)c2[nH]c(-c3cnc(N)s3)nc2n(C)c1=O] |
| Ia-79 | 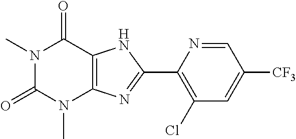 [SMILES: Cn1c(=O)c2[nH]c(-c3ncc(C(F)(F)F)cc3Cl)nc2n(C)c1=O] |
| Ia-80 | 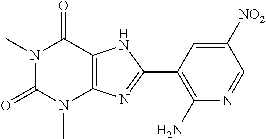 [SMILES: Cn1c(=O)c2[nH]c(-c3cc([N+](=O)[O-])cnc3N)nc2n(C)c1=O] |
| Ia-81 | 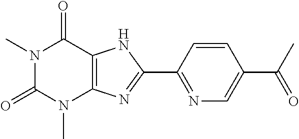 [SMILES: CC(=O)c1ccc(-c2nc3c([nH]2)c(=O)n(C)c(=O)n3C)nc1] |
| Ia-82 | 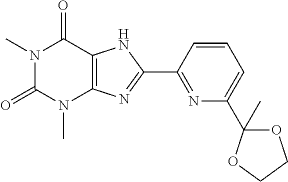 [SMILES: Cn1c(=O)c2[nH]c(-c3cccc(C4(C)OCCO4)n3)nc2n(C)c1=O] |
| Ia-83 | 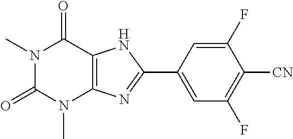 [SMILES: Cn1c(=O)c2[nH]c(-c3cc(F)c(C#N)c(F)c3)nc2n(C)c1=O] |
| Ia-84 | 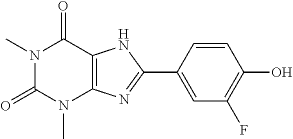 [SMILES: Cn1c(=O)c2[nH]c(-c3ccc(O)c(F)c3)nc2n(C)c1=O] |
| Ia-85 | 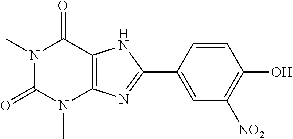 [SMILES: Cn1c(=O)c2[nH]c(-c3ccc(O)c([N+](=O)[O-])c3)nc2n(C)c1=O] |

or a pharmaceutically acceptable salt, solvate and/or prodrug thereof.

In some embodiments, the present application also includes the novel compounds listed in Table 1.

TABLE 1

| Compound No. | Structure |
|---|---|
| Ia-1 | 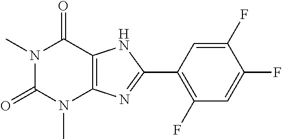 [SMILES: Cn1c(=O)c2[nH]c(-c3cc(F)c(F)cc3F)nc2n(C)c1=O] |
| Ia-2 | 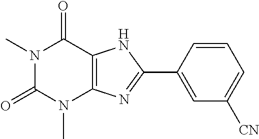 [SMILES: Cn1c(=O)c2[nH]c(-c3cccc(C#N)c3)nc2n(C)c1=O] |
| Ia-3 | 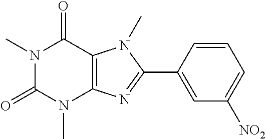 [SMILES: Cn1c(=O)c2c(nc(-c3cccc([N+](=O)[O-])c3)n2C)n(C)c1=O] |
| Ia-4 | 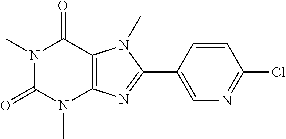 [SMILES: Cn1c(=O)c2c(nc(-c3ccc(Cl)nc3)n2C)n(C)c1=O] |
| Ia-5 | 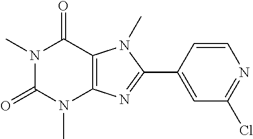 [SMILES: Cn1c(=O)c2c(nc(-c3ccnc(Cl)c3)n2C)n(C)c1=O] |
| Ia-6 | 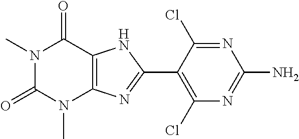 [SMILES: Cn1c(=O)c2[nH]c(-c3c(Cl)nc(N)nc3Cl)nc2n(C)c1=O] |
| Ia-7 | 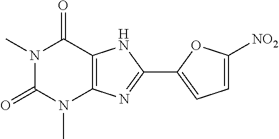 [SMILES: Cn1c(=O)c2[nH]c(-c3ccc([N+](=O)[O-])o3)nc2n(C)c1=O] |
| Ia-8 | 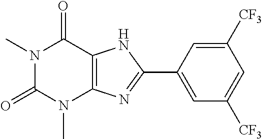 [SMILES: Cn1c(=O)c2[nH]c(-c3cc(C(F)(F)F)cc(C(F)(F)F)c3)nc2n(C)c1=O] |
| Ia-9 | 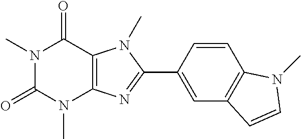 |
| Ia-10 | 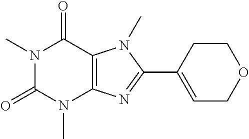 [SMILES: Cn1c(=O)c2c(nc(C3=CCOCC3)n2C)n(C)c1=O] |
| Ia-11 | 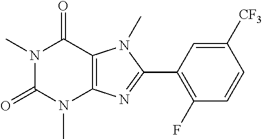 [SMILES: Cn1c(=O)c2c(nc(-c3cc(C(F)(F)F)ccc3F)n2C)n(C)c1=O] |
| Ia-12 | 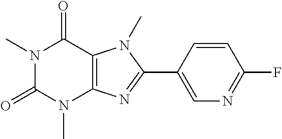 [SMILES: Cn1c(=O)c2c(nc(-c3ccc(F)nc3)n2C)n(C)c1=O] |
| Ia-13 | 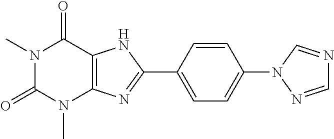 [SMILES: Cn1c(=O)c2[nH]c(-c3ccc(-n4cncn4)cc3)nc2n(C)c1=O] |
| Ia-14 | 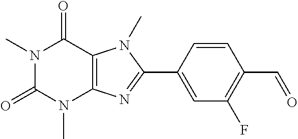 [SMILES: Cn1c(=O)c2c(nc(-c3ccc(C=O)c(F)c3)n2C)n(C)c1=O] |
| Ia-15 | 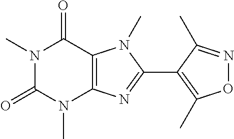 [SMILES: Cc1noc(C)c1-c1nc2c(c(=O)n(C)c(=O)n2C)n1C] |
| Ia-16 | 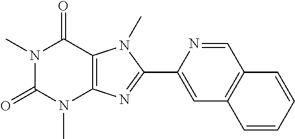 [SMILES: Cn1c(=O)c2c(nc(-c3cc4ccccc4cn3)n2C)n(C)c1=O] |
| Ia-17 | 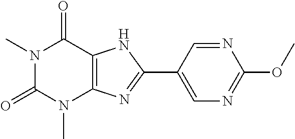 |
| Ia-18 | 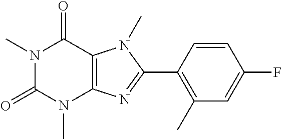 [SMILES: Cc1cc(F)ccc1-c1nc2c(c(=O)n(C)c(=O)n2C)n1C] |
| Ia-19 | 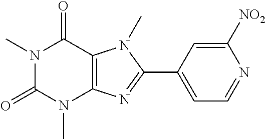 [SMILES: Cn1c(=O)c2c(nc(-c3ccnc([N+](=O)[O-])c3)n2C)n(C)c1=O] |
| Ia-20 | 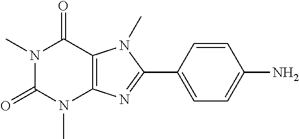 [SMILES: Cn1c(=O)c2c(nc(-c3ccc(N)cc3)n2C)n(C)c1=O] |
| Ia-21 | 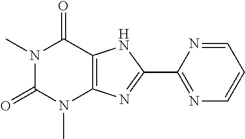 [SMILES: Cn1c(=O)c2[nH]c(-c3ncccn3)nc2n(C)c1=O] |
| Ia-22 | 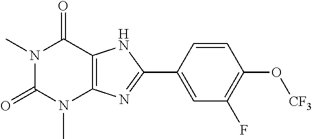 [SMILES: Cn1c(=O)c2[nH]c(-c3ccc(OC(F)(F)F)c(F)c3)nc2n(C)c1=O] |
| Ia-23 | 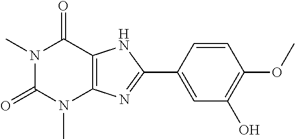 |
| Ia-24 | 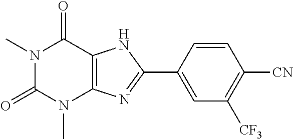 [SMILES: Cn1c(=O)c2[nH]c(-c3ccc(C#N)c(C(F)(F)F)c3)nc2n(C)c1=O] |
| Ia-25 | 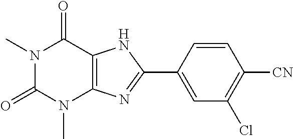 [SMILES: Cn1c(=O)c2[nH]c(-c3ccc(C#N)c(Cl)c3)nc2n(C)c1=O] |
| Ia-26 | 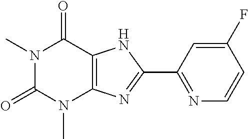 [SMILES: Cn1c(=O)c2[nH]c(-c3cc(F)ccn3)nc2n(C)c1=O] |
| Ia-27 | 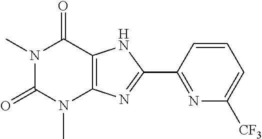 [SMILES: Cn1c(=O)c2[nH]c(-c3cccc(C(F)(F)F)n3)nc2n(C)c1=O] |
| Ia-28 | 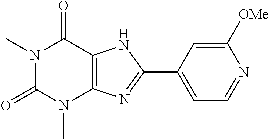 [SMILES: COc1cc(-c2nc3c([nH]2)c(=O)n(C)c(=O)n3C)ccn1] |
| Ia-29 | 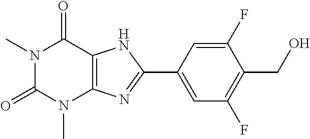 [SMILES: Cn1c(=O)c2[nH]c(-c3cc(F)c(CO)c(F)c3)nc2n(C)c1=O] |
| Ia-31 | 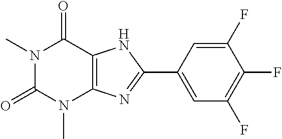 [SMILES: Cn1c(=O)c2[nH]c(-c3cc(F)c(F)c(F)c3)nc2n(C)c1=O] |
| Ia-32 | 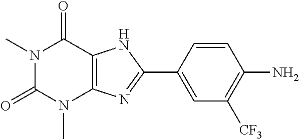 [SMILES: Cn1c(=O)c2[nH]c(-c3ccc(N)c(C(F)(F)F)c3)nc2n(C)c1=O] |
| Ia-33 | 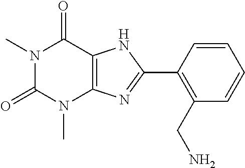 [SMILES: Cn1c(=O)c2[nH]c(-c3ccccc3CN)nc2n(C)c1=O] |
| Ia-34 | 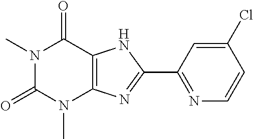 [SMILES: Cn1c(=O)c2[nH]c(-c3cc(Cl)ccn3)nc2n(C)c1=O] |
| Ia-35 | 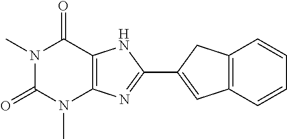 [SMILES: Cn1c(=O)c2[nH]c(C3=Cc4ccccc4C3)nc2n(C)c1=O] |
| Ia-36 | 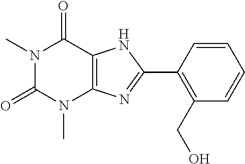 [SMILES: Cn1c(=O)c2[nH]c(-c3ccccc3CO)nc2n(C)c1=O] |
| Ia-37 | 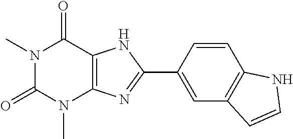 [SMILES: Cn1c(=O)c2[nH]c(-c3ccc4[nH]ccc4c3)nc2n(C)c1=O] |
| Ia-38 | 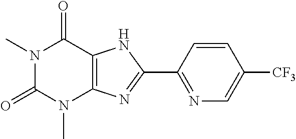 [SMILES: Cn1c(=O)c2[nH]c(-c3ccc(C(F)(F)F)cn3)nc2n(C)c1=O] |
| Ia-39 | 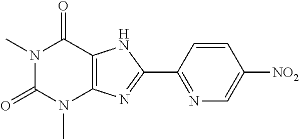 [SMILES: Cn1c(=O)c2[nH]c(-c3ccc([N+](=O)[O-])cn3)nc2n(C)c1=O] |
| Ia-40 | 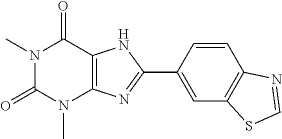 [SMILES: Cn1c(=O)c2[nH]c(-c3ccc4ncsc4c3)nc2n(C)c1=O] |
| Ia-41 | 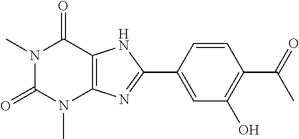 [SMILES: CC(=O)c1ccc(-c2nc3c([nH]2)c(=O)n(C)c(=O)n3C)cc1O] |
| Ia-42 | 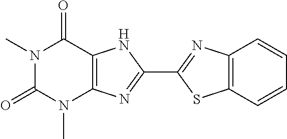 [SMILES: Cn1c(=O)c2[nH]c(-c3nc4ccccc4s3)nc2n(C)c1=O] |
| Ia-43 | 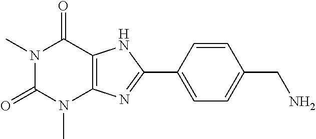 [SMILES: Cn1c(=O)c2[nH]c(-c3ccc(CN)cc3)nc2n(C)c1=O] |
| Ia-44 | 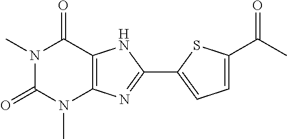 [SMILES: CC(=O)c1ccc(-c2nc3c([nH]2)c(=O)n(C)c(=O)n3C)s1] |
| Ia-45 | 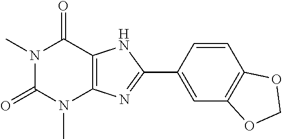 [SMILES: Cn1c(=O)c2[nH]c(-c3ccc4c(c3)OCO4)nc2n(C)c1=O] |
| Ia-47 | 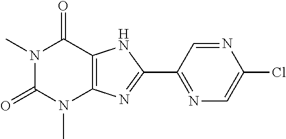 [SMILES: Cn1c(=O)c2[nH]c(-c3cnc(Cl)cn3)nc2n(C)c1=O] |
| Ia-50 | 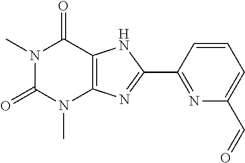 [SMILES: Cn1c(=O)c2[nH]c(-c3cccc(C=O)n3)nc2n(C)c1=O] |
| Ia-51 | 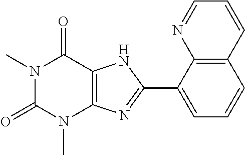 [SMILES: Cn1c(=O)c2[nH]c(-c3cccc4cccnc34)nc2n(C)c1=O] |
| Ia-53 | 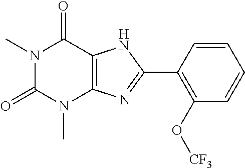 [SMILES: Cn1c(=O)c2[nH]c(-c3ccccc3OC(F)(F)F)nc2n(C)c1=O] |
| Ia-55 | 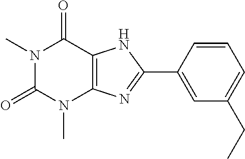 [SMILES: CCc1cccc(-c2nc3c([nH]2)c(=O)n(C)c(=O)n3C)c1] |
| Ia-56 | 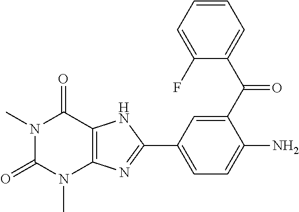 [SMILES: Cn1c(=O)c2[nH]c(-c3ccc(N)c(C(=O)c4ccccc4F)c3)nc2n(C)c1=O] |
| Ia-57 | 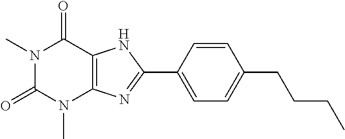 [SMILES: CCCCc1ccc(-c2nc3c([nH]2)c(=O)n(C)c(=O)n3C)cc1] |
| Ia-58 | 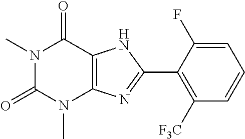 [SMILES: Cn1c(=O)c2[nH]c(-c3c(F)cccc3C(F)(F)F)nc2n(C)c1=O] |
| Ia-59 | 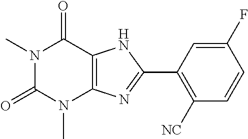 [SMILES: Cn1c(=O)c2[nH]c(-c3cc(F)ccc3C#N)nc2n(C)c1=O] |
| Ia-60 | 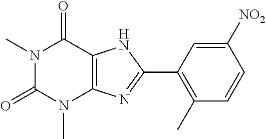 [SMILES: Cc1ccc([N+](=O)[O-])cc1-c1nc2c([nH]1)c(=O)n(C)c(=O)n2C] |
| Ia-61 | 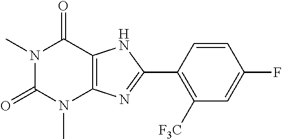 [SMILES: Cn1c(=O)c2[nH]c(-c3ccc(F)cc3C(F)(F)F)nc2n(C)c1=O] |
| Ia-62 | 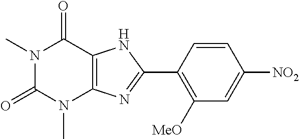 [SMILES: COc1cc([N+](=O)[O-])ccc1-c1nc2c([nH]1)c(=O)n(C)c(=O)n2C] |
| Ia-63 | 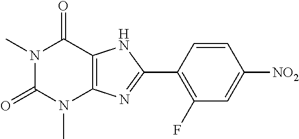 [SMILES: Cn1c(=O)c2[nH]c(-c3ccc([N+](=O)[O-])cc3F)nc2n(C)c1=O] |
| Ia-64 | 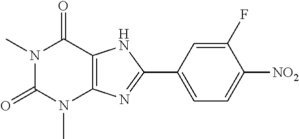 [SMILES: Cn1c(=O)c2[nH]c(-c3ccc([N+](=O)[O-])c(F)c3)nc2n(C)c1=O] |
| Ia-65 | 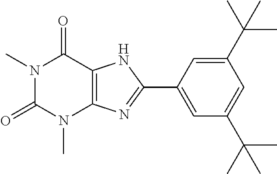 [SMILES: Cn1c(=O)c2[nH]c(-c3cc(C(C)(C)C)cc(C(C)(C)C)c3)nc2n(C)c1=O] |
| Ia-66 | 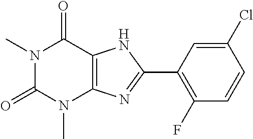 [SMILES: Cn1c(=O)c2[nH]c(-c3cc(Cl)ccc3F)nc2n(C)c1=O] |
| Ia-67 | 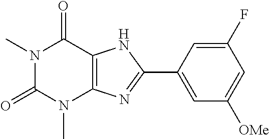 [SMILES: COc1cc(F)cc(-c2nc3c([nH]2)c(=O)n(C)c(=O)n3C)c1] |
| Ia-68 | 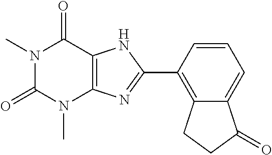 [SMILES: Cn1c(=O)c2[nH]c(-c3cccc4c3CCC4=O)nc2n(C)c1=O] |
| Ia-69 | 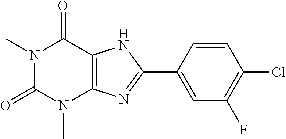 [SMILES: Cn1c(=O)c2[nH]c(-c3ccc(Cl)c(F)c3)nc2n(C)c1=O] |
| Ia-70 | 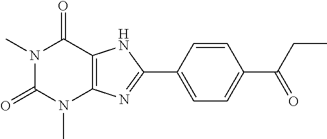 [SMILES: CCC(=O)c1ccc(-c2nc3c([nH]2)c(=O)n(C)c(=O)n3C)cc1] |
| Ia-71 | 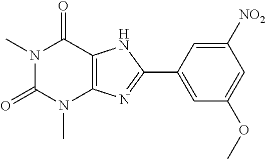 [SMILES: COc1cc(-c2nc3c([nH]2)c(=O)n(C)c(=O)n3C)cc([N+](=O)[O-])c1] |
| Ia-72 | 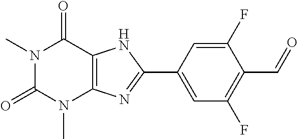 [SMILES: Cn1c(=O)c2[nH]c(-c3cc(F)c(C=O)c(F)c3)nc2n(C)c1=O] |
| Ia-73 | 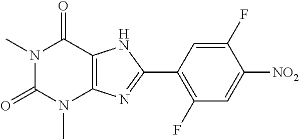 [SMILES: Cn1c(=O)c2[nH]c(-c3cc(F)c([N+](=O)[O-])cc3F)nc2n(C)c1=O] |
| Ia-74 | 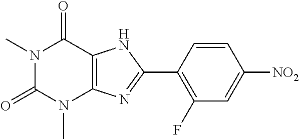 [SMILES: Cn1c(=O)c2[nH]c(-c3ccc([N+](=O)[O-])cc3F)nc2n(C)c1=O] |
| Ia-75 | 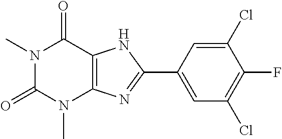 [SMILES: Cn1c(=O)c2[nH]c(-c3cc(Cl)c(F)c(Cl)c3)nc2n(C)c1=O] |
| Ia-76 | 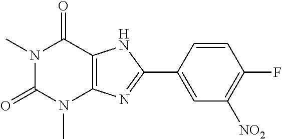 [SMILES: Cn1c(=O)c2[nH]c(-c3ccc(F)c([N+](=O)[O-])c3)nc2n(C)c1=O] |
| Ia-77 | 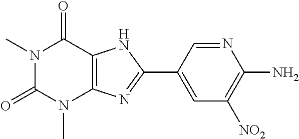 [SMILES: Cn1c(=O)c2[nH]c(-c3cnc(N)c([N+](=O)[O-])c3)nc2n(C)c1=O] |
| Ia-78 | 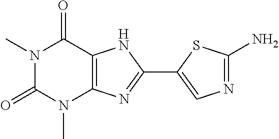 [SMILES: Cn1c(=O)c2[nH]c(-c3cnc(N)s3)nc2n(C)c1=O] |
| Ia-79 | 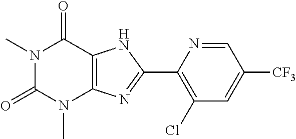 [SMILES: Cn1c(=O)c2[nH]c(-c3ncc(C(F)(F)F)cc3Cl)nc2n(C)c1=O] |
| Ia-80 | 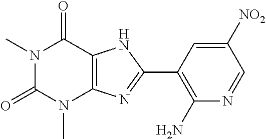 [SMILES: Cn1c(=O)c2[nH]c(-c3cc([N+](=O)[O-])cnc3N)nc2n(C)c1=O] |
| Ia-81 | 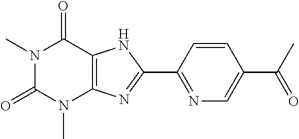 [SMILES: CC(=O)c1ccc(-c2nc3c([nH]2)c(=O)n(C)c(=O)n3C)nc1] |
| Ia-82 | 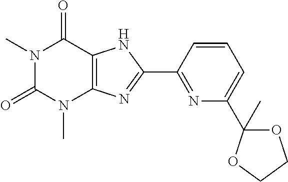 [SMILES: Cn1c(=O)c2[nH]c(-c3cccc(C4(C)OCCO4)n3)nc2n(C)c1=O] |
| Ia-83 | 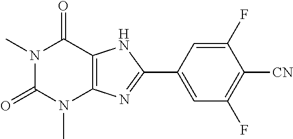 [SMILES: Cn1c(=O)c2[nH]c(-c3cc(F)c(C#N)c(F)c3)nc2n(C)c1=O] |
| Ia-84 | 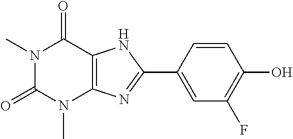 [SMILES: Cn1c(=O)c2[nH]c(-c3ccc(O)c(F)c3)nc2n(C)c1=O] |
| Ia-85 | 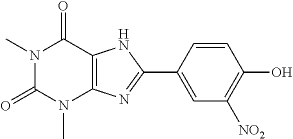 [SMILES: Cn1c(=O)c2[nH]c(-c3ccc(O)c([N+](=O)[O-])c3)nc2n(C)c1=O] |
| Ia-87 | 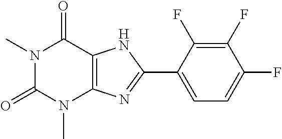 [SMILES: Cn1c(=O)c2[nH]c(-c3ccc(F)c(F)c3F)nc2n(C)c1=O] |
| Ia-88 | 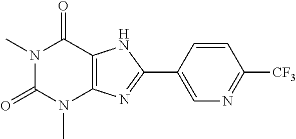 [SMILES: Cn1c(=O)c2[nH]c(-c3ccc(C(F)(F)F)nc3)nc2n(C)c1=O] |
| Ia-93 | 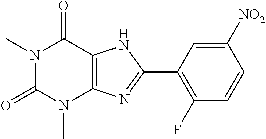 [SMILES: Cn1c(=O)c2[nH]c(-c3cc([N+](=O)[O-])ccc3F)nc2n(C)c1=O] |
| Ia-94 | 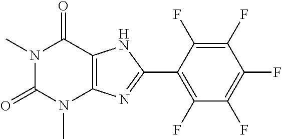 [SMILES: Cn1c(=O)c2[nH]c(-c3c(F)c(F)c(F)c(F)c3F)nc2n(C)c1=O] |
| Ia-97 | 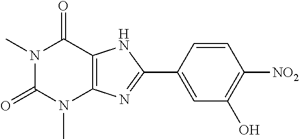 [SMILES: Cn1c(=O)c2[nH]c(-c3ccc([N+](=O)[O-])c(O)c3)nc2n(C)c1=O] |
| Ia-99 | 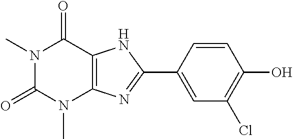 [SMILES: Cn1c(=O)c2[nH]c(-c3ccc(O)c(Cl)c3)nc2n(C)c1=O] |
| Ia-100 | 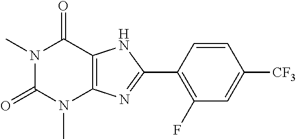 [SMILES: Cn1c(=O)c2[nH]c(-c3ccc(C(F)(F)F)cc3F)nc2n(C)c1=O] |
| Ia-102 | 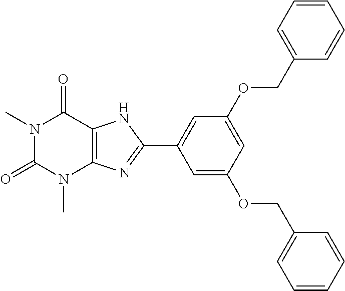 [SMILES: Cn1c(=O)c2[nH]c(-c3cc(OCc4ccccc4)cc(OCc4ccccc4)c3)nc2n(C)c1=O] |
| Ia-103 | 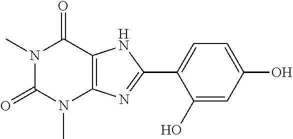 [SMILES: Cn1c(=O)c2[nH]c(-c3ccc(O)cc3O)nc2n(C)c1=O] |
| Ia-104 | 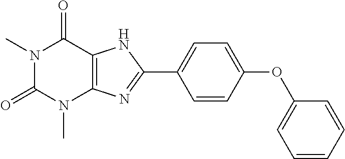 [SMILES: Cn1c(=O)c2[nH]c(-c3ccc(Oc4ccccc4)cc3)nc2n(C)c1=O] |
| Ia-105 | 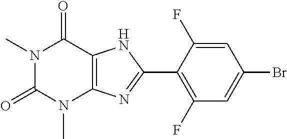 [SMILES: Cn1c(=O)c2[nH]c(-c3c(F)cc(Br)cc3F)nc2n(C)c1=O] |
| Ia-106 | 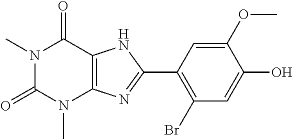 |
| Ia-108 | 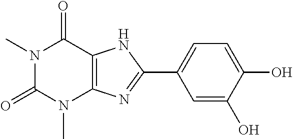 [SMILES: Cn1c(=O)c2[nH]c(-c3ccc(O)c(O)c3)nc2n(C)c1=O] |
| Ia-110 | 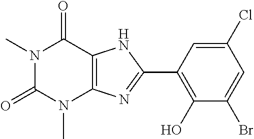 [SMILES: Cn1c(=O)c2[nH]c(-c3cc(Cl)cc(Br)c3O)nc2n(C)c1=O] |
| Ia-111 | 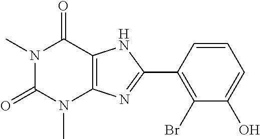 [SMILES: Cn1c(=O)c2[nH]c(-c3cccc(O)c3Br)nc2n(C)c1=O] |
| Ia-113 | 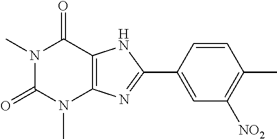 |
| Ia-115 | 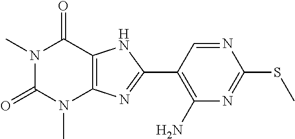 |
| Ia-116 | 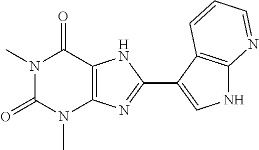 [SMILES: Cn1c(=O)c2[nH]c(-c3c[nH]c4ncccc34)nc2n(C)c1=O] |
| Ia-117 | 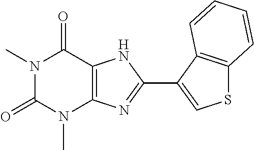 [SMILES: Cn1c(=O)c2[nH]c(-c3csc4ccccc34)nc2n(C)c1=O] |
| Ia-120 | 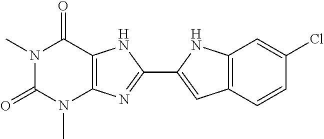 [SMILES: Cn1c(=O)c2[nH]c(-c3cc4ccc(Cl)cc4[nH]3)nc2n(C)c1=O] |
| Ia-121 | 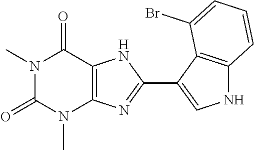 [SMILES: Cn1c(=O)c2[nH]c(-c3c[nH]c4cccc(Br)c34)nc2n(C)c1=O] |
| Ia-123 | 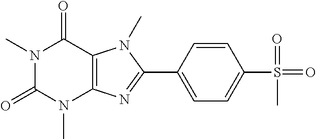 [SMILES: Cn1c(=O)c2c(nc(-c3ccc(S(C)(=O)=O)cc3)n2C)n(C)c1=O] |
| Ia-124 | 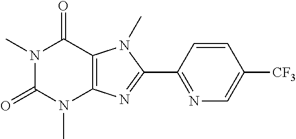 [SMILES: Cn1c(=O)c2c(nc(-c3ccc(C(F)(F)F)cn3)n2C)n(C)c1=O] |
| Ia-127 | 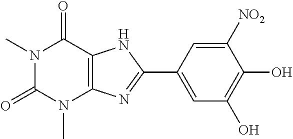 [SMILES: Cn1c(=O)c2[nH]c(-c3cc(O)c(O)c([N+](=O)[O-])c3)nc2n(C)c1=O] |
| Ia-129 | 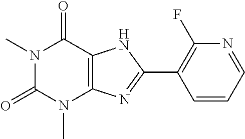 [SMILES: Cn1c(=O)c2[nH]c(-c3cccnc3F)nc2n(C)c1=O] |
| Ia-130 | 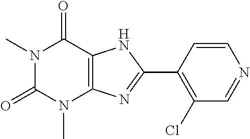 [SMILES: Cn1c(=O)c2[nH]c(-c3ccncc3Cl)nc2n(C)c1=O] |
| Ia-132 | 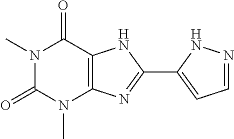 [SMILES: Cn1c(=O)c2[nH]c(-c3ccn[nH]3)nc2n(C)c1=O] |
| Ia-134 | 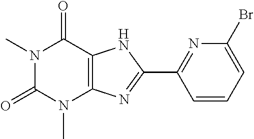 [SMILES: Cn1c(=O)c2[nH]c(-c3cccc(Br)n3)nc2n(C)c1=O] |
| Ia-135 | 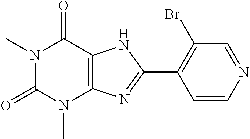 [SMILES: Cn1c(=O)c2[nH]c(-c3ccncc3Br)nc2n(C)c1=O] |
| Ia-136 | 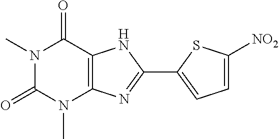 [SMILES: Cn1c(=O)c2[nH]c(-c3ccc([N+](=O)[O-])s3)nc2n(C)c1=O] |
| Ia-137 | 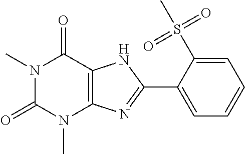 [SMILES: Cn1c(=O)c2[nH]c(-c3ccccc3S(C)(=O)=O)nc2n(C)c1=O] |
| Ia-138 | 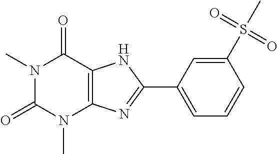 [SMILES: Cn1c(=O)c2[nH]c(-c3cccc(S(C)(=O)=O)c3)nc2n(C)c1=O] |
| Ia-139 | 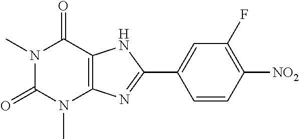 [SMILES: Cn1c(=O)c2[nH]c(-c3ccc([N+](=O)[O-])c(F)c3)nc2n(C)c1=O] |
| Ia-140 | 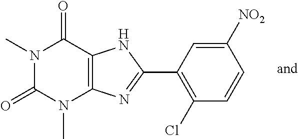 [SMILES: Cn1c(=O)c2[nH]c(-c3cc([N+](=O)[O-])ccc3Cl)nc2n(C)c1=O] |
| Ia-142 | 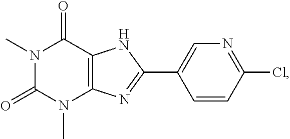 [SMILES: Cn1c(=O)c2[nH]c(-c3ccc(Cl)nc3)nc2n(C)c1=O] |

or a pharmaceutically acceptable salt, solvate and/or prodrug thereof.

In some embodiments of the present application, the compounds of the application, including compounds of Formula (I) and (Ia) may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

In some embodiments, the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like.

Prodrugs of the compounds of the present application may be, for example, conventional esters formed with available hydroxy, thiol, amino or carboxyl groups. Some common esters which have been utilized as prodrugs are phenyl esters, aliphatic (C1-C24) esters, acyloxymethyl esters, carbamates and amino acid esters.

### (b) Methods of Treatment

The present application also includes a method of treating a disease, disorder or condition treatable by blocking SREBP2 activation and/or PCSK9 gene expression, the method comprising administering an effect amount of one or more compounds of Formula (I), or a pharmaceutically acceptable salt, solvate and/or prodrug thereof, to a subject in need thereof:

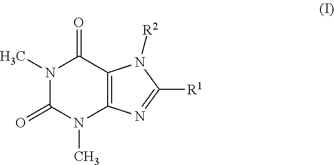

wherein
R¹ is selected from
   (i) phenyl substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆ alkyleneNH(C₁₋₆alkyl), C₁₋₆alkyleneN(C₁₋₆alkyl)(C₁₋₆alkyl), X¹—C₁₋₆alkyl, X¹—C₁₋₆ fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₅fluoroalkyl and NH₂;
   (ii) 9-10-membered bicyclic aryl wherein the second ring is phenyl or C₅₋₆cycloalkyl and the 9-10 membered bicyclic aryl is optionally substituted with one or more substituents independently selected from ═O, halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iii) monocyclic C₃₋₆cycloalkyl or C₅₋₆cycloalkenyl optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iv) 8-10-membered bicyclic cycloalkyl or cycloalkenyl wherein the second ring is C₅₋₆ heterocycloalkyl, phenyl, C₅₋₆cycloalkyl or C₅₋₆heteroaryl and the 8-10-membered bicyclic cycloalkyl or cycloalkenyl is optionally substituted with one or more substituents independently selected from halo, CN, NH₂, OH, NO₂, C₁₋₆alkyl, C₁₋₆ fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, X¹-phenyl, C₁₋₆alkylene-phenyl, X¹—C₁₋₆ alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (v) monocyclic C₅₋₆heterocycloalkyl optionally substituted with one or more substituents independently selected from halo, CN, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vi) 8-10-membered bicyclic heterocycloalkyl wherein the second ring is C₅₋₆ heterocycloalkyl, phenyl, C₅₋₆cycloalkyl or C₅₋₆heteroaryl and the 8-10-membered bicyclic heterocycloalkyl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vii) monocyclic C₅₋₆heteroaryl, optionally substituted with one or more substituents independently selected from halo, CN, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X—C₁₋₆fluoroalkyl, CO₂H, C(O)H, C₅₋₆heteroaryl, C₅₋₆ heterocycloalkyl, phenyl, X¹-phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂; and
   (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₅fluoroalkyl and NH₂;
R² is selected from H, C₁₋₆alkyl and C₁₋₆alkyl substituted with one or more substituents independently selected from OH and halo; and
X¹ and X² are independently selected from O, NH, N(C₁₋₆alkyl), N(C₁₋₆fluoroalkyl), C(O), C(O)O, OC(O), C(O)NH, C(O)N(C₁₋₆alkyl), C(O)N(C₁₋₆fluoroalkyl), NHC(O), N(C₁₋₆alkyl)C(O), N(C₁₋₆fluoroalkyl)C(O), S, S(O) and SO₂.

In accordance with another aspect of the present application, there is also provided a method of treating a disease, disorder or condition treatable by blocking SREBP2 activation and/or PCSK9 gene expression, the method comprising administering a therapeutically effective amount of one or more compounds of Formula (I), or a pharmaceutically acceptable salt, solvate and/or prodrug thereof, to a subject in need thereof:

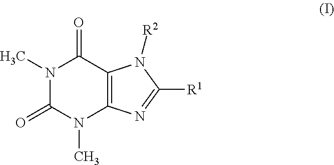

wherein
R¹ is selected from
   (i) phenyl substituted with one or more substituents independently selected from halo, NH₂, OH, NO₂, C₁₋₆alkyl, C₁₋₆fluoroalkyl, C₁₋₆alkyleneOH, C₁₋₆alkyleneNH₂, C₁₋₆ alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (ii) bicyclic C₉₋₁₀-aryl wherein the second ring in the bicyclic C₉₋₁₀aryl is phenyl or C₅₋₆cycloalkyl and the C₉₋₁₀-aryl is optionally substituted with one or more substituents independently selected from ═O, halo, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (iii) monocyclic C₃₋₆cycloalkyl or C₅₋₆cycloalkenyl optionally substituted with one or more substituents independently selected from halo, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆ heteroaryl, C₅₋₆heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₅ fluoroalkyl and NH₂;
   (iv) bicyclic C₅₋₁₀cycloalkyl or C₅₋₁₀cycloalkenyl wherein the second ring in the bicyclic C₅₋₁₀cycloalkyl or C₅₋₁₀cycloalkenyl is C₅₋₆heterocycloalkyl, phenyl, C₅₋₆cycloalkyl or C₅₋₆heteroaryl and the C₅₋₁₀cycloalkyl or C₅₋₁₀cycloalkenyl is optionally substituted with one or more substituents independently selected from halo, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, C₃₋₆cycloalkyl and C₃₋₆-cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₅ fluoroalkyl and NH₂;
   (v) monocyclic C₅₋₆heterocycloalkyl optionally substituted with one or more substituents independently selected from halo, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vi) bicyclic C₅₋₁₀heterocycloalkyl wherein the second ring in the bicyclic heterocycloalkyl is C₅₋₆heterocycloalkyl, phenyl, C₅₋₆cycloalkyl or C₅₋₆heteroaryl and the C₉₋₁₀heterocycloalkyl is optionally substituted with one or more substituents independently selected from halo, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
   (vii) monocyclic C₅₋₆heteroaryl, optionally substituted with one or more substituents independently selected from halo, NH₂, OH, NO₂, C₁₋₆alkyl, C₁₋₆fluoroalkyl, C₁₋₆ alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆ heterocycloalkyl, phenyl, C₃₋₆cycloalkyl and C₃₋₆cycloalkenyl, the latter 5 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂; and
   (viii) bicyclic C₅₋₁₀heteroaryl wherein the second ring in the bicyclic heteroaryl is C₅₋₆ heterocycloalkyl, phenyl, C₅₋₆cycloalkyl or C₅₋₆heteroaryl and the bicyclic C₉₋₁₀heteroaryl is optionally substituted with one or more substituents independently selected from halo, NH₂, OH, NO₂, C₁₋₆alkyl, C₁₋₆fluoroalkyl, C₁₋₆alkyleneOH, C₁₋₆ alkyleneNH₂, C₁₋₆alkyleneNH(C₁₋₆alkyl), C₁₋₆alkyleneN(C₁₋₆alkyl)(C₁₋₆alkyl), X¹—C₁₋₆ alkyl, X¹—C₁₋₆fluoroalkyl, CO₂H, C(O)H, C₅₋₈heteroaryl, C₅₋₆heterocycloalkyl, phenyl, C₃₋₆cycloalkyl and C₃₋₆cycloalkenyl, the latter 5 groups being optionally substituted with one or more substituents independently selected from halo, OH, C₁₋₆ alkyl, C₁₋₆fluoroalkyl, X²—C₁₋₆alkyl, X²—C₁₋₆fluoroalkyl and NH₂;
R² is selected from H, C₁₋₆alkyl and C₁₋₆alkyl substituted with one or more substituents independently selected from OH and halo; and
X¹ and X² are independently selected from O, NH, N(C₁₋₆alkyl), N(C₁₋₆fluoroalkyl), C(O), C(O)O, OC(O), C(O)NH, C(O)N(C₁₋₆alkyl), C(O)N(C₁₋₆fluoroalkyl), NHC(O), N(C₁₋₆alkyl)C(O), N(C₁₋₆fluoroalkyl)C(O), S, S(O) and SO₂

In some embodiments, R¹ is (i) phenyl substituted with one to five substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆ alkylene-phenyl, X¹—C₁₋₆alkylenephenyl, C₃₋₆cycloalkyl and C₃₋₆-cycloalkenyl, the latter 8 groups being optionally substituted with one to five substituents independently selected from halo, OH, C₁₋₆alkyl, C₁₋₆fluoroalkyl, X²—C₁₋₆alkyl, X²—C₁₋₆fluoroalkyl and NH₂. In some embodiments, R¹ is (i) phenyl substituted with one to five substituents independently selected from halo, CN, NH₂, OH, NO₂, C₁₋₄alkyl, C₁₋₄fluoroalkyl, C₁₋₄alkyleneOH, C₁₋₄alkyleneNH₂, C₁₋₄alkyleneNH(C₁₋₆alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆ heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, R¹ is (i) phenyl substituted with one to five substituents independently selected from F, Cl, Br, CN, NH₂, OH, NO₂, C₁₋₄alkyl, C₁₋₄fluoroalkyl, C₁₋₂ alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), X¹—Cl-2alkyl, X¹—C₁₋₄fluoroalkyl, CO₂H, C(O)H, C₅₋₆heteroaryl, C₅₋₆heterocycloalkyl, phenyl, X¹-phenyl, C₁₋₂alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (i) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (i) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ is selected from O, C(O), C(O)O, OC(O), C(O)NH, NHC(O), S and SO₂

In some embodiments, X² in (i) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (i) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (i) is selected from O, NH, C(O), C(O)O, OC(O), C(O)NH, NHC(O), S, S(O) and SO₂. In some embodiments, X² n (i) is selected from O, NH and C(O). In some embodiments, X² in (i) selected from O and C(O).

In some embodiments, X¹ in (i) is selected from O, C(O), C(O)O, OC(O), C(O)NH, NHC(O), and SO₂ and X² in (i) is selected from O and C(O), and R¹ is (i) phenyl substituted with one to five substituents independently selected from F, Cl, Br, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), O—C₁₋₂alkyl, O—C₁₋₂fluoroalkyl, NHC(O)—C₁₋₂alkyl, NHC(O)C₁₋₄ fluoroalkyl, C(O)C₁₋₄alkyl, C(O)C₁₋₄fluoroalkyl C(O)OC₁₋₄alkyl, C(O)OC₁₋₄fluoroalkyl, OC(O)C₁₋₂alkyl, OC(O)C₁₋₄fluoroalkyl, SO₂C₁₋₄alkyl, SO₂C₁₋₄fluoroalkyl, CO₂H, C(O)H, C₅₋₆ heteroaryl, C₅₋₆heterocycloalkyl, phenyl, O-phenyl, C(O)-phenyl, C₁₋₂alkylene-phenyl, O—C₁₋₂alkylenephenyl, C(O)—C₁₋₂alkylenephenyl, C₃₋₆cycloalkyl and C₃₋₆cycloalkenyl, the latter 10 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂.

In some embodiments, R¹ is (i) phenyl substituted with one to four substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, NHC(O)CH₃, NHC(O)CF₃, C(O)CH₃, C(O)CF₃, OC(O)CH₃, OC(O)CF₃, SO₂CH₃, SO₂CF₃, CO₂H, C(O)H, C₅₋₆heteroaryl, C₅₋₆heterocycloalkyl, phenyl, O-phenyl, C(O)-phenyl, CH₂-phenyl, O—CH₂phenyl, C(O)—CH₂pheny, C₃₋₆cycloalkyl and C₃₋₆cycloalkenyl, the latter 10 groups being optionally substituted with one to five substituents independently selected from F, Cl, OH, CH₃, CF₃, CH₃O, CF₃O, and NH₂.

In some embodiments, R¹ is (i) phenyl substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, NHC(O)CH₃, NHC(O)CF₃, C(O)CH₃, C(O)CF₃, OC(O)CH₃, OC(O)CF₃, SO₂CH₃, SO₂CF₃, CO₂H, C(O)H. In some embodiments, R¹ is (i) phenyl substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CH₃O, CF₃O, CH₂OH, CH₂NH₂, NHC(O)CH₃, NHC(O)CF₃, C(O)CH₃, C(O)CF₃, OC(O)CH₃, OC(O)CF₃, SO₂CH₃, SO₂CF₃, CO₂H and C(O)H.

In some embodiments, R¹ is (i) phenyl substituted with one or two substituents independently selected from C₅₋₆heteroaryl, C₅₋₆heterocycloalkyl, phenyl, O-phenyl, C(O)-phenyl, CH₂-phenyl, O-CH₂phenyl, C(O)—CH₂pheny, C₃₋₆cycloalkyl and C₃₋₆cycloalkenyl, the latter 10 groups being optionally substituted with one to four substituents independently selected from F, Cl, OH, CH₃, CF₃, CH₃O, CF₃O, and NH₂. In some embodiments, R¹ is phenyl substituted with one to two substituents independently selected from C₅₋₆heteroaryl, phenyl, O-phenyl, C(O)-phenyl, O—CH₂phenyl and C(O)—CH₂phenyl, the latter 6 groups being optionally substituted with one to three substituents independently selected from F, Cl, OH, CH₃, CF₃, CH₃O, CF₃O, and NH₂.

In some embodiments, R¹ is (ii) 9-10-membered bicyclic aryl wherein the second ring is phenyl or C₅₋₆cycloalkyl and the 9-10 membered bicyclic aryl is optionally substituted with one o five substituents independently selected from ═O, halo, CN, NH₂, OH, NO₂, C₁₋₄alkyl, C₁₋₄ fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₆alkyl), X¹—C₁₋₄alkyl, X¹—C₁₋₄fluoroalkyl, CO₂H, C(O)H, C₅₋₆heteroaryl, C₅₋₆ heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one o five substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (ii) 9-10-membered bicyclic aryl wherein the second ring is phenyl or C₅₋₆cycloalkyl and the 9-10 membered bicyclic aryl is optionally substituted with one to five substituents independently selected from ═O, halo, CN, NH₂, OH, NO₂, C₁₋₄alkyl, C₁₋₄ fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₆alkyl), X¹—C₁₋₄alkyl, X¹—C₁₋₄fluoroalkyl, CO₂H, C(O)H, C₅₋₆heteroaryl, C₅₋₆ heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (ii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (ii) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (ii) is selected from O, C(O), C(O)O, OC(O), C(O)NH, NHC(O), S and SO₂ In some embodiments, X¹ in (ii) is O,

In some embodiments, X² in (ii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (ii) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (ii) is selected from O, NH, C(O), C(O)O, OC(O), C(O)NH, NHC(O), S, S(O) and SO₂. In some embodiments, X² in (ii is selected from O, NH and C(O). In some embodiments, X² in (ii) is O.

In some embodiments, X¹ in (ii) is O and X² in (ii) is O, and R¹ is (ii) 9-10-membered bicyclic aryl wherein the second ring is phenyl or C₅₋₆cycloalkyl and the 9-10 membered bicyclic aryl is optionally substituted with one to four substituents independently selected from ═O, halo, CN, NH₂, OH, NO₂, C₁₋₄alkyl, C₁₋₄fluoroalkyl, C₁₋₂alkyleneOH, C₁₋₂alkyleneNH₂, C₁₋₂ alkyleneNH(C₁₋₄alkyl), C₁₋₂alkyleneN(C₁₋₄alkyl)(C₁₋₆alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₄alkylene-phenyl, O—C₁₋₄ alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄ fluoroalkyl, O—C₁₋₄alkyl, O—C₁₋₄fluoroalkyl and NH₂.

In some embodiments, X¹ in (ii) is O and X² in (ii) is O, and R¹ is (ii) 9-10-membered bicyclic aryl wherein the second ring is phenyl or C₅₋₆cycloalkyl and the 9-10 membered bicyclic aryl is optionally substituted with one to three substituents independently selected from ═O, F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂phenyl, O CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂.

In some embodiments, R¹ is (ii) 9-10-membered bicyclic aryl wherein the second ring is phenyl or C₅₋₆cycloalkyl and the 9-10 membered bicyclic aryl is optionally substituted with one to three substituents independently selected from ═O, F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H and C(O)H. In some embodiments, R¹ is (ii) 9-10-membered bicyclic aryl wherein the second ring is phenyl or C₅₋₆cycloalkyl and the 9-10 membered bicyclic aryl is optionally substituted with one to three substituents independently selected from ═O, F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H and C(O)H.

In some embodiments, R¹ is (ii) 9-10-membered bicyclic aryl wherein the second ring is phenyl and 9-10 membered bicyclic aryl is optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H and C(O)H. In some embodiments, R¹ is (ii) 9-10-membered bicyclic aryl wherein the second ring is phenyl and 9-10 membered bicyclic aryl is optionally substituted with one to three substituents independently selected from F, Cl, Br and CH₃. In some embodiments, R¹ is (ii) 9-10-membered bicyclic aryl wherein the second ring is C₅₋₆cycloalkyl and the 9-10 membered bicyclic aryl is optionally substituted with one to three substituents independently selected from ═O, F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H and C(O)H. In some embodiments, R¹ is (ii) 9-10-membered bicyclic aryl wherein the second ring is C₅₋₆cycloalkyl and the 9-10 membered bicyclic aryl is optionally substituted with one to three substituents independently selected from ═O and F, Cl, Br, CH₃. In some embodiments, R¹ is (ii) 9-10-membered bicyclic aryl wherein the second ring is C₅₋₆ cycloalkyl and the 9-10 membered bicyclic aryl is optionally substituted with one to two ═O.

In some embodiments, R¹ is (ii) 9-10-membered bicyclic aryl wherein the second ring is phenyl or C₅₋₆cycloalkyl and the 9-10 membered bicyclic aryl is optionally substituted with one to three substituents independently selected from, C₅₋₆heteroaryl, C₅₋₆heterocycloalkyl, phenyl, O-phenyl, CH₂phenyl, O CH₂phenyl, C₃₋₆cycloalkyl and C₃₋₆cycloalkenyl, the latter 8 groups being optionally substituted with one to three substituents independently selected from halo, OH, C₁₋₄ alkyl, C₁₋₄fluoroalkyl, O—C₁₋₄alkyl, O—C₁₋₄fluoroalkyl and NH₂.

In some embodiments, R¹ is (iii) monocyclic C₃₋₆cycloalkyl or C₅₋₆cycloalkenyl optionally substituted with one to five substituents independently selected from halo, CN, NH₂, OH, NO₂, C₁₋₄alkyl, C₁₋₄fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆ heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (iii) monocyclic C₃₋₆cycloalkyl or C₅₋₆cycloalkenyl optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂ alkyleneNH(C₁₋₆alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₈heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₆ fluoroalkyl, X²—C₁₋₄alkyl, X²—C₁₋₄fluoroalkyl and NH₂

In some embodiments, X¹ in (iii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (iii) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ is selected from O, C(O), C(O)O, OC(O), C(O)NH, NHC(O), S and SO₂ In some embodiments, X¹ in (iii) is O.

In some embodiments, X² in (iii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (ii) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (iii) is selected from O, NH, C(O), C(O)O, OC(O), C(O)NH, NHC(O), S, S(O) and SO₂. In some embodiments, X² in (iii) is selected from O, NH and C(O). In some embodiments, X² in (iii) is O.

In some embodiments, X¹ in (iii) is O and X² in (iii) is O, and R¹ is (iii) monocyclic C₃₋₆cycloalkyl or C₅₋₆cycloalkenyl optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₄ alkylene-phenyl, O—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₆fluoroalkyl, O—C₁₋₄alkyl, O—C₁₋₄fluoroalkyl and NH₂ In some embodiments, R¹ is (iii) monocyclic C₅₋₆cycloalkyl or C₅₋₆cycloalkenyl optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆ heterocycloalkyl, phenyl, O-phenyl, C₁₋₄alkylene-phenyl, O—C₁₋₄alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₆fluoroalkyl, O—C₁₋₄alkyl, O—C₁₋₄fluoroalkyl and NH₂ In some embodiments, R¹ is (iii) monocyclic C₅₋₆cycloalkyl optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆ heterocycloalkyl, phenyl, O-phenyl, C₁₋₄alkylene-phenyl, O—C₁₋₄alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂ In some embodiments, R¹ is (iii) monocyclic C₅₋₆cycloalkenyl optionally substituted with one to three substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₄alkylene-phenyl, O—C₁₋₄alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₆fluoroalkyl, O—C₁₋₄alkyl, O—C₁₋₄ fluoroalkyl and NH₂

In some embodiments, R¹ is (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one to five substituents independently selected from halo, CN, NH₂, OH, NO₂, C₁₋₄alkyl, C₁₋₄ fluoroalkyl, C₁₋₄alkyleneOH, C₁₋₄alkyleneNH₂, C₁₋₄alkyleneNH(C₁₋₆alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆ heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one to five substituents independently selected from halo, CN, NH₂, OH, NO₂, C₁₋₄alkyl, C₁₋₄fluoroalkyl, C₁₋₂ alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₂alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (iv) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (iv) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (iv) is selected from O, C(O), C(O)O, OC(O), C(O)NH, NHC(O), S and SO₂ In some embodiments, X¹ in (iv) is O,

In some embodiments, X² in (iv) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (iv) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (iv) is selected from O, NH, C(O), C(O)O, OC(O), C(O)NH, NHC(O), S, S(O) and SO₂. In some embodiments, X² in (iv) is selected from O, NH and C(O). In some embodiments, X² in (iv) is O.

In some embodiments, X¹ in (iv) is O and X² in (iv) is O, and R¹ is (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂ alkyleneNH(C₁₋₆alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₂alkylene-phenyl, O—C₁₋₄ alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄ fluoroalkyl, O—C₁₋₄alkyl, O—C₁₋₄fluoroalkyl and NH₂.

In some embodiments, X¹ in (iv) is O and X² in (iv) is O, and R¹ is (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O-CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂.

In some embodiments, R¹ is (iv) 8-10-membered bicyclic cycloalkyl wherein the second ring is C₅₋₆heterocycloalkyl, phenyl, C₅₋₆cycloalkyl or C₅₋₆heteroaryl and the 8-10-membered bicyclic cycloalkyl is optionally substituted with one to five substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O—CH₂phenyl, C₃₋₆cycloalkyl and C₃₋₆cycloalkenyl, the latter 8 groups being optionally substituted with one to five substituents independently selected from halo, OH, C₁₋₄alkyl, C₁₋₄ fluoroalkyl, O—C₁₋₄alkyl, O—C₁₋₄fluoroalkyl and NH₂. In some embodiments, R¹ is (iv) 8-10-membered bicyclic cycloalkyl wherein the second ring is phenyl or C₅₋₆heteroaryl and the 8-10-membered bicyclic cycloalkyl is optionally substituted with one to five substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O—CH₂phenyl, C₃₋₆cycloalkyl and C₃₋₆cycloalkenyl, the latter 8 groups being optionally substituted with one to five substituents independently selected from halo, OH, C₁₋₄alkyl, C₁₋₄ fluoroalkyl, O—C₁₋₄alkyl, O—C₁₋₄fluoroalkyl and NH₂. In some embodiments, R¹ is (iv) 8-10-membered bicyclic cycloalkyl wherein the second ring is phenyl and the 8-10-membered bicyclic cycloalkyl or cycloalkenyl is optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O—CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄ fluoroalkyl, O—C₁₋₄alkyl, O—C₁₋₄fluoroalkyl and NH₂.

In some embodiments, R¹ is (iv) 8-10-membered bicyclic cycloalkenyl wherein the second ring is C₅₋₆heterocycloalkyl, phenyl, C₅₋₆cycloalkyl or C₅₋₆heteroaryl and the 8-10-membered bicyclic cycloalkenyl is optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O—CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (iv) 8-10-membered bicyclic cycloalkenyl wherein the second ring is phenyl or C₅₋₆heteroaryl and the 8-10-membered bicyclic cycloalkenyl is optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O—CH₂phenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (iv) 8-10-membered bicyclic cycloalkenyl wherein the second ring is phenyl and the 8-10-membered bicyclic cycloalkenyl is optionally substituted with one to three substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, CH₂-phenyl, O—CH₂phenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (iv) 8-10-membered bicyclic cycloalkenyl wherein the second ring is phenyl.

In some embodiments, R¹ is (v) monocyclic C₅₋₆heterocycloalkyl optionally substituted with one to five substituents independently selected from halo, CN, NH₂, OH, NO₂, C₁₋₄ alkyl, C₁₋₄fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄ fluoroalkyl and NH₂. In some embodiments, R¹ is (v) monocyclic C₅₋₆heterocycloalkyl optionally substituted with one five substituents independently selected from halo, CN, NH₂, OH, NO₂, C₁₋₄ alkyl, C₁₋₄fluoroalkyl, C₁₋₂alkyleneOH, C₁₋₂alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₄alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄ fluoroalkyl and NH₂.

In some embodiments, X¹ in (v) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (v) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (v) is selected from O, C(O), C(O)O, OC(O), C(O)NH, NHC(O), S and SO₂ In some embodiments, X¹ in (v) is O,

In some embodiments, X² in (v) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (v) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (v) is selected from O, NH, C(O), C(O)O, OC(O), C(O)NH, NHC(O), S, S(O) and SO₂. In some embodiments, X² in (v) is selected from O, NH and C(O). In some embodiments, X² in (v) is O.

In some embodiments, X¹ in (v) is O and X² in (iv) is O, and R¹ is (v) monocyclic C₅₋₆heterocycloalkyl optionally substituted with one to five substituents independently selected from F, Br, Cl, CN, NH₂, OH, NO₂, C₁₋₄alkyl, C₁₋₄fluoroalkyl, C₁₋₂alkyleneOH, C₁₋₂alkyleneNH₂, C₁₋₄alkyleneNH(C₁₋₄alkyl), C₁₋₄alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₄alkylene-phenyl, O—C₁₋₂ alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄ fluoroalkyl, O—C₁₋₄alkyl, O—C₁₋₄fluoroalkyl and NH₂. In some embodiments, R¹ is (v) monocyclic C₅₋₆heterocycloalkyl optionally substituted with one to four substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₄ alkylene-phenyl, O—C₁₋₂alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂.

In some embodiments, the monocyclic C₅₋₆heterocycloalkyl in R¹ is selected from tetrahydrofuranyl, tetrahydrothiophenyl, pyrrolidinyl, imidazolidinyl, pyrazolidinyl, 2-oxopiperazinyl, 2-oxopiperdinyl, 2-oxopyrrolidinyl, isoxthiolidinyl, thiazolidinyl, isothiazolidinyl, piperidinyl, dihydropyranyl, tetrahydropyranyl, diazinanyl (e.g, piperazinyl), piperidinyl, morpholinyl, thiomorpholinyl, thiomorpholinyl sulfoxide, dioxanyl and dithianyl each of which is optionally substituted with one to three substituents independently selected from F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₄alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄ fluoroalkyl, O—C₁₋₄alkyl, O—C₁₋₄fluoroalkyl and NH₂. In some embodiments, the monocyclic C₅₋₆ heterocycloalkyl in R¹ is dihydropyranyl.

In some embodiments, R¹ is (vi) 8-10-membered bicyclic heterocycloalkyl wherein the second ring is C₅₋₆heterocycloalkyl, phenyl, C₅₋₆cycloalkyl or C₅₋₆heteroaryl and the 8-10-membered bicyclic heterocycloalkyl is optionally substituted with one to five substituents independently selected from halo, CN, NH₂, OH, NO₂, C₁₋₄alkyl, C₁₋₄fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₄ alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (vi) 8-10-membered bicyclic heterocycloalkyl wherein the second ring is C₅₋₆heterocycloalkyl, phenyl, C₅₋₆cycloalkyl or C₅₋₆heteroaryl and the 8-10-membered bicyclic heterocycloalkyl is optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₂alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (vi) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (vi) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (vi) is selected from O, C(O), C(O)O, OC(O), C(O)NH, NHC(O), S and SO₂ In some embodiments, X¹ in (vi) is O,

In some embodiments, X² in (v) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (vi) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (vi) is selected from O, NH, C(O), C(O)O, OC(O), C(O)NH, NHC(O), S, S(O) and SO₂. In some embodiments, X² in (vi) is selected from O, NH and C(O). In some embodiments, X² in (vi) is O.

In some embodiments, X¹ in (vi) is O and X² in (vi) is O, and R¹ is (v) 8-10-membered bicyclic heterocycloalkyl wherein the second ring is C₅₋₆heterocycloalkyl, phenyl, C₅₋₆ cycloalkyl or C₅₋₆heteroaryl and the 8-10-membered bicyclic heterocycloalkyl is optionally substituted with one to five substituents independently selected from halo, CN, NH₂, OH, NO₂, C₁₋₄ alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆ heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (v) 8-10-membered bicyclic heterocycloalkyl wherein the second ring is C₅₋₆heterocycloalkyl, phenyl, C₅₋₆cycloalkyl or C₅₋₆heteroaryl and the 8-10-membered bicyclic heterocycloalkyl is optionally substituted with one to four substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₂ alkylene-phenyl, O—C₁₋₂alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (v) 9-10-membered bicyclic heterocycloalkyl wherein the second ring is C₅₋₆heterocycloalkyl, phenyl, C₅₋₆cycloalkyl or C₅₋₆heteroaryl and the 9-10-membered bicyclic heterocycloalkyl is optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄ fluoroalkyl and NH₂. In some embodiments, R¹ is (vi) 9-10-membered bicyclic heterocycloalkyl wherein the second ring is phenyl and the 9-10-membered bicyclic heterocycloalkyl is optionally substituted with one o three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CHF₂O, CH₂OH, CH₂NH₂, CO₂H, C(O)H, and phenyl, the phenyl being optionally substituted with one to three substituents independently selected from halo, OH, C₁₋₄alkyl, C₁₋₄fluoroalkyl, O—C₁₋₄alkyl, O—C₁₋₄fluoroalkyl and NH₂.

In some embodiments, R¹ is (vii) monocyclic C₅₋₆heteroaryl, optionally substituted with one to five substituents independently selected from halo, CN, NH₂, OH, NO₂, C₁₋₄alkyl, C₁₋₄ fluoroalkyl, C₁₋₄alkyleneOH, C₁₋₄alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆ heterocycloalkyl, phenyl, X¹-phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂. In some embodiments, R¹ is (vii) monocyclic C₅₋₆heteroaryl, optionally substituted with one to five substituents independently selected from halo, CN, NH₂, OH, NO₂, C₁₋₄ alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, X¹-phenyl, C₁₋₂alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (vii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (vii) is selected from O, C(O), C(O)O, OC(O), C(O)NH, C(O)N(C₁₋₄alkyl), C(O)N(C₁₋₄ fluoroalkyl), NHC(O), N(C₁₋₄alkyl)C(O), N(C₁₋₄fluoroalkyl)C(O), S and SO₂. In some embodiments, X¹ in (vii) is selected from O, C(O), C(O)O, OC(O), C(O)NH, NHC(O), S and SO₂ In some embodiments, X¹ in (vii) is selected from O and S.

In some embodiments, X² in (vii) is selected from O, NH, N(C₁₋₄alkyl), N(C₁₋₄ fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (vii) is selected from O, NH, C(O), C(O)O, OC(O), C(O)NH, NHC(O), N(C₁₋₄alkyl)C(O), N(C₁₋₄ fluoroalkyl)C(O), S, S(O) and SO₂. In some embodiments, X² in (vii) is selected from O, NH, C(O), C(O)O, OC(O), C(O)NH, NHC(O), S, S(O) and SO₂. In some embodiments, X² in (vii) is selected from O, NH and C(O). In some embodiments, X² in (vii) is O.

In some embodiments, X¹ in (vii) is selected from O and S and X² in (vii) is O and R¹ is (vi) monocyclic C₅₋₆heteroaryl, optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), O—C₁₋₄alkyl, S—C₁₋₄alkyl, O—C₁₋₄fluoroalkyl, S—C₁₋₄fluoroalkyl, CO₂H, C(O)H, C₅₋₆heteroaryl, C₅₋₆ heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄ fluoroalkyl and NH₂. In some embodiments, R¹ is (vii) monocyclic C₅₋₆heteroaryl, optionally substituted with one to four substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, CH₃S, CF₃S, CH₂OH, CH₂NH₂, CO₂H, C(O)H, C₅₋₆heteroaryl, C₅₋₆ heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to four substituents independently selected from halo, OH, C₁₋₄alkyl, C₁₋₄fluoroalkyl, O—C₁₋₄alkyl, O—C₁₋₄ fluoroalkyl and NH₂. In some embodiments, R¹ is (vii) monocyclic C₅₋₆heteroaryl, optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CH₃S, CF₃S, CH₂OH, CH₂NH₂, CO₂H, C(O)H, C₅₋₆heteroaryl, C₅₋₆heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂.

In some embodiments, the monocyclic C₅₋₆heteroaryl in R¹ is selected from furanyl, thiophenyl, pyridyl, triazolyl, pyrrolyl, tetrazolyl, imidazolyl, pyrazolyl, oxazolyl, thiazolyl, pyrimidinyl, isoxazolyl and isothiazolyl, optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CH₃S, CF₃S, CH₂OH, CH₂NH₂, CO₂H, C(O)H, C₅₋₆heteroaryl, C₅₋₆heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂. In some embodiments, the monocyclic C₅₋₆heteroaryl in R¹ is selected from thiophenyl, pyridyl, pyrazolyl, oxazolyl, pyrimidinyl, and isothiazolyl, optionally substituted with one or more substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CH₃S, CF₃S, CH₂OH, CH₂NH₂, CO₂H, C(O)H, C₅₋₆heteroaryl, C₅₋₆ heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄ fluoroalkyl and NH₂. In some embodiments, the monocyclic C₅₋₆heteroaryl in R¹ is selected from pyridyl, and pyrimidinyl optionally substituted with one to three substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₃O, CF₃O, CH₃S, CF₃S, CH₂OH, CH₂NH₂, CO₂H, C(O)H, C₅₋₆heteroaryl, C₅₋₆ heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄ fluoroalkyl and NH₂.

In some embodiments, R¹ is (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one to five substituents independently selected from halo, CN, NH₂, OH, NO₂, C₁₋₆alkyl, C₁₋₄fluoroalkyl, C₁₋₄alkyleneOH, C₁₋₄ alkyleneNH₂, C₁₋₄alkyleneNH(C₁₋₄alkyl), C₁₋₄alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆heterocycloalkyl, phenyl, X¹-phenyl, C₁₋₄alkylene-phenyl, X¹—C₁₋₄alkylenephenyl, C₃₋₆cycloalkyl and C₃₋₆cycloalkenyl, the latter 8 groups being optionally substituted with one to five substituents independently selected from halo, OH, C₁₋₄ alkyl, C₁₋₄fluoroalkyl, X²—C₁₋₄alkyl, X²—C₁₋₄fluoroalkyl and NH₂. In some embodiments, R¹ is (viii) 8-10-membered bicyclic heteroaryl wherein the second ring is C₅₋₆heterocycloalkyl, phenyl, C₅₋₆ cycloalkyl or C₅₋₆heteroaryl and the 8-10-membered bicyclic heteroaryl is optionally substituted with one to five substituents independently selected from halo, CN, NH₂, OH, NO₂, C₁₋₆alkyl, C₁₋₄fluoroalkyl, C₁₋₄alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), X¹—C₁₋₄alkyl, X¹—C₁₋₄fluoroalkyl, CO₂H, C(O)H, C₅₋₆heteroaryl, C₅₋₆ heterocycloalkyl, phenyl, X¹-phenyl, C₁₋₂alkylene-phenyl, X¹—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, X²—C₁₋₄alkyl, X²—C₁₋₄fluoroalkyl and NH₂.

In some embodiments, X¹ in (viii) is selected from O, NH, N(C₁₋₄alkyl), N(C₁₋₄fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X¹ in (viii) is selected from O, C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O) and SO₂. In some embodiments, X¹ in (viii) is selected from O, C(O), C(O)O, OC(O), C(O)NH, NHC(O), and SO₂ In some embodiments, X¹ in (viii) is O,

In some embodiments, X² in (viii) is selected from O, NH, N(C₁₋₄alkyl), N(C₁₋₄fluoroalkyl), C(O), C(O)O, OC(O), C(O)NH, C(O)N(C₁₋₄alkyl), C(O)N(C₁₋₄fluoroalkyl), NHC(O), N(C₁₋₄alkyl)C(O), N(C₁₋₄fluoroalkyl)C(O), S, S(O) and SO₂. In some embodiments, X² in (viii) is selected from O, NH, C(O), C(O)O, OC(O), C(O)NH, NHC(O), N(C₁₋₄alkyl)C(O), N(C₁₋₄fluoroalkyl)C(O), S, S(O) and SO₂. In some embodiments, X² in (viii) is selected from O, NH, C(O), C(O)O, OC(O), C(O)NH, NHC(O), S, S(O) and SO₂. In some embodiments, X² in (viii) is selected from O, NH and C(O). In some embodiments, X² in (viii) is O.

In some embodiments, X¹ in (viii) is selected from O and S and X² in (viii) is O and R¹ is (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one to five substituents independently selected from halo, CN, NH₂, OH, NO₂, C₁₋₆alkyl, C₁₋₄fluoroalkyl, C₁₋₄alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₈heteroaryl, C₅₋₆ heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂. In some embodiments, R¹ is (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one to four substituents independently selected F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, CO₂H, C(O)H, C₅₋₆heteroaryl, C₅₋₆heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to four substituents independently selected from halo, OH, C₁₋₄ alkyl, C₁₋₄fluoroalkyl, O—C₁₋₄alkyl, O—C₁₋₄fluoroalkyl and NH₂. In some embodiments, R¹ is (viii) 9-10-membered bicyclic heteroaryl wherein the second ring is C₅₋₆heterocycloalkyl, phenyl, C₅₋₆ cycloalkyl or C₅₋₆heteroaryl and the 9-10-membered bicyclic heteroaryl is optionally substituted with one to three substituents independently selected F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, C(CF₃)₃, CH(CH₃)₂O, CH₃CH₂O, CH₃O, CF₃O, CHF₂O, CH₂OH, CH₂NH₂, CO₂H, C(O)H, C₅₋₆heteroaryl, C₅₋₆ heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂.

In some embodiments, the 9-10-membered bicyclic heteroaryl in R¹ is selected from benzofuranyl, indolyl, isoindolyl, benzodioxolyl, benzothiazolyl, quinolinyl, isoquinolinyl and benzothiophenyl each of which is optionally substituted with one to three substituents independently selected F, Cl, Br, CN, NH₂, OH, NO₂, CH₃, CH₂CH₃, CH₂CH₂CH₃, CH₂CH₂CH₂CH₃, CH(CH₃)₂, C(CH₃)₃, CF₃, CF₂H, C(CF₃)₃, CH(CH₃)₂O, CH₃CH₂O, CH₃O, CF₃O, CHF₂O, CH₂OH, CH₂NH₂, CO₂H, C(O)H, C₅₋₆heteroaryl, C₅₋₆heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to three substituents independently selected from halo, OH, C₁₋₄alkyl, C₁₋₄fluoroalkyl, O—C₁₋₄alkyl, O—C₁₋₄fluoroalkyl and NH₂.

In some embodiments, each C₅₋₆heteroaryl in the substituents on R¹ is independently selected from furanyl, thiophenyl, pyridyl, triazolyl, pyrrolyl, tetrazolyl, imidazolyl, pyrazolyl, oxazolyl, thiazolyl, pyrimidinyl, isoxazolyl and isothiazolyl.

In some embodiments, the C₅₋₆heteroaryl in the substituents on R¹ in (i) is selected from triazolyl and pyridyl. In some embodiments, the triazole is a 1, 2, 4 triazolyl. In some embodiments, the substituent C₅₋₆heteroaryl in R¹ in (i) is pyridyl.

In some embodiments, the C₅₋₆heterocycloalkyl in the substituents on R¹ is independently selected from tetrahydrofuranyl, tetrahydrothiophenyl, pyrrolidinyl, imidazolidinyl, pyrazolidinyl, 2-oxopiperazinyl, 2-oxopiperdinyl, 2-oxopyrrolidinyl, isoxthiolidinyl, thiazolidinyl, isothiazolidinyl, piperidinyl, dihydropyranyl, tetrahydropyranyl, diazinanyl (e.g, piperazinyl), piperidinyl, morpholinyl, thiomorpholinyl, thiomorpholinyl sulfoxide, dioxanyl and dithianyl.

In some embodiments, each C₃₋₆cycloalkyl in the substituents on R¹ is independently selected from, cyclopropyl, cyclobutyl, cyclopentyl and cyclohexyl,

In some embodiments, each C₃₋₆cycloalkenyl in the substituents on R¹ is independently selected from, cyclopropenyl, cyclobutenyl, cyclopentenyl and cyclohexenyl.

In some embodiments, R² is selected from H, C₁₋₄alkyl and C₁₋₄alkyl substituted with one or more substituents independently selected from OH and halo. In some embodiments, R² is selected from H, CH₃, CF₃, CF₂H, CH₃O, CF₃O, CHF₂O and CH₂OH. In some embodiments, R² is H.

In some embodiments, “one or more” is one to five. In some embodiments, “one or more” is one to four. In some embodiments, “one or more” is one to three. In some embodiments, “one or more” is “one or two”.

In some embodiments, the compound of Formula (I), or pharmaceutically acceptable salt, solvate and/or prodrug thereof, is a compound of Formula (Ia), or pharmaceutically acceptable salt, solvate and/or prodrug thereof or is a compound listed in Table 1 or pharmaceutically acceptable salt, solvate and/or prodrug thereof. In some embodiments, the compound of Formula (I), or pharmaceutically acceptable salt, solvate and/or prodrug thereof, is a compound of Formula (Ia), or pharmaceutically acceptable salt, solvate and/or prodrug thereof. In some embodiments, the compound of Formula (I), or pharmaceutically acceptable salt, solvate and/or prodrug thereof, is a compound listed in Table 1 or pharmaceutically acceptable salt, solvate and/or prodrug thereof.

Therefore, in some embodiments, the compound of Formula (I), or pharmaceutically acceptable salt, solvate and/or prodrug thereof, including the compound of Formula (Ia) or pharmaceutically acceptable salt, solvate and/or prodrug thereof and the compounds listed in Table 1, or pharmaceutically acceptable salt, solvate and/or prodrug thereof, is selected from the compounds listed in Table 2:

TABLE 2

| Compound No. | Structure |
|---|---|
| I-1 | 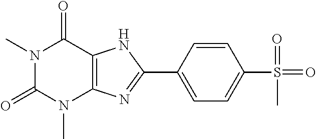 [SMILES: Cn1c(=O)c2[nH]c(-c3ccc(S(C)(=O)=O)cc3)nc2n(C)c1=O] |
| I-2 | 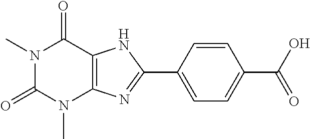 [SMILES: Cn1c(=O)c2[nH]c(-c3ccc(C(=O)O)cc3)nc2n(C)c1=O] |
| I-5 | 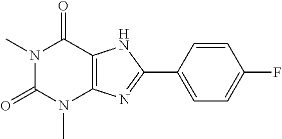 [SMILES: Cn1c(=O)c2[nH]c(-c3ccc(F)cc3)nc2n(C)c1=O] |
| I-6 | 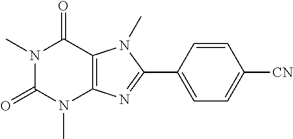 [SMILES: Cn1c(=O)c2c(nc(-c3ccc(C#N)cc3)n2C)n(C)c1=O] |
| I-7 | 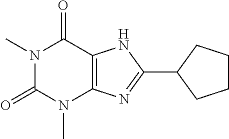 [SMILES: Cn1c(=O)c2[nH]c(C3CCCC3)nc2n(C)c1=O] |
| I-8 | 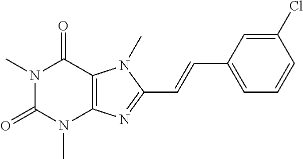 [SMILES: Cn1c(=O)c2c(nc(/C=C/c3cccc(Cl)c3)n2C)n(C)c1=O] |
| I-10 | 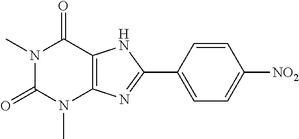 [SMILES: Cn1c(=O)c2[nH]c(-c3ccc([N+](=O)[O-])cc3)nc2n(C)c1=O] |
| I-11 | 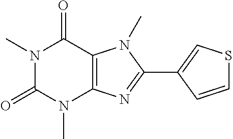 [SMILES: Cn1c(=O)c2c(nc(-c3ccsc3)n2C)n(C)c1=O] |
| I-12 | 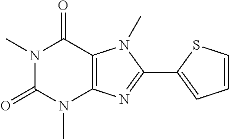 [SMILES: Cn1c(=O)c2c(nc(-c3cccs3)n2C)n(C)c1=O] |
| Ia-1 | 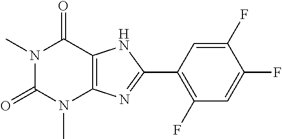 [SMILES: Cn1c(=O)c2[nH]c(-c3cc(F)c(F)cc3F)nc2n(C)c1=O] |
| Ia-2 | 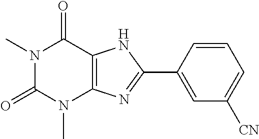 [SMILES: Cn1c(=O)c2[nH]c(-c3cccc(C#N)c3)nc2n(C)c1=O] |
| Ia-3 | 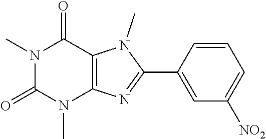 [SMILES: Cn1c(=O)c2c(nc(-c3cccc([N+](=O)[O-])c3)n2C)n(C)c1=O] |
| Ia-4 | 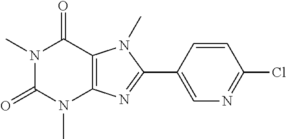 [SMILES: Cn1c(=O)c2c(nc(-c3ccc(Cl)nc3)n2C)n(C)c1=O] |
| Ia-5 | 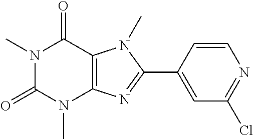 [SMILES: Cn1c(=O)c2c(nc(-c3ccnc(Cl)c3)n2C)n(C)c1=O] |
| Ia-6 | 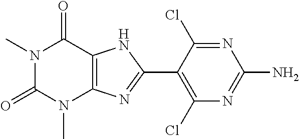 [SMILES: Cn1c(=O)c2[nH]c(-c3c(Cl)nc(N)nc3Cl)nc2n(C)c1=O] |
| Ia-7 | 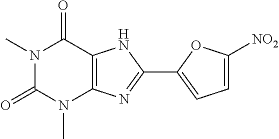 [SMILES: Cn1c(=O)c2[nH]c(-c3ccc([N+](=O)[O-])o3)nc2n(C)c1=O] |
| Ia-8 | 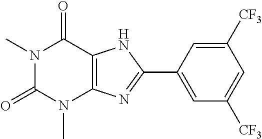 [SMILES: Cn1c(=O)c2[nH]c(-c3cc(C(F)(F)F)cc(C(F)(F)F)c3)nc2n(C)c1=O] |
| Ia-9 | 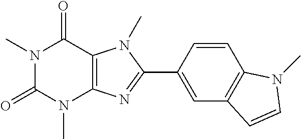 |
| Ia-10 | 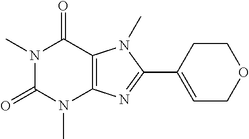 [SMILES: Cn1c(=O)c2c(nc(C3=CCOCC3)n2C)n(C)c1=O] |
| Ia-11 | 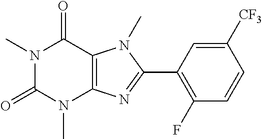 [SMILES: Cn1c(=O)c2c(nc(-c3cc(C(F)(F)F)ccc3F)n2C)n(C)c1=O] |
| Ia-12 | 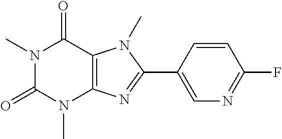 [SMILES: Cn1c(=O)c2c(nc(-c3ccc(F)nc3)n2C)n(C)c1=O] |
| Ia-13 | 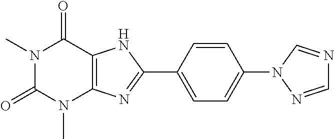 [SMILES: Cn1c(=O)c2[nH]c(-c3ccc(-n4cncn4)cc3)nc2n(C)c1=O] |
| Ia-14 | 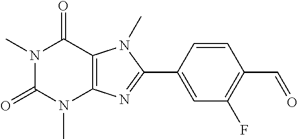 [SMILES: Cn1c(=O)c2c(nc(-c3ccc(C=O)c(F)c3)n2C)n(C)c1=O] |
| Ia-15 | 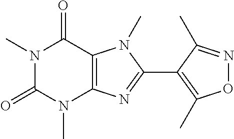 [SMILES: Cc1noc(C)c1-c1nc2c(c(=O)n(C)c(=O)n2C)n1C] |
| Ia-16 | 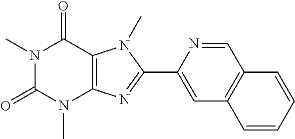 [SMILES: Cn1c(=O)c2c(nc(-c3cc4ccccc4cn3)n2C)n(C)c1=O] |
| Ia-17 | 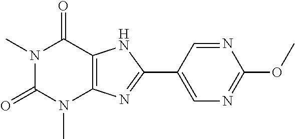 |
| Ia-18 | 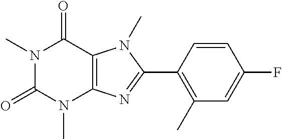 [SMILES: Cc1cc(F)ccc1-c1nc2c(c(=O)n(C)c(=O)n2C)n1C] |
| Ia-19 | 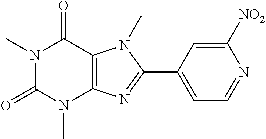 [SMILES: Cn1c(=O)c2c(nc(-c3ccnc([N+](=O)[O-])c3)n2C)n(C)c1=O] |
| Ia-20 | 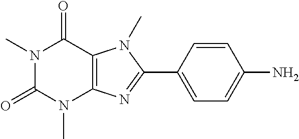 [SMILES: Cn1c(=O)c2c(nc(-c3ccc(N)cc3)n2C)n(C)c1=O] |
| Ia-21 | 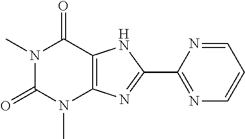 [SMILES: Cn1c(=O)c2[nH]c(-c3ncccn3)nc2n(C)c1=O] |
| Ia-22 | 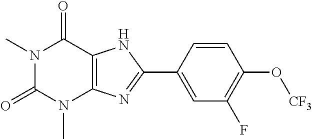 [SMILES: Cn1c(=O)c2[nH]c(-c3ccc(OC(F)(F)F)c(F)c3)nc2n(C)c1=O] |
| Ia-23 | 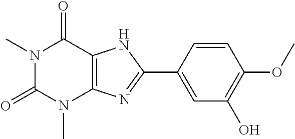 |
| Ia-24 | 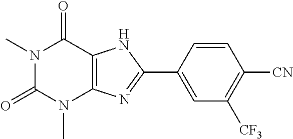 [SMILES: Cn1c(=O)c2[nH]c(-c3ccc(C#N)c(C(F)(F)F)c3)nc2n(C)c1=O] |
| Ia-25 | 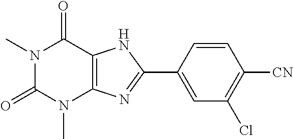 [SMILES: Cn1c(=O)c2[nH]c(-c3ccc(C#N)c(Cl)c3)nc2n(C)c1=O] |
| Ia-26 | 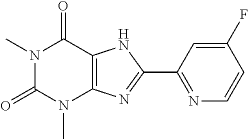 [SMILES: Cn1c(=O)c2[nH]c(-c3cc(F)ccn3)nc2n(C)c1=O] |
| Ia-27 | 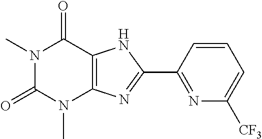 [SMILES: Cn1c(=O)c2[nH]c(-c3cccc(C(F)(F)F)n3)nc2n(C)c1=O] |
| Ia-28 | 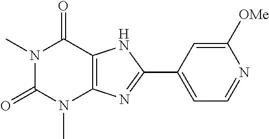 [SMILES: COc1cc(-c2nc3c([nH]2)c(=O)n(C)c(=O)n3C)ccn1] |
| Ia-29 | 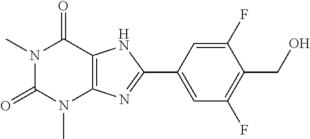 [SMILES: Cn1c(=O)c2[nH]c(-c3cc(F)c(CO)c(F)c3)nc2n(C)c1=O] |
| Ia-30 | 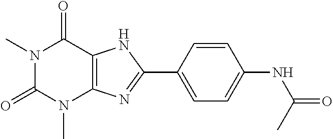 [SMILES: CC(=O)Nc1ccc(-c2nc3c([nH]2)c(=O)n(C)c(=O)n3C)cc1] |
| Ia-31 | 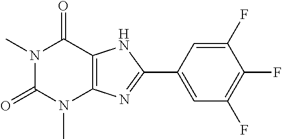 [SMILES: Cn1c(=O)c2[nH]c(-c3cc(F)c(F)c(F)c3)nc2n(C)c1=O] |
| Ia-32 | 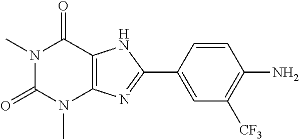 [SMILES: Cn1c(=O)c2[nH]c(-c3ccc(N)c(C(F)(F)F)c3)nc2n(C)c1=O] |
| Ia-33 | 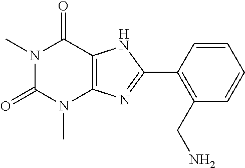 [SMILES: Cn1c(=O)c2[nH]c(-c3ccccc3CN)nc2n(C)c1=O] |
| Ia-34 | 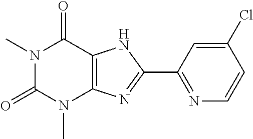 [SMILES: Cn1c(=O)c2[nH]c(-c3cc(Cl)ccn3)nc2n(C)c1=O] |
| Ia-35 | 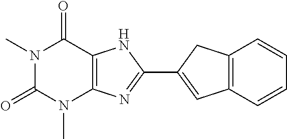 [SMILES: Cn1c(=O)c2[nH]c(C3=Cc4ccccc4C3)nc2n(C)c1=O] |
| Ia-36 | 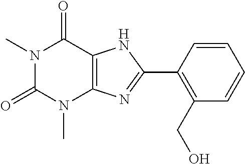 [SMILES: Cn1c(=O)c2[nH]c(-c3ccccc3CO)nc2n(C)c1=O] |
| Ia-37 | 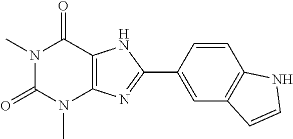 [SMILES: Cn1c(=O)c2[nH]c(-c3ccc4[nH]ccc4c3)nc2n(C)c1=O] |
| Ia-38 | 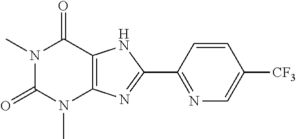 [SMILES: Cn1c(=O)c2[nH]c(-c3ccc(C(F)(F)F)cn3)nc2n(C)c1=O] |
| Ia-39 | 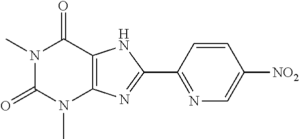 [SMILES: Cn1c(=O)c2[nH]c(-c3ccc([N+](=O)[O-])cn3)nc2n(C)c1=O] |
| Ia-40 | 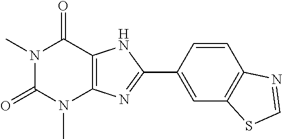 [SMILES: Cn1c(=O)c2[nH]c(-c3ccc4ncsc4c3)nc2n(C)c1=O] |
| Ia-41 | 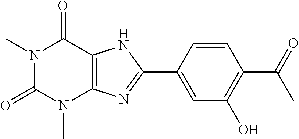 [SMILES: CC(=O)c1ccc(-c2nc3c([nH]2)c(=O)n(C)c(=O)n3C)cc1O] |
| Ia-42 | 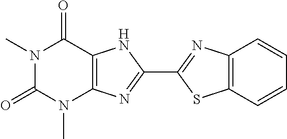 [SMILES: Cn1c(=O)c2[nH]c(-c3nc4ccccc4s3)nc2n(C)c1=O] |
| Ia-43 | 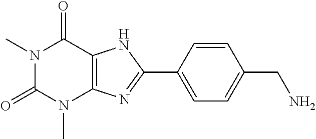 [SMILES: Cn1c(=O)c2[nH]c(-c3ccc(CN)cc3)nc2n(C)c1=O] |
| Ia-44 | 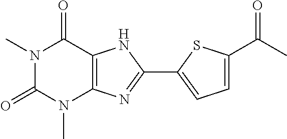 [SMILES: CC(=O)c1ccc(-c2nc3c([nH]2)c(=O)n(C)c(=O)n3C)s1] |
| Ia-45 | 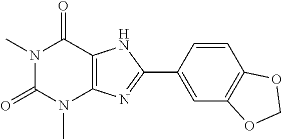 [SMILES: Cn1c(=O)c2[nH]c(-c3ccc4c(c3)OCO4)nc2n(C)c1=O] |
| I-46 | 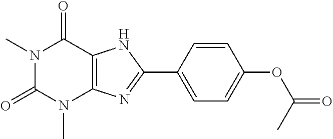 [SMILES: CC(=O)Oc1ccc(-c2nc3c([nH]2)c(=O)n(C)c(=O)n3C)cc1] |
| Ia-47 | 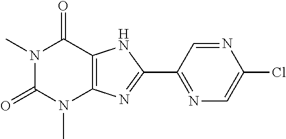 [SMILES: Cn1c(=O)c2[nH]c(-c3cnc(Cl)cn3)nc2n(C)c1=O] |
| I-48 | 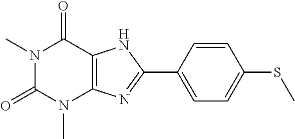 |
| I-49 | 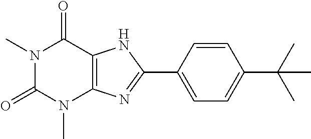 [SMILES: Cn1c(=O)c2[nH]c(-c3ccc(C(C)(C)C)cc3)nc2n(C)c1=O] |
| Ia-50 | 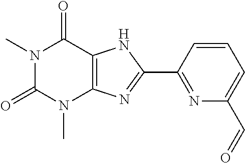 [SMILES: Cn1c(=O)c2[nH]c(-c3cccc(C=O)n3)nc2n(C)c1=O] |
| Ia-51 | 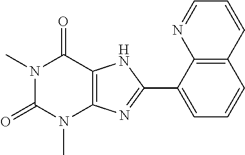 [SMILES: Cn1c(=O)c2[nH]c(-c3cccc4cccnc34)nc2n(C)c1=O] |
| I-52 | 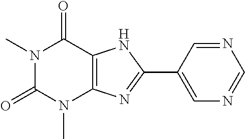 [SMILES: Cn1c(=O)c2[nH]c(-c3cncnc3)nc2n(C)c1=O] |
| Ia-53 | 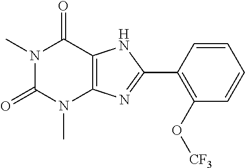 [SMILES: Cn1c(=O)c2[nH]c(-c3ccccc3OC(F)(F)F)nc2n(C)c1=O] |
| I-54 | 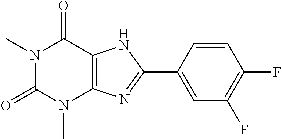 [SMILES: Cn1c(=O)c2[nH]c(-c3ccc(F)c(F)c3)nc2n(C)c1=O] |
| Ia-55 | 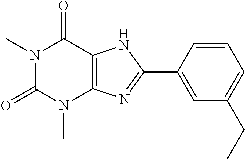 [SMILES: CCc1cccc(-c2nc3c([nH]2)c(=O)n(C)c(=O)n3C)c1] |
| Ia-56 | 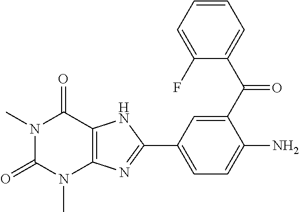 [SMILES: Cn1c(=O)c2[nH]c(-c3ccc(N)c(C(=O)c4ccccc4F)c3)nc2n(C)c1=O] |
| Ia-57 | 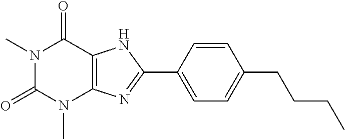 [SMILES: CCCCc1ccc(-c2nc3c([nH]2)c(=O)n(C)c(=O)n3C)cc1] |
| Ia-58 | 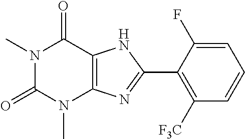 [SMILES: Cn1c(=O)c2[nH]c(-c3c(F)cccc3C(F)(F)F)nc2n(C)c1=O] |
| Ia-59 | 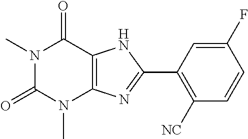 [SMILES: Cn1c(=O)c2[nH]c(-c3cc(F)ccc3C#N)nc2n(C)c1=O] |
| Ia-60 | 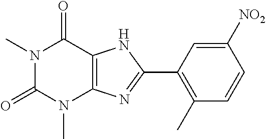 [SMILES: Cc1ccc([N+](=O)[O-])cc1-c1nc2c([nH]1)c(=O)n(C)c(=O)n2C] |
| Ia-61 | 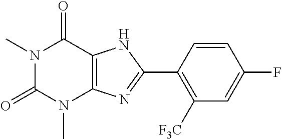 [SMILES: Cn1c(=O)c2[nH]c(-c3ccc(F)cc3C(F)(F)F)nc2n(C)c1=O] |
| Ia-62 | 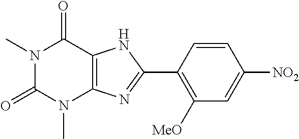 [SMILES: COc1cc([N+](=O)[O-])ccc1-c1nc2c([nH]1)c(=O)n(C)c(=O)n2C] |
| Ia-63 | 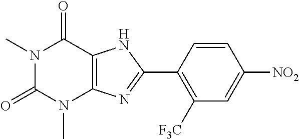 [SMILES: Cn1c(=O)c2[nH]c(-c3ccc([N+](=O)[O-])cc3C(F)(F)F)nc2n(C)c1=O] |
| Ia-64 | 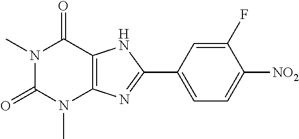 [SMILES: Cn1c(=O)c2[nH]c(-c3ccc([N+](=O)[O-])c(F)c3)nc2n(C)c1=O] |
| Ia-65 | 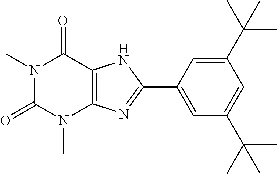 [SMILES: Cn1c(=O)c2[nH]c(-c3cc(C(C)(C)C)cc(C(C)(C)C)c3)nc2n(C)c1=O] |
| Ia-66 | 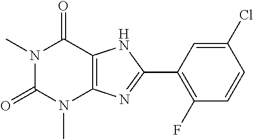 [SMILES: Cn1c(=O)c2[nH]c(-c3cc(Cl)ccc3F)nc2n(C)c1=O] |
| Ia-67 | 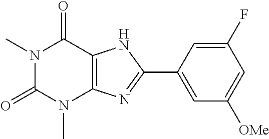 [SMILES: COc1cc(F)cc(-c2nc3c([nH]2)c(=O)n(C)c(=O)n3C)c1] |
| Ia-68 | 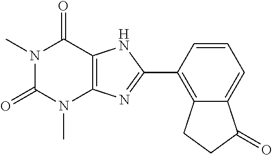 [SMILES: Cn1c(=O)c2[nH]c(-c3cccc4c3CCC4=O)nc2n(C)c1=O] |
| Ia-69 | 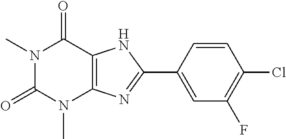 [SMILES: Cn1c(=O)c2[nH]c(-c3ccc(Cl)c(F)c3)nc2n(C)c1=O] |
| Ia-70 | 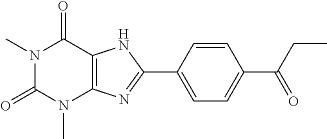 [SMILES: CCC(=O)c1ccc(-c2nc3c([nH]2)c(=O)n(C)c(=O)n3C)cc1] |
| Ia-71 | 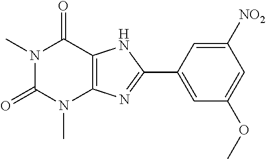 [SMILES: COc1cc(-c2nc3c([nH]2)c(=O)n(C)c(=O)n3C)cc([N+](=O)[O-])c1] |
| Ia-72 | 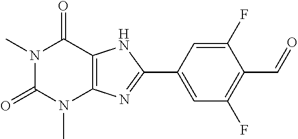 [SMILES: Cn1c(=O)c2[nH]c(-c3cc(F)c(C=O)c(F)c3)nc2n(C)c1=O] |
| Ia-73 | 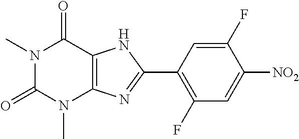 [SMILES: Cn1c(=O)c2[nH]c(-c3cc(F)c([N+](=O)[O-])cc3F)nc2n(C)c1=O] |
| Ia-74 | 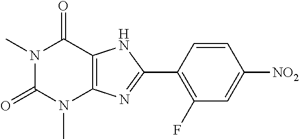 [SMILES: Cn1c(=O)c2[nH]c(-c3ccc([N+](=O)[O-])cc3F)nc2n(C)c1=O] |
| Ia-75 | 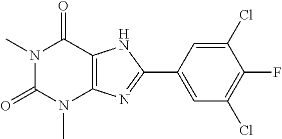 [SMILES: Cn1c(=O)c2[nH]c(-c3cc(Cl)c(F)c(Cl)c3)nc2n(C)c1=O] |
| Ia-76 | 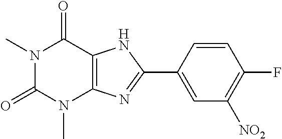 [SMILES: Cn1c(=O)c2[nH]c(-c3ccc(F)c([N+](=O)[O-])c3)nc2n(C)c1=O] |
| Ia-77 | 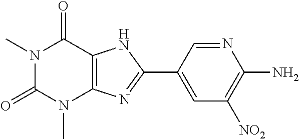 [SMILES: Cn1c(=O)c2[nH]c(-c3cnc(N)c([N+](=O)[O-])c3)nc2n(C)c1=O] |
| Ia-78 | 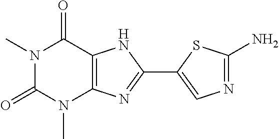 [SMILES: Cn1c(=O)c2[nH]c(-c3cnc(N)s3)nc2n(C)c1=O] |
| Ia-79 | 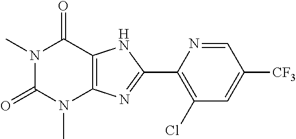 [SMILES: Cn1c(=O)c2[nH]c(-c3ncc(C(F)(F)F)cc3Cl)nc2n(C)c1=O] |
| Ia-80 | 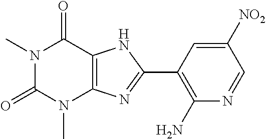 [SMILES: Cn1c(=O)c2[nH]c(-c3cc([N+](=O)[O-])cnc3N)nc2n(C)c1=O] |
| Ia-81 | 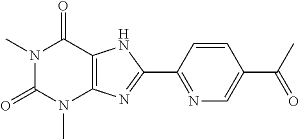 [SMILES: CC(=O)c1ccc(-c2nc3c([nH]2)c(=O)n(C)c(=O)n3C)nc1] |
| Ia-82 | 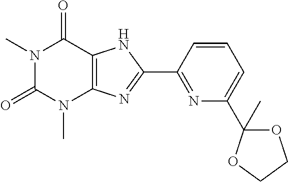 [SMILES: Cn1c(=O)c2[nH]c(-c3cccc(C4(C)OCCO4)n3)nc2n(C)c1=O] |
| Ia-83 | 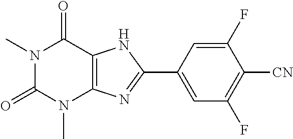 [SMILES: Cn1c(=O)c2[nH]c(-c3cc(F)c(C#N)c(F)c3)nc2n(C)c1=O] |
| Ia-84 | 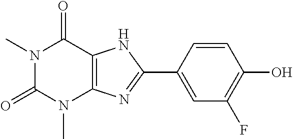 [SMILES: Cn1c(=O)c2[nH]c(-c3ccc(O)c(F)c3)nc2n(C)c1=O] |
| Ia-85 | 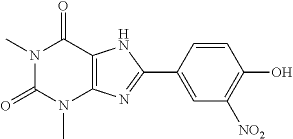 [SMILES: Cn1c(=O)c2[nH]c(-c3ccc(O)c([N+](=O)[O-])c3)nc2n(C)c1=O] |
| I-86 | 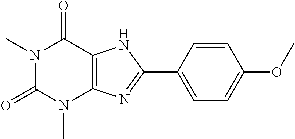 |
| Ia-87 | 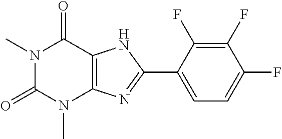 [SMILES: Cn1c(=O)c2[nH]c(-c3ccc(F)c(F)c3F)nc2n(C)c1=O] |
| Ia-88 | 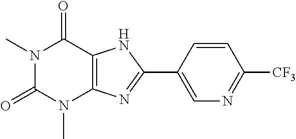 [SMILES: Cn1c(=O)c2[nH]c(-c3ccc(C(F)(F)F)nc3)nc2n(C)c1=O] |
| I-89 | 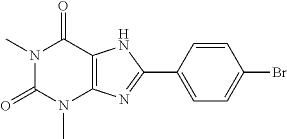 [SMILES: Cn1c(=O)c2[nH]c(-c3ccc(Br)cc3)nc2n(C)c1=O] |
| I-90 | 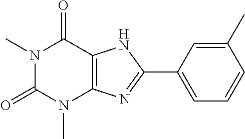 |
| I-91 | 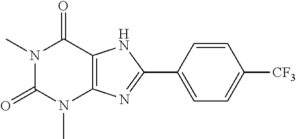 [SMILES: Cn1c(=O)c2[nH]c(-c3ccc(C(F)(F)F)cc3)nc2n(C)c1=O] |
| I-92 | 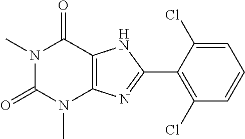 [SMILES: Cn1c(=O)c2[nH]c(-c3c(Cl)cccc3Cl)nc2n(C)c1=O] |
| Ia-93 | 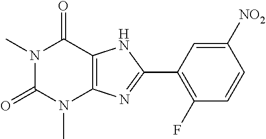 [SMILES: Cn1c(=O)c2[nH]c(-c3cc([N+](=O)[O-])ccc3F)nc2n(C)c1=O] |
| Ia-94 | 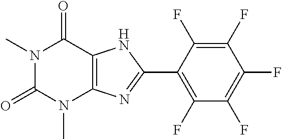 [SMILES: Cn1c(=O)c2[nH]c(-c3c(F)c(F)c(F)c(F)c3F)nc2n(C)c1=O] |
| I-95 | 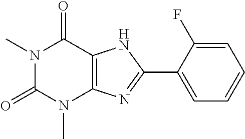 [SMILES: Cn1c(=O)c2[nH]c(-c3ccccc3F)nc2n(C)c1=O] |
| I-96 | 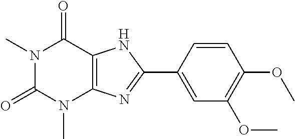 |
| Ia-97 | 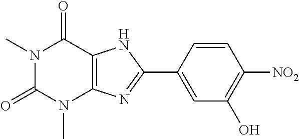 [SMILES: Cn1c(=O)c2[nH]c(-c3ccc([N+](=O)[O-])c(O)c3)nc2n(C)c1=O] |
| I-98 | 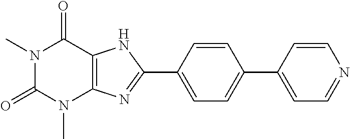 [SMILES: Cn1c(=O)c2[nH]c(-c3ccc(-c4ccncc4)cc3)nc2n(C)c1=O] |
| Ia-99 | 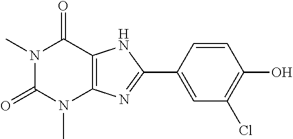 [SMILES: Cn1c(=O)c2[nH]c(-c3ccc(O)c(Cl)c3)nc2n(C)c1=O] |
| Ia-100 | 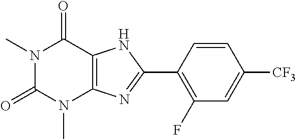 [SMILES: Cn1c(=O)c2[nH]c(-c3ccc(C(F)(F)F)cc3F)nc2n(C)c1=O] |
| I-101 | 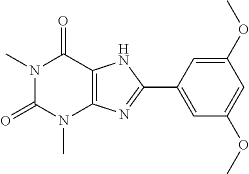 [SMILES: COc1cc(OC)cc(-c2nc3c([nH]2)c(=O)n(C)c(=O)n3C)c1] |
| Ia-102 | 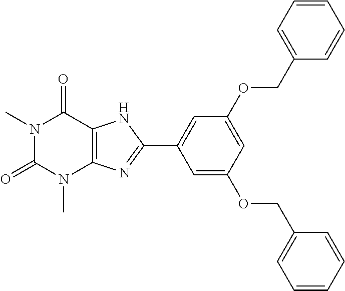 [SMILES: Cn1c(=O)c2[nH]c(-c3cc(OCc4ccccc4)cc(OCc4ccccc4)c3)nc2n(C)c1=O] |
| Ia-103 | 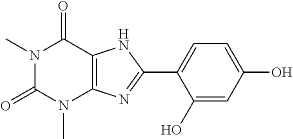 [SMILES: Cn1c(=O)c2[nH]c(-c3ccc(O)cc3O)nc2n(C)c1=O] |
| Ia-104 | 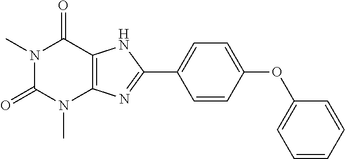 [SMILES: Cn1c(=O)c2[nH]c(-c3ccc(Oc4ccccc4)cc3)nc2n(C)c1=O] |
| Ia-105 | 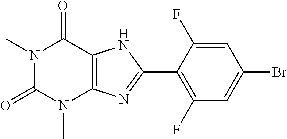 [SMILES: Cn1c(=O)c2[nH]c(-c3c(F)cc(Br)cc3F)nc2n(C)c1=O] |
| Ia-106 | 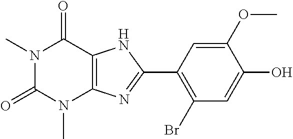 |
| I-107 | 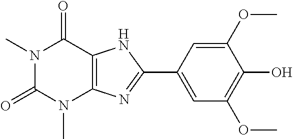 |
| Ia-108 | 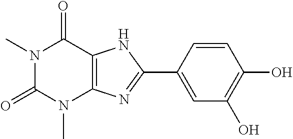 [SMILES: Cn1c(=O)c2[nH]c(-c3ccc(O)c(O)c3)nc2n(C)c1=O] |
| I-109 | 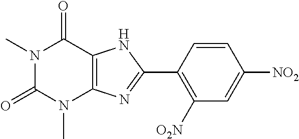 [SMILES: Cn1c(=O)c2[nH]c(-c3ccc([N+](=O)[O-])cc3[N+](=O)[O-])nc2n(C)c1=O] |
| Ia-110 | 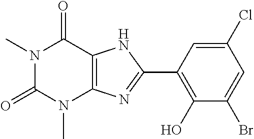 [SMILES: Cn1c(=O)c2[nH]c(-c3cc(Cl)cc(Br)c3O)nc2n(C)c1=O] |
| Ia-111 | 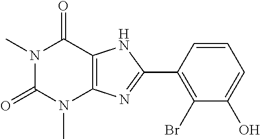 [SMILES: Cn1c(=O)c2[nH]c(-c3cccc(O)c3Br)nc2n(C)c1=O] |
| I-112 | 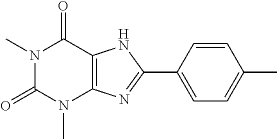 |
| Ia-113 | 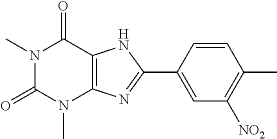 |
| I-114 | 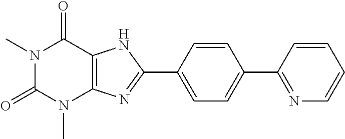 [SMILES: Cn1c(=O)c2[nH]c(-c3ccc(-c4ccccn4)cc3)nc2n(C)c1=O] |
| Ia-115 | 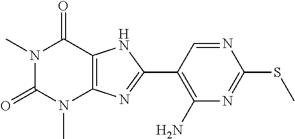 |
| Ia-116 | 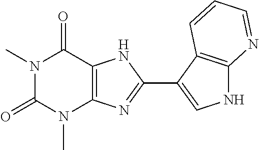 [SMILES: Cn1c(=O)c2[nH]c(-c3c[nH]c4ncccc34)nc2n(C)c1=O] |
| Ia-117 | 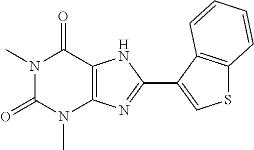 [SMILES: Cn1c(=O)c2[nH]c(-c3csc4ccccc34)nc2n(C)c1=O] |
| I-118 | 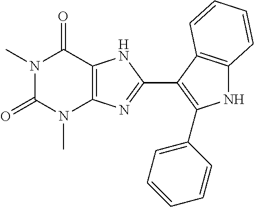 [SMILES: Cn1c(=O)c2[nH]c(-c3c(-c4ccccc4)[nH]c4ccccc34)nc2n(C)c1=O] |
| I-119 | 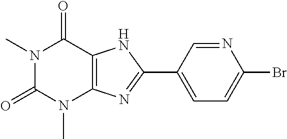 [SMILES: Cn1c(=O)c2[nH]c(-c3ccc(Br)nc3)nc2n(C)c1=O] |
| Ia-120 | 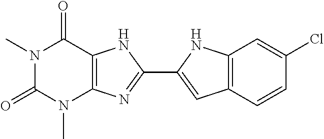 [SMILES: Cn1c(=O)c2[nH]c(-c3cc4ccc(Cl)cc4[nH]3)nc2n(C)c1=O] |
| Ia-121 | 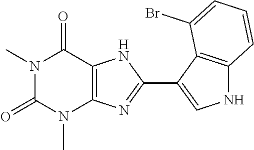 [SMILES: Cn1c(=O)c2[nH]c(-c3c[nH]c4cccc(Br)c34)nc2n(C)c1=O] |
| I-122 | 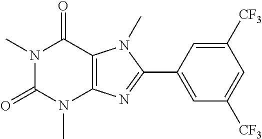 [SMILES: Cn1c(=O)c2c(nc(-c3cc(C(F)(F)F)cc(C(F)(F)F)c3)n2C)n(C)c1=O] |
| Ia-123 | 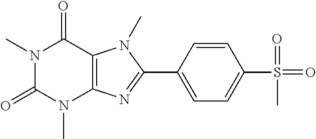 [SMILES: Cn1c(=O)c2c(nc(-c3ccc(S(C)(=O)=O)cc3)n2C)n(C)c1=O] |
| Ia-124 | 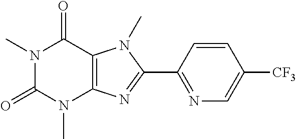 [SMILES: Cn1c(=O)c2c(nc(-c3ccc(C(F)(F)F)cn3)n2C)n(C)c1=O] |
| I-125 | 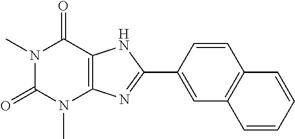 [SMILES: Cn1c(=O)c2[nH]c(-c3ccc4ccccc4c3)nc2n(C)c1=O] |
| I-126 | 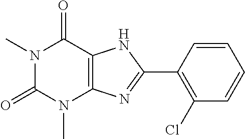 [SMILES: Cn1c(=O)c2[nH]c(-c3ccccc3Cl)nc2n(C)c1=O] |
| Ia-127 | 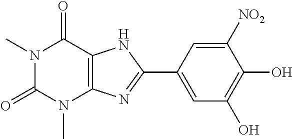 [SMILES: Cn1c(=O)c2[nH]c(-c3cc(O)c(O)c([N+](=O)[O-])c3)nc2n(C)c1=O] |
| I-128 | 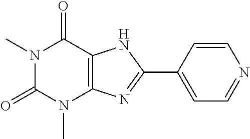 [SMILES: Cn1c(=O)c2[nH]c(-c3ccncc3)nc2n(C)c1=O] |
| Ia-129 | 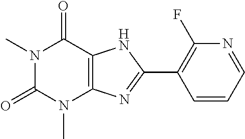 [SMILES: Cn1c(=O)c2[nH]c(-c3cccnc3F)nc2n(C)c1=O] |
| Ia-130 | 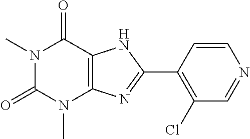 [SMILES: Cn1c(=O)c2[nH]c(-c3ccncc3Cl)nc2n(C)c1=O] |
| I-131 | 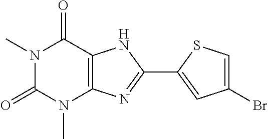 [SMILES: Cn1c(=O)c2[nH]c(-c3cc(Br)cs3)nc2n(C)c1=O] |
| Ia-132 | 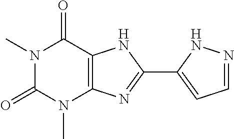 [SMILES: Cn1c(=O)c2[nH]c(-c3ccn[nH]3)nc2n(C)c1=O] |
| I-133 | 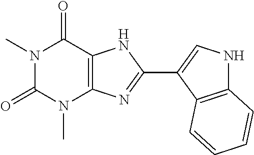 [SMILES: Cn1c(=O)c2[nH]c(-c3c[nH]c4ccccc34)nc2n(C)c1=O] |
| Ia-134 | 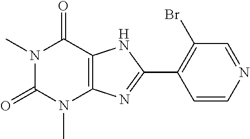 [SMILES: Cn1c(=O)c2[nH]c(-c3ccncc3Br)nc2n(C)c1=O] |
| Ia-135 | 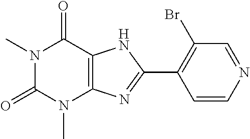 [SMILES: Cn1c(=O)c2[nH]c(-c3ccncc3Br)nc2n(C)c1=O] |
| Ia-136 | 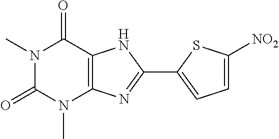 [SMILES: Cn1c(=O)c2[nH]c(-c3ccc([N+](=O)[O-])s3)nc2n(C)c1=O] |
| Ia-137 | 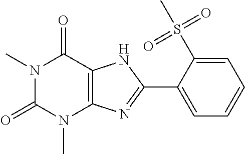 [SMILES: Cn1c(=O)c2[nH]c(-c3ccccc3S(C)(=O)=O)nc2n(C)c1=O] |
| Ia-138 | 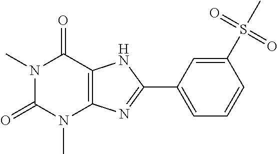 [SMILES: Cn1c(=O)c2[nH]c(-c3cccc(S(C)(=O)=O)c3)nc2n(C)c1=O] |
| Ia-139 | 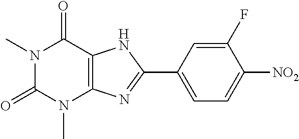 [SMILES: Cn1c(=O)c2[nH]c(-c3ccc([N+](=O)[O-])c(F)c3)nc2n(C)c1=O] |
| Ia-140 | 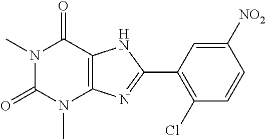 [SMILES: Cn1c(=O)c2[nH]c(-c3cc([N+](=O)[O-])ccc3Cl)nc2n(C)c1=O] |
| I-141 | 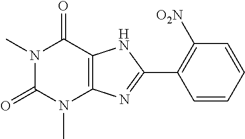 [SMILES: Cn1c(=O)c2[nH]c(-c3ccccc3[N+](=O)[O-])nc2n(C)c1=O] |
| Ia-142 | 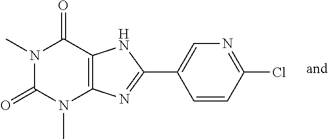 [SMILES: Cn1c(=O)c2[nH]c(-c3ccc(Cl)nc3)nc2n(C)c1=O] |
| I-143 | 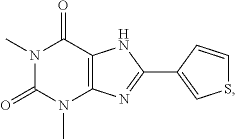 [SMILES: Cn1c(=O)c2[nH]c(-c3ccsc3)nc2n(C)c1=O] |

or pharmaceutically acceptable salt, solvate and/or prodrug thereof.

In some embodiments, the compound of Formula (I) is selected from:

| Compound | |
|---|---|
| No. | Structure |
| Ia-1 | 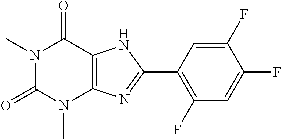 [SMILES: Cn1c(=O)c2[nH]c(-c3cc(F)c(F)cc3F)nc2n(C)c1=O] |
| Ia-2 | 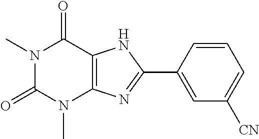 [SMILES: Cn1c(=O)c2[nH]c(-c3cccc(C#N)c3)nc2n(C)c1=O] |
| Ia-3 | 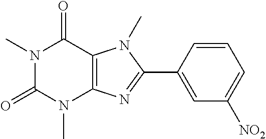 [SMILES: Cn1c(=O)c2c(nc(-c3cccc([N+](=O)[O-])c3)n2C)n(C)c1=O] |
| Ia-4 | 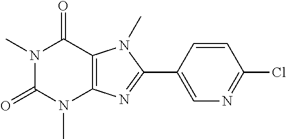 [SMILES: Cn1c(=O)c2c(nc(-c3ccc(Cl)nc3)n2C)n(C)c1=O] |
| Ia-5 | 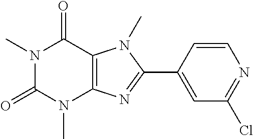 [SMILES: Cn1c(=O)c2c(nc(-c3ccnc(Cl)c3)n2C)n(C)c1=O] |
| Ia-6 | 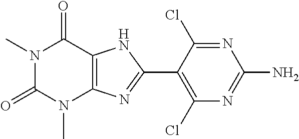 [SMILES: Cn1c(=O)c2[nH]c(-c3c(Cl)nc(N)nc3Cl)nc2n(C)c1=O] |
| Ia-7 | 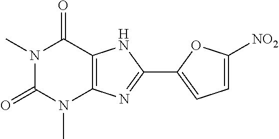 [SMILES: Cn1c(=O)c2[nH]c(-c3ccc([N+](=O)[O-])o3)nc2n(C)c1=O] |
| Ia-8 | 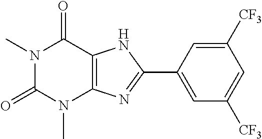 [SMILES: Cn1c(=O)c2[nH]c(-c3cc(C(F)(F)F)cc(C(F)(F)F)c3)nc2n(C)c1=O] |
| Ia-9 | 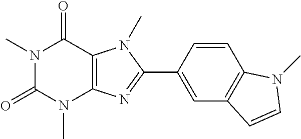 |
| Ia-10 | 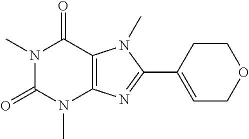 [SMILES: Cn1c(=O)c2c(nc(C3=CCOCC3)n2C)n(C)c1=O] |
| Ia-11 | 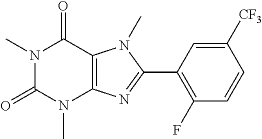 [SMILES: Cn1c(=O)c2c(nc(-c3cc(C(F)(F)F)ccc3F)n2C)n(C)c1=O] |
| Ia-12 | 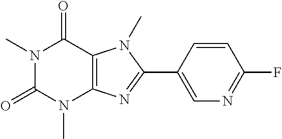 [SMILES: Cn1c(=O)c2c(nc(-c3ccc(F)nc3)n2C)n(C)c1=O] |
| Ia-13 | 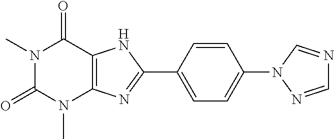 [SMILES: Cn1c(=O)c2[nH]c(-c3ccc(-n4cncn4)cc3)nc2n(C)c1=O] |
| Ia-14 | 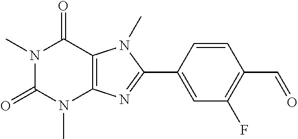 [SMILES: Cn1c(=O)c2c(nc(-c3ccc(C=O)c(F)c3)n2C)n(C)c1=O] |
| Ia-15 | 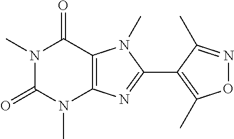 [SMILES: Cc1noc(C)c1-c1nc2c(c(=O)n(C)c(=O)n2C)n1C] |
| Ia-16 | 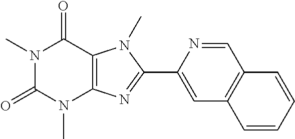 [SMILES: Cn1c(=O)c2c(nc(-c3cc4ccccc4cn3)n2C)n(C)c1=O] |
| Ia-17 | 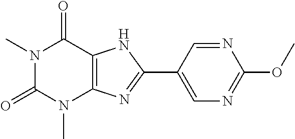 |
| Ia-18 | 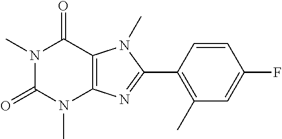 [SMILES: Cc1cc(F)ccc1-c1nc2c(c(=O)n(C)c(=O)n2C)n1C] |
| Ia-19 | 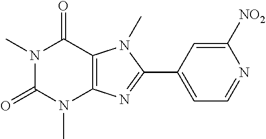 [SMILES: Cn1c(=O)c2c(nc(-c3ccnc([N+](=O)[O-])c3)n2C)n(C)c1=O] |
| Ia-20 | 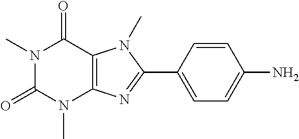 [SMILES: Cn1c(=O)c2c(nc(-c3ccc(N)cc3)n2C)n(C)c1=O] |
| I-1 | 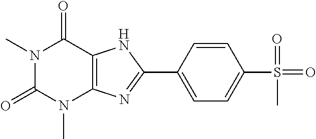 [SMILES: Cn1c(=O)c2[nH]c(-c3ccc(S(C)(=O)=O)cc3)nc2n(C)c1=O] |
| I-2 | 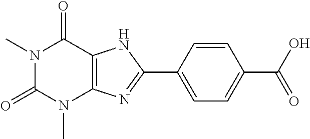 [SMILES: Cn1c(=O)c2[nH]c(-c3ccc(C(=O)O)cc3)nc2n(C)c1=O] |
| I-5 | 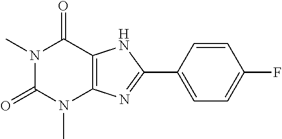 [SMILES: Cn1c(=O)c2[nH]c(-c3ccc(F)cc3)nc2n(C)c1=O] |
| I-6 | 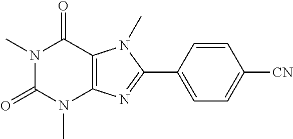 [SMILES: Cn1c(=O)c2c(nc(-c3ccc(C#N)cc3)n2C)n(C)c1=O] |
| I-7 | 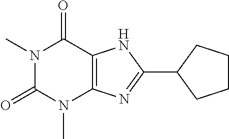 [SMILES: Cn1c(=O)c2[nH]c(C3CCCC3)nc2n(C)c1=O] |
| I-10 | 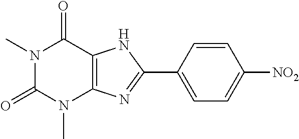 [SMILES: Cn1c(=O)c2[nH]c(-c3ccc([N+](=O)[O-])cc3)nc2n(C)c1=O] |
| 11 | 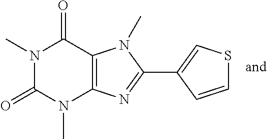 [SMILES: Cn1c(=O)c2c(nc(-c3ccsc3)n2C)n(C)c1=O] |
| 12 | 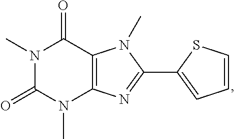 [SMILES: Cn1c(=O)c2c(nc(-c3cccs3)n2C)n(C)c1=O] |

or a pharmaceutically acceptable salt, solvate and/or prodrug thereof.

The present application also includes a method of treating a disease, disorder or condition treatable by blocking SREBP2 activation and/or PCSK9 gene expression, the method comprising administering an effect amount of one or more compound I-8

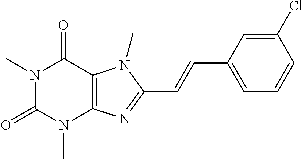 [SMILES: Cn1c(=O)c2c(nc(/C=C/c3cccc(Cl)c3)n2C)n(C)c1=O]

or a pharmaceutically acceptable salt, solvate and/or prodrug thereof, to a subject in need thereof.

In some embodiments, the disease, disorder or condition is caused and/or exacerbated by increased SREBP2 and/or PCSK9 function or activity.

In some embodiments, the disease, disorder or condition is elevated cholesterol levels, liver disease or chronic kidney disease. In some embodiments, elevated cholesterol levels are associated with cardiovascular disease. In some embodiments, the liver disease is NAFLD or NASH.

In some embodiments, the therapeutically effective amount of the one or more compounds is administered in combination with one or more other therapeutic agents. When used in combination with other agents or therapies, it is an embodiment that the compounds of the application are administered contemporaneously with those agents or therapies. As used herein, “contemporaneous administration” of two substances or therapies to a subject means providing each of the two substances or therapies so that they are both biologically active in the individual at the same time. The exact details of the administration will depend on the pharmacokinetics of the two substances or therapies in the presence of each other, and can include administering the two substances or therapies within a few hours of each other, or even administering one substance or therapy within 24 hours of administration of the other, if the pharmacokinetics are suitable. Design of suitable dosing regimens is routine for one skilled in the art. In some embodiments, the substances or therapies will be administered substantially simultaneously, i.e., within minutes of each other, or in a single composition in the case of administration of two substances. It is a further embodiment of the present application that a combination of agents or therapies is administered to a subject in a non-contemporaneous fashion.

Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom.

In some embodiments, the subject is a mammal. In some embodiments, the subject is a human.

The present application also includes a method of blocking SREBP2 activation and/or PCSK9 gene expression in a cell, either in a biological sample or in a subject, comprising administering a therapeutically effective amount of one or more compounds of Formula (I), or a pharmaceutically acceptable salt, solvate and/or prodrug thereof, to a cell in need thereof.

Also provided is a method of increasing endoplasmic reticulum calcium levels in a cell, either in a biological sample or in a subject, comprising administering a therapeutically effective amount of one or more compounds of Formula (I), or a pharmaceutically acceptable salt, solvate and/or prodrug thereof, to a cell in need thereof.

Further provided is a method of lowering serum LDL cholesterol levels comprising administering a therapeutically effective amount of one or more compounds of Formula (I), or a pharmaceutically acceptable salt, solvate and/or prodrug thereof, to a subject in need thereof.

The application also includes a method of treating or preventing a disease, disorder or condition treatable by lowering serum LDL cholesterol levels comprising administering a therapeutically effective amount of one or more compounds of Formula (I), or a pharmaceutically acceptable salt, solvate and/or prodrug thereof, to a subject in need thereof.

In some embodiments, serum LDL cholesterol levels are lowered compared to pre-dose serum LDL cholesterol levels in the subject.

Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom.

In some embodiments, the therapeutically effective amount of the one or more compounds is administered in combination with one or more other therapeutic agents.

In some embodiments, the one or more other therapeutic agents elevates serum LDLR cholesterol levels. In some embodiments, the one or more other therapeutic agents lowers serum LDL cholesterol levels.

In some embodiments, the one or more other therapeutic agents is a statin. The statin may be selected from, but not limited to, the group consisting of atorvastatin, fluvastatin, lovastatin, mevastatin, pitavastatin, pravastatin, rosuvastatin and simvastatin, and combinations thereof.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Compounds of the application may be administered in a single daily, weekly or monthly dose or the total daily dose may be divided into two, three or four daily doses.

In some embodiments, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

Also provided is a use of one or more compounds of the application for blocking SREBP2 activation and/or PCSK9 gene expression, for treating a disease, disorder or condition treatable by blocking SREBP2 activation and/or PCSK9 gene expression as well as for the preparation of a medicament for treating a disease, disorder or condition treatable by blocking SREBP2 activation and/or PCSK9 gene expression.

### (c) Compositions of the Application

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

A compound of the application including salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the composition will comprise from about 0.05 wt % to about 99 wt % or about 0.10 wt % to about 70 wt %, of the active ingredient, and from about 1 wt % to about 99.95 wt % or about 30 wt % to about 99.90 wt % of an acceptable carrier, all percentages by weight being based on the total composition.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, tale or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In an embodiment, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

In some embodiments, depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of one or more pharmaceutically acceptable carriers, all percentages by weight being based on the total composition.

In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g. a compound of Formula (I) or (Ia)), an additional therapeutic agent, and a pharmaceutically acceptable carrier. In some embodiments, the additional therapeutic agents is one or more other cholesterol lowering agents.

To be clear, in the above, the term “a compound” also includes embodiments wherein one or more compounds are referenced.

### III. Methods of Preparation

Compounds of the present application can be prepared by various synthetic processes. The choice of particular structural features and/or substituents may influence the selection of one process over another. The selection of a particular process to prepare a given compound the application is within the purview of the person of skill in the art. Some starting materials for preparing compounds of the present application are available from commercial chemical sources. Other starting materials, for example as described below, are readily prepared from available precursors using straightforward transformations that are well known in the art. In the Schemes below showing the preparation of compounds of the application, all variables are as defined in Formula (I), unless otherwise stated.

The compounds of the application generally can be prepared according to the processes illustrated in the Schemes below. In the structural formulae shown below the variables are as defined in Formula (I) or (Ia) unless otherwise stated. A person skilled in the art would appreciate that many of the reactions depicted in the Schemes below would be sensitive to oxygen and water and would know to perform the reaction under an anhydrous, inert atmosphere if needed. Reaction temperatures and times are presented for illustrative purposes only and may be varied to optimize yield as would be understood by a person skilled in the art.

Accordingly, in some embodiments, the compounds of Formula (I) or (Ia), wherein R² is H are prepared as shown in Scheme 1:

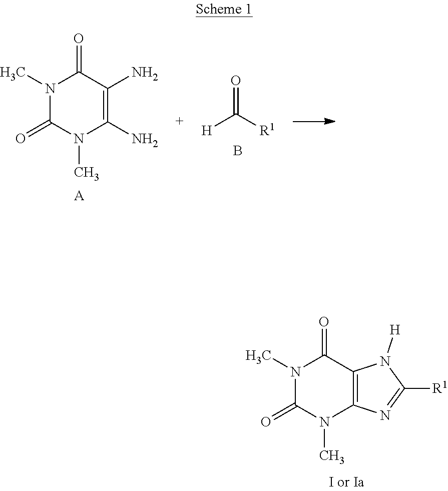

Therefore, in some embodiments, 5,6-diamino-1,3-dimethyluracil of Formula A (1 equiv), a compound of Formula B (1 equiv), azobisisobutyronitrile (AIBN, 0.02 equiv), and N-bromosuccinimide (0.7 equiv) are combined in a suitable solvent and reacted under conditions to provide the compounds of Formula (I) or (Ia). In some embodiments, the conditions comprise stirring the combined compounds at a temperature of about 10° C. to about 60° C. for about 1 hour to about 12 hours.

In some embodiments, the compounds of Formula (I) or (Ia), wherein R² is CH₃ are prepared as shown in Scheme 2:

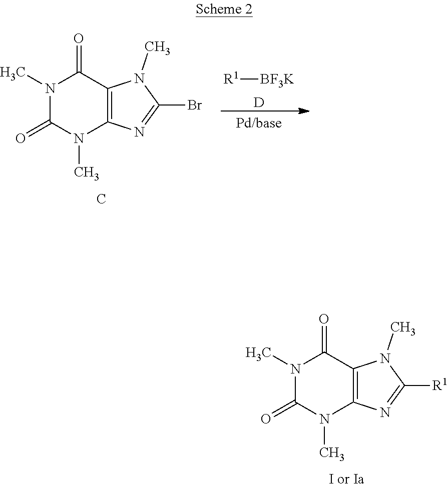

Therefore in some embodiments, 8-bromocaffeine of Formula C (1 equiv), potassium trifluoroborate salt of Formula D (1.5 equiv), a suitable inorganic base such as K₂CO₃ (13 equiv), and a suitable Pd catalyst such as Pd(amphos)Cl₂ (5 mol %,) are combine in a suitable solvent and reacted under conditions to provide the compounds of Formula (I) or (Ia). In some embodiments, the conditions comprise reacting the combined reagents in Monowave reactor for about 10 minutes to about 90 minutes at a temperature of about 100° C. to about 200° C.

In some embodiments, the compounds of Formula (I) or (Ia), wherein R² is CH₃ and are prepared as shown in Scheme 3.

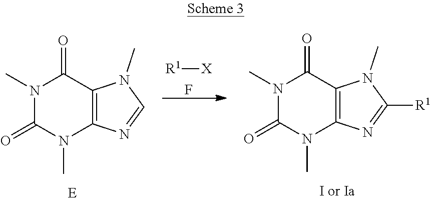

Therefore, in some embodiments, a compound of Formula E (caffeine), is combined with a suitable compound of Formula F wherein X is a suitable leaving group such as halide (e.g Br) under suitable cross coupling conditions such as in the presence of a suitable catalyst such as a palladium catalyst, (e.g Pd(OAc)₂) suitable base such as a cesium salts (e.g. Cs₂CO₃) and suitable ligand such as tricyclohexylphosphonium tetrafluoroborate (Cy₃P·HBF₄) in a suitable solvent such as polyvinyl alcohol (PvOH) to provide the compound of Formula I or Ia.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C1-C24) esters, acyloxymethyl esters, carbamates and amino acid esters.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### EXAMPLES

The following non-limiting examples are illustrative of the present application:

### Example 1. Effects of Caffeine on PCSK9

### Materials and Methods

### Cell Culture, Treatments and Transfections

HuH7 and HepG2 cells were routinely grown in complete Dulbecco's Modified Eagle's Medium (Gibco, Thermofisher Scientific) supplemented with 10% fetal bovine serum (Sigma-Aldrich) and 100 U/ml of penicillin and streptomycin (Sigma-Aldrich). Caffeine (CF), ryanodine, 2 APB, CDN, theobromine, paraxanthine, 8-cyclopentyl-1,3-dimethylxanthine (8CD), 8-(3-Chlorostyryl) CF (8CC), PSB603, cyclopiazonic acid and U18666A were purchased from Tocris Bioscience. All cell treatments were carried out for 24 h unless otherwise stated. Cells were transfected with a cocktail consisting of plasmid DNA (1 μg), X-tremeGENE HP (3 μl; Thermofisher Scientific) and opti-MEM (100 μl; Thermofischer Scientific) per 1 ml complete medium containing plated cells. Human PCSK9 was overexpressed using the bicistronic pIRES-EGFP plasmid; calnexin using the mPA-GFP-N1 plasmid. To block the expression of GRP78 and CD36, siGENOME smartpool siRNA was purchased from GE Dharmacon (M-008198-02 and L-010206-00-0005 respectively) and transfected using lipofectamine RNAiMAX as per manufacturer's protocol.

### Ca²⁺ Studies: Fluorogenic Dyes and Genetically Encoded FRET-Based Sensors

Intracellular Ca²⁺ in Huh7 and HepG2 cells was measured using a high-affinity Ca²⁺ indicator, Fura-2-AM (Thermofisher Scientific). ER Ca²⁺ levels were assessed using the low-affinity Ca²⁺ indicator, Mag-Fluo-4, and via transfection of cells with D1ER. The D1ER plasmid encodes an ER-resident calcium binding protein linked to a fluorescent protein and increases in fluorescence intensity upon Ca²⁺ binding (7). For assessment using indicators, cells were plated in black clear-bottom 96-well plates to a confluence of 70-75% and treated with Ca²⁺ modulating agents for 24 h (n=6). Cells were then washed and incubated with Fura-2-AM (2 μM) or Mag-Fluo-4 (2 μM) for 45 minutes at 37° C. in HBSS containing 20 mM HEPES and 2% pluronic acid v/v (Thermofisher Scientific). Fluorescence intensity of intracellular Fura-2-AM was measured at two distinct wavelengths (ex 340/em 515 and ex 380/em 515), following three consecutive washes, to assess bound and unbound states using a SpectraMax® GeminiEM fluorescent spectrophotometer (Molecular Devices, Sunnyvale, California, USA). Fluorescence intensity of Mag-Fluo-4 was quantified at a single wavelength (ex 495/em 515). For assessment using D1ER, HuH7 cells were plated in black clear-bottom 96-well plates to a confluence of 70-75% and transfected (n=6). Twenty-four h later, cells were treated with Ca²⁺ modulating agents for an additional 24 h and quantified (ex 495/em 515) using a SpectraMax® GeminiEM fluorescent spectrophotometer.

### Immunoblot Analysis

Cells were washed in phosphate-buffered saline (PBS), lysed in 4× SDS-PAGE lysis buffer and separated on 7-10% polyacrylamide gels in denaturing conditions. Gels were transferred to nitrocellulose membranes using the BioRad mini trans-blot system, blocked in 5% skim milk in tris-buffered saline (TBS) for 1 h and incubated in primary antibody overnight for 16 h at 4° C. Membranes were then exposed to horse radish peroxidase conjugated secondary antibodies and visualized using EZ-ECL chemiluminescent reagent (Froggabio). Band intensities were quantified using ImageJ software (BioRad) against membranes reprobed for housekeeping proteins. A list of antibodies used for immunoblot analysis is presented in Table 3.

### Immunoprecipitations

Cells were grown in 10 cm dishes were resuspended in ice cold non-denaturing immunoprecipitation buffer containing 20 mM tris HCL, 137 mM NaCl, 1% NP-40, 2 mM EDTA and protease inhibitor (Roche). Total cell protein was normalized using a protein assay (BioRad) and 1 mg of protein from each sample was incubated with 2 μg of capture antibody targeted against GRP78 (Santa Cruz Biotechnology; SC-1050) and rotated on a platform for 24 h at 4° C. Following incubation, samples were exposed to 100 μl of Protein G magnetic Surebeads® (BioRad) for an additional 2 h on a rotating platform at 4° C. Beads conjugated to the anti-GRP78 antibody were subsequently isolated and the remaining sample was collected and labelled “input” for use as controls. The magnetic beads underwent 4 consecutive washes using the non-denaturing IP buffer and resuspended and boiled in 100 μl of 4× SDS-PAGE sample buffer.

### Immunofluorescent Staining

Cells were plated in 4-well chamber slides and incubated in complete DMEM for 24 h and exposed to treatments 24 h later for an additional 24 h. Cells were fixed with 4% paraformaldehyde for 30 minutes and washed with either non-permeabilizing, or permeabilizing PBS containing 0.025% Triton-X. Cells were then blocked with 1% bovine serum albumin (BSA) for 30 minutes and stained with anti-GFP antibody for 1 h in PBS containing 1% BSA. Afterwards, cells were washed and incubated with Alexa 488 fluorescently-labelled secondary antibodies (Thermofisher Scientific), as well as the DAPI nuclear stain. Slides were then mounted with permafluor and visualized using the EVOS FL colour imaging system at either 20 or 40× magnification. A list of antibodies used for immunofluorescence staining is presented in Table 3.

### Thioflavin-T Staining

Following treatment, live cells were incubated in complete DMEM containing 5 μM Thioflavin-T (ThT; Thermofisher Scientific) for 15 minutes. Cells were then fixed in 4% paraformaldehyde and mounted with permafluor. Fluorescent staining was visualized using the EVOS FL colour system at either 20 or 40× magnification.

### Immunohistochemical Staining

Liver tissues were fixed in formaldehyde and subsequently embedded in paraffin for sectioning. 4 μM thick sections underwent epitope retrieval and were subsequently stained with primary antibodies for 16 h at 4° C. Slides were then exposed to biotin-conjugated secondary antibodies for 45 minutes and then streptavidin peroxidase for 10 minutes. Staining was visualized using the Nova Red HRP Substrate (Vector Laboratories). A list of antibodies used for immunohistochemical analysis is presented in Table 3.

### Quantitative Real-Time PCR

RNA purification/isolation was performed using RNeasy mini kits (Qiagen) and normalized to 2 μg RNA using a NanoDrop® spectrophotometer. Samples were then reverse transcribed into cDNA using Superscript Vilo cDNA Synthesis kit (Thermofisher Scientific). Real-time PCR was executed with Fast SYBR Green (Thermo Fisher Scientific) using the ΔΔct method on the ViiA7 real-time PCR platform (Thermofisher Scientific). A list of primers used for PCR analysis is presented in Table 4.

### ELISAs

Secreted PCSK9 levels were assessed directly in cell culture medium of cells grown in FBS-free medium for 24 h or in the serum isolated from either mice or human subjects. Mouse PCSK9 was measured using the Quantikinine® ELISA kit (#MCP900, R&D Systems) and human PCSK9 using the PCSK9 Quantikinine® ELISA kit (#DCP900, R&D Systems). Serum ApoB levels were also quantified using ELISAs (#DAPB00, R&D Systems). Serum samples were diluted as per manufacturer's instructions.

### Mouse Studies and Primary Hepatocyte Isolation

All animal studies were carried out in 8 h-fasted male wild-type, Pcsk9^{-/-} or Ampkβ1^{-/-} mice on the C57BL/6J background. CF (25-100 mg/kg-8 h) and CDN (50 mg/kg) treatments were administered via intraperitoneal injection unless specified otherwise. Primary mouse hepatocytes were isolated using a two-step process with EGTA (500 M in HEPES buffer, Sigma Aldrich) and collagenase (0.05% in HEPES buffer, Sigma Aldrich) in 12-week-old male mice on the C57BL/6J background. Cells were then washed, centrifuged, and plated following isolation in cell strainers. Hepatosure® 100-donor pooled primary human hepatocytes were purchased from Xenotech. Primary hepatocytes were regularly grown in William's E medium (Gibco, Thermo Fisher Scientific) containing 10% fetal bovine serum, 100 IU/ml penicillin, and 100 g/ml streptomycin.

### DiI-LDL Uptake Assay

Cells were seeded plated in black clear-bottom 96-well plates for 24 h and treated with experimental agents for an additional 24 h. During the last 5 h of treatment (h 19 to 24) cells were exposed to DiI-LDL (10 μg/ml) and then washed with two changes of pre-warmed (37° C.) HBSS containing 20 mM HEPES prior to analysis. The intracellular fluorescence intensity of DiI was then quantified using the SpectraMax GeminiEM fluorescent spectrophotometer (Molecular Devices; ex 554/em 571).

### CF Studies in Healthy Human Subjects

Healthy human subjects between the ages of 22 and 45 underwent fasting for 12 h prior to oral administration of 400 mg CF (˜5 mg/kg). Blood was collected prior to administration and at h 2 and 4 following administration.

### Statistics

Statistical analysis for differences between experimental groups was performed using two-tailed unpaired Student's t-test. The paired Student's t-test was used to compared pre- and post-treatment values in human subjects. Differences between groups were considered significant at p<0.05 and all values are expressed as mean±SD.

### Results

### CF Blocks PCSK9 Expression and Secretion in Hepatocytes

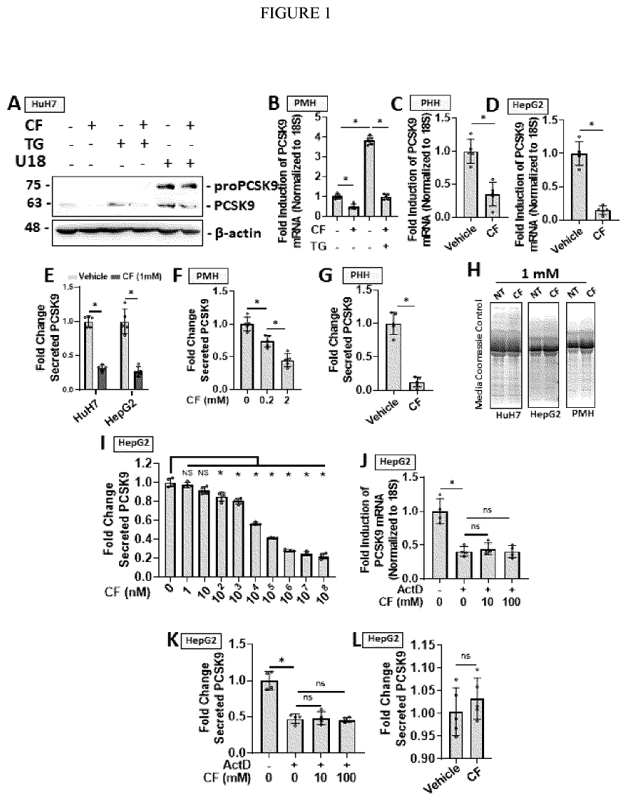

Cultured immortalized hepatocytes known to express and secrete PCSK9 (Lebeau, P., et al., 2017), including HuH7 and HepG2 cells, as well as primary mouse- and human-hepatocytes (PMH and PHH, respectively), were treated with CF for 24 h and assessed for PCSK9 expression via immunoblots and real-time PCR (FIG. 1 A to D). These initial experiments revealed that CF reduced protein and mRNA transcript levels of PCSK9. CF also attenuated PCSK9 expression resulting from the SERCA antagonist and established ER stress-inducing agent, thapsigargin (TG; FIGS. 1A and 1B) (Lebeau, P., et al., 2017). Given that sterol deprivation represents another well-established promoter of SREBP2 activation (Lebeau, P., et al., 2017), cells were also treated with CF in the presence and absence of U18666A (U18) that depletes intracellular sterols. Like TG-treated cells, CF attenuated U18-induced PCSK9 expression (FIG. 1 A); see Table 5 for a list of compounds and mechanisms of action. CF also blocked the secretion of PCSK9 from HuH7 and HepG2 cultured hepatocytes, as well as from PMH and PHH (FIG. 1 E to G). A Coomassie stain of electrophoretically-resolved media harvested from these cells was also used to confirm that CF was not affecting global protein secretion (FIG. 1 H). Next, HepG2 cells were treated with an increasing dose of CF. Results from this experiment demonstrate that PCSK9 elicited a dynamic response to caffeine from the 102 to the 108 nM range in hepatocytes (FIG. 1 I). To determine whether caffeine was affecting PCSK9 expression and secretion on a transcriptional or post-transcriptional manner, HepG2 cells were pre-treated with a transcription blocker (actinomycin D [ActD]) and subsequently exposed to CF. The failure of CF to block PCSK9 mRNA (FIG. 1 J) and secreted protein (FIG. 1 K) in the presence of ActD suggest that CF exerts its effect on PCSK9 in a transcription-dependent manner. In support of these findings, CF also failed to block the secretion of PCSK9 in cells transfected with a CMV-driven PCSK9 vector (FIG. 1 L). These data demonstrate that CF, down to the nanomolar range, can cause a significant reduction of PCSK9 expression at the mRNA and protein levels, in a variety of cultured hepatocyte cell models and that the inhibition likely occurs at the transcriptional level.

### CF Blocks SREBP2 Activation in Hepatocytes

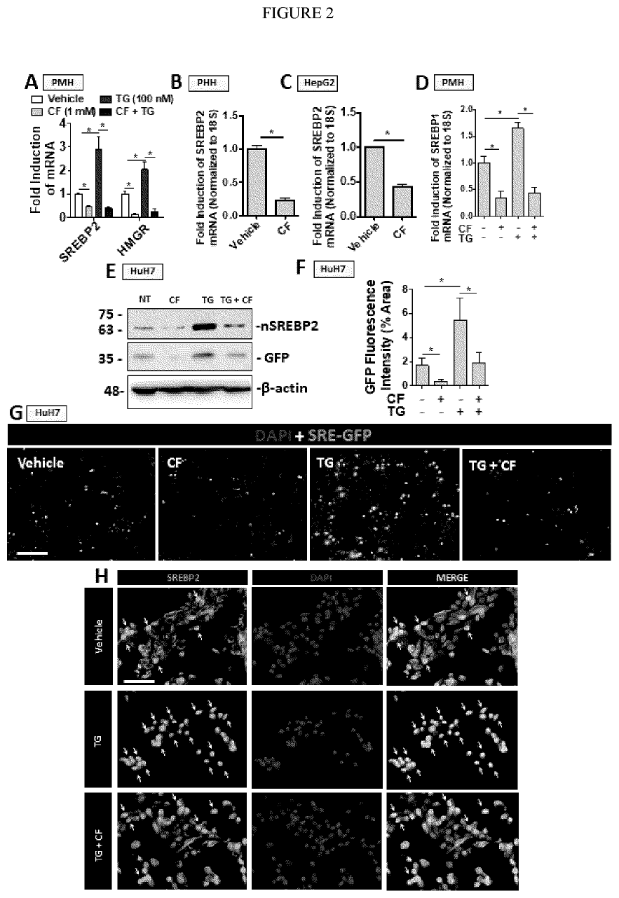

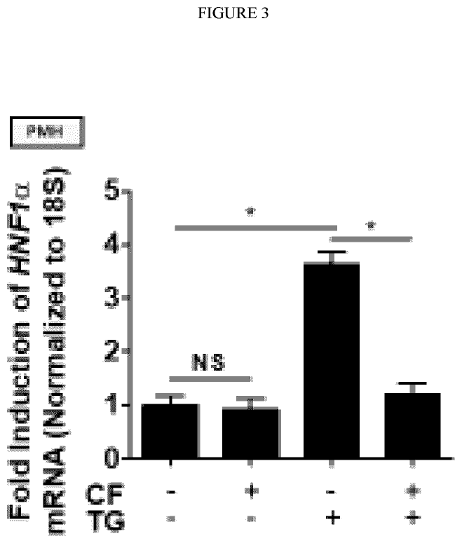

It has been previously demonstrated that ER stress, specifically resulting from the depletion of ER Ca²⁺, promotes the activation of SREBP2 and expression of PCSK9 (Lebeau, P., et al., 2017; Werstuck, G. H.; et al., 2001; Colgan, S. M., et al., 2007). Therefore, the effect of CF on TG-; induced SREBP2 activation was examined. Consistent with previous studies (Quan, H. Y., et al., 2013), it was observed that CF blocked the expression of SREBP2 in PMHs and PHHs, as well as in HepG2 cells (FIG. 2 A to C). CF also blocked the expression of a downstream target of SREBP2 transcriptional activity in mouse hepatocytes, HMGR (FIG. 2 A), as well as SREBP1, the isoform known to regulate fatty acid synthesis (FIG. 2 D). The effect of CF on the expression of hepatocyte nuclear factor 1α, a liver-expressed transcription factor also known to regulate PCSK9 expression (Dong, B., et al., 2010), was assessed but did not yield a significant difference in the absence of TG (FIG. 3A). SREBP2 activity was then examined at the protein level in HuH7 cells transfected with a plasmid encoding GFP driven by the sterol regulatory element (SRE-GFP; FIG. 2E to G). Consistent with the real-time PCR data, it was observed that CF blocked the nuclear/activated isoform of SREBP2 (nSREBP2; ˜60 kDa) and the expression of SRE-driven GFP in the presence and absence of TG. GFP expression was also visualized via immunofluorescent staining and quantified using ImageJ Software. Immunofluorescent staining of SREBP2 in cells treated with TG in the presence and absence of CF also demonstrated that CF attenuated the re-localization of SREBP2 from the perinuclear region of the cell to the nucleus (FIG. 2 H; nuclei containing activated SREBP2 are indicated by white arrows). Collectively, given the well-established role of SREBP2 in the transcriptional regulation of PCSK9, these data suggest that CF reduces PCSK9 expression and secretion by antagonizing de novo synthesis.

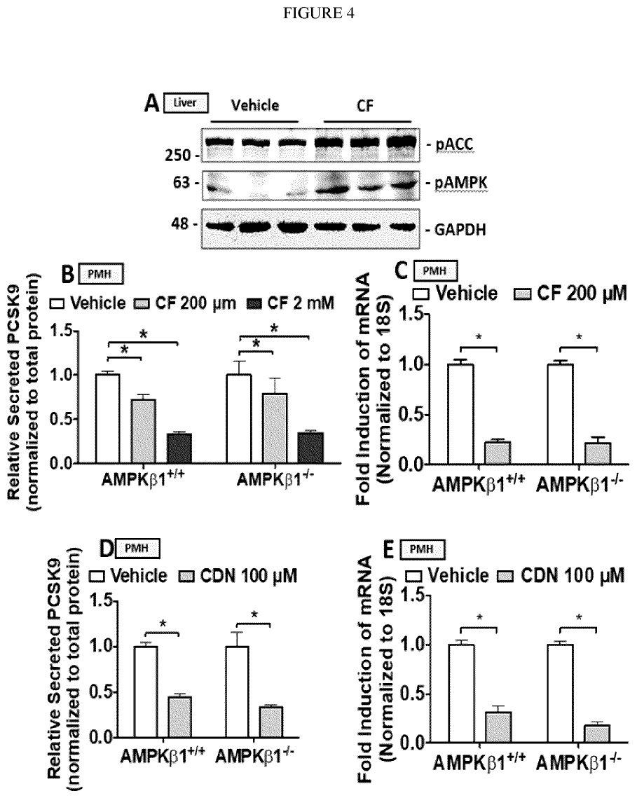

Previous studies have also shown that CF can promote the phosphorylation and activation of AMPK (Quan, H. Y., et al., 2013; Tsuda, S., et al., 2015), a liver-expressed kinase known to induce the inhibitory phosphorylation of SREBP1c (Li, Y., et al., 2011). Similar results were observed in this study, whereby CF treatment induced the phosphorylation and activation of AMPK (pAMPK) and subsequent induction of a downstream marker (phosphorylated acetyl CoA carboxylase [pACC]) in the livers of C57BL/6J mice (FIG. 4 A). Primary mouse hepatocytes were then isolated from wildtype (WT) and Ampkβ1−/− mice (Dzamko, N., et al., 2010). Treatment of these hepatocytes with CF led to a reduction in PCSK9 expression and secretion (FIG. 4 B and C), suggesting that AMPK is not directly involved in CF-mediated PCSK9 inhibition. A similar result was observed in hepatocytes treated with CDN1163 (CDN), a pharmacologic agent known to increase ER Ca2+ levels by inducing SERCA pump activation (FIG. 4 D and E) (Kang, S., et al., 2016).

### ER Ca² Modulates PCSK9 Expression and Secretion

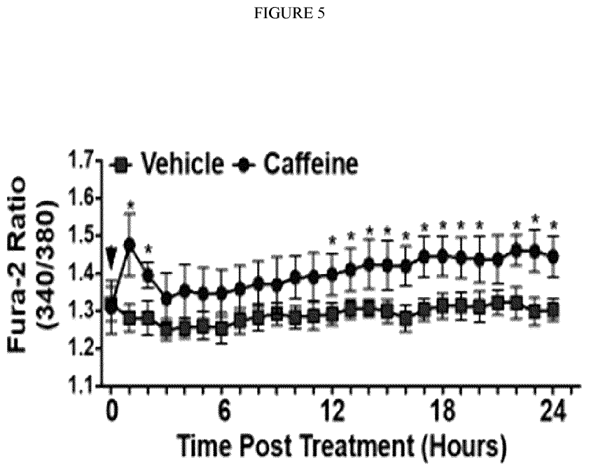

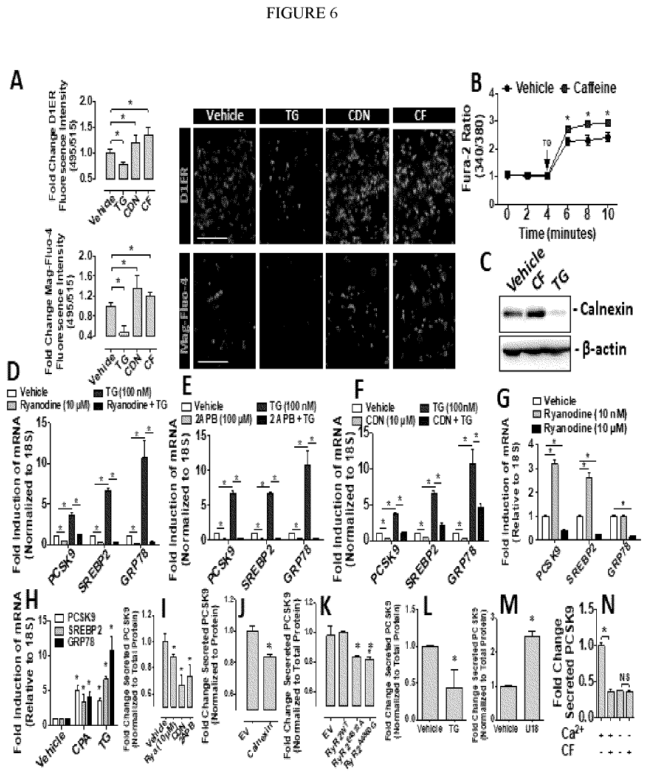

Among the many intracellular effects of CF on the cell, its ability to increase intracellular Ca²⁺ levels is well-studied (Echeverri, D., et al., 2010). Given that it has been previously demonstrated that ER Ca²⁺ depletion induced SREBP2 activation (Lebeau, P., et al., 2017), in the present study it was investigated whether (a) CF may increase ER Ca²⁺ levels, and (b) other agents known to increase ER Ca²⁺ levels may also block SREBP2 activation and PCSK9 expression. To test this hypothesis, cytosolic Ca²⁺ levels in CF-treated cells were first examined using the high-affinity fluorescent Ca²⁺ indicator, Fura-2-AM. Consistent with previous studies, CF significantly increased cytosolic Ca²⁺ levels in immortalized hepatocytes (FIG. 5). ER Ca²⁺ levels were then examined in cells transfected with D1ER; a genetically-encoded ER-resident fluorescence resonance energy transfer (FRET)-based calreticulin chameleon Ca²⁺ sensor, which increases in fluorescence intensity upon Ca²⁺ binding (Palmer, A. E., et al., 2004). The low-affinity Ca²⁺ indicator, Mag-Fluo-4, was also utilized for the direct assessment of ER Ca²⁺ and increases in fluorescence intensity upon Ca²⁺ binding (Diercks, B. P., et al., 2017; Lebeau, P., et al., 2021). The fluorescence intensity of cells treated with CF and control agents, TG and CDN, was assessed using a fluorescent spectrophotometer and visualized using a fluorescent microscope (FIG. 6 A). In addition to heightened cytosolic Ca²⁺ levels, it was also observed that CF increased ER Ca²⁺ levels. As expected, control agent CDN increased ER Ca²⁺ levels, whereas TG reduced ER Ca²⁺ levels. ER Ca²⁺ content was also assessed indirectly with the high affinity Ca²⁺ dye, fura-2-AM (FIG. 6 B). HuH7 cells were pretreated with CF for 24 h and subsequently exposed to a high dose of TG, which causes a spontaneous loss of ER Ca²⁺. In response to this treatment, cells pretreated with CF exhibited increased ER Ca²⁺ efflux compared to cells treated with the vehicle control when exposed to TG. It was also observed that the protein expression of calnexin, an ER-resident protein with high capacity for Ca²⁺ binding (Williams, D. B., 2006), was induced by CF and blocked by TG (FIG. 6 C).

To further test the hypothesis that increasing ER Ca²⁺ blocks PCSK9 expression, cells with a variety of well-established Ca²⁺-modulating agents were treated. At low dose (10 nM), ryanodine is known to facilitate ER Ca²⁺ loss by enhancing RyR-mediated Ca² transients, whereas high dose ryanodine (10 μM) is known to block RyR-mediated ER Ca²⁺ leakage (Chen, W., et al., 2014). The compound 2-APB also blocks the exit of ER Ca²⁺ by antagonizing IP3Rs (Bootman, M. D., et al., 2002). In contrast to these two agents that modulate ER Ca²⁺ release, CDN is an established allosteric activator of the SERCA pump and thus increases the entry of Ca²⁺ into the ER (Robinson, J. G., et al., 2019). Consistent with the hypothesis, it was observed that high-dose ryanodine, 2 APB and CDN, blocked SREBP2 and PCSK9 at the mRNA transcript level in the presence or absence of TG (FIG. 6 D to F). These agents also blocked TG-induced expression of the Ca²⁺-dependent chaperone, and ER stress marker, GRP78. Consistent with previous studies, it was also observed that ER Ca² depletion via TG and cyclopiazonic acid (CPA) treatment increased PCSK9 and SREBP2 expression (FIG. 6 G and H). As expected, these established ER stress-inducing agents also increased the expression of GRP78.

Secreted PCSK9 levels in the media harvested from cells treated with Ca²⁺-modulating agents were then assessed using ELISAs. Consistent with real-time PCR findings, it was observed that high-dose ryanodine, CDN and 2 APB blocked PCSK9 secretion (FIG. 6 I). Overexpression of calnexin and loss-of function ryanodine receptor variants (RyR2^{E4872A} and RyR2^{A4860}G), which were previously shown to increase ER Ca²⁺ levels ((Williams, D. B., 2006; Bootman, M. D., et al., 2002), also blocked PCSK9 secretion (FIG. 6 J and K). In contrast to its effect on PCSK9 mRNA transcript levels, it was also observed that TG blocked PCSK9 secretion (FIG. 3 L); an observation consistent with a previous study (Lebeau, P., et al., 2017). Sterol deprivation via treatment with U18, which is not known to affect ER Ca²⁺ levels, also yielded findings consistent with previous observations (Lebeau, P., et al., 2017) and increased PCSK9 secretion (FIG. 6 M). Finally, to confirm that CF blocked PCSK9 secretion in a Ca²⁺ dependent manner, experiments were repeated in HepG2 cells incubated in Ca²⁺-deficient medium for 48 h (FIG. 6 N). It was previously demonstrated that this treatment causes robust ER stress and likely explains the observed reduction of secreted PCSK9 levels in the absence of CF. Importantly, however, these data reveal that CF failed to antagonize PCSK9 secretion in cells that have been deprived of Ca²⁺. Overall, these data provide strong evidence that ER Ca²⁺ levels not only affect the expression of ER stress markers, but also regulate the cholesterol-regulatory proteins, PCSK9 and SREBP2.

Ca² Increases the Binding Capacity of GRP78 for ER-Resident SREBP2 and Prevents its Exit from the ER

GRP78 is among a number of Ca²⁺-dependent chaperones that play a central role facilitating a chemical equilibrium that favors elevated Ca²⁺ levels in the ER lumen relative to the cytosol via direct binding/sequestration and buffering (Coe, H. and Michalak, M., 2009). It is estimated that GRP78 increases the Ca²⁺-retaining ability of the ER by 25% (Lievremont, J. P., et al., 1997). Given that chaperones increase ER Ca²⁺ levels but are also Ca²⁺-dependent in their capacity to bind and fold polypeptides, it was investigated whether ER Ca²⁺ could modulate the ability of GRP78 to interact with ER-resident pre-mature SREBP2 (˜125 kDa). Previous studies have demonstrated that (a) GRP78 is highly promiscuous in its client specificity (Flynn, G C., et al., 1991), capable of binding to one site every 36 amino acids of a randomly generated peptide (Blond-Elguindi, S., et al., 1993); (b) Ca²⁺ and ATP bind to GRP78 in a cooperative manner and that ATP is necessary for the peptide binding and folding abilities of this chaperone (Yang, J., et al., 2015); and (c) overexpression of GRP78 can attenuate the activation SREBPs in response to ER stress (Werstuck, G. H.; et al., 2001).

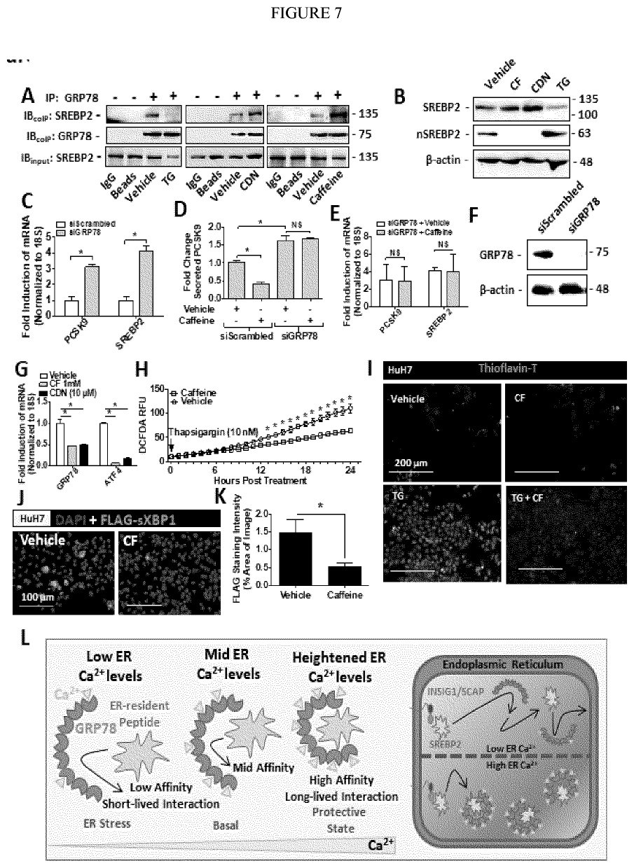

To determine whether increasing ER Ca²⁺ levels enhance GRP78 peptide binding capacity, HuH7 cells were treated with either CDN, which increases ER Ca²⁺ levels, or TG which causes ER Ca²⁺ depletion. Following treatment, the interaction taking place between GRP78 and SREBP2 was examined via immunoprecipitation of the former. By affecting ER Ca²⁺ levels however, these agents also directly impact the expression and abundance of GRP78 compared to untreated cells. Therefore, assessment of the relative binding capacity GRP78 for SREBP2 between treatments required normalization of immunoprecipitations to equivalent GRP78 protein levels (FIG. 7 A). Following conditions of TG-induced ER Ca²⁺ depletion and stress, GRP78 lost its intrinsic ability to interact with pre-mature SREBP2. Conversely, increasing ER Ca²⁺ levels via CDN treatment enhanced the ability of GRP78 to interact with and sequester pre-mature SREBP2. Strikingly, it was observed that CF-treated cells behaved much like that of CDN-treated cells, suggesting that CF also increases the SREBP2-binding capacity of GRP78. Consistent with immunoprecipitations, immunoblots of whole-cell lysates demonstrate that CF and CDN antagonized the activation and nuclear localization of SREBP2, whereas TG had the opposite effect (FIG. 7 B).

To confirm that CF blocked SREBP2 activation in a manner dependent on GRP78, cells transfected with siRNA targeted against GRP78 (siGRP78) were also treated with CF. These findings demonstrate that siGRP78 treatment significantly increased the mRNA and secreted forms of PCSK9, as well as the mRNA levels of SREBP2 (FIG. 7 C and D). Consistent with these findings, it was also observed that CF failed to attenuate PCSK9 and SREBP2 mRNA expression or secreted PCSK9 levels in the presence of siGRP78 (FIG. 7 D and E). The siRNA-mediated knockdown of GRP78 was confirmed via immunoblotting (FIG. 7 F).

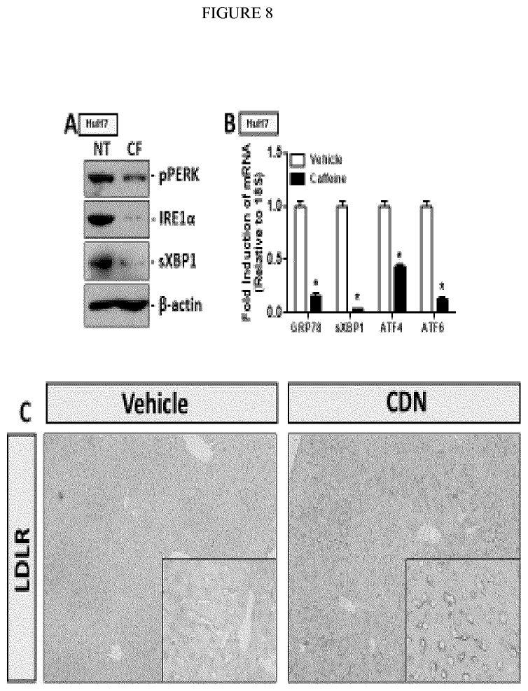

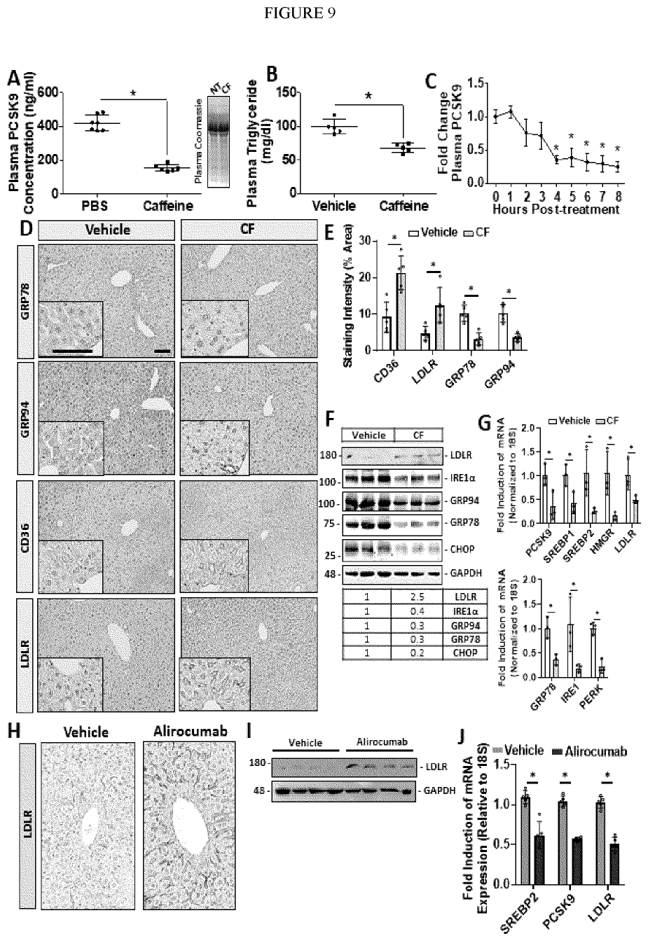

Because ER Ca²⁺ depletion induces a compensatory unfolded protein response (UPR), it was also postulated that CF may attenuate UPR marker expression by increasing ER Ca²⁺ levels. Upon assessment of PHH treated with CF, a reduction in mRNA transcript levels of ER stress markers GRP78 and activating transcription factor 4 was observed (ATF4; FIG. 7 G). Similar experiments were also carried out in cultured HuH7 cells (FIG. 8 A and B), in which a CF-mediated reduction in the expression of pPERK, IRE1α, sXBP1, ATF4 and ATF6 was also observed via immunoblotting and real-time PCR. Reactive oxygen species production, a process known to occur during conditions of ER stress, was also attenuated by CF (FIG. 9 H). Consistent with its effect on ER stress markers, CF blocked the accumulation of thioflavin-T-stained misfolded protein aggregates and the expression of FLAG-sXBP1 in cells transfected with the ER activated indicator plasmid (FIG. 7 I-K). Collectively, these data support a model in which heightened ER Ca²⁺ levels promote chaperone function and efficiency, thereby leading to a reduction in chaperone abundance. While increasing the protein binding ability of chaperones, such as GRP78, CF also attenuates SREBP2-driven gene expression (FIG. 7 L).

### CF Blocks Hepatic ER Chaperone Expression and Attenuates PCSK9 Secretion in Mice

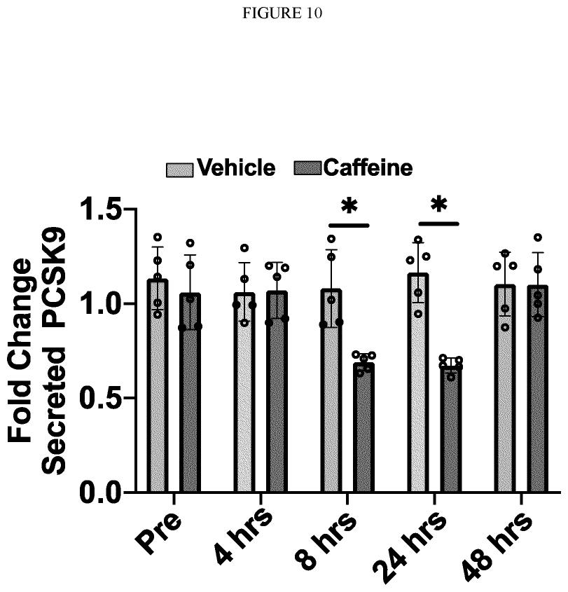

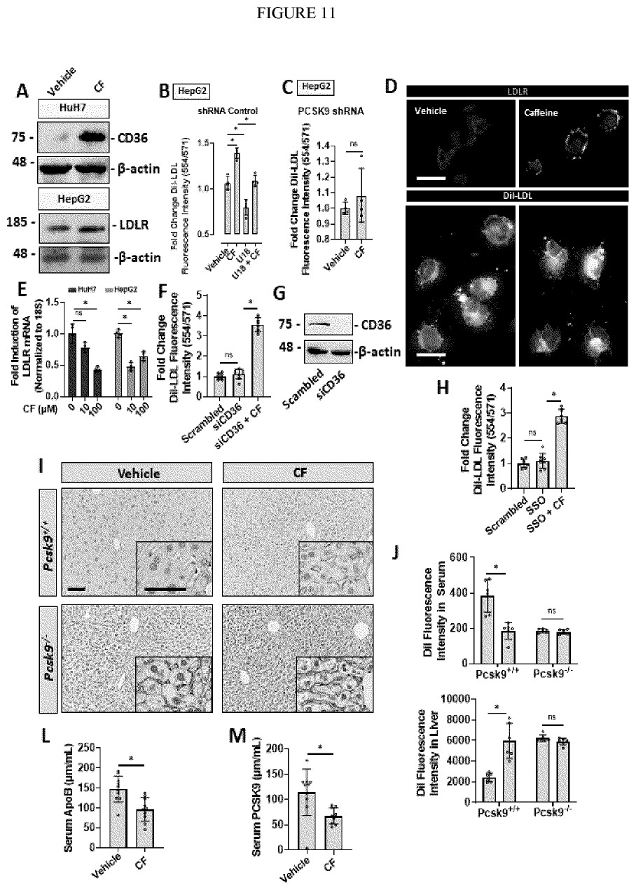

Next, the effect of CF on PCSK9 expression/secretion and ER stress marker expression was assessed in mice. Following IP injection of CF (50 mg/kg-8 h), a significant reduction of circulating PCSK9 and triglyceride levels was observed (FIG. 9 A and B). A time-course experiment also revealed that CF treatment required 4 h to significantly reduce plasma PCSK9 levels in mice (FIG. 9 C). Consistent with these observations, administration of CF via oral-gavage (20 mg/kg) also reduced circulating PCSK9 levels (FIG. 10) The protein and mRNA expression of UPR chaperones, GRP78, GRP94, IRE1α and CHOP was also assessed via immunohistochemical staining, immunoblots and real-time PCR in the livers of these mice. Consistent with the findings in cultured cells, CF reduced the expression of UPR markers (FIG. 9 D to G). Likewise, in a manner similar to previous reports, an inverse correlation between plasma PCSK9 levels and the expression of hepatic cell-surface LDLR and CD36 protein was observed (FIG. 9D to E) (Abifadel, M., et al., 2003; Benjannet, S., et al., 2004; Demers, A., et al., 2015). Immunohistochemical staining intensities were quantified using ImageJ Software (FIG. 9 E). Despite the increase in hepatic cell-surface LDLR levels, RT-PCR data revealed that LDLR transcript levels were reduced by CF; a result that is consistent with other SREBP-2 regulated genes. Similar to CF, CDN also increased hepatic LDLR expression in mice (FIG. 8 C. 50 mg/kg; IP; 8 h).

To confirm that this mouse model was responding to treatments in a manner consistent with previous studies, mice were treated with alirocumab; a well-established clinically approved anti-PCSK9 monoclonal antibody (Kuhnast, S., et al., 2014). Treatment with alirocumab led to a significant increase in hepatic LDLR expression (FIG. 9 H and I). A reduction in the mRNA levels of SREBP2, PCSK9 and the LDLR was also observed (FIG. 9 J). Overall, these studies demonstrate that CF blocks the secretion of PCSK9 and increases the expression of the LDLR in vivo.

### CF Increases Hepatic LDL Uptake

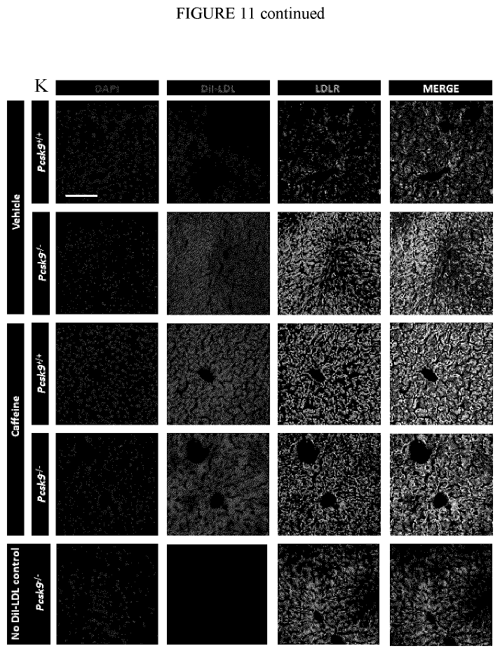

It is well-established that PCSK9 enhances the degradation of the LDLR and reduces the capacity of hepatocytes to bind and internalized extracellular LDL cholesterol (Seidah, N. G., et al., 2017). Therefore, it was examined if CF and other agents that increase ER Ca²⁺ levels, may also increase LDLc clearance. This started by confirming that CF increased the expression of PCSK9-regulated receptors in the cultured cell models using immunoblots (FIG. 11 A). Next, an assay was developed whereby HepG2 cells plated in black clear-bottom 96-well plates were treated with agents for 24 h and subsequently exposed to fluorescently-labelled DiI-LDL for 5 h in FBS-free medium prior to analysis. The uptake and accumulation of DiI-LDL was then quantified using a fluorescent spectrophotometer (Molecular Devices). It was observed that CF increased LDLc uptake and that U18, an agent that increased secreted PCSK9 levels (FIG. 6 M), reduced LDLc uptake (FIG. 11B). To confirm that CF increased LDL uptake in cultured hepatocytes in a manner dependent on PCSK9 inhibition, this experiment was repeated in HepG2 cells stably transfected with PCSK9 shRNA (FIG. 11 C). CF treatment failed to significantly increase LDLc uptake in conditions of reduced PCSK9 levels. Live-cell staining of the LDLR was also performed in HepG2 cells exposed to DiI-LDL (FIG. 11 D). Increased cell-surface LDLR, as well as intracellular DiI-LDLc, was observed in CF-treated HepG2 cells compared to vehicle-treated cells using a fluorescent microscope. Despite the increased protein abundance of the LDLR, as well as increased uptake of LDLc, CF reduced mRNA transcript levels of the LDLR in HuH7 and HepG2 cells (FIG. 11 E). Given the observed increase in CD36 receptor levels, additional experiments were conducted to determine whether CD36 played a role in LDL uptake in response to CF. Results from these experiments demonstrate that the knockdown of CD36 via siRNA, as well as the pharmacologic inhibition using sulfosuccinimidyl oleate (SSO), failed to affect CF-mediated DiI-LDL uptake (FIG. 11 F and G). The knockdown of CD36 via siRNA was confirmed via immunoblotting (FIG. 11 G).

The effect of CF on hepatic LDLc uptake was next examined in mice. Accordingly, Pcsk9+/+ and Pcsk9−/− mice were treated with either CF or PBS-vehicle for 8 h, as well as fluorescently labeled DiI-LDL cholesterol for one h prior to sacrifice. In support of the in vitro studies, it was observed that CF increased hepatic cell-surface LDLR expression in the Pcsk9+/+ mice but did not increase LDLR expression in Pcsk9−/− mice (FIG. 11 I). CF also increased hepatic DiI fluorescence intensity and reduced serum DiI fluorescence intensity in Pcsk9+/+ mice but failed to affect these parameters in Pcsk9−/− mice (FIG. 11 J). Hepatic cell-surface LDLR staining and DiI-LDL uptake were also examined using a fluorescent microscope (FIG. 11 K). Finally, native LDLc was also examined in 18-week-old male C57BL/6J mice treated with CF (30 mg/kg) every 24 hours for 14 days (FIG. 11 L and M). Using ELISAs, a reduction of the surrogate marker of LDLc, Apolipoprotein B (ApoB), as well as PCSK9 was observed in response to a daily dose of CF. Collectively, these data suggest that CF increases hepatic LDLc clearance by increasing LDLR expression in a manner dependent on its ability to block PCSK9 secretion from hepatocytes.

### CF Reduces Plasma PCSK9 Levels in Healthy Human Subjects

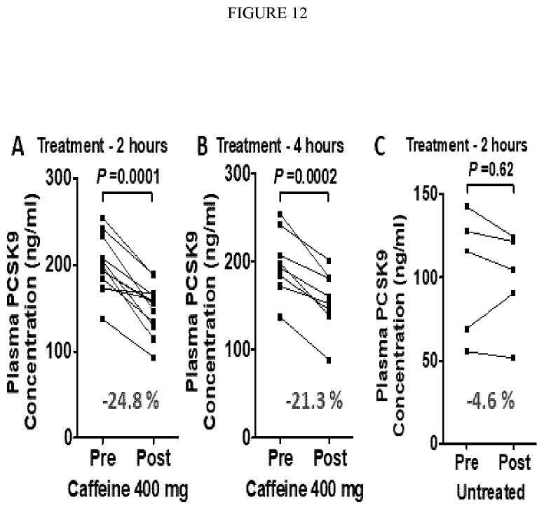

Given that CF is among the most commonly consumed pharmacologically active compounds in the world (8), its ability to affect PCSK9 levels in fasted healthy volunteers was assessed. Serum was collected prior to, as well as 2- and 4-h post CF treatment (400 mg orally; ˜5 mg/kg). Consistent with the observations in cultured hepatocytes and in mice, CF reduced plasma PCSK9 levels in healthy subjects by 25% (n=12) and 21% (n=8) at the 2- and 4-h time points, respectively (FIG. 11 A and B). Plasma PCSK9 levels were also examined control subjects that did not consume CF, to verify whether the additional 2 h of fasting during the course of the experiment would alter PCSK9 levels. No significant difference was observed in this group (n=5; FIG. 12 C).

### Example 2. Xanthine Derivatives as Inhibitors of PCSK9

### Materials and Methods

### Synthesis of Exemplary Xanthine Derivatives

One of four procedures (Procedure A, Procedure B or Procedure C) was used to synthesize various xanthine derivatives as follows:

Procedure A: 5,6-diamino-1,3-dimethyluracil (213.0 mg, 1.25 mmol, 1 equiv), arylbenzaldehyde (1.25 mmol, 1 equiv), azobisisobutyronitrile (AIBN, 4.1 mg, 0.025 mmol, 0.02 equiv), and n-bromosuccinimide (NBS, 155.7 mg, 0.875 mmol, 0.7 equiv) were added to a solution of MeCN/H₂O (9:1, 5 mL). The reaction mixture was stirred at 30° C. for 3 hours. The resulting precipitate was filtered and rinsed with ethanol to afford the desired product.

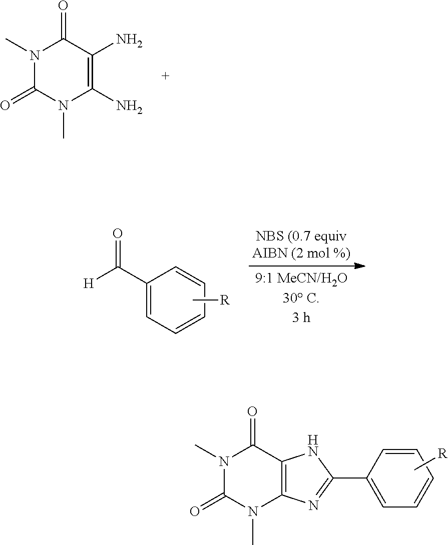

Procedure B: 8-bromocaffeine (0.366 mmol, 100 mg, 1 equiv), potassium trifluoroborate salt (0.549 mmol, 1.5 equiv), K₂CO₃ (1.10 mmol, 152 mg, 3 equiv), and Pd(amphos)Cl₂ (5 mol %, 13 mg) was added to a Monowave tube of 6:1 THF:H₂O (3 mL) and reacted in the Monowave for 30 minutes at 160° C. The crude reaction was filtered through Celite, concentrated under reduced pressure, and separated via column chromatography. Products were solids obtained in 12-72% yield (10 mg-150 mg).

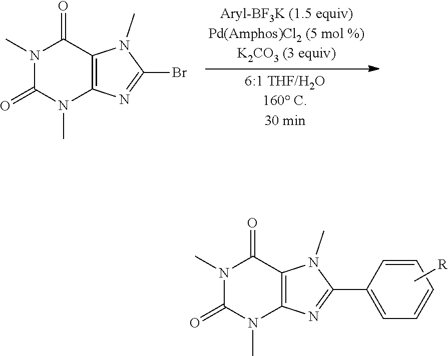

Procedure C: Caffeine (0.5 mmol), Cs₂CO₃ (1.5 mmol), Pd(OAc)₂ (0.025 mmol), Cy₃P·HBF₄ (0.05 mmol) and PvOH (0.1 mmol) were added to an oven-dried vial. The vial was then sealed with a rubber septum and evacuated under nitrogen. Dry DMF (3 mL) and the aryl-bromide (0.75 mmol) were then injected into the vial. The mixture was stirred for 20 h at 130° C. The resulting mixture was vacuum filtered through celite, and then extracted three times with DCM, washed three times with water, and washed once with 10% LiCl solution. The organic phase was dried with Na₂SO₄ and concentrated via rotary evaporation. The crude mixture was separated via column chromatography to yield the arylated xanthine product (20-80% yield).

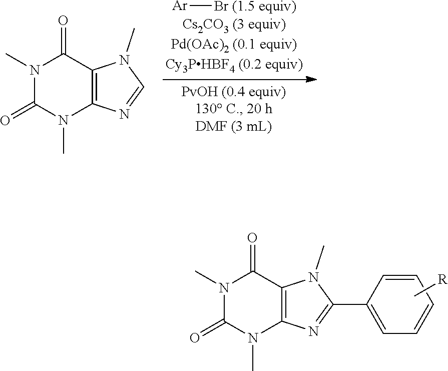

### Characterization of Exemplary Xanthine Derivatives

Procedure A was followed with isonicotinaldehyde to obtain I-9 as a light brown solid in 18% yield (30 mg). ¹H NMR (700 MHz, DMSO-d₆): δ (ppm) 14.28 (s, br, 1H, NH), 8.72 (d, J=5.7 Hz, 2H), 8.03 (d, J=5.6 Hz, 2H), 3.50 (s, 3H, CH3), 3.26 (s, 3H, CH3).

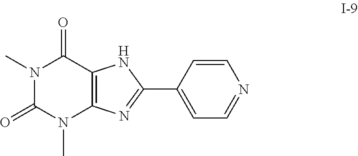 [SMILES: Cn1c(=O)c2[nH]c(-c3ccncc3)nc2n(C)c1=O]

Procedure A was followed with 3,5-bis(trifluoromethyl)benzaldehyde to obtain Ja-8 as a white solid in 40% yield (196 mg). Mp: 329-330° C. ¹H NMR (700 MHz, DMSO-d₆): δ (ppm) 14.38 (s, br, 1H, NH), 8.76 (s, 1H), 8.24 (s, 1H), 3.53 (s, 3H, CH3), 3.28 (s, 3H, CH3). ¹³C NMR DEPTQ (176 MHz, CDCl₃): δ (ppm) 154.32, 151.09, 148.14, 146.17, 131.38-130.81, 126.39, 125.41-120.76, q (J=273.1 Hz), 123.31, 29.86, 27.84. FT-IR (cm⁻¹): 3052.09, 2943.32, 2759.41, 1704.53, 1649.52, 1601.04. LCMS (ESI) m/z: 393.07807 calculated for ([M+H]⁺); 393.07684 observed.

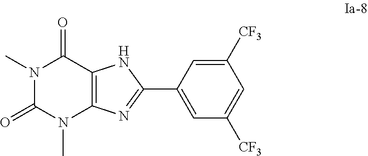 [SMILES: Cn1c(=O)c2[nH]c(-c3cc(C(F)(F)F)cc(C(F)(F)F)c3)nc2n(C)c1=O]

Procedure A was followed with 2,4,5-trifluorobenzaldehyde to obtain Ia-1 as a light brown solid in 30% yield (117 mg). Mp: 361-363° C. ¹H NMR (700 MHz, DMSO-d₆): δ (ppm) 13.86 (s, br, 1H, NH), 8.05-8.01 (dd, J=17.1, 9.0 Hz, 1H), 7.83-7.79 (td, J=10.4, 6.8 Hz, 1H), 3.49 (s, 3H, CH3), 3.27 (s, 3H, CH3). FT-IR (cm⁻¹): 3281.65, 3039.61, 1695.87, 1651.37, 1602.14. LCMS (ESI) m/z: 311.07504 calculated for ([M+H]⁺); 311.07481 observed.

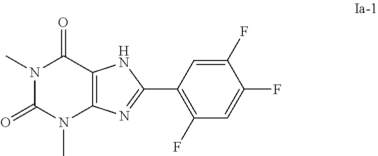 [SMILES: Cn1c(=O)c2[nH]c(-c3cc(F)c(F)cc3F)nc2n(C)c1=O]

Procedure A was followed with 5-nitro-2-furaldehyde to obtain Ia-7 as a yellow solid in 40% yield (35 mg). ¹H NMR (400 MHz, DMSO-d₆): δ (ppm) 14.61 (s, 1H), 7.86 (d, J=4.0 Hz, 1H), 7.46 (d, J=4.0 Hz, 1H), 3.48 (s, 3H), 3.26 (s, 3H).

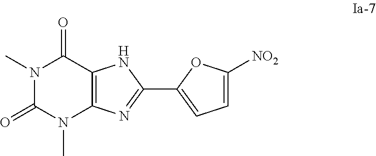 [SMILES: Cn1c(=O)c2[nH]c(-c3ccc([N+](=O)[O-])o3)nc2n(C)c1=O]

Procedure A was followed with 4-(1H-1,2,4-triazol-1-yl)benzaldehyde to obtain Ia-13 as a white solid in 25% yield (24 mg). ¹H NMR (400 MHz, DMSO-d₆): δ (ppm) 13.95 (s, 1H), 9.39 (s, 1H), 8.29 (dd, J=6.9, 1.9 Hz, 3H), 8.08-7.96 (m, 2H), 3.51 (s, 3H), 3.26 (s, 3H).

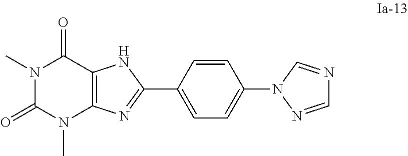 [SMILES: Cn1c(=O)c2[nH]c(-c3ccc(-n4cncn4)cc3)nc2n(C)c1=O]

Procedure A was followed with 2-methoxypyrimidine-5-carbaldehyde to obtain Ia-17 as a beige solid in 6% yield (5 mg). ¹H NMR (400 MHz, DMSO-d₆): δ (ppm) 14.05 (s, 1H), 9.21 (s, 2H), 4.00 (s, 3H), 3.50 (s, 3H), 3.27 (s, 3H).

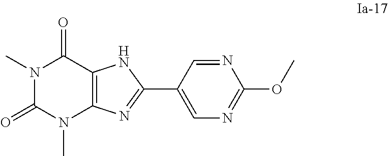

Procedure A was followed with isovanillin to obtain Ia-23 as a brown solid in 14% yield (13 mg). ¹H NMR (400 MHz, DMSO-d₆): δ (ppm) 13.54 (s, 1H), 9.29 (s, 1H), 7.59 (d, J=8.0 Hz, 2H), 7.03 (d, J=8.3 Hz, 1H), 3.83 (s, 3H), 3.48 (s, 3H), 3.25 (s, 3H).

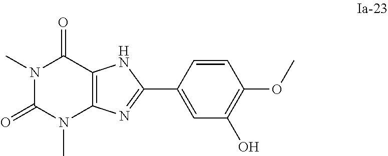

Procedure A was followed with pyrimidine-5-carbaldehyde to obtain I-52 as a beige solid in 25% yield (20 mg). ¹H NMR (400 MHz, DMSO-d₆): δ (ppm) 14.34 (s, 1H), 9.42 (s, 2H), 9.28 (s, 1H), 3.52 (s, 3H), 3.28 (s, 3H).

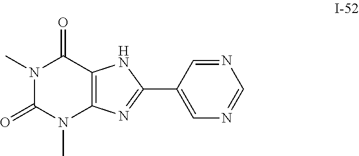 [SMILES: Cn1c(=O)c2[nH]c(-c3cncnc3)nc2n(C)c1=O]

Procedure A was followed with 2,3,4-trifluorobenzaldehyde to obtain Ia-87 as a white solid in 86% yield (80 mg). ¹H NMR (400 MHz, DMSO-d₆): δ (ppm) 13.95 (s, 1H), 7.93-7.70 (m, 1H), 7.51 (m, 1H), 3.27 (s, 3H).

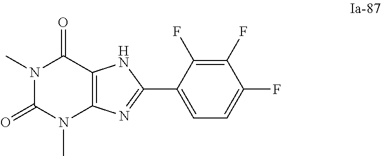 [SMILES: Cn1c(=O)c2[nH]c(-c3ccc(F)c(F)c3F)nc2n(C)c1=O]

Procedure A was followed with 6-(trifluoromethyl)nicotinaldehyde to obtain Ia-88 as a white solid in 25% yield (24 mg). ¹H NMR (400 MHz, DMSO-d₆): δ (ppm) 14.38 (s, 1H), 9.42 (d, J=2.1 Hz, 1H), 8.69 (ddd, J=8.3, 2.2, 0.8 Hz, 1H), 8.07 (dd, J=8.4, 0.8 Hz, 1H), 3.51 (s, 3H), 3.27 (s, 3H).

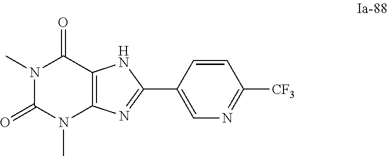 [SMILES: Cn1c(=O)c2[nH]c(-c3ccc(C(F)(F)F)nc3)nc2n(C)c1=O]

Procedure A was followed with 4-bromobenzaldehyde to obtain 1-89 as a white solid in 50% yield (50 mg). ¹H NMR (400 MHz, DMSO-d₆): δ (ppm) 13.97 (s, 1H), 8.12-7.99 (m, 2H), 7.79-7.69 (m, 2H), 3.50 s 3H). 3.27 (s. 3H).

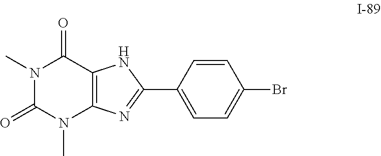 [SMILES: Cn1c(=O)c2[nH]c(-c3ccc(Br)cc3)nc2n(C)c1=O]

Procedure A was followed with 3-methylbenzaldehyde to obtain Ia-90 as a white solid in 41% yield (33 mg). ¹H NMR (400 MHz, DMSO-d₆): δ (ppm) 13.77 (s, 1H), 7.97 (d, J=1.8 Hz, 1H), 7.93 (dd, J=7.9, 1.6 Hz, 1H), 7.39 (t, J=7.7 Hz, 1H), 7.32-7.26 (m, 1H), 3.49 (s, 3H), 3.26 (s, 3H), 2.37 (s, 3H).

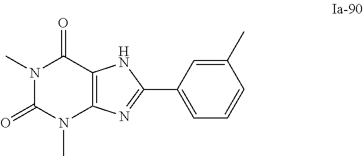

Procedure A was followed with 4-(trifluoromethyl)benzaldehyde to obtain Ia-91 as a white solid in 74% yield (72 mg). ¹H NMR (400 MHz, DMSO-d₆): δ (ppm) 14.17 (s, 1H), 8.34 (d, J=8.2 Hz, 2H), 7.93-7.86 (m, 2H), 3.52 (s, 3H), 3.28 (s, 3H).

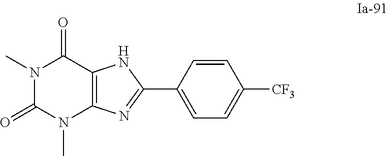 [SMILES: Cn1c(=O)c2[nH]c(-c3ccc(C(F)(F)F)cc3)nc2n(C)c1=O]

Procedure A was followed with 2,6-dichlorobenzaldehyde to obtain Ia-92 as a white solid in 20% yield (20 mg). ¹H NMR (400 MHz, DMSO-d₆): δ (ppm) 13.95 (s, 1H), 7.69-7.58 (m, 3H), 3.47 (s, 3H), 3.27 (s, 3H).

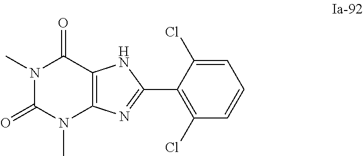 [SMILES: Cn1c(=O)c2[nH]c(-c3c(Cl)cccc3Cl)nc2n(C)c1=O]

Procedure A was followed with 2-fluoro-5-nitrobenzaldehyde to obtain Ia-93 as a white solid in 94% yield (90 mg). ¹H NMR (400 MHz, DMSO-d₆): δ (ppm) 14.11 (s, 1H), 8.85 (dd, J=6.3, 2.9 Hz, 1H), 8.41 (ddd, J=9.1, 4.1, 2.9 Hz, 1H), 7.72 (dd, J=10.0, 9.1 Hz, 1H), 3.51 (s, 3H), 3.28 (s, 3H).

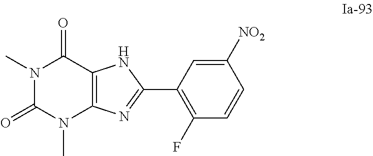 [SMILES: Cn1c(=O)c2[nH]c(-c3cc([N+](=O)[O-])ccc3F)nc2n(C)c1=O]

Procedure A was followed with 2,3,4,5,6-pentafluorobenzaldehyde to obtain Ia-94 as a white solid in 60% yield (62 mg). ¹H NMR (400 MHz, DMSO-d₆): δ (ppm) 14.38 (s, 1H), 3.47 (s, 3H), 3.27 (s, 3H).

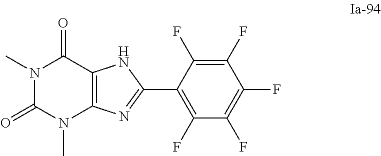 [SMILES: Cn1c(=O)c2[nH]c(-c3c(F)c(F)c(F)c(F)c3F)nc2n(C)c1=O]

Procedure A was followed with 2-fluorobenzaldehyde to obtain I-95 as a light gray solid in 60% yield (49 mg). ¹H NMR (400 MHz, DMSO-d₆): δ (ppm) 13.71 (s, 1H), 7.96 (t, J=7.5 Hz, 1H), 7.57 (q, J=6.9 Hz, 1H), 7.38 (dt, J=14.8, 8.8 Hz, 2H), 3.50 (s, 3H), 3.27 (s, 3H).

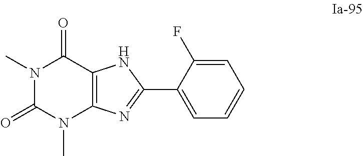 [SMILES: Cn1c(=O)c2[nH]c(-c3ccccc3F)nc2n(C)c1=O]

Procedure A was followed with 3,4-dimethoxybenzaldehyde to obtain I-96 as a brown solid in 31% yield (29 mg). ¹H NMR (400 MHz, DMSO-d₆): δ (ppm) 13.63 (s, 1H), 7.79-7.66 (m, 2H), 7.09 (d, J=8.2 Hz, 1H), 3.85 (s, 3H), 3.82 (s, 3H), 3.50 (s, 3H), 3.27 (s, 3H).

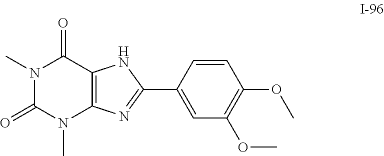

Procedure A was followed with 3-hydroxy-4-nitrobenzaldehyde to obtain Ia-97 as a light brown solid in 61% yield (58 mg). ¹H NMR (400 MHz, DMSO-d₆): δ (ppm) 14.21 (s, 1H), 11.26 (s, 1H), 8.02 (d, J=8.6 Hz, 1H), 7.88 (d, J=1.8 Hz, 1H), 7.72 (dd, J=8.7, 1.8 Hz, 1H), 3.50 (s, 3H), 3.27 (s, 3H).

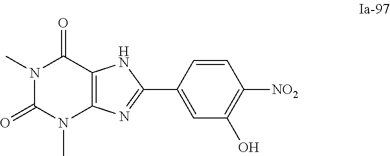 [SMILES: Cn1c(=O)c2[nH]c(-c3ccc([N+](=O)[O-])c(O)c3)nc2n(C)c1=O]

Procedure A was followed with 4-(pyridin-4-yl)benzaldehyde to obtain I-98 as a yellow solid in 82% yield (82 mg). ¹H NMR (400 MHz, DMSO-d₆): δ (ppm) 14.06 (s, 1H), 8.93 (d, J=5.9 Hz, 2H), 8.37-8.28 (m, 4H), 8.20-8.12 (m, 2H), 3.52 (s, 3H), 3.26 (s, 3H).

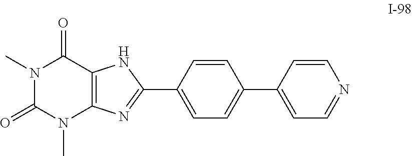 [SMILES: Cn1c(=O)c2[nH]c(-c3ccc(-c4ccncc4)cc3)nc2n(C)c1=O]

Procedure A was followed with 4-hydroxy-3-nitrobenzaldehyde to obtain Ia-99 as a light brown solid in 41% yield (38 mg). ¹H NMR (400 MHz, DMSO-d₆): δ (ppm) 13.63 (s, 1H), 10.82 (s, 1H), 8.13 (d, J=2.2 Hz, 1H), 7.91 (dd, J=8.5, 2.2 Hz, 1H), 7.06 (d, J=8.5 Hz, 1H), 3.47 (d, J=1.8 Hz, 3H), 3.25 (d, J=1.8 Hz, 3H).

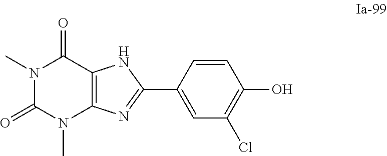 [SMILES: Cn1c(=O)c2[nH]c(-c3ccc(O)c(Cl)c3)nc2n(C)c1=O]

Procedure A was followed with 2-fluoro-4-(trifluoromethyl)benzaldehyde to obtain Ia-100 as a white solid in 47% yield (48 mg). ¹H NMR (400 MHz, DMSO-d₆): δ (ppm) 13.97 (s, 1H), 8.18 (td, J=7.7, 1.1 Hz, 1H), 7.90 (dd, J=10.7, 1.8 Hz, 1H), 7.79-7.64 (m, 1H), 3.49 (s, 3H), 3.27 (s, 3H).

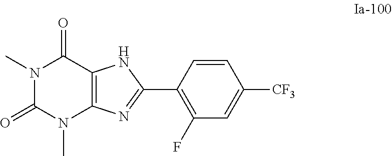 [SMILES: Cn1c(=O)c2[nH]c(-c3ccc(C(F)(F)F)cc3F)nc2n(C)c1=O]

Procedure A was followed with 3,5-dimethoxybenzaldehyde to obtain I-101 as a beige solid in 13% yield (12 mg). ¹H NMR (400 MHz, DMSO-d₆): δ (ppm) 13.83 (s, 1H), 7.34 (d, J=2.3 Hz, 2H), 6.60 (t, J=2.3 Hz, 1H), 3.81 (s, 6H), 3.50 (s, 3H), 3.27 (s, 3H).

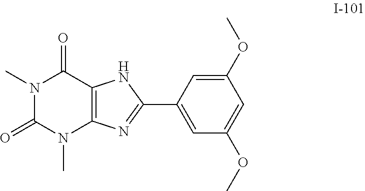 [SMILES: COc1cc(OC)cc(-c2nc3c([nH]2)c(=O)n(C)c(=O)n3C)c1]

Procedure A was followed with 3,5-dibenzyloxybenzaldehyde to obtain Ia-102 as a beige solid in 31% yield (44 mg). ¹H NMR (400 MHz, DMSO-d₆): δ (ppm) 13.81 (s, 1H), 7.50-7.31 (m, 14H), 5.15 (s, 4H), 3.49 (s, 3H), 3.26 (s, 3H).

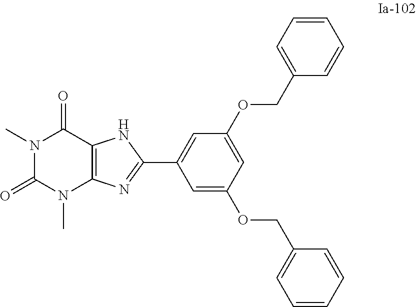 [SMILES: Cn1c(=O)c2[nH]c(-c3cc(OCc4ccccc4)cc(OCc4ccccc4)c3)nc2n(C)c1=O]

Procedure A was followed with 2,4-dihydroxybenzaldehyde to obtain Ia-103 as a dark brown-red solid in 27% yield (23 mg). ¹H NMR (400 MHz, DMSO-d₆): δ (ppm) 13.42 (s, 1H), 11.61 (s, 1H), 9.98 (d, J=53.1 Hz, 1H), 7.89 (d, J=8.6 Hz, 1H), 6.45-6.21 (m, 2H), 3.46 (d, J=1.7 Hz, 3H), 3.25 (d, J=2.8 Hz, 3H).

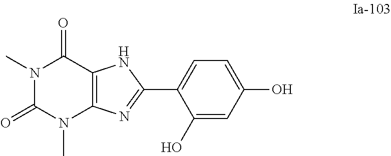 [SMILES: Cn1c(=O)c2[nH]c(-c3ccc(O)cc3O)nc2n(C)c1=O]

Procedure A was followed with 4-benzyloxybenzaldehyde to obtain Ia-104 as a white solid in 31% yield (27 mg). ¹H NMR (400 MHz, DMSO-d₆): δ (ppm) 13.64 (s, 1H), 8.14-7.96 (m, 2H), 7.57-7.28 (m, 5H), 7.19-7.03 (m, 2H), 3.50 (s, 3H), 3.26 (s, 3H).

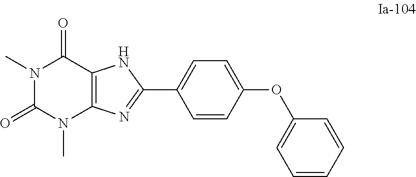 [SMILES: Cn1c(=O)c2[nH]c(-c3ccc(Oc4ccccc4)cc3)nc2n(C)c1=O]

Procedure A was followed with 4-bromo-2,6-difluorobenzaldehyde to obtain Ia-105 as a white solid in 86% yield (96 mg). ¹H NMR (400 MHz, DMSO-d₆): δ (ppm) 14.10 (s, 1H), 7.74 (d, J=8.3 Hz, 2H), 3.46 (s, 3H), 3.27 (s, 3H).

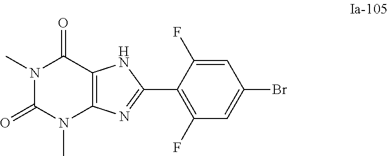 [SMILES: Cn1c(=O)c2[nH]c(-c3c(F)cc(Br)cc3F)nc2n(C)c1=O]

Procedure A was followed with 6-bromovanillin to obtain Ia-106 as a beige solid in 15% yield (17 mg). ¹H NMR (400 MHz, DMSO-d₆): δ (ppm) 13.57 (s, 1H), 10.06 (s, 1H), 7.23 (d, J=10.3 Hz, 1H), 7.11 (s, 1H), 3.82 (s, 3H), 3.48 (d, J=1.2 Hz, 3H), 3.27 (s, 3H).

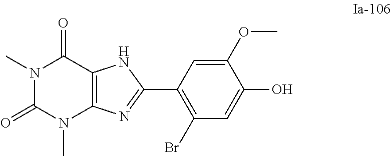

Procedure A was followed with 3,5-dimethoxy-4-hydroxybenzaldehyde to obtain I-107 as a light brown solid in 5% yield (5 mg). ¹H NMR (400 MHz, DMSO-d₆): δ (ppm) 7.45 (s, 2H), 7.23 (s, 1H), 7.10 (s, 1H), 3.84 (s, 6H), 3.50 (s, 3H), 3.26 (s, 3H).

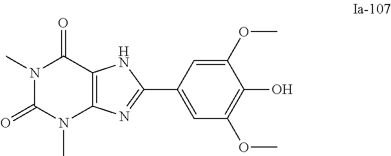

Procedure A was followed with 3,4-dihydroxybenzaldehyde to obtain Ia-108 as a brown solid in 22% yield (19 mg). ¹H NMR (400 MHz, DMSO-d₆): δ (ppm) 13.45 (s, 1H), 9.51 (s, 1H), 9.23 (s, 1H), 7.56 (d, J=2.2 Hz, 1H), 7.47 (dd, J=8.3, 2.2 Hz, 1H), 6.94-6.74 (m, 1H), 3.48 (s, 3H), 3.25 (s, 3H).

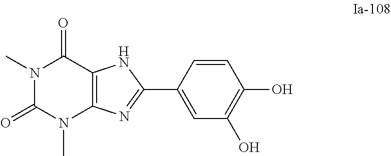 [SMILES: Cn1c(=O)c2[nH]c(-c3ccc(O)c(O)c3)nc2n(C)c1=O]

Procedure A was followed with 2,4-dinitrobenzaldehyde to obtain 1-109 as a yellow solid in 56% yield (58 mg). ¹H NMR (400 MHz, DMSO-d₆): δ (ppm) 14.54 (s, 1H), 8.82 (d, J=2.3 Hz, 1H), 8.62 (dd, J=8.7, 2.3 Hz, 1H), 8.25 (d, J=8.6 Hz, 1H), 3.40 (s, 3H), 3.26 (s, 3H).

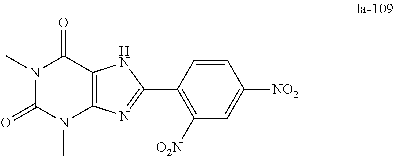 [SMILES: Cn1c(=O)c2[nH]c(-c3ccc([N+](=O)[O-])cc3[N+](=O)[O-])nc2n(C)c1=O]

Procedure A was followed with 3-bromo-5-chlorosalicylaldehyde to obtain Ia-110 as a beige solid in 22% yield (25 mg). ¹H NMR (400 MHz, DMSO-d₆): δ (ppm) 14.31 (d, J=55.9 Hz, 1H), 12.69 (s, 1H), 8.26 (s, 1H), 7.80 (s, 1H), 3.50 (s, 3H), 3.27 (s, 3H).

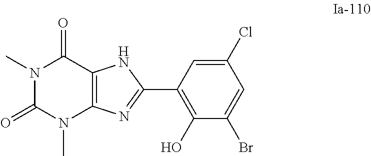 [SMILES: Cn1c(=O)c2[nH]c(-c3cc(Cl)cc(Br)c3O)nc2n(C)c1=O]

Procedure A was followed with 2-bromo-3-hydroxybenzaldehyde to obtain Ia-111 as a white solid in 40% yield (42 mg). ¹H NMR (400 MHz, DMSO-d₆): δ (ppm) 13.68 (s, 1H), 10.56 (s, 1H), 7.29 (t, J=7.8 Hz, 1H), 7.06 (ddd, J=28.2, 7.9, 1.5 Hz, 2H), 3.47 (s, 3H), 3.27 (s, 3H).

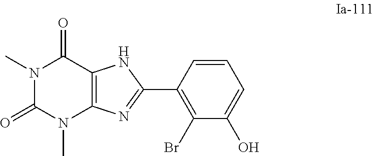 [SMILES: Cn1c(=O)c2[nH]c(-c3cccc(O)c3Br)nc2n(C)c1=O]

Procedure A was followed with p-tolualdehyde to obtain 1-112 as a white solid in 40% yield (32 mg). ¹H NMR (400 MHz, DMSO-d₆): δ (ppm) 13.74 (s, 1H), 8.06-7.98 (m, 2H), 7.37-7.27 (m, 2H), 3.50 (s, 3H), 3.27 (s, 3H), 2.36 (s, 3H).

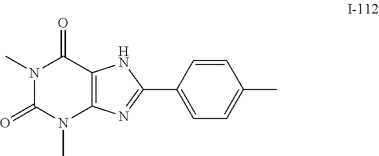

Procedure A was followed with 4-methyl-3-nitrobenzaldehyde to obtain Ia-113 as a white solid in 74% yield (70 mg). ¹H NMR (400 MHz, DMSO-d₆): δ (ppm) 14.16 (s, 1H), 8.75 (s, 1H), 8.33 (d, J=7.3 Hz, 1H), 7.67 (d, J=7.7 Hz, 1H), 3.51 (s, 3H), 3.27 (s, 3H), 2.58 (s, 3H).

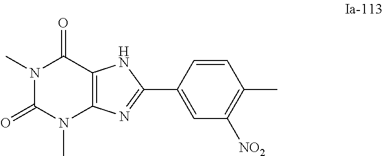

Procedure A was followed with 4-(pyridin-2-yl)benzaldehyde to obtain I-114 as an orange-brown solid in 84% yield (84 mg). ¹H NMR (400 MHz, DMSO-d₆): δ (ppm) 8.83 (d, J=5.4 Hz, 1H), 8.39-8.03 (m, 7H), 7.79-7.59 (m, 1H), 3.52 (s, 3H), 3.27 (s, 3H).

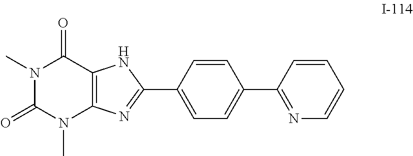 [SMILES: Cn1c(=O)c2[nH]c(-c3ccc(-c4ccccn4)cc3)nc2n(C)c1=O]

Procedure A was followed with 4-amino-2-methylsulfanylpyrimidine-5-carbaldehyde to obtain Ia-115 as a beige solid in 8% yield (8 mg). ¹H NMR (400 MHz, DMSO-d₆): δ (ppm) 13.54 (s, 1H), 9.29 (s, 1H), 7.59 (d, J=7.9 Hz, 2H), 3.83 (s, 3H), 3.48 (s, 3H), 3.25 (s, 3H).

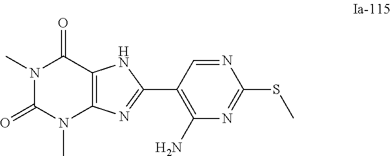

Procedure A was followed with 1H-pyrrolo[2,3-b]pyridine-3-carbaldehyde to obtain Ia-116 as a light brown solid in 13% yield (12 mg). ¹H NMR (400 MHz, DMSO-d₆): δ (ppm) 13.43 (s, 1H), 12.25 (s, 1H), 8.70 (d, J=6.9 Hz, 1H), 8.38-8.23 (m, 2H), 7.32-7.17 (m, 1H), 3.56 (s, 3H), 3.27 (s, 3H).

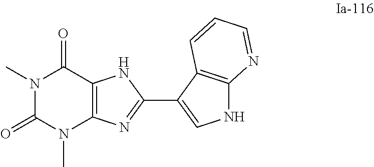 [SMILES: Cn1c(=O)c2[nH]c(-c3c[nH]c4ncccc34)nc2n(C)c1=O]

Procedure A was followed with benzothiophene-3-carboxaldehyde to obtain Ia-117 as a brownish solid in 26% yield (24 mg). ¹H NMR (400 MHz, DMSO-d₆): δ (ppm) 13.90 (s, 1H), 8.98 (dt, J=8.2, 1.2 Hz, 1H), 8.61 (s, 1H), 8.10 (dt, J=8.0, 1.0 Hz, 1H), 7.52 (dddd, J=27.1, 8.3, 7.1, 1.2 Hz, 2H), 3.58 (s, 3H), 3.29 (s, 3H).

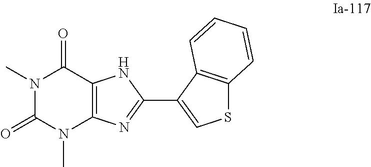 [SMILES: Cn1c(=O)c2[nH]c(-c3csc4ccccc34)nc2n(C)c1=O]

Procedure A was followed with 2-phenyl-1H-indole-3-carbaldehyde to obtain I-118 as a light brown solid in 18% yield (20 mg). ¹H NMR (400 MHz, DMSO-d₆): δ (ppm) 12.93 (s, 1H), 11.92 (s, 1H), 7.75 (d, J=7.9 Hz, 1H), 7.61-7.54 (m, 2H), 7.50-7.35 (m, 4H), 7.18 (dddd, J=34.7, 8.0, 7.1, 1.2 Hz, 2H), 3.50 (s, 3H), 3.26 (s, 3H).

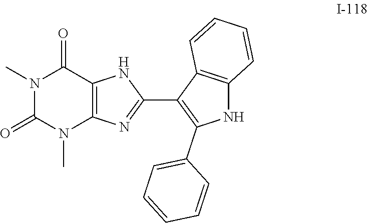 [SMILES: Cn1c(=O)c2[nH]c(-c3c(-c4ccccc4)[nH]c4ccccc34)nc2n(C)c1=O]

Procedure A was followed with 6-bromopyridine-3-carbaldehyde to obtain 1-119 as a beige solid in 44% yield (44 mg). ¹H NMR (400 MHz, DMSO-d₆): δ (ppm) 14.19 (s, 1H), 9.07 (d, J=2.5 Hz, 1H), 8.37 (dd, J=8.4, 2.5 Hz, 1H), 7.83 (d, J=8.4 Hz, 1H), 3.50 (s, 3H), 3.27 (s, 3H).

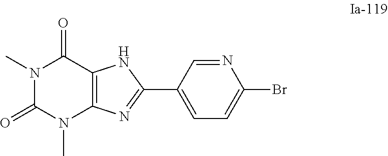 [SMILES: Cn1c(=O)c2[nH]c(-c3ccc(Br)nc3)nc2n(C)c1=O]

Procedure A was followed with 6-chloro-1H-indole-2-carbaldehyde to obtain Ia-120 as a brown solid in 33% yield (33 mg). ¹H NMR (400 MHz, DMSO-d₆): δ (ppm) 13.68 (s, 1H), 12.04 (d, J=11.1 Hz, 1H), 7.72-6.80 (m, 4H), 3.51 (d, J=4.2 Hz, 3H), 3.27 (s, 3H).

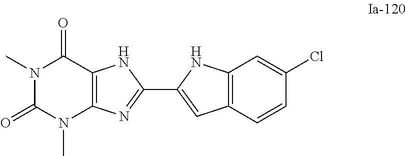 [SMILES: Cn1c(=O)c2[nH]c(-c3cc4ccc(Cl)cc4[nH]3)nc2n(C)c1=O]

Procedure A was followed with 4-bromo-1H-indole-3-carbaldehyde to obtain Ia-121 as a beige solid in 8% yield (9 mg). ¹H NMR (400 MHz, DMSO-d₆): δ (ppm) 11.99 (d, J=2.8 Hz, 1H), 7.83 (d, J=2.8 Hz, 1H), 7.53 (d, J=8.1 Hz, 1H), 7.32 (d, J=7.5 Hz, 1H), 7.12 (t, J=7.9 Hz, 1H), 3.49 (s, 3H), 3.27 (s, 3H).

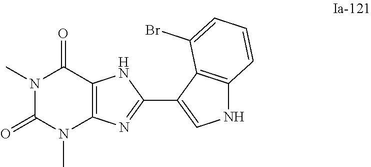 [SMILES: Cn1c(=O)c2[nH]c(-c3c[nH]c4cccc(Br)c34)nc2n(C)c1=O]

Procedure A was followed with 2-naphthaldehyde to obtain I-125 in 32% yield (29 mg) as a white solid. ¹H NMR (400 MHz, DMSO-d₆): δ (ppm) 14.01 (s, 1H, NH), 8.75 (s, 1H), 8.27 (dd, J=8.6, 1.8 Hz, 1H), 8.09-8.00 (m, 2H), 7.98 (dd, J=6.1, 3.4 Hz, 1H), 7.61 (dt, J=6.2, 3.4 Hz, 2H), 3.57 (s, 3H, CH3), 3.30 (s, 3H, CH3).

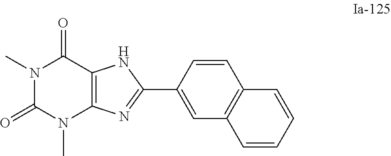 [SMILES: Cn1c(=O)c2[nH]c(-c3ccc4ccccc4c3)nc2n(C)c1=O]

Procedure A was followed with 2-chlorobenzaldehyde to obtain I-126 in 9% yield (8 mg) as a yellow solid. ¹H NMR (400 MHz, DMSO-d₆): δ (ppm) 13.81 (s, 1H, NH), 7.73 (dd, J=7.6, 1.8 Hz, 1H), 7.64 (dd, J=8.0, 1.3 Hz, 1H), 7.60-7.45 (m, 2H), 3.49 (s, 3H, CH3), 3.28 (s, 3H, CH3).

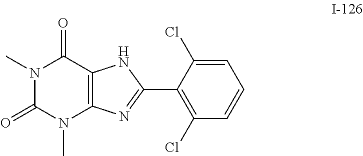 [SMILES: Cn1c(=O)c2[nH]c(-c3c(Cl)cccc3Cl)nc2n(C)c1=O]

Procedure A was followed with 3,4-dihydroxy-5-nitrobenzaldehyde to obtain Ia-127 in 7% yield (7 mg) as a light orange solid. ¹H NMR (400 MHz, DMSO-d₆): δ (ppm) 13.9 (s, 1H, NH), 10.64 (s, 2H, OH), 8.23 (d, J=2.1 Hz, 1H), 7.85 (d, J=2.2 Hz, 1H), 3.49 (s, 3H, CH3), 3.28 (s, 3H, CH3).

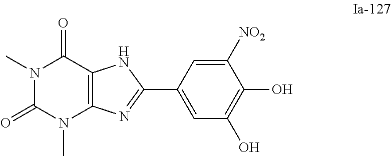 [SMILES: Cn1c(=O)c2[nH]c(-c3cc(O)c(O)c([N+](=O)[O-])c3)nc2n(C)c1=O]

Procedure A was followed with 4-pyridinecarboxaldehyde to obtain I-128 in 47% yield (36 mg) as a yellow solid. ¹H NMR (400 MHz, DMSO-d₆): δ (ppm) 13.96 (s, 1H, NH), 9.02-8.95 (m, 2H), 8.50-8.44 (m, 2H), 3.51 (s, 3H, CH3), 3.28 (s, 3H, CH3).

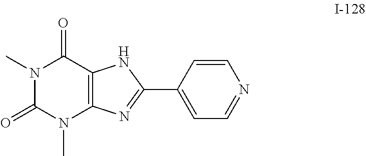 [SMILES: Cn1c(=O)c2[nH]c(-c3ccncc3)nc2n(C)c1=O]

Procedure A was followed with 2-fluoropyridine-3-carbaldehyde to obtain Ia-129 in 14% yield (11 mg) as a light yellow solid. ¹H NMR (400 MHz, DMSO-d₆): δ (ppm) 13.94 (s, 1H, NH), 8.50 (ddd, J=9.7, 7.6, 2.0 Hz, 1H), 8.38 (ddd, J=4.9, 2.0, 1.1 Hz, 1H), 7.56 (ddd, J=7.7, 4.8, 1.8 Hz, 1H), 3.51 (s, 3H, CH3), 3.28 (s, 3H, CH3).

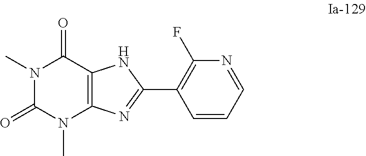 [SMILES: Cn1c(=O)c2[nH]c(-c3cccnc3F)nc2n(C)c1=O]

Procedure A was followed with 3-chloro-4-pyridinecarbaldehyde to obtain Ia-130 in 22% yield (20 mg) as a white solid. ¹H NMR (400 MHz, DMSO-d₆): δ (ppm) 14.19 (s, 1H), 8.83 (s, 1H), 8.68 (d, J=5.0 Hz, 1H), 7.82 (d, J=4.9 Hz, 1H), 3.50 (s, 3H, CH3), 3.28 (s, 3H, CH3).

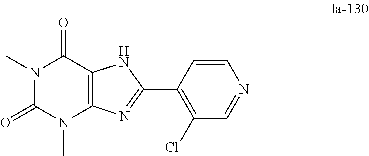 [SMILES: Cn1c(=O)c2[nH]c(-c3ccncc3Cl)nc2n(C)c1=O]

Procedure A was followed with 4-bromothiophene-2-carbaldehyde to obtain I-131 in 17% yield (17 mg) as a white solid. ¹H NMR (400 MHz, DMSO-d₆): δ (ppm) 14.05 (s, 1H, NH), 7.86 (s, 1H), 7.10 (s, 1H), 3.45 (s, 3H, CH3), 3.26 (s, 3H, CH3).

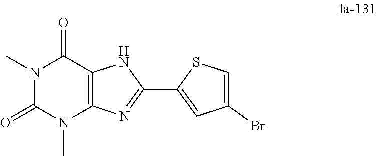 [SMILES: Cn1c(=O)c2[nH]c(-c3cc(Br)cs3)nc2n(C)c1=O]

Procedure A was followed with 1H-pyrazole-5-carbaldehyde to obtain Ia-132 in 44% yield (32 mg) as a white solid. ¹H NMR (400 MHz, DMSO-d₆): δ (ppm) 13.72 (s, 1H, NH), 13.34 (s, 1H, NH), 7.89 (s, 1H), 6.9 (s, 1H), 3.50 (s, 3H, CH3), 3.26 (s, 3H, CH3).

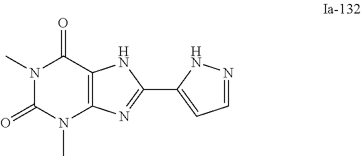 [SMILES: Cn1c(=O)c2[nH]c(-c3ccn[nH]3)nc2n(C)c1=O]

Procedure A was followed with indole-3-carboxaldehyde to obtain 1-133 in 11% yield (9 mg) as a dark yellow solid. ¹H NMR (400 MHz, DMSO-d₆): δ (ppm) 12.13 (s, 1H, NH), 9.93 (s, 1H, NH), 8.28 (d, J=3.1 Hz, 1H), 8.12-8.05 (m, 1H), 7.51 (dt, J=8.0, 1.0 Hz, 1H), 7.31-7.11 (m, 2H), 3.58 (s, 3H, CH3), 3.35 (s, 3H, CH3).

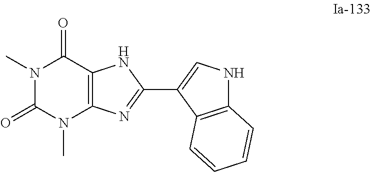 [SMILES: Cn1c(=O)c2[nH]c(-c3c[nH]c4ccccc34)nc2n(C)c1=O]

Procedure A was followed with 6-bromopyridine-2-carbaldehyde to obtain Ia-134 in 44% yield (44 mg) as a white solid. ¹H NMR (400 MHz, DMSO-d₆): δ (ppm) 14.27 (s, 1H, NH), 8.15 (dd, J=7.7, 1.7 Hz, 1H), 7.92 (td, J=7.8, 1.6 Hz, 1H), 7.75 (d, J=8.0 Hz, 1H), 3.52 (s, 3H, CH3), 3.27 (s, 3H, CH3).

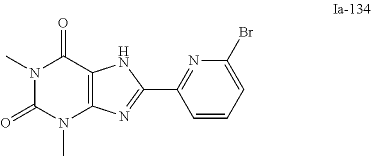 [SMILES: Cn1c(=O)c2[nH]c(-c3cccc(Br)n3)nc2n(C)c1=O]

Procedure A was followed with 3-bromopyridine-4-carbaldehyde to obtain Ia-135 in 49% yield (49 mg) as a white solid. ¹H NMR (400 MHz, DMSO-d₆): δ (ppm) 13.9 (s, 1H, NH), 8.95 (s, 1H), 8.71 (d, J=5.0 Hz, 1H), 7.77 (d, J=5.0 Hz, 1H), 3.50 (s, 3H, CH3), 3.28 (s, 3H, CH3).

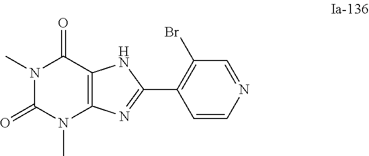 [SMILES: Cn1c(=O)c2[nH]c(-c3ccncc3Br)nc2n(C)c1=O]

Procedure A was followed with 4-bromo-5-nitrothiophene to obtain Ia-136 in 42% yield (39 mg) as a light orange solid. ¹H NMR (400 MHz, DMSO-d₆): δ (ppm) 14.54 (s, 1H, NH), 8.20 (d, J=4.4 Hz, 1H), 7.86 (d, J=4.4 Hz, 1H), 3.46 (s, 3H, CH3), 3.26 (s, 3H, CH3).

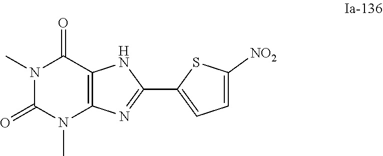 [SMILES: Cn1c(=O)c2[nH]c(-c3ccc([N+](=O)[O-])s3)nc2n(C)c1=O]

Procedure A was followed with 2-methylsulfonylbenzaldehyde to obtain Ia-137 in 52% yield (52 mg) as a white solid. ¹H NMR (400 MHz, DMSO-d₆): δ (ppm) 13.94 (s, 1H, NH), 8.14 (dd, J=7.6, 1.6 Hz, 1H), 7.93-7.79 (m, 2H), 7.74 (dd, J=7.4, 1.6 Hz, 1H), 3.60 (s, 3H, CH3), 3.48 (s, 3H, CH3), 3.29 (s, 3H, CH3).

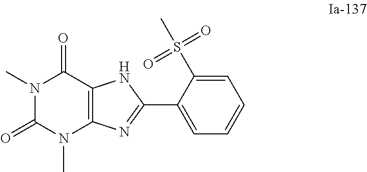 [SMILES: Cn1c(=O)c2[nH]c(-c3ccccc3S(C)(=O)=O)nc2n(C)c1=O]

Procedure A was followed with 3-methylsulfonylbenzaldehyde to obtain Ia-138 in 12% yield (12 mg) as a white solid. ¹H NMR (400 MHz, DMSO-d₆): δ (ppm) 14.18 (s, 1H, NH), 8.68 (t, J=1.8 Hz, 1H), 8.43 (dt, J=8.1, 1.3 Hz, 1H), 8.03 (ddd, J=7.9, 1.8, 1.1 Hz, 1H), 7.81 (t, J=7.9 Hz, 1H), 3.52 (s, 3H, CH3), 3.29 (s, 3H, CH3), 3.27 (s, 3H, CH3).

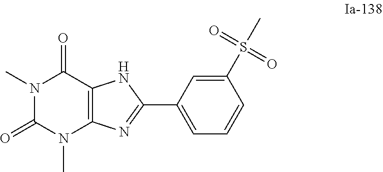 [SMILES: Cn1c(=O)c2[nH]c(-c3cccc(S(C)(=O)=O)c3)nc2n(C)c1=O]

Procedure A was followed with 3-fluoro-4-nitrobenzaldehyde to obtain Ia-139 in 28% yield (27 mg) as a yellow solid. ¹H NMR (400 MHz, DMSO-d₆): δ (ppm) 14.32 (s, 1H, NH), 8.29 (t, J=8.3 Hz, 1H), 8.17 (dd, J=12.5, 1.8 Hz, 1H), 8.11 (dd, J=8.6, 1.8 Hz, 1H), 3.49 (s, 3H, CH3), 3.26 (s, 3H, CH3).

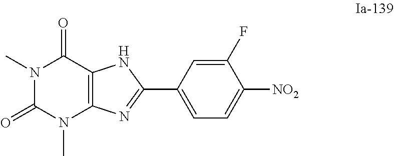 [SMILES: Cn1c(=O)c2[nH]c(-c3ccc([N+](=O)[O-])c(F)c3)nc2n(C)c1=O]

Procedure A was followed with 2-2chloro-5-nitrobenzaldehyde to obtain Ia-140 in 15% yield (15 mg) as a white solid. ¹H NMR (400 MHz, DMSO-d₆): δ (ppm) 14.15 (s, 1H, NH), 8.59 (d, J=2.7 Hz, 1H), 8.35 (dd, J=8.8, 2.8 Hz, 1H), 7.95 (d, J=8.9 Hz, 1H), 3.50 (s, 3H, CH3), 3.29 (s, 3H, CH3).

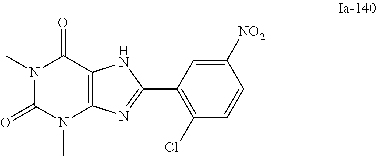 [SMILES: Cn1c(=O)c2[nH]c(-c3cc([N+](=O)[O-])ccc3Cl)nc2n(C)c1=O]

Procedure B was followed with (6-chloropyridin-3-yl)boronic acid to obtain Ia-4 as a yellowish powder in 21% yield (23 mg). ¹H NMR (700 MHz, Chloroform-d) δ 8.53 (s, 1H), 8.31 (d, J=8.5 Hz, 1H), 8.00 (d, J=8.6 Hz, 1H), 4.12 (s, 3H), 3.78 (s, 3H), 3.43 (s, 3H).

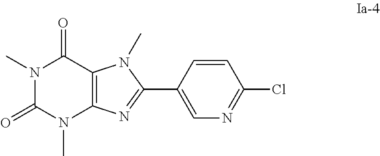 [SMILES: Cn1c(=O)c2c(nc(-c3ccc(Cl)nc3)n2C)n(C)c1=O]

Procedure B was followed with thiophen-3-ylboronic acid to obtain I-11 as a light-yellow solid in 24% yield (24 mg). ¹H NMR (700 MHz, Chloroform-d) δ 8.02 (s, 1H), 7.90 (d, J=8.0 Hz, 1H), 7.39 (d, J=7.9 Hz, 1H), 4.18 (s, 3H), 3.76 (s, 3H), 3.41 (s, 3H).

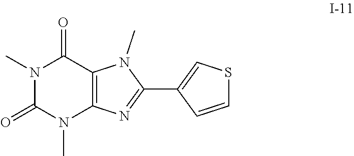 [SMILES: Cn1c(=O)c2c(nc(-c3ccsc3)n2C)n(C)c1=O]

Procedure B was followed with (3-nitrophenyl)boronic acid to obtain Ia-3 as a yellowish powder in 23% yield (27 mg). ¹H NMR (700 MHz, Chloroform-d) δ 8.71 (s, 1H), 8.64-8.51 (m, 2H), 7.88 (dd, J=8.4 Hz, 1H), 4.08 (s, 3H), 3.72 (s, 3H), 3.39 (s, 3H).

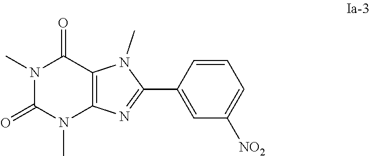 [SMILES: Cn1c(=O)c2c(nc(-c3cccc([N+](=O)[O-])c3)n2C)n(C)c1=O]

Procedure C was followed with 1-bromo-3,5-bis(trifluoromethyl)benzene to obtain I-122 as a white powder in 60% yield (122 mg). ¹H NMR (400 MHz, Chloroform-d) δ 8.21-8.16 (m, 2H), 8.03 (tt, J=1.6, 0.8 Hz, 1H), 4.14 (s, 3H), 3.64 (s, 3H), 3.44 (s, 3H).

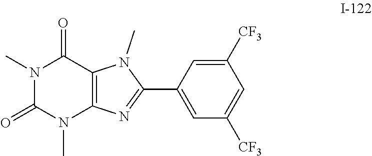 [SMILES: Cn1c(=O)c2c(nc(-c3cc(C(F)(F)F)cc(C(F)(F)F)c3)n2C)n(C)c1=O]

Procedure C was followed with 1-bromo-4-(methylsulfonyl)benzene to obtain Ia-123 as a white powder in 57% yield (100 mg). ¹H NMR (400 MHz, Chloroform-d) δ 8.14-8.08 (m, 2H), 7.97-7.91 (m, 2H), 4.12 (s, 3H), 3.63 (s, 3H), 3.45 (s, 3H), 3.11 (s, 3H).

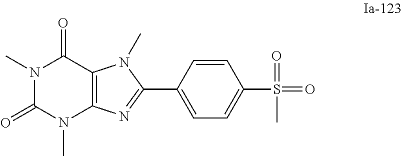 [SMILES: Cn1c(=O)c2c(nc(-c3ccc(S(C)(=O)=O)cc3)n2C)n(C)c1=O]

Procedure C was followed with 2-bromo-5-(trifluoromethyl)pyridine to obtain Ia-124 as a yellow powder in 50% yield (85 mg). ¹H NMR (400 MHz, Chloroform-d) δ 8.94 (dq, J=2.5, 0.9 Hz, 1H), 8.41 (dt, J=8.4, 0.8 Hz, 1H), 8.09-8.01 (m, 1H), 4.52 (s, 3H), 3.65 (s, 3H), 3.45 (s, 3H).

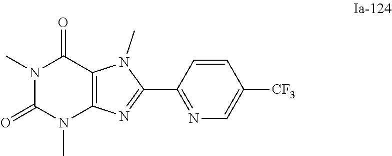 [SMILES: Cn1c(=O)c2c(nc(-c3ccc(C(F)(F)F)cn3)n2C)n(C)c1=O]

Using the methods described above, the following additional compounds of Formula (I)/Ia were prepared:

| Compound | |
|---|---|
| No. | Structure |
| Ia-2 | 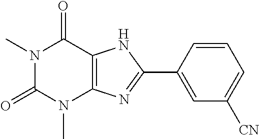 [SMILES: Cn1c(=O)c2[nH]c(-c3cccc(C#N)c3)nc2n(C)c1=O] |
| Ia-5 | 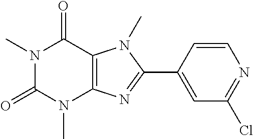 [SMILES: Cn1c(=O)c2c(nc(-c3ccnc(Cl)c3)n2C)n(C)c1=O] |
| Ia-6 | 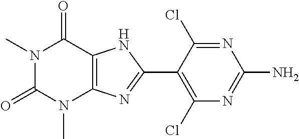 [SMILES: Cn1c(=O)c2[nH]c(-c3c(Cl)nc(N)nc3Cl)nc2n(C)c1=O] |
| Ia-7 | 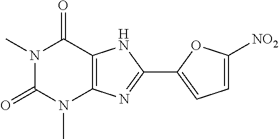 [SMILES: Cn1c(=O)c2[nH]c(-c3ccc([N+](=O)[O-])o3)nc2n(C)c1=O] |
| Ia-9 | 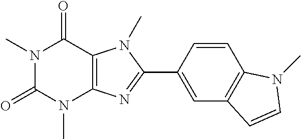 |
| Ia-10 | 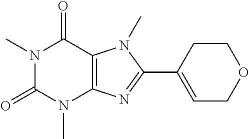 [SMILES: Cn1c(=O)c2c(nc(C3=CCOCC3)n2C)n(C)c1=O] |
| Ia-11 | 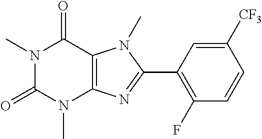 [SMILES: Cn1c(=O)c2c(nc(-c3cc(C(F)(F)F)ccc3F)n2C)n(C)c1=O] |
| Ia-12 | 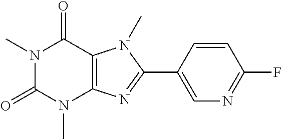 [SMILES: Cn1c(=O)c2c(nc(-c3ccc(F)nc3)n2C)n(C)c1=O] |
| Ia-13 | 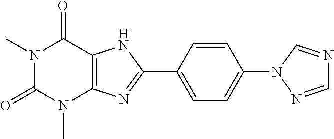 [SMILES: Cn1c(=O)c2[nH]c(-c3ccc(-n4cncn4)cc3)nc2n(C)c1=O] |
| Ia-14 | 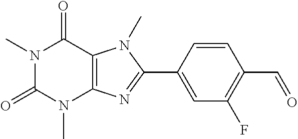 [SMILES: Cn1c(=O)c2c(nc(-c3ccc(C=O)c(F)c3)n2C)n(C)c1=O] |
| Ia-15 | 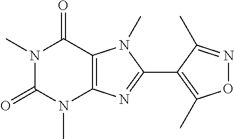 [SMILES: Cc1noc(C)c1-c1nc2c(c(=O)n(C)c(=O)n2C)n1C] |
| Ia-16 | 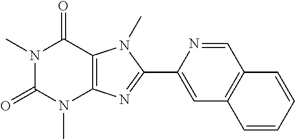 [SMILES: Cn1c(=O)c2c(nc(-c3cc4ccccc4cn3)n2C)n(C)c1=O] |
| Ia-17 | 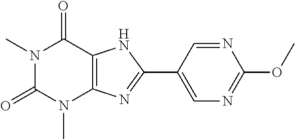 |
| Ia-18 | 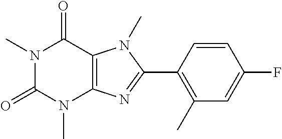 [SMILES: Cc1cc(F)ccc1-c1nc2c(c(=O)n(C)c(=O)n2C)n1C] |
| Ia-19 | 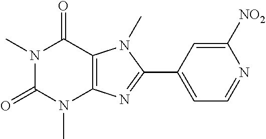 [SMILES: Cn1c(=O)c2c(nc(-c3ccnc([N+](=O)[O-])c3)n2C)n(C)c1=O] |
| Ia-20 | 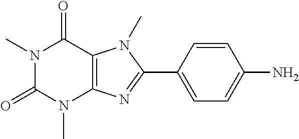 [SMILES: Cn1c(=O)c2c(nc(-c3ccc(N)cc3)n2C)n(C)c1=O] |
| Ia-21 | 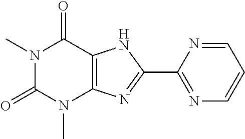 [SMILES: Cn1c(=O)c2[nH]c(-c3ncccn3)nc2n(C)c1=O] |
| Ia-22 | 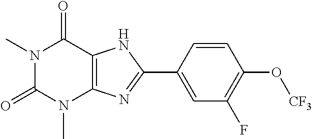 [SMILES: Cn1c(=O)c2[nH]c(-c3ccc(OC(F)(F)F)c(F)c3)nc2n(C)c1=O] |
| Ia-23 | 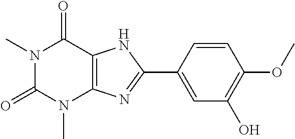 |
| Ia-24 | 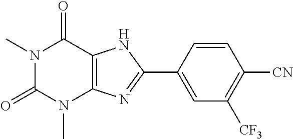 [SMILES: Cn1c(=O)c2[nH]c(-c3ccc(C#N)c(C(F)(F)F)c3)nc2n(C)c1=O] |
| Ia-25 | 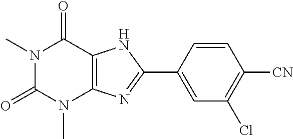 [SMILES: Cn1c(=O)c2[nH]c(-c3ccc(C#N)c(Cl)c3)nc2n(C)c1=O] |
| Ia-26 | 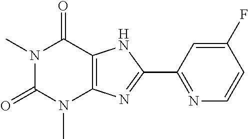 [SMILES: Cn1c(=O)c2[nH]c(-c3cc(F)ccn3)nc2n(C)c1=O] |
| Ia-27 | 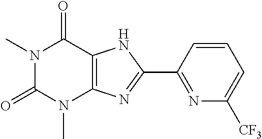 [SMILES: Cn1c(=O)c2[nH]c(-c3cccc(C(F)(F)F)n3)nc2n(C)c1=O] |
| Ia-28 | 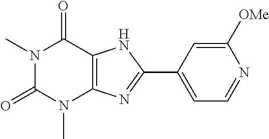 [SMILES: COc1cc(-c2nc3c([nH]2)c(=O)n(C)c(=O)n3C)ccn1] |
| Ia-29 | 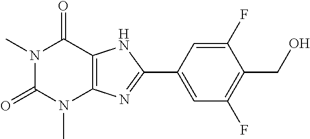 [SMILES: Cn1c(=O)c2[nH]c(-c3cc(F)c(CO)c(F)c3)nc2n(C)c1=O] |
| Ia-30 | 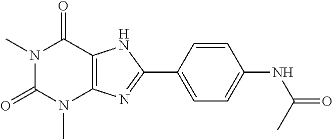 [SMILES: CC(=O)Nc1ccc(-c2nc3c([nH]2)c(=O)n(C)c(=O)n3C)cc1] |
| Ia-31 | 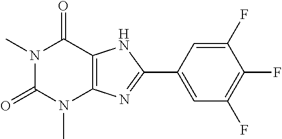 [SMILES: Cn1c(=O)c2[nH]c(-c3cc(F)c(F)c(F)c3)nc2n(C)c1=O] |
| Ia-32 | 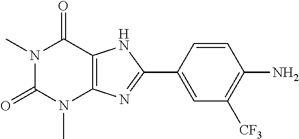 [SMILES: Cn1c(=O)c2[nH]c(-c3ccc(N)c(C(F)(F)F)c3)nc2n(C)c1=O] |
| Ia-33 | 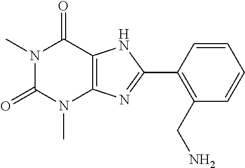 [SMILES: Cn1c(=O)c2[nH]c(-c3ccccc3CN)nc2n(C)c1=O] |
| Ia-34 | 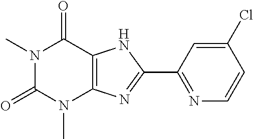 [SMILES: Cn1c(=O)c2[nH]c(-c3cc(Cl)ccn3)nc2n(C)c1=O] |
| Ia-35 | 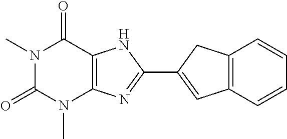 [SMILES: Cn1c(=O)c2[nH]c(C3=Cc4ccccc4C3)nc2n(C)c1=O] |
| Ia-36 | 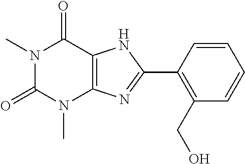 [SMILES: Cn1c(=O)c2[nH]c(-c3ccccc3CO)nc2n(C)c1=O] |
| Ia-37 | 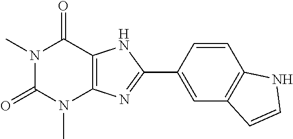 [SMILES: Cn1c(=O)c2[nH]c(-c3ccc4[nH]ccc4c3)nc2n(C)c1=O] |
| Ia-38 | 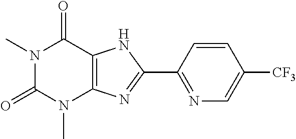 [SMILES: Cn1c(=O)c2[nH]c(-c3ccc(C(F)(F)F)cn3)nc2n(C)c1=O] |
| Ia-39 | 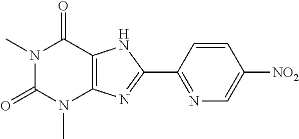 [SMILES: Cn1c(=O)c2[nH]c(-c3ccc([N+](=O)[O-])cn3)nc2n(C)c1=O] |
| Ia-40 | 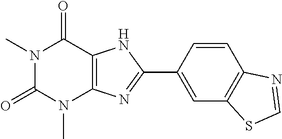 [SMILES: Cn1c(=O)c2[nH]c(-c3ccc4ncsc4c3)nc2n(C)c1=O] |
| Ia-41 | 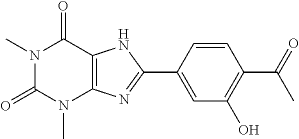 [SMILES: CC(=O)c1ccc(-c2nc3c([nH]2)c(=O)n(C)c(=O)n3C)cc1O] |
| Ia-42 | 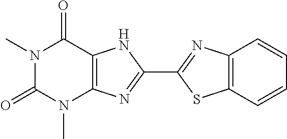 [SMILES: Cn1c(=O)c2[nH]c(-c3nc4ccccc4s3)nc2n(C)c1=O] |
| Ia-43 | 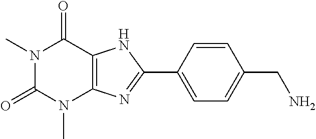 [SMILES: Cn1c(=O)c2[nH]c(-c3ccc(CN)cc3)nc2n(C)c1=O] |
| Ia-44 | 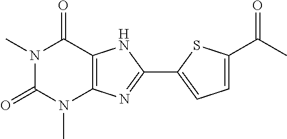 [SMILES: CC(=O)c1ccc(-c2nc3c([nH]2)c(=O)n(C)c(=O)n3C)s1] |
| Ia-45 | 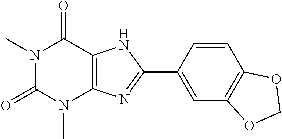 [SMILES: Cn1c(=O)c2[nH]c(-c3ccc4c(c3)OCO4)nc2n(C)c1=O] |
| I-46 | 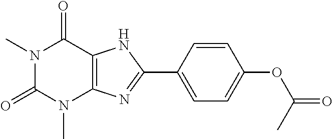 [SMILES: CC(=O)Oc1ccc(-c2nc3c([nH]2)c(=O)n(C)c(=O)n3C)cc1] |
| Ia-47 | 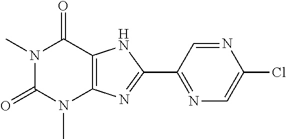 [SMILES: Cn1c(=O)c2[nH]c(-c3cnc(Cl)cn3)nc2n(C)c1=O] |
| I-48 | 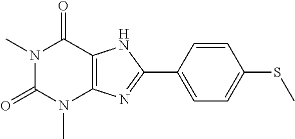 |
| I-49 | 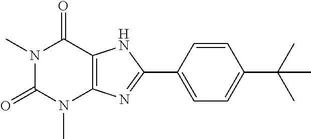 [SMILES: Cn1c(=O)c2[nH]c(-c3ccc(C(C)(C)C)cc3)nc2n(C)c1=O] |
| Ia-50 | 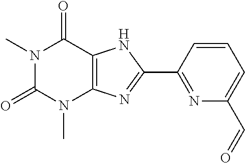 [SMILES: Cn1c(=O)c2[nH]c(-c3cccc(C=O)n3)nc2n(C)c1=O] |
| Ia-51 | 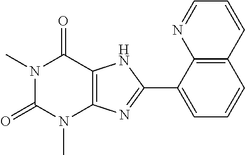 [SMILES: Cn1c(=O)c2[nH]c(-c3cccc4cccnc34)nc2n(C)c1=O] |
| I-52 | 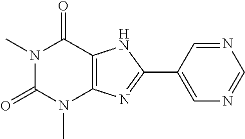 [SMILES: Cn1c(=O)c2[nH]c(-c3cncnc3)nc2n(C)c1=O] |
| Ia-53 | 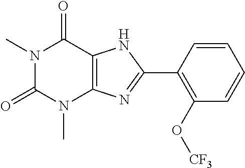 [SMILES: Cn1c(=O)c2[nH]c(-c3ccccc3OC(F)(F)F)nc2n(C)c1=O] |
| I-54 | 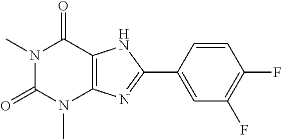 [SMILES: Cn1c(=O)c2[nH]c(-c3ccc(F)c(F)c3)nc2n(C)c1=O] |
| Ia-55 | 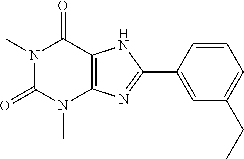 [SMILES: CCc1cccc(-c2nc3c([nH]2)c(=O)n(C)c(=O)n3C)c1] |
| Ia-56 | 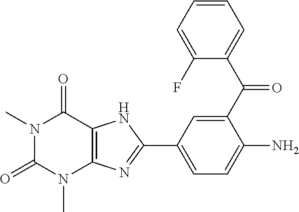 [SMILES: Cn1c(=O)c2[nH]c(-c3ccc(N)c(C(=O)c4ccccc4F)c3)nc2n(C)c1=O] |
| Ia-57 | 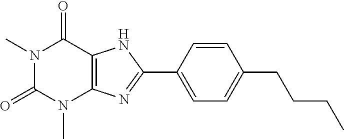 [SMILES: CCCCc1ccc(-c2nc3c([nH]2)c(=O)n(C)c(=O)n3C)cc1] |
| Ia-58 | 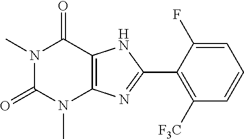 [SMILES: Cn1c(=O)c2[nH]c(-c3c(F)cccc3C(F)(F)F)nc2n(C)c1=O] |
| Ia-59 | 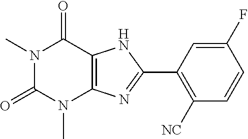 [SMILES: Cn1c(=O)c2[nH]c(-c3cc(F)ccc3C#N)nc2n(C)c1=O] |
| Ia-60 | 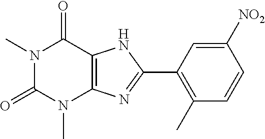 [SMILES: Cc1ccc([N+](=O)[O-])cc1-c1nc2c([nH]1)c(=O)n(C)c(=O)n2C] |
| Ia-61 | 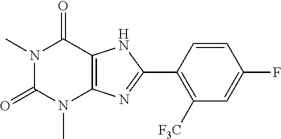 [SMILES: Cn1c(=O)c2[nH]c(-c3ccc(F)cc3C(F)(F)F)nc2n(C)c1=O] |
| Ia-62 | 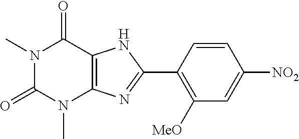 [SMILES: COc1cc([N+](=O)[O-])ccc1-c1nc2c([nH]1)c(=O)n(C)c(=O)n2C] |
| Ia-63 | 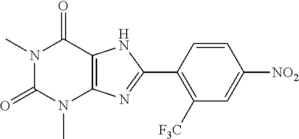 [SMILES: Cn1c(=O)c2[nH]c(-c3ccc([N+](=O)[O-])cc3C(F)(F)F)nc2n(C)c1=O] |
| Ia-64 | 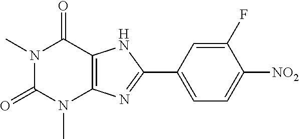 [SMILES: Cn1c(=O)c2[nH]c(-c3ccc([N+](=O)[O-])c(F)c3)nc2n(C)c1=O] |
| Ia-65 | 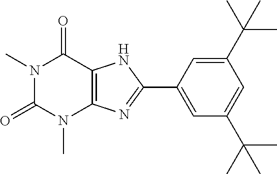 [SMILES: Cn1c(=O)c2[nH]c(-c3cc(C(C)(C)C)cc(C(C)(C)C)c3)nc2n(C)c1=O] |
| Ia-66 | 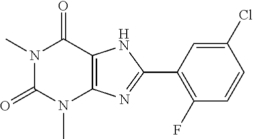 [SMILES: Cn1c(=O)c2[nH]c(-c3cc(Cl)ccc3F)nc2n(C)c1=O] |
| Ia-67 | 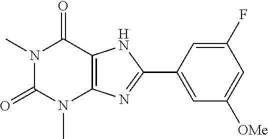 [SMILES: COc1cc(F)cc(-c2nc3c([nH]2)c(=O)n(C)c(=O)n3C)c1] |
| Ia-68 | 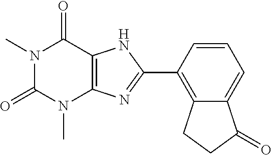 [SMILES: Cn1c(=O)c2[nH]c(-c3cccc4c3CCC4=O)nc2n(C)c1=O] |
| Ia-69 | 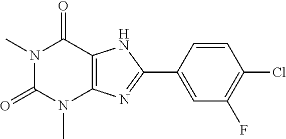 [SMILES: Cn1c(=O)c2[nH]c(-c3ccc(Cl)c(F)c3)nc2n(C)c1=O] |
| Ia-70 | 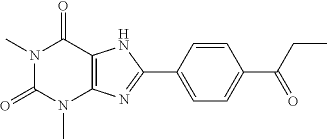 [SMILES: CCC(=O)c1ccc(-c2nc3c([nH]2)c(=O)n(C)c(=O)n3C)cc1] |
| Ia-71 | 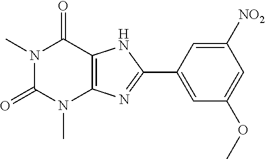 [SMILES: COc1cc(-c2nc3c([nH]2)c(=O)n(C)c(=O)n3C)cc([N+](=O)[O-])c1] |
| Ia-72 | 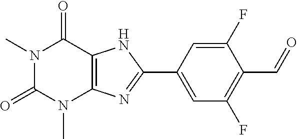 [SMILES: Cn1c(=O)c2[nH]c(-c3cc(F)c(C=O)c(F)c3)nc2n(C)c1=O] |
| Ia-73 | 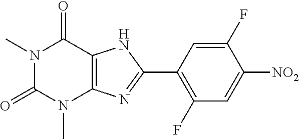 [SMILES: Cn1c(=O)c2[nH]c(-c3cc(F)c([N+](=O)[O-])cc3F)nc2n(C)c1=O] |
| Ia-74 | 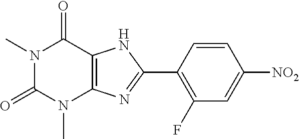 [SMILES: Cn1c(=O)c2[nH]c(-c3ccc([N+](=O)[O-])cc3F)nc2n(C)c1=O] |
| Ia-75 | 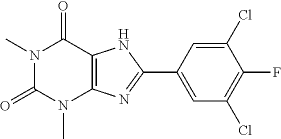 [SMILES: Cn1c(=O)c2[nH]c(-c3cc(Cl)c(F)c(Cl)c3)nc2n(C)c1=O] |
| Ia-76 | 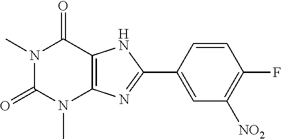 [SMILES: Cn1c(=O)c2[nH]c(-c3ccc(F)c([N+](=O)[O-])c3)nc2n(C)c1=O] |
| Ia-77 | 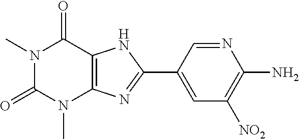 [SMILES: Cn1c(=O)c2[nH]c(-c3cnc(N)c([N+](=O)[O-])c3)nc2n(C)c1=O] |
| Ia-78 | 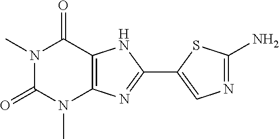 [SMILES: Cn1c(=O)c2[nH]c(-c3cnc(N)s3)nc2n(C)c1=O] |
| Ia-79 | 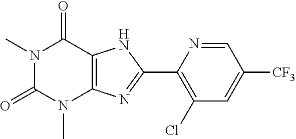 [SMILES: Cn1c(=O)c2[nH]c(-c3ncc(C(F)(F)F)cc3Cl)nc2n(C)c1=O] |
| Ia-80 | 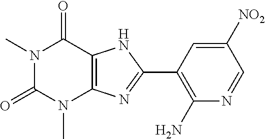 [SMILES: Cn1c(=O)c2[nH]c(-c3cc([N+](=O)[O-])cnc3N)nc2n(C)c1=O] |
| Ia-81 | 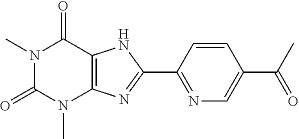 [SMILES: CC(=O)c1ccc(-c2nc3c([nH]2)c(=O)n(C)c(=O)n3C)nc1] |
| Ia-82 | 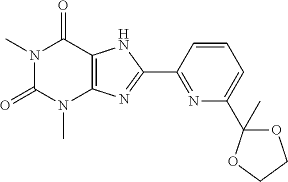 [SMILES: Cn1c(=O)c2[nH]c(-c3cccc(C4(C)OCCO4)n3)nc2n(C)c1=O] |
| Ia-83 | 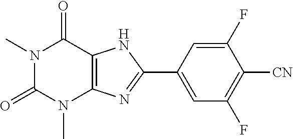 [SMILES: Cn1c(=O)c2[nH]c(-c3cc(F)c(C#N)c(F)c3)nc2n(C)c1=O] |
| Ia-84 | 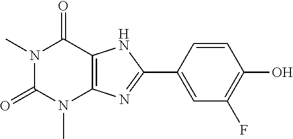 [SMILES: Cn1c(=O)c2[nH]c(-c3ccc(O)c(F)c3)nc2n(C)c1=O] |
| Ia-85 | 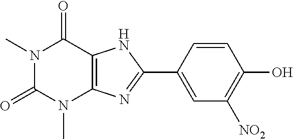 [SMILES: Cn1c(=O)c2[nH]c(-c3ccc(O)c([N+](=O)[O-])c3)nc2n(C)c1=O] |
| I-86 | 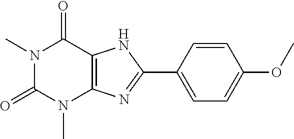 |
| Ia-87 | 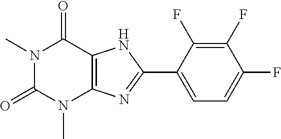 [SMILES: Cn1c(=O)c2[nH]c(-c3ccc(F)c(F)c3F)nc2n(C)c1=O] |
| Ia-88 | 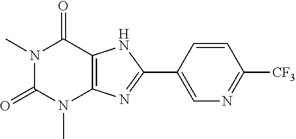 [SMILES: Cn1c(=O)c2[nH]c(-c3ccc(C(F)(F)F)nc3)nc2n(C)c1=O] |
| I-89 | 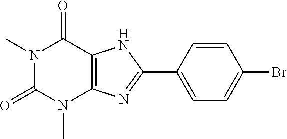 [SMILES: Cn1c(=O)c2[nH]c(-c3ccc(Br)cc3)nc2n(C)c1=O] |
| I-90 | 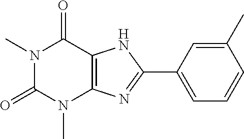 |
| I-91 | 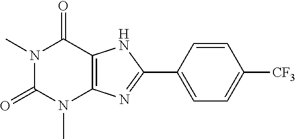 [SMILES: Cn1c(=O)c2[nH]c(-c3ccc(C(F)(F)F)cc3)nc2n(C)c1=O] |
| I-92 | 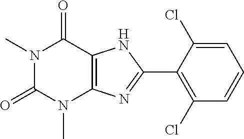 [SMILES: Cn1c(=O)c2[nH]c(-c3c(Cl)cccc3Cl)nc2n(C)c1=O] |
| Ia-93 | 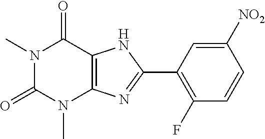 [SMILES: Cn1c(=O)c2[nH]c(-c3cc([N+](=O)[O-])ccc3F)nc2n(C)c1=O] |
| Ia-94 | 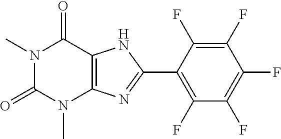 [SMILES: Cn1c(=O)c2[nH]c(-c3c(F)c(F)c(F)c(F)c3F)nc2n(C)c1=O] |
| I-95 | 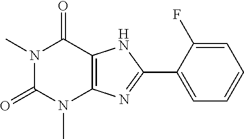 [SMILES: Cn1c(=O)c2[nH]c(-c3ccccc3F)nc2n(C)c1=O] |
| I-96 | 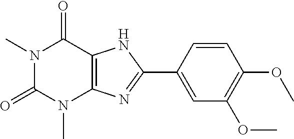 |
| Ia-97 | 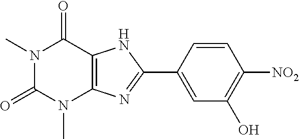 [SMILES: Cn1c(=O)c2[nH]c(-c3ccc([N+](=O)[O-])c(O)c3)nc2n(C)c1=O] |
| I-98 | 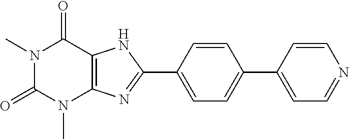 [SMILES: Cn1c(=O)c2[nH]c(-c3ccc(-c4ccncc4)cc3)nc2n(C)c1=O] |
| Ia-99 | 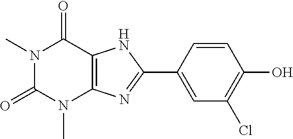 [SMILES: Cn1c(=O)c2[nH]c(-c3ccc(O)c(Cl)c3)nc2n(C)c1=O] |
| Ia-100 | 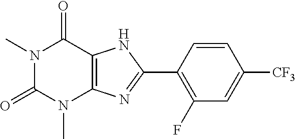 [SMILES: Cn1c(=O)c2[nH]c(-c3ccc(C(F)(F)F)cc3F)nc2n(C)c1=O] |
| I-101 | 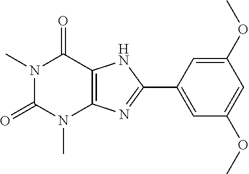 [SMILES: COc1cc(OC)cc(-c2nc3c([nH]2)c(=O)n(C)c(=O)n3C)c1] |
| Ia-102 | 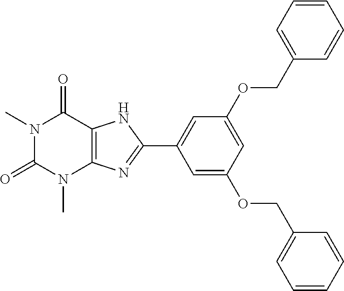 [SMILES: Cn1c(=O)c2[nH]c(-c3cc(OCc4ccccc4)cc(OCc4ccccc4)c3)nc2n(C)c1=O] |
| Ia-103 | 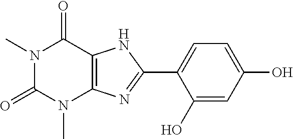 [SMILES: Cn1c(=O)c2[nH]c(-c3ccc(O)cc3O)nc2n(C)c1=O] |
| Ia-104 | 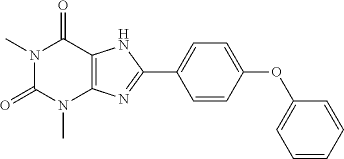 [SMILES: Cn1c(=O)c2[nH]c(-c3ccc(Oc4ccccc4)cc3)nc2n(C)c1=O] |
| Ia-105 | 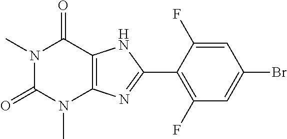 [SMILES: Cn1c(=O)c2[nH]c(-c3c(F)cc(Br)cc3F)nc2n(C)c1=O] |
| Ia-106 | 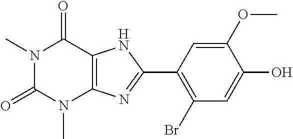 |
| I-107 | 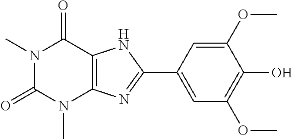 |
| Ia-108 | 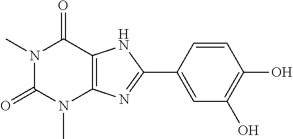 [SMILES: Cn1c(=O)c2[nH]c(-c3ccc(O)c(O)c3)nc2n(C)c1=O] |
| I-109 | 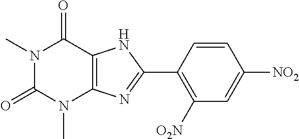 [SMILES: Cn1c(=O)c2[nH]c(-c3ccc([N+](=O)[O-])cc3[N+](=O)[O-])nc2n(C)c1=O] |
| Ia-110 | 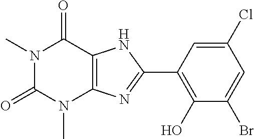 [SMILES: Cn1c(=O)c2[nH]c(-c3cc(Cl)cc(Br)c3O)nc2n(C)c1=O] |
| Ia-111 | 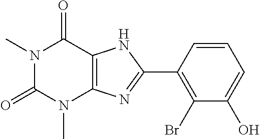 [SMILES: Cn1c(=O)c2[nH]c(-c3cccc(O)c3Br)nc2n(C)c1=O] |
| I-112 | 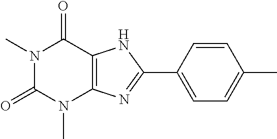 |
| Ia-113 | 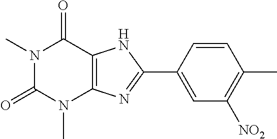 |
| I-114 | 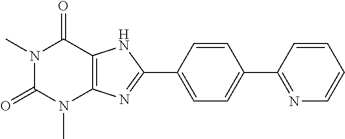 [SMILES: Cn1c(=O)c2[nH]c(-c3ccc(-c4ccccn4)cc3)nc2n(C)c1=O] |
| Ia-115 | 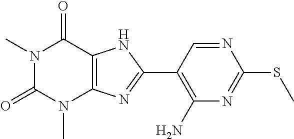 |
| Ia-116 | 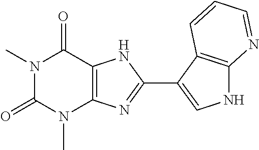 [SMILES: Cn1c(=O)c2[nH]c(-c3c[nH]c4ncccc34)nc2n(C)c1=O] |
| Ia-117 | 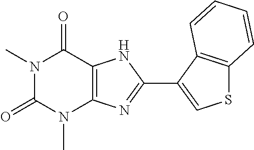 [SMILES: Cn1c(=O)c2[nH]c(-c3csc4ccccc34)nc2n(C)c1=O] |
| I-118 | 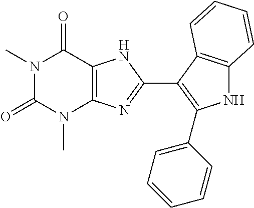 [SMILES: Cn1c(=O)c2[nH]c(-c3c(-c4ccccc4)[nH]c4ccccc34)nc2n(C)c1=O] |
| I-119 | 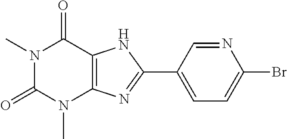 [SMILES: Cn1c(=O)c2[nH]c(-c3ccc(Br)nc3)nc2n(C)c1=O] |
| Ia-120 | 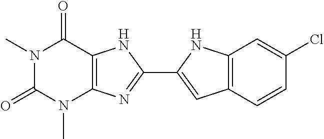 [SMILES: Cn1c(=O)c2[nH]c(-c3cc4ccc(Cl)cc4[nH]3)nc2n(C)c1=O] |
| Ia-121 | 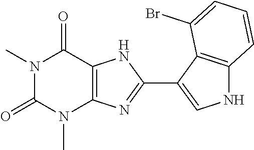 [SMILES: Cn1c(=O)c2[nH]c(-c3c[nH]c4cccc(Br)c34)nc2n(C)c1=O] |
| I-122 | 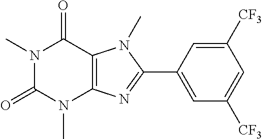 [SMILES: Cn1c(=O)c2c(nc(-c3cc(C(F)(F)F)cc(C(F)(F)F)c3)n2C)n(C)c1=O] |
| Ia-123 | 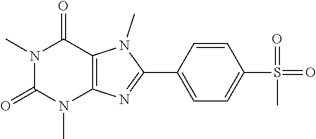 [SMILES: Cn1c(=O)c2c(nc(-c3ccc(S(C)(=O)=O)cc3)n2C)n(C)c1=O] |
| Ia-124 | 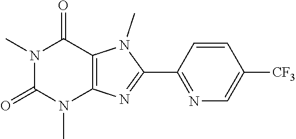 [SMILES: Cn1c(=O)c2c(nc(-c3ccc(C(F)(F)F)cn3)n2C)n(C)c1=O] |
| I-125 | 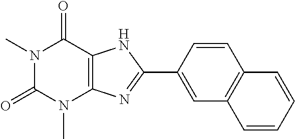 [SMILES: Cn1c(=O)c2[nH]c(-c3ccc4ccccc4c3)nc2n(C)c1=O] |
| I-126 | 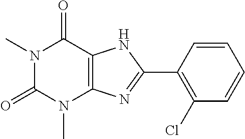 [SMILES: Cn1c(=O)c2[nH]c(-c3ccccc3Cl)nc2n(C)c1=O] |
| Ia-127 | 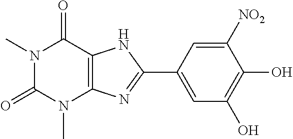 [SMILES: Cn1c(=O)c2[nH]c(-c3cc(O)c(O)c([N+](=O)[O-])c3)nc2n(C)c1=O] |
| I-128 | 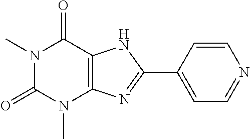 [SMILES: Cn1c(=O)c2[nH]c(-c3ccncc3)nc2n(C)c1=O] |
| Ia-129 | 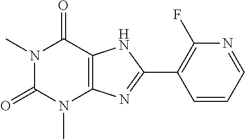 [SMILES: Cn1c(=O)c2[nH]c(-c3cccnc3F)nc2n(C)c1=O] |
| Ia-130 | 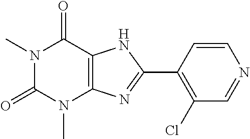 [SMILES: Cn1c(=O)c2[nH]c(-c3ccncc3Cl)nc2n(C)c1=O] |
| I-131 | 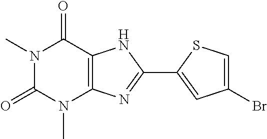 [SMILES: Cn1c(=O)c2[nH]c(-c3cc(Br)cs3)nc2n(C)c1=O] |
| Ia-132 | 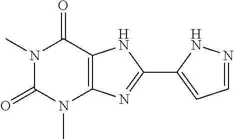 [SMILES: Cn1c(=O)c2[nH]c(-c3ccn[nH]3)nc2n(C)c1=O] |
| I-133 | 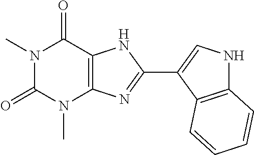 [SMILES: Cn1c(=O)c2[nH]c(-c3c[nH]c4ccccc34)nc2n(C)c1=O] |
| Ia-134 | 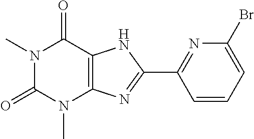 [SMILES: Cn1c(=O)c2[nH]c(-c3cccc(Br)n3)nc2n(C)c1=O] |
| Ia-135 | 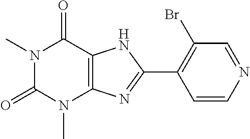 [SMILES: Cn1c(=O)c2[nH]c(-c3ccncc3Br)nc2n(C)c1=O] |
| Ia-136 | 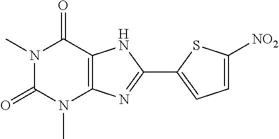 [SMILES: Cn1c(=O)c2[nH]c(-c3ccc([N+](=O)[O-])s3)nc2n(C)c1=O] |
| Ia-137 | 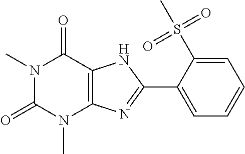 [SMILES: Cn1c(=O)c2[nH]c(-c3ccccc3S(C)(=O)=O)nc2n(C)c1=O] |
| Ia-138 | 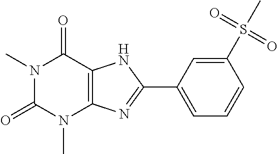 [SMILES: Cn1c(=O)c2[nH]c(-c3cccc(S(C)(=O)=O)c3)nc2n(C)c1=O] |
| Ia-139 | 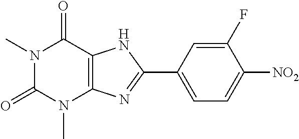 [SMILES: Cn1c(=O)c2[nH]c(-c3ccc([N+](=O)[O-])c(F)c3)nc2n(C)c1=O] |
| Ia-140 | 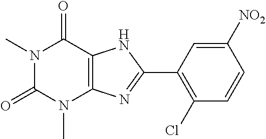 [SMILES: Cn1c(=O)c2[nH]c(-c3cc([N+](=O)[O-])ccc3Cl)nc2n(C)c1=O] |
| I-141 | 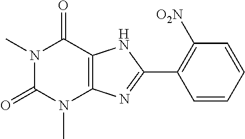 [SMILES: Cn1c(=O)c2[nH]c(-c3ccccc3[N+](=O)[O-])nc2n(C)c1=O] |
| Ia-142 | 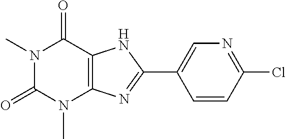 [SMILES: Cn1c(=O)c2[nH]c(-c3ccc(Cl)nc3)nc2n(C)c1=O] |
| I-143 | 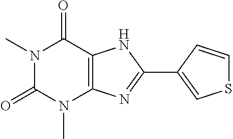 [SMILES: Cn1c(=O)c2[nH]c(-c3ccsc3)nc2n(C)c1=O] |
| I-1 | 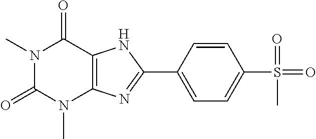 [SMILES: Cn1c(=O)c2[nH]c(-c3ccc(S(C)(=O)=O)cc3)nc2n(C)c1=O] |
| I-2 | 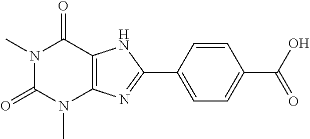 [SMILES: Cn1c(=O)c2[nH]c(-c3ccc(C(=O)O)cc3)nc2n(C)c1=O] |
| I-3 | 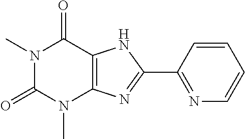 [SMILES: Cn1c(=O)c2[nH]c(-c3ccccn3)nc2n(C)c1=O] |
| I-4 | 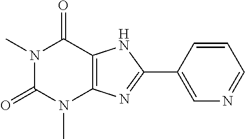 [SMILES: Cn1c(=O)c2[nH]c(-c3cccnc3)nc2n(C)c1=O] |
| I-5 | 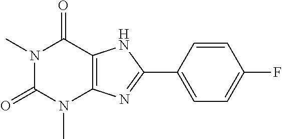 [SMILES: Cn1c(=O)c2[nH]c(-c3ccc(F)cc3)nc2n(C)c1=O] |
| I-6 | 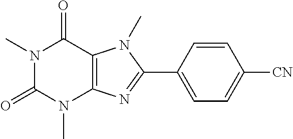 [SMILES: Cn1c(=O)c2c(nc(-c3ccc(C#N)cc3)n2C)n(C)c1=O] |
| I-7 | 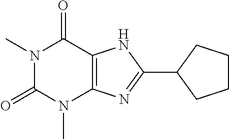 [SMILES: Cn1c(=O)c2[nH]c(C3CCCC3)nc2n(C)c1=O] |
| I-10 | 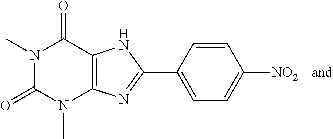 [SMILES: Cn1c(=O)c2[nH]c(-c3ccc([N+](=O)[O-])cc3)nc2n(C)c1=O ; "and"] |
| I-12 | 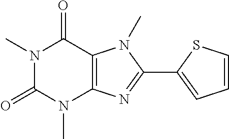 [SMILES: Cn1c(=O)c2c(nc(-c3cccs3)n2C)n(C)c1=O] |

### Results

### Characterization of Exemplary Xanthine Derivatives as Antagonists of PCSK9

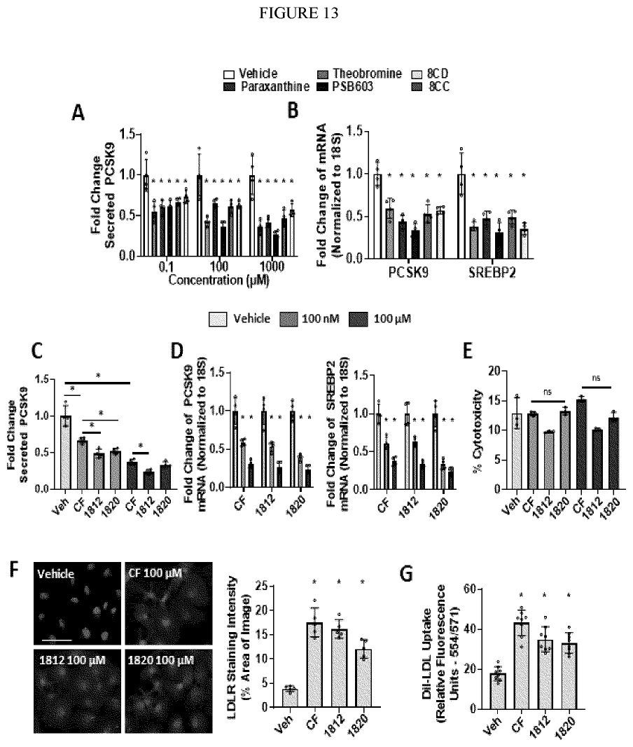

These data demonstrated that CF antagonized secreted PCSK9 levels in pre-clinical models, as well as in humans. CF, however, is a well-characterized compound having several health benefits with few known adverse effects. Achieving an optimal level of PCSK9 inhibition absent of the neuro-excitatory effect of CF, however, may be a challenge for the long-term clinical application of these findings. To address this concern, a variety of known caffeine derivatives, as well as novel compounds, were screened as potential CF alternatives to CF that may achieve significant PCSK9 inhibition while avoiding the undesired neuro-excitatory effect. CF metabolites including theobromine and paraxanthine, as well as other xanthine-derived compounds, such as PSB603, 8CD and 8CC, exhibited a dose-dependent reduction of mRNA expression and secreted levels of PCSK9 (FIG. 13 A and B). See Table 6 for the structure activity relationships of several other known xanthine-based compounds explored at varying doses in HepG2 cells for 24 h. Table 7 shows the structure activity relationships of the newly synthesized xanthine derivatives using either 1 uM or 100 uM.

Initial screening of novel xanthine derivatives optimized for anti-PCSK9 activity yielded compounds with significantly greater efficacy for PCSK9 inhibition than caffeine, for example, Compound No. Ia-7 and Ia-1. Experiments done in HepG2 cells demonstrate that treatment with Compound No. Ia-7 and Ia-1 (denoted 1812 and 1820, respectively, in FIG. 13) yield a 2-fold reduction of secreted PCSK9 levels compared to CF treatment at the same dose (FIG. 13 C). Moreover, at a concentration of 100 nM, these novel compounds achieved similar levels of inhibition as CF at a dose of 100 μM. Assessment at the mRNA level revealed a similar mode of action, whereby antagonism of SREBP2 reduced de novo synthesis of PCSK9 (FIG. 13 D). Like CF, it was also observed that Compound No. Ja-7 and Ia-1 did not exhibit cytotoxic properties on cultured HepG2 hepatocytes (FIG. 13 E). Next, the impact of Compound No. Ia-7 and Ia-1 on DiI-LDL uptake was examined. Consistent with the observed reduction in secreted PCSK9 levels, treatment of HepG2 cells with these novel compounds led to a significant increase in LDLR expression (FIG. 13F) and DiI-LDL cholesterol uptake (FIG. 13 G). Overall, these data demonstrate that a variety of xanthine-derived compounds exhibit potency for the antagonism of PCSK9 expression.

### Discussion

PCSK9 enhances the degradation of the LDLR and promotes the onset and progression of CVD, which represents one of most challenging and costly health care problems that society faces today. Developing an understanding of the regulatory mechanisms that modulate the expression and secretion of PCSK9 from hepatocytes may aid in the development of novel anti-PCSK9 therapies that are more cost-effective than those that currently exist. Overall, results in this application provide evidence that small molecules like CF, capable of increasing ER Ca²⁺ levels, can block the activation of SREBP2 by enhancing GRP78 chaperone function and binding capacity. It is also reported herein that CF potently blocks the expression of PCSK9, a downstream target of SREBP2 transcriptional activity, in cultured hepatocytes, in mice and in healthy human subjects. By extension, it was also observed that CF induced the expression of cell-surface hepatic LDLR and increased the uptake of LDL cholesterol. These findings delineate a novel mechanism by which ER Ca²⁺ and its modulators can affect the expression and activity of proteins that play a central role CVD, liver disease and CKD.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### Tables

TABLE 3
Antibodies used for immunoblotting and immunohistochemical staining.

| | | | Dilution and |
|---|---|---|---|
| Antibody | Catalog no. | Application | Protocol |
| β-actin | A2228, Sigma-Aldrich | IB | 1:5000 |
| CD36 | NB400-144, Novus Biologicals | IB | 1:500 |
| PCSK9 | NB300-959, Novus Biologicals | IB | 1:500 |
| SREBP-2 | 557037, BD Biosciences | IB | 1:500 |
| GFP | NB600-308, Novus Biologicals | IB | 1:1000 |
| Calnexin | C5C9, Cell Signaling | IB | 1:1000 |
| Flag | F3165, Sigma-Aldrich | IB | 1:1000 |
| GRP78 | Ab21685, Abcam | IHC | 1:1000 HIER |
| GRP94 | ADI-SPA-850, Enzo Life Sciences | IHC | 1:1000, HIER |
| LDLR | AF2255, R and D Systems | IHC | 1:100, HIER |
| CD36 | NB400-144, Novus Biologicals | IHC | 1:100, HIER |

IB, immunoblot; IHC, immunohistochemistry; HIER, heat-induced epitope retrieval

TABLE 4
Primers used for real-time PCR.

| Gene | Species | Forward | Reverse |
|---|---|---|---|
| GRP78 | Mouse | GTCCTGCATCATCAGCGA | GGTAGCCACATACTGAACA |
| | | AAG (SEQ ID NO: 1) | CCA (SEQ ID NO: 2) |
| GRP78 | Human | CATCACGCCGTCCTATGT | CGTCAAAGACCGTGTTCTCG |
| | | CG (SEQ ID NO: 3) | (SEQ ID NO: 4) |
| SREBP1 | Human | CAGGTACCGAGTTCTGGT | ACTGCTAGCCGCGCTGCCGC |
| | | GTGTTGGGCCA (SEQ ID | CTCGCTAG (SEQ ID NO: 6) |
| | | NO: 5) | |
| SREBP1 | Mouse | ACCCTGGTGAGTGGAGG | CTTTGCTTCAGTGCCCACCA |
| | | GACCATCTTGG (SEQ ID | CCAGGTCTTT |
| | | NO: 7) | (SEQ ID NO: 8) |
| SREBP2 | Mouse | GCAGCAACGGGACCATT | CCCCATGACTAAGTCCTTCA |
| | | CT (SEQ ID NO: 9) | ACT (SEQ ID NO: 10) |
| SREBP2 | Human | CCTGGGAGACATCGACG | TGAATGACCGTTGCACTGAA |
| | | AGAT (SEQ ID NO: 11) | G (SEQ ID NO: 12) |
| HMGR | Mouse | CTTTCAGAAACGAACTGT | CTAGTGGAAGATGAATGGA |
| | | AGCTCAC (SEQ ID NO: 13) | CATGAT (SEQ ID NO: 14) |
| HMGR | Human | TCTGGAGGATCCAAGGA | ACCAAGTGGCTGTCTCAGTG |
| | | TTCTG (SEQ ID NO: 15) | AT (SEQ ID NO: 16) |
| IRE1α | Mouse | TGAAACACCCCTTCTTCT | CCTCCTTTTCTATTCGGTCAC |
| | | GG (SEQ ID NO: 17) | TT (SEQ ID NO: 18) |
| PCSK9 | Mouse | TGCAAAATCAAGGAGCA | CAGGGAGCACATTGCATCC |
| | | TGGG (SEQ ID NO: 19) | (SEQ ID NO: 20) |
| PERK | Mouse | CCTTGGTTTCATCTAGCC | ATCCAGGGAGGGGATGAT |
| | | TCA (SEQ ID NO: 21) | (SEQ ID NO: 22) |

TABLE 5
Compounds used to study blocking the expression and secretion of PCSK9 from hepatocytes.

| Compound | Effect on ER Ca²⁺ Levels | Concentration | | Mechanism of Action |
|---|---|---|---|---|
| thapsigargin (TG) | decrease | 100 | nM | irreversible SERCA antagonist |
| CDN1163 (CDN) | increase | 10 | μM | allosteric SERCA agonist |
| 2 APB | increase | 100 | μM | IP3R antagonist |
| ryanodine | decrease/increase | 10 nM-10 μM | | RyR agonist at nM and RyR |
| | | | | antagonist at μM |
| Caffeine (CF) | hypothesized increase | 10 nM-1 mM | | multiple |
| 8CC | hypothesized increase | 100 nM-1 mM | | adenosine receptor antagonist |
| 8CD | hypothesized increase | 100 nM-1 mM | | adenosine receptor antagonist |
| PSB 603 | hypothesized increase | 100 nM-1 mM | | adenosine receptor antagonist |
| paraxanthine | hypothesized increase | 100 nM-1 mM | | multiple |
| theophylline | hypothesized increase | 100 nM-1 mM | | multiple |
| theobromine | hypothesized increase | 100 nM-1 mM | | multiple |
| Cyclopiazonic acid | decrease | 100 | μM | SERCA antagonist |
| (CPA) | | | | |
| U18666A | N/A | 10 | μM | Intracellular sterol depletion |

TABLE 6
Known xanthine derivatives evaluated in the application.

| | | PCSK9 |
|---|---|---|
| | | reduction |
| | | (1 um) |
| CAS Number | Structure | (or 100 uM*) |
| 1332135-42-8 (Cpd. No. I-1) | 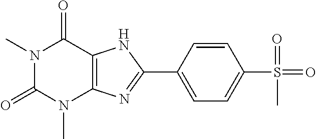 [SMILES: Cn1c(=O)c2[nH]c(-c3ccc(S(C)(=O)=O)cc3)nc2n(C)c1=O] | 0.42 |
| 85872-58-8 (Cpd. No. I-2) | 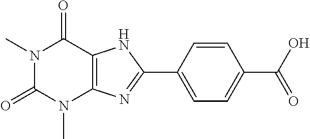 [SMILES: Cn1c(=O)c2[nH]c(-c3ccc(C(=O)O)cc3)nc2n(C)c1=O] | 0.57 |
| 1088-65-9 (Cpd. No. I-3) | 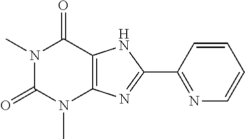 [SMILES: Cn1c(=O)c2[nH]c(-c3ccccn3)nc2n(C)c1=O] | 0.58 |
| 1029-62-5 (Cpd. No. I-4) | 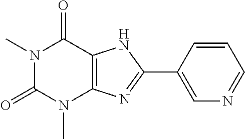 [SMILES: Cn1c(=O)c2[nH]c(-c3cccnc3)nc2n(C)c1=O] | 0.68 |
| 57281-09-01 (Cpd. No. I-5) | 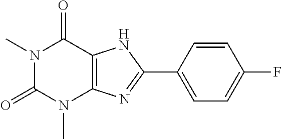 [SMILES: Cn1c(=O)c2[nH]c(-c3ccc(F)cc3)nc2n(C)c1=O] | 0.73 |
| 1092351-10-4 | 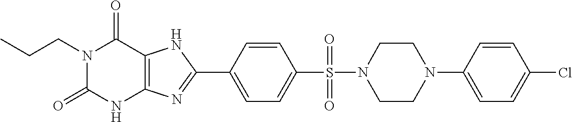 [SMILES: CCCn1c(=O)[nH]c2nc(-c3ccc(S(=O)(=O)N4CCN(c5ccc(Cl)cc5)CC4)cc3)[nH]c2c1=O] | 0.82 |
| 1137486-80-6 (Cpd. No. I-6) | 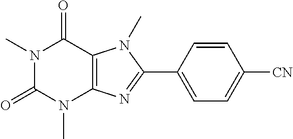 [SMILES: Cn1c(=O)c2c(nc(-c3ccc(C#N)cc3)n2C)n(C)c1=O] | 0.89 |
| 35873-49-5 (Cpd. No. I-7) | 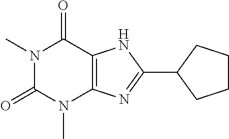 [SMILES: Cn1c(=O)c2[nH]c(C3CCCC3)nc2n(C)c1=O] | 0.94 |
| 147700-11-6 (Cpd. No. I-8) | 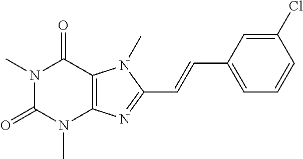 [SMILES: Cn1c(=O)c2c(nc(/C=C/c3cccc(Cl)c3)n2C)n(C)c1=O] | 0.78* |
| 4114-46-5 | 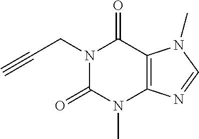 [SMILES: C#CCn1c(=O)c2c(ncn2C)n(C)c1=O] | 0.59* |
| 58-08-2 (caffeine; CF) | 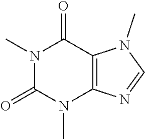 | 0.48* |
| 611-59-6 (paraxanthine) | 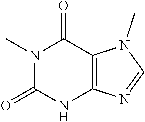 | 0.68* |
| 63906-63-7 | 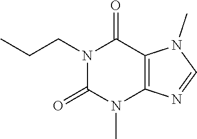 [SMILES: CCCn1c(=O)c2c(ncn2C)n(C)c1=O] | 0.38* |
| 80288-49-9 | 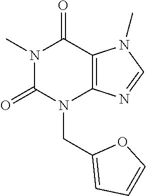 [SMILES: Cn1c(=O)c2c(ncn2C)n(Cc2ccco2)c1=O] | 0.41* |
| 83-67-0 (theobromine) | 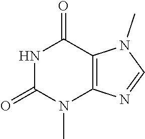 | 0.67* |
| 58-55-9 (theophylline) | 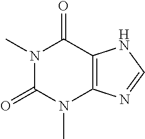 | 0.42* |
| 1088-64-8 (Cpd. No. I-9) | 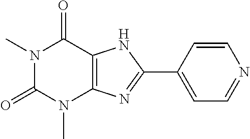 [SMILES: Cn1c(=O)c2[nH]c(-c3ccncc3)nc2n(C)c1=O] | 0.86 (100 nM) |
| 1094-63-9 (Cpd. No. I-10) | 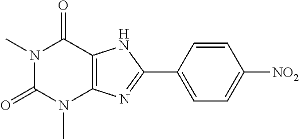 [SMILES: Cn1c(=O)c2[nH]c(-c3ccc([N+](=O)[O-])cc3)nc2n(C)c1=O] | 0.93 (100 nM) |
| 2059817-16-0 (Cpd. No. I-11) | 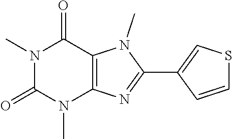 [SMILES: Cn1c(=O)c2c(nc(-c3ccsc3)n2C)n(C)c1=O] | 0.71 |
| 249929-54-2 (Cpd. No. I-12) | 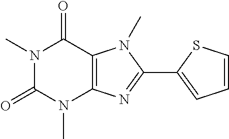 [SMILES: Cn1c(=O)c2c(nc(-c3cccs3)n2C)n(C)c1=O] | 0.79 |
| Cpd No I-90 | 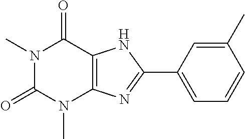 | 0.82 |
| Cpd No I-91 | 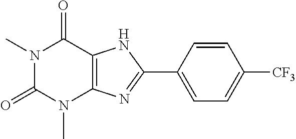 [SMILES: Cn1c(=O)c2[nH]c(-c3ccc(C(F)(F)F)cc3)nc2n(C)c1=O] | 0.83 |
| Cpd No I-92 | 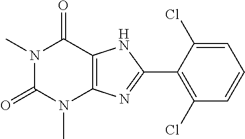 [SMILES: Cn1c(=O)c2[nH]c(-c3c(Cl)cccc3Cl)nc2n(C)c1=O] | 0.78 |
| Cpd No I-95 | 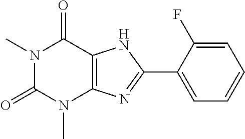 [SMILES: Cn1c(=O)c2[nH]c(-c3ccccc3F)nc2n(C)c1=O] | 0.78 |
| Cpd No I-98 | 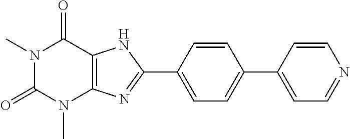 [SMILES: Cn1c(=O)c2[nH]c(-c3ccc(-c4ccncc4)cc3)nc2n(C)c1=O] | 0.79 |

TABLE 7
Exemplary xanthine derivatives of the application.

| | | PCSK9 reduction (1 uM) |
|---|---|---|
| Compound No. | Structure | (or 100 uM*) |
| Ia-1 | 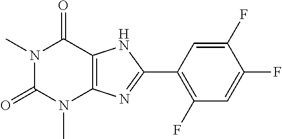 [SMILES: Cn1c(=O)c2[nH]c(-c3cc(F)c(F)cc3F)nc2n(C)c1=O] | 0.54 |
| Ia-2 | 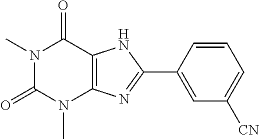 [SMILES: Cn1c(=O)c2[nH]c(-c3cccc(C#N)c3)nc2n(C)c1=O] | 0.62 |
| Ia-3 | 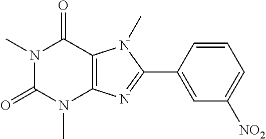 [SMILES: Cn1c(=O)c2c(nc(-c3cccc([N+](=O)[O-])c3)n2C)n(C)c1=O] | 0.66 |
| Ia-4 | 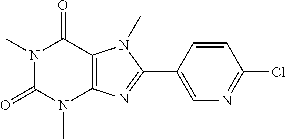 [SMILES: Cn1c(=O)c2c(nc(-c3ccc(Cl)nc3)n2C)n(C)c1=O] | 0.68 |
| Ia-5 | 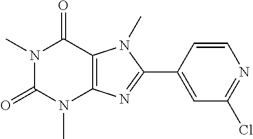 [SMILES: Cn1c(=O)c2c(nc(-c3ccnc(Cl)c3)n2C)n(C)c1=O] | 0.68 |
| Ia-6 | 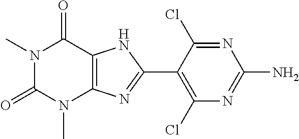 [SMILES: Cn1c(=O)c2[nH]c(-c3c(Cl)nc(N)nc3Cl)nc2n(C)c1=O] | 0.71 |
| Ia-7 | 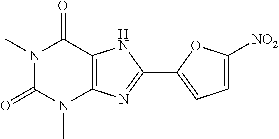 [SMILES: Cn1c(=O)c2[nH]c(-c3ccc([N+](=O)[O-])o3)nc2n(C)c1=O] | 0.72 |
| Ia-8 | 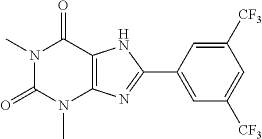 [SMILES: Cn1c(=O)c2[nH]c(-c3cc(C(F)(F)F)cc(C(F)(F)F)c3)nc2n(C)c1=O] | 0.75 |
| Ia-9 | 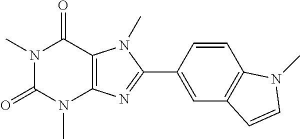 | 0.77 |
| Ia-10 | 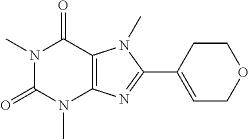 [SMILES: Cn1c(=O)c2c(nc(C3=CCOCC3)n2C)n(C)c1=O] | 0.80 |
| Ia-11 | 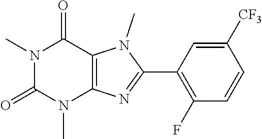 [SMILES: Cn1c(=O)c2c(nc(-c3cc(C(F)(F)F)ccc3F)n2C)n(C)c1=O] | 0.81 |
| Ia-12 | 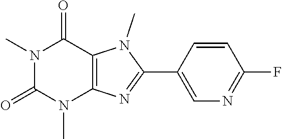 [SMILES: Cn1c(=O)c2c(nc(-c3ccc(F)nc3)n2C)n(C)c1=O] | 0.88 |
| Ia-13 | 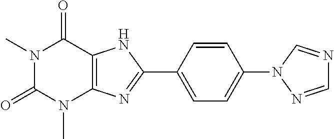 [SMILES: Cn1c(=O)c2[nH]c(-c3ccc(-n4cncn4)cc3)nc2n(C)c1=O] | 0.89 |
| Ia-14 | 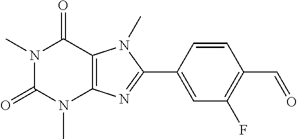 [SMILES: Cn1c(=O)c2c(nc(-c3ccc(C=O)c(F)c3)n2C)n(C)c1=O] | 0.89 |
| Ia-15 | 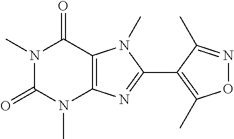 [SMILES: Cc1noc(C)c1-c1nc2c(c(=O)n(C)c(=O)n2C)n1C] | 0.91 |
| Ia-16 | 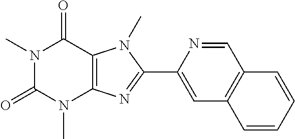 [SMILES: Cn1c(=O)c2c(nc(-c3cc4ccccc4cn3)n2C)n(C)c1=O] | 0.91 |
| Ia-17 | 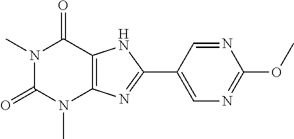 | 0.92 |
| Ia-18 | 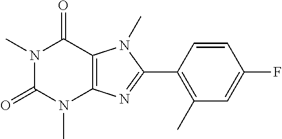 [SMILES: Cc1cc(F)ccc1-c1nc2c(c(=O)n(C)c(=O)n2C)n1C] | 0.98 |
| Ia-19 | 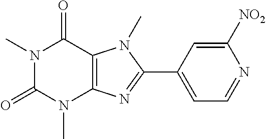 [SMILES: Cn1c(=O)c2c(nc(-c3ccnc([N+](=O)[O-])c3)n2C)n(C)c1=O] | 0.50* |
| Ia-20 | 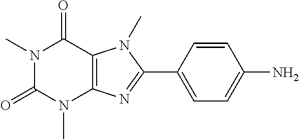 [SMILES: Cn1c(=O)c2c(nc(-c3ccc(N)cc3)n2C)n(C)c1=O] | 0.62* |
| Ia-47 | 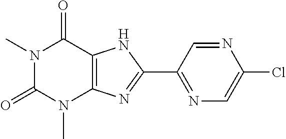 [SMILES: Cn1c(=O)c2[nH]c(-c3cnc(Cl)cn3)nc2n(C)c1=O] | 0.74 |
| Ia-87 | 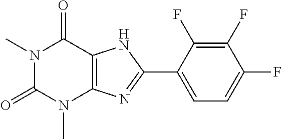 [SMILES: Cn1c(=O)c2[nH]c(-c3ccc(F)c(F)c3F)nc2n(C)c1=O] | 0.79 |
| Ia-88 | 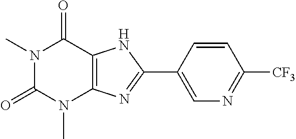 [SMILES: Cn1c(=O)c2[nH]c(-c3ccc(C(F)(F)F)nc3)nc2n(C)c1=O] | 0.70 |
| Ia-93 | 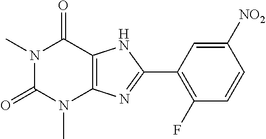 [SMILES: Cn1c(=O)c2[nH]c(-c3cc([N+](=O)[O-])ccc3F)nc2n(C)c1=O] | 0.84 |
| Ia-94 | 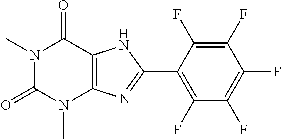 [SMILES: Cn1c(=O)c2[nH]c(-c3c(F)c(F)c(F)c(F)c3F)nc2n(C)c1=O] | 0.89 |
| Ia-99 | 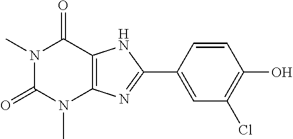 [SMILES: Cn1c(=O)c2[nH]c(-c3ccc(O)c(Cl)c3)nc2n(C)c1=O] | 0.80 |
| Ia-100 | 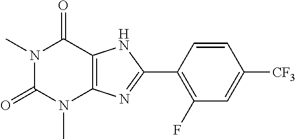 [SMILES: Cn1c(=O)c2[nH]c(-c3ccc(C(F)(F)F)cc3F)nc2n(C)c1=O] | 0.75 |

### REFERENCES

Abifadel M., Varret M., Rabes J. P., Allard D., Ouguerram K., Devillers M., Cruaud C., Benjannet S., Wickham L., Erlich D., Derre A., Villeger L., Farnier M., Beucler I., Bruckert E., Chambaz J., Chanu B., Lecerf J. M., Luc G., Moulin P., Weissenbach J., Prat A., Krempf M., Junien C., Seidah N. G., Boileau C., Mutations in PCSK9 cause autosomal dominant hypercholesterolemia. Nat Genet 34, 154-156 (2003).
Ajoolabady A., Wang S., Kroemer G., Klionsky D. J., Uversky V. N., Sowers J. R., Aslkhodapasandhokmabad H., Bi Y., Ge J., Ren J., ER stress in Cardiometabolic Diseases: from Molecular Mechanisms to Therapeutics. Endocr Rev bnab006 (2021).
Benjannet S., Rhainds D., Essalmani R., Mayne J., Wickham L., Jin W., Asselin M. C., Hamelin J., Varret M., Allard D., Trillard M., Abifadel M., Tebon A., Attie A. D., Rader D. J., Boileau C., Brissette L., Chretien M., Prat A., Seidah N. G., NARC-1/PCSK9 and its natural mutants: zymogen cleavage and effects on the low density lipoprotein (LDL) receptor and LDL cholesterol. The Journal of biological chemistry 279, 48865-48875 (2004).
Blond-Elguindi S., Cwirla S. E., Dower W. J., Lipshutz R. J., Sprang S. R., Sambrook J. F., Gething M. J., Affinity panning of a library of peptides displayed on bacteriophages reveals the binding specificity of BiP. Cell 75, 717-728 (1993).
Bootman M. D., Collins T. J., Mackenzie L., Roderick H. L., Berridge M. J., Peppiatt C. M., 2-aminoethoxydiphenyl borate (2-APB) is a reliable blocker of store-operated Ca2+ entry but an inconsistent inhibitor of InsP3-induced Ca2+ release. FASEB J 16, 1145-1150 (2002).
Chen W., Wang R., Chen B., Zhong X., Kong H., Bai Y., Zhou Q., Xie C., Zhang J., Guo A., Tian X., Jones P. P., O'Mara M. L., Liu Y., Mi T., Zhang L., Bolstad J., Semeniuk L., Cheng H., Zhang J., Chen J., Tieleman D. P., Gillis A. M., Duff H. J., Fill M., Song L. S., Chen S. R., The ryanodine receptor store-sensing gate controls Ca2+ waves and Ca2+-triggered arrhythmias. Nat Med 20, 184-192 (2014).
Coe H., Michalak M., Calcium binding chaperones of the endoplasmic reticulum. Gen Physiol Biophys 28, F96-F103 (2009).
Colgan S. M., Tang D., Werstuck G. H., Austin R. C., Endoplasmic reticulum stress causes the activation of sterol regulatory element binding protein-2. Int J Biochem Cell Biol 39, 1843-1851 (2007).
Dara L., Ji C., Kaplowitz N., The contribution of endoplasmic reticulum stress to liver diseases. Hepatology 5, 1752-1763 (2011).
Demers A., Samami S., Lauzier B., Des Rosiers C., Ngo Sock E. T., Ong H., Mayer G., PCSK9 Induces CD36 Degradation and Affects Long-Chain Fatty Acid Uptake and Triglyceride Metabolism in Adipocytes and in Mouse Liver. Arterioscler Thromb Vase Biol 35, 2517-2525 (2015).
Diercks B. P., Fliegert R., Guse A. H., Mag-Fluo4 in T cells: Imaging of intra-organelle free Ca(2+) concentrations. Biochim Biophys Acta Mol Cell Res 1864, 977-986 (2017).
Ding M., Bhupathiraju S. N., Satija A., van Dam R. M., Hu F. B., Long-term coffee consumption and risk of cardiovascular disease: a systematic review and a dose-response meta-analysis of prospective cohort studies. Circulation 129, 643-659 (2014).
Dong B., Wu M., Li H., Kraemer F. B., Adeli K., Seidah N. G., Park S. W., Liu J., Strong induction of PCSK9 gene expression through HNFlalpha and SREBP2: mechanism for the resistance to LDL-cholesterol lowering effect of statins in dyslipidemic hamsters. J Lipid Res 51, 1486-1495 (2010).
Dzamko N., van Denderen B. J., Hevener A. L., Jorgensen S. B., Honeyman J., Galic S., Chen Z. P., Watt M. J., D. Campbell J., Steinberg G. R., Kemp B. E., AMPK beta1 deletion reduces appetite, preventing obesity and hepatic insulin resistance. The Journal of biological chemistry 285, 115-122 (2010).
Echeverri D., Montes F. R., Cabrera M., Galan A., Prieto A., Caffeine's Vascular Mechanisms of Action. Int J Vase Med 2010, 834060 (2010).
Flynn G. C., Pohl J., Flocco M. T., Rothman J. E., Peptide-binding specificity of the molecular chaperone BiP. Nature 353, 726-730 (1991).
Horton J. D., Shah N. A., Warrington J. A., Anderson N. N., Park S. W., Brown M. S., Goldstein J. L., Combined analysis of oligonucleotide microarray data from transgenic and knockout mice identifies direct SREBP target genes. Proc Natl Acad Sci USA 100, 12027-12032 (2003).
Jo H., Choe S. S., Shin K. C., Jang H., Lee J. H., Seong J. K., Back S. H., Kim J. B., Endoplasmic reticulum stress induces hepatic steatosis via increased expression of the hepatic very low-density lipoprotein receptor. Hepatology 4, 1366-1377 (2013).
Kaplowitz N., Than T. A., Shinohara M., Ji C., Endoplasmic reticulum stress and liver injury. Semin Liver Dis 4, 367-377 (2013).
Kang S., Dahl R., Hsieh W., Shin A., Zsebo K. M., Buettner C., Hajjar R. J., Lebeche D., Small Molecular Allosteric Activator of the Sarco/Endoplasmic Reticulum Ca2+-ATPase (SERCA) Attenuates Diabetes and Metabolic Disorders. The Journal of biological chemistry 291, 5185-5198 (2016).
Khazaei M., Moien-Afshari F., Laher I., Vascular endothelial function in health and diseases. Pathophysiology 15, 49-67 (2008).
Kuhnast S., van der Hoorn J. W., Pieterman E. J., van den Hoek A. M., Sasiela W. J., Gusarova V., Peyman A., Schafer H. L., Schwahn U., Jukema J. W., Princen H. M., Alirocumab inhibits atherosclerosis, improves the plaque morphology, and enhances the effects of a statin. J Lipid Res 55, 2103-2112 (2014).
Lebeau P., Al-Hashimi A., Sood S., Lhotak S., Yu P., Gyulay G., Pare G., Chen S. R., Trigatti B., Prat A., Seidah N. G., Austin R. C., Endoplasmic Reticulum Stress and Ca²⁺ Depletion Differentially Modulate the Sterol Regulatory Protein PCSK9 to Control Lipid Metabolism. The Journal of biological chemistry 292, 1510-1523 (2017).
Lebeau P. F., Byun J. H., Platko K., Al-Hashimi A. A., Lhotik S., MacDonald M. E., Mejia-Benitez A., Prat A., Igdoura S. A., Trigatti B., Maclean K. N., Seidah N. G., Austin R. C., Pcsk9 knockout exacerbates diet-induced non-alcoholic steatohepatitis, fibrosis and liver injury in mice. JHEP Rep 6, 418-429 (2019).
Lievremont J. P., Rizzuto R., Hendershot L., Meldolesi J., BiP, a major chaperone protein of the endoplasmic reticulum lumen, plays a direct and important role in the storage of the rapidly exchanging pool of Ca2+. The Journal of biological chemistry 272, 30873-30879 (1997).
Li Y., Xu S., Mihaylova M. M., Zheng B., Hou X., Jiang B., Park O., Luo Z., Lefai E., Shyy J. Y., Gao B., Wierzbicki M., Verbeuren T. J., Shaw R. J., Cohen R. A., Zang M., AMPK phosphorylates and inhibits SREBP activity to attenuate hepatic steatosis and atherosclerosis in diet-induced insulin-resistant mice. Cell Metab 13, 376-388 (2011).
Maxwell K. N., Breslow J. L, Adenoviral-mediated expression of Pcsk9 in mice results in a low-density lipoprotein receptor knockout phenotype. Proc Natl Acad Sci USA 101, 7100-7105 (2004).
Moslehi A., Hamidi-Zad Z., Role of SREBPs in Liver Diseases: A Mini-review. J Clin Transl Hepatol 3, 332-338 (2018).
Mustafa M., Wang T. N., Chen X., Gao B., Krepinsky J. C., American Journal of Physiology-Renal Physiology 311, F614-F625 (2016).
Palmer A. E., Jin C., Reed J. C., Tsien R. Y., Bel-2-mediated alterations in endoplasmic reticulum Ca2+ analyzed with an improved genetically encoded fluorescent sensor. Proc Natl Acad Sci USA 101, 17404-17409 (2004).
Quan H. Y., Kim D. Y., Chung S. H., Caffeine attenuates lipid accumulation via activation of AMP-activated protein kinase signaling pathway in HepG2 cells. BMB Rep 46, 207-212 (2013).
Robinson J. G., Jayanna M. B., Brown A. S., Aspry K., Orringer C., Gill E. A., Goldberg A., Jones L. K., Maki K., Dixon D. L., Saseen J. J., Soffer D., Enhancing the value of PCSK9 monoclonal antibodies by identifying patients most likely to benefit. A consensus statement from the National Lipid Association. J Clin Lipidol 4, 525-537 (2019).
Sabatine M. S., Giugliano R. P., Keech A. C., Honarpour N., Wiviott S. D., Murphy S. A., Kuder J. F., Wang H., Liu T., Wasserman S. M., Sever P. S., Pedersen T. R., F. S. Committee, Investigators, Evolocumab and Clinical Outcomes in Patients with Cardiovascular Disease. N Engl J Med 376, 1713-1722 (2017).
Seidah N. G., Abifadel M., Prost S., Boileau C., Prat A., The Proprotein Convertases in Hypercholesterolemia and Cardiovascular Diseases: Emphasis on Proprotein Convertase Subtilisin/Kexin 9. Pharmacol Rev 69, 33-52 (2017).
Seidah N. G., Benjannet S., Wickham L., Marcinkiewicz J., Jasmin S. B., Stifani S., Basak A., Prat A., Chretien M., The secretory proprotein convertase neural apoptosis-regulated convertase 1 (NARC-1): liver regeneration and neuronal differentiation. Proc Natl Acad Sci USA 100, 928-933 (2003).
Tsuda S., Egawa T., Kitani K., Oshima R., Ma X., Hayashi T., Caffeine and contraction synergistically stimulate 5′-AMP-activated protein kinase and insulin-independent glucose transport in rat skeletal muscle. Physiol Rep 3, (2015).
Turnbull D., Rodricks J. V., Mariano G. F., Chowdhury F., Caffeine and cardiovascular health. Regul Toxicol Pharmacol 89, 165-185 (2017).
Wang W. A., Liu W. X., Durnaoglu S., Lee S. K., Lian J., Lehner R., Ahnn J., Agellon L. B., Michalak M., Loss of Calreticulin Uncovers a Critical Role for Calcium in Regulating Cellular Lipid Homeostasis. Sci Rep 7, 5941 (2017).
Wang L., Chen J., Ning C., Lei D., Ren J., Endoplasmic reticulum stress related molecular mechanisms in nonalcoholic fatty liver disease (NAFLD). Curr Drug Targets 19, 1087-1094 (2018).
Werstuck G. H., Lentz S. R., Dayal S., Hossain G. S., Sood S. K., Shi Y. Y., Zhou J., Maeda N., Krisans S. K., Malinow M. R., Austin R. C., Homocysteine-induced endoplasmic reticulum stress causes dysregulation of the cholesterol and triglyceride biosynthetic pathways. J Clin Invest 107, 1263-1273 (2001).
Williams D. B., Beyond lectins: the calnexin/calreticulin chaperone system of the endoplasmic reticulum. J Cell Sci 119, 615-623 (2006).
Yang J., Nune M., Zong Y., Zhou L., Liu Q., Close and Allosteric Opening of the Polypeptide-Binding Site in a Human Hsp70 Chaperone BiP. Structure 23, 2191-2203 (2015).
Yeganeh B., Moghadam A. R., Alizadeh J., Wiechec E., Alavian S. M., Hashemi M., Geramizadeh B., Samali A., Lankarani K. B., Post M., Peymani P., Coombs K. M., Ghavami S., Hepatitis B and C virus-induced hepatitis: Apoptosis, autophagy, and unfolded protein response. World J Gastroenterol 21, 13225-13239 (2015).
Zucchi R., Ronca-Testoni S., The sarcoplasmic reticulum Ca²⁺ channel/ryanodine receptor: modulation by endogenous effectors, drugs and disease states. Pharmacol Rev 49, 1-51 (1997).

1. A compound of Formula (Ia): 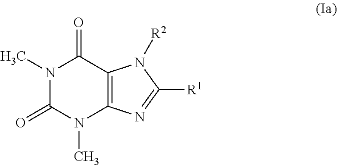
or a pharmaceutically acceptable salt, solvate and/or prodrug thereof;
wherein
R¹ is selected from
(i) phenyl optionally substituted with one to five substituents independently selected from CN, halo, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H and C(O)H, and substituted with one or two of C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(ii) 9-10-membered bicyclic aryl wherein the second ring is phenyl or C₅₋₆cycloalkyl and the 9-10 membered bicyclic aryl is optionally substituted with one or more substituents independently selected from ═O, CN, halo, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(iii) monocyclic C₃₋₆cycloalkyl or C₅₋₆cycloalkenyl optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one or more substituents independently selected from halo, CN NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(v) monocyclic C₅₋₆heterocycloalkyl optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(vi) 8-10-membered bicyclic heterocycloalkyl wherein the second ring is C₅₋₆heterocycloalkyl, phenyl, C₅₋₆cycloalkyl or C₅₋₆heteroaryl and the 8-10-membered bicyclic heterocycloalkyl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(vii) monocyclic C₅₋₆heteroaryl, optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂; and
(viii) 8-10-membered bicyclic heteroaryl wherein the second ring C₅₋₆heterocycloalkyl, phenyl, C₅₋₆cycloalkyl or C₅₋₆heteroaryl and the 8-10-membered bicyclic heteroaryl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
R² is selected from H, C₁₋₆alkyl and C₁₋₆alkyl substituted with one or more substituents independently selected from OH and halo; and
X¹ and X² are independently selected from O, NH, N(C₁₋₆alkyl), N(C₁₋₆fluoroalkyl), C(O), C(O)O, OC(O), C(O)NH, C(O)N(C₁₋₆alkyl), C(O)N(C₁₋₆fluoroalkyl), NHC(O), N(C₁₋₆alkyl)C(O), N(C₁₋₆fluoroalkyl)C(O), S, S(O) and SO₂;
provided that R¹ is not:
(a) phenyl monosubstituted with unsubstituted phenyl, unsubstituted pyridinyl and unsubstituted pyrazolyl;
(b) unsubstituted thiophenyl or thiophenyl substituted with Br, NO₂, CN, CH₃, C(O)H, C(O)CH₃ or OCH₃;
(c) unsubstituted furanyl or furanyl substituted with Cl, Br, CH₃, CF₃, C(O)H or phenyl;
(d) unsubstituted pyrimidinyl, unsubstituted pyridinyl or pyridinyl monosubstituted with NHCH₃, Br, CH₃ or pyridinyl;
(e) 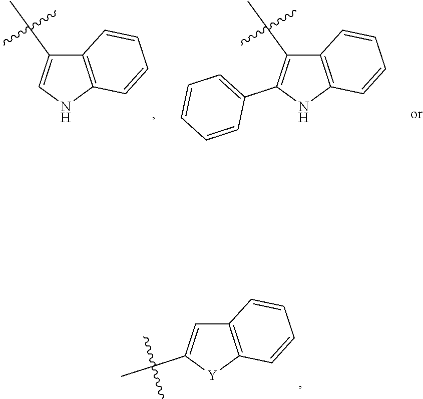
wherein Y is NH, O or S, and when Y is, the phenyl ring is unsubstituted or mono-substituted with OCH₃ or Cl; and
(f)

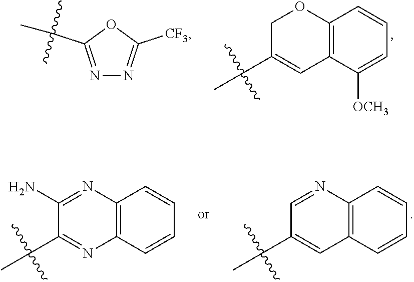 [SMILES: *c1nnc(C(F)(F)F)o1 ; *C1=Cc2c(OC)cccc2OC1 ; *c1nc2ccccc2nc1N ; *c1cnc2ccccc2c1]

2. (canceled)

3. The compound of claim 1, wherein X¹ is selected from O, C(O), C(O)O, OC(O), C(O)NH, NHC(O) and SO₂ and X² is selected from O and C(O), and R¹ is (i) phenyl optionally substituted with one to five substituents independently selected from F, Cl, Br, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), O—C₁₋₂alkyl, O—C₁₋₂fluoroalkyl, NHC(O)—C₁₋₂alkyl, NHC(O)C₁₋₄fluoroalkyl, C(O)C₁₋₄alkyl, C(O)C₁₋₄fluoroalkyl C(O)OC₁₋₄alkyl, C(O)OC₁₋₄fluoroalkyl, OC(O)C₁₋₂alkyl, OC(O)C₁₋₄fluoroalkyl, SO₂C₁₋₄alkyl, SO₂C₁₋₄fluoroalkyl, CO₂H, C(O)H, and substituted with one or two of C₅₋₆heteroaryl, C₅₋₆heterocycloalkyl, phenyl, O-phenyl, C(O)-phenyl, C₁₋₂alkylene-phenyl, O—C₁₋₂alkylenephenyl, C(O)—C₁₋₂alkylenephenyl, C₃₋₆cycloalkyl and C₃₋₆cycloalkenyl, the latter 10 groups being optionally substituted with one or more substituents independently selected from halo, OH, C₁₋₄alkyl, C₁₋₄fluoroalkyl, O—C₁₋₄alkyl, O—C₁₋₄fluoroalkyl and NH₂.

4. The compound of claim 3, wherein R¹ is (i) phenyl substituted with one to four substituents independently selected from F, Cl, Br, CN, NH₂, OH, NO₂, CH₃, CH₂CH₃, CH₂CH₂CH₃, CH(CH₃)₂, C(CH₃)₃, CF₃, CF₂H, CH₂CF₂H, CH₂CF₃, CH₂CFH₂, CH(CF₃)₂, C(CF₃)₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, (CF₃)₃CO, CH₂OH, CH₂NH₂, NHC(O)CH₃, NHC(O)CF₃, C(O)CH₃, C(O)CF₃, OC(O)CH₃, OC(O)CF₃, SO₂CH₃, SO₂CF₃, CO₂H, C(O)H, and substituted with one or two of C₅₋₆heteroaryl, C₅₋₆heterocycloalkyl, phenyl, O-phenyl, C(O)-phenyl, CH₂-phenyl, O-CH₂phenyl, C(O)—CH₂phenyl, C₃₋₆cycloalkyl and C₃₋₆cycloalkenyl, the latter 10 groups being optionally substituted with one or more substituents independently selected from F, Cl, OH, CH₃, CF₃, CH₃O, CF₃O, and NH₂.

5.-6. (canceled)

7. The compound of claim 1, wherein X¹ is O and X² is O, and R¹ is (ii) 9-10-membered bicyclic aryl wherein the second ring is phenyl or C₅₋₆cycloalkyl and the 9-10 membered bicyclic aryl is optionally substituted with one to five substituents independently selected from ═O, halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₆alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₄alkylene-phenyl, O—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂.

8.-11. (canceled)

12. The compound of claim 1, wherein X¹ is O and X² is O, and R¹ is (iii) monocyclic C₃₋₆cycloalkyl or C₅₋₆cycloalkenyl optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₄alkylene-phenyl, O—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₆fluoroalkyl, O—C₁₋₄alkyl, O—C₁₋₄fluoroalkyl and NH₂.

13.-14. (canceled)

15. The compound of claim 1, wherein X¹ is O and X² is O, and R¹ is (iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₆alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₂alkylene-phenyl, O—C₁₋₄alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂.

16.-18. (canceled)

19. The compound of claim 1, wherein X¹ in (v) is O and X² in (v) is O, and R¹ is (v) monocyclic C₅₋₆heterocycloalkyl optionally substituted with one to five substituents independently selected from F, Br, Cl, CN, NH₂, OH, NO₂, C₁₋₄alkyl, C₁₋₄fluoroalkyl, C₁₋₂alkyleneOH, C₁₋₂alkyleneNH₂, C₁₋₄alkyleneNH(C₁₋₄alkyl), C₁₋₄alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₄alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂.

20. (canceled)

21. (canceled)

22. The compound of claim 1, wherein X¹ is O and X² is O, and R¹ is (vi) 8-10-membered bicyclic heterocycloalkyl wherein the second ring is C₅₋₆heterocycloalkyl, phenyl, C₅₋₆cycloalkyl or C₅₋₆heteroaryl and the 8-10-membered bicyclic heterocycloalkyl is optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂.

23.-24. (canceled)

25. The compound of claim 1, wherein X¹ in (vii) is selected from O and S and X² in (vii) is O and R¹ is (vi) monocyclic C₅₋₆heteroaryl, optionally substituted with one to five substituents independently selected from halo, CN, NH₂, OH, NO₂, C₁₋₄alkyl, C₁₋₄fluoroalkyl, C₁₋₂alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), O—C₁₋₄alkyl, S—C₁₋₄alkyl, O—C₁₋₄fluoroalkyl, S—C₁₋₄fluoroalkyl, CO₂H, C(O)H, C₅₋₆heteroaryl, C₅₋₆heterocycloalkyl, phenyl, O-phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂.

26.-27. (canceled)

28. The compound of claim 1, wherein X¹ is selected from O and S and X² is O and R¹ is (viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one to five substituents independently selected from halo, CN, NH₂, OH, NO₂, C₁₋₆alkyl, C₁₋₄fluoroalkyl, C₁₋₄alkyleneOH, C₁₋₂alkyleneNH₂, C₁₋₂alkyleneNH(C₁₋₄alkyl), C₁₋₂alkyleneN(C₁₋₄alkyl)(C₁₋₄alkyl), O—C₁₋₄alkyl, O—C₁₋₄fluoroalkyl, CO₂H, C(O)H, C₅₋₆heteroaryl, C₅₋₆heterocycloalkyl, phenyl, O-phenyl, C₁₋₂alkylene-phenyl, O—C₁₋₂alkylenephenyl, C₃₋₆cycloalkyl and C₃₋₆cycloalkenyl, the latter 8 groups being optionally substituted with one to five substituents independently selected from halo, OH, C₁₋₄alkyl, C₁₋₄fluoroalkyl, O—C₁₋₄alkyl, O—C₁₋₄fluoroalkyl and NH₂.

29.-30. (canceled)

31. The compound of claim 1, wherein R² is selected from H, CH₃, CF₃, CF₂H, CH₃O, CF₃O, CHF₂O and CH₂OH.

32. The compound of claim 31, wherein R² is H.

33. A compound selected from the compounds listed in Table 1 or a pharmaceutically acceptable salt, solvate and/or prodrug thereof.

34. A pharmaceutical composition comprising one or more compounds of claim 1 or a pharmaceutically acceptable salt, solvate and/or hydrate thereof, and one or more pharmaceutically acceptable carriers.

35. The composition of claim 34, further comprising one or more cholesterol lowering agents.

36. A method of treating a disease, disorder or condition treatable by blocking SREBP2 activation and/or PCSK9 gene expression, the method comprising administering a therapeutically effective amount of one or more compounds of Formula (I), or a pharmaceutically acceptable salt, solvate and/or prodrug thereof, to a subject in need thereof: 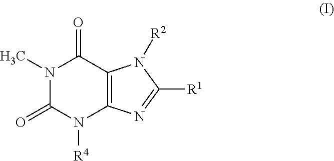
wherein
R¹ is selected from
(i) phenyl substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(ii) 9-10-membered bicyclic aryl wherein the second ring is phenyl or C₅₋₆cycloalkyl and the 9-10 membered bicyclic aryl is optionally substituted with one or more substituents independently selected from ═O, halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(iii) monocyclic C₃₋₆cycloalkyl or C₅₋₆cycloalkenyl optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(iv) 8-10-membered bicyclic cycloalkyl or cycloalkenyl wherein the second ring is C₅₋₆heterocycloalkyl, phenyl, C₅₋₆cycloalkyl or C₅₋₆heteroaryl and the 8-10-membered bicyclic cycloalkyl or cycloalkenyl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(v) monocyclic C₅₋₆heterocycloalkyl optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(vi) 8-10-membered bicyclic heterocycloalkyl wherein the second ring is C₅₋₆heterocycloalkyl, phenyl, C₅₋₆cycloalkyl or C₅₋₆heteroaryl and the 8-10-membered bicyclic heterocycloalkyl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
(vii) monocyclic C₅₋₆heteroaryl, optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂; and
(viii) 8-10-membered bicyclic heteroaryl wherein the second ring is C₅₋₆heterocycloalkyl, phenyl, C₅₋₆cycloalkyl or C₅₋₆heteroaryl and the 8-10-membered bicyclic heteroaryl is optionally substituted with one or more substituents independently selected from halo, CN, NH₂, OH, NO₂, C₁₋₆alkyl, C₁₋₆fluoroalkyl, C₁₋₆alkyleneOH, C₁₋₆alkyleneNH₂, C₁₋₆alkyleneNH(C₁₋₆alkyl), C₁₋₆alkyleneN(C₁₋₆alkyl)(C₁₋₆alkyl), X¹—C₁₋₆alkyl, X¹—C₁₋₆fluoroalkyl, CO₂H, C(O)H, C₅₋₆heteroaryl, C₅₋₆heterocycloalkyl, phenyl, X¹-phenyl, C₁₋₆alkylene-phenyl, X¹—C₁₋₆alkylenephenyl, C₃₋₆cycloalkyl and C₃₋₆cycloalkenyl, the latter 8 groups being optionally substituted with one or more substituents independently selected from halo, OH, C₁₋₆alkyl, C₁₋₆fluoroalkyl, X²—C₁₋₆alkyl, X²—C₁₋₆fluoroalkyl and NH₂;
R² is selected from H, C₁₋₆alkyl and C₁₋₆alkyl substituted with one or more substituents independently selected from OH and halo; and
X¹ and X² are independently selected from O, NH, N(C₁₋₆alkyl), N(C₁₋₆fluoroalkyl), C(O), C(O)O, OC(O), C(O)NH, C(O)N(C₁₋₆alkyl), C(O)N(C₁₋₆fluoroalkyl), NHC(O), N(C₁₋₆alkyl)C(O), N(C₁₋₆fluoroalkyl)C(O), S, S(O) and SO₂.

37.-69. (canceled)

70. The method of claim 36, wherein the disease, disorder or condition is caused and/or exacerbated by increased SREBP2 and/or PCSK9 function or activity.

71. The method of claim 36, wherein the disease, disorder or condition is elevated cholesterol levels, liver disease or chronic kidney disease.

72. The method of claim 36, wherein the therapeutically effective amount of the one or more compounds is administered in combination with one or more other therapeutic agents.

73.-80. (canceled)

81. The method of claim 36, wherein the one or more other therapeutic agents is a statin.

82. (canceled)
